(19) United States
(12) Patent Application Publication
(10) Pub. No.: US 2005/0234046 A1
(43) Pub. Date: Oct. 20, 2005

(54) Aryl sulfonamide and sulfonyl compounds as modulators of PPAR and methods of treating metabolic disorders

(75) Inventors: Cunxiang Zhao, San Diego, CA (US); James W. Malecha, San Diego, CA (US); Stewart A. Noble, San Diego, CA (US); Sergio Gonzalez Duron, San Diego, CA (US); Andrew K. Lindstrom, San Diego, CA (US); Andrew K. Shiau, San Diego, CA (US)

Correspondence Address: PILLSBURY WINTHROP SHAW PITTMAN LLP, ATTENTION: DOCKETING DEPARTMENT, 11682 EL CAMINO REAL, SUITE 200, SAN DIEGO, CA, 92130, US

(73) Assignee: Kalypsys, Inc., San Diego, CA (US)

(21) Appl. No.: 11/102,356

(22) Filed: Apr. 7, 2005

Related U.S. Application Data
(60) Provisional application No. 60/560,579, filed on Apr. 7, 2004.
(60) Provisional application No. 60/656,157, filed on Feb. 24, 2005.

(51) Int. Cl. A61K 31/551; A61K 31/496; A61K 31/495; A61K 31/445; A61K 31/407; C07D 417/02; C07D 43/04

(52) U.S. Cl. 514/218; 514/253.01; 514/254.02; 514/412; 514/255.02; 514/317; 514/252.14; 540/575; 544/295; 544/360; 544/368; 544/383; 546/216

(57) ABSTRACT

Aryl sulfonamide and sulfonyl compounds as modulators of peroxisome proliferator activated receptors, pharmaceutical compositions comprising the same, and methods of treating disease using the same are disclosed.

### CROSS-REFERENCE TO RELATED APPLICATIONS

This application claims the benefit of U.S. provisional application No. 60/560,579, filed Apr. 7, 2004 and U.S. Provisional Application No. 60/656,157, filed Feb. 24, 2005.

### FIELD OF THE INVENTION

The present invention is in the field of medicinal chemistry. More specifically, the present invention relates to novel aryl sulfonamide and sulfonyl compounds and methods for treating various diseases by modulation of nuclear receptor mediated processes using these compounds, and in particular processes mediated by peroxisome proliferator activated receptors (PPARs).

### BACKGROUND OF THE INVENTION

Peroxisome proliferators are a structurally diverse group of compounds which, when administered to certain mammals (e.g., rodents), have been shown to elicit dramatic increases in the size and number of hepatic and renal peroxisomes, as well as concomitant increases in the capacity of peroxisomes to metabolize fatty acids via increased expression of the enzymes required for the β-oxidation cycle (Lazarow and Fujiki, Ann. Rev. Cell Biol. 1:489-530 (1985); Vamecq and Draye, Essays Biochem. 24:1115-225 (1989); and Nelali et al., Cancer Res. 48:5316-5324 (1988)). Compounds that activate or otherwise interact with one or more of the PPARs have been implicated in the regulation of triglyceride and cholesterol levels in animal models. Compounds included in this group are the fibrate class of hypolipidermic drugs, herbicides, and phthalate plasticizers (Reddy and Lalwani, Crit. Rev. Toxicol. 12:1-58 (1983)). Peroxisome proliferation can also be elicited by dietary or physiological factors such as a high-fat diet and cold acclimatization.

Biological processes modulated by PPAR are those modulated by receptors, or receptor combinations, which are responsive to the PPAR receptor ligands. These processes include, for example, plasma lipid transport and fatty acid catabolism, regulation of insulin sensitivity and blood glucose levels, which are involved in hypoglycemia/hyperinsulinemia (resulting from, for example, abnormal pancreatic beta cell function, insulin secreting tumors and/or autoimmune hypoglycemia due to autoantibodies to insulin, the insulin receptor, or autoantibodies that are stimulatory to pancreatic beta cells), macrophage differentiation which lead to the formation of atherosclerotic plaques, inflammatory response, carcinogenesis, hyperplasia, and adipocyte differentiation.

Subtypes of PPAR include PPAR-alpha, PPAR-delta (also known as NUCl, PPAR-beta, and FAAR) and two isoforms of PPAR-gamma. These PPARs can regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis (H. Keller and W. Wahli, Trends Endoodn. Met. 291-296, 4 (1993)).

Insight into the mechanism whereby peroxisome proliferators exert their pleiotropic effects was provided by the identification of a member of the nuclear hormone receptor superfamily activated by these chemicals (Isseman and Green, Nature 347-645-650 (1990)). The receptor, termed PPAR-alpha (or alternatively, PPARα), was subsequently shown to be activated by a variety of medium and long-chain fatty acids and to stimulate expression of the genes encoding rat acyl-CoA oxidase and hydratase-dehydrogenase (enzymes required for peroxisomal β-oxidation), as well as rabbit cytochrome P450 4A6, a fatty acid ω-hydroxylase (Gottlicher et al., Proc. Natl. Acad. Sci. USA 89:4653-4657 (1992); Tugwood et al., EMBO J 11:433-439 (1992); Bardot et al., Biochem. Biophys. Res. Comm. 192:37-45 (1993); Muerhoff et al., J Biol. Chem. 267:19051-19053 (1992); and Marcus et al., Proc. Natl. Acad Sci. USA 90(12):5723-5727 (1993).

Activators of the nuclear receptor PPAR-gamma (or alternatively, PPARγ), for example troglitazone, have been clinically shown to enhance insulin-action, to reduce serum glucose and to have small but significant effects on reducing serum triglyceride levels in patients with Type 2 diabetes. See, for example, D. E. Kelly et al., Curr. Opin. Endocrinol. Diabetes, 90-96, 5 (2), (1998); M. D. Johnson et al., Ann. Pharmacother., 337-348, 32 (3), (1997); and M. Leutenegger et al., Curr. Ther. Res., 403-416, 58 (7), (1997).

PPAR-delta (or alternatively, PPARδ) is broadly expressed in the body and has been shown to be a valuable molecular target for treatment of dyslipedimia and other diseases. For example, in a recent study in insulin-resistant obese rhesus monkeys, a potent and selective PPAR-delta compound was shown to decrease VLDL and increase HDL in a dose response manner (Oliver et al., Proc. Natl. Acad. Sci. U.S.A.98: 5305, 2001).

Because there are three isoforms of PPAR and all of them have been shown to play important roles in energy homeostasis and other important biological processes in human body and have been shown to be important molecular targets for treatment of metabolic and other diseases (see Willson, et al. J. Med. Chem. 43: 527-550 (2000)), it is desired in the art to identify compounds which are capable of selectively interacting with only one of the PPAR isoforms or compounds which are capable of interacting with multiple PPAR isoforms. Such compounds would find a wide variety of uses, such as, for example, in the treatment or prevention of obesity, for the treatment or prevention of diabetes, dyslipidemia, metabolic syndrome X and other uses.

### SUMMARY OF THE INVENTION

The present invention relates to aryl sulfonamide and sulfonyl compounds, useful as modulators of PPAR and methods of treating metabolic disorders. One embodiment of the invention are compounds having structural Formula (I)

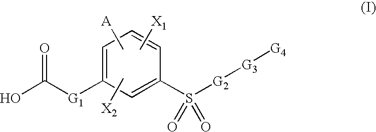

or a pharmaceutically acceptable N-oxide, pharmaceutically acceptable prodrug, pharmaceutically acceptable metabolite, pharmaceutically acceptable salt, pharmaceutically acceptable ester, pharmaceutically acceptable amide, or pharmaceutically acceptable solvate thereof;

wherein:

G₁ is selected from the group consisting of —CR₁R₂)ₙ, -Z(CR₁R₂)ₙ,

—(CR₁R₂)ₙZ-, and —CR₁R₂)ᵣZ(CR₁R₂)ₛ—, wherein Z is O, S, or NR₃;

n is 1-5; r and s are each independently 0 or 1 wherein each R₁ and each R₂ are each independently hydrogen, halogen, optionally substituted lower alkyl, optionally substituted lower heteroalkyl, optionally substituted lower alkoxy, or together may form an optionally substituted cycloalkyl; r and s are not both 0; each R₃ is selected from the group consisting of hydrogen, optionally substituted lower alkyl, and optionally substituted heteroalkyl; A, X₁ and X₂ are each independently selected from the group consisting of hydrogen, optionally substituted lower alkyl, optionally substituted cycloalkyl, halogen, optionally substituted heteroalkyl, optionally substituted cycloheteroalkyl, optionally substituted lower alkynyl, perhaloalkyl, perhaloalkoxy, hydroxy, optionally substituted lower alkoxy, nitro, cyano, and NH₂;

G₂ is a 5, 6, or 7-membered cyclic moiety having the structure

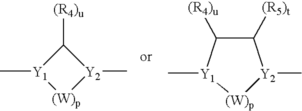

wherein Y₁ is C—R₆ or N and Y₂ is C—R₆ or N;

each R₄ and each R₅ are each independently selected from the group consisting of hydrogen, optionally substituted lower alkyl, halogen, lower perhaloalkyl, hydroxy, optionally substituted heteroalkyl, optionally substituted cycloalkyl, optionally substituted lower alkoxy, nitro, cyano, lower perhaloalkoxy, NH₂,

and —C(O)—O—R₁₁ wherein R₁₁ is hydrogen or optionally substituted lower alkyl, provided that R₄ is not hydroxy or NH₂ when Y₁ is N and R₅ is not hydroxy or NH₂ when Y₂ is N;

W is independently selected from the group consisting of —CR₇R₈—, and a moiety —CR₇— joined together with Y₁ or Y₂ by a double bond;

R₆ is selected from the group consisting of hydrogen, optionally substituted lower alkyl, hydroxy, and lower perhaloalkyl, or is null when Y₁ or Y₂ is joined to W by a double bond; each u is 1 or 2, and each t is 1 or 2, provided that when both Y₁ and Y₂ are N, one of R₄ or R₅ may be taken together with one of W to form an optionally substituted 1- or 2-carbon bridge moiety;

each R₇ and each R₈ are each independently selected from the group consisting of hydrogen, optionally substituted lower alkyl, optionally substituted cycloalkyl, optionally substituted heteroalkyl, hydroxy, optionally substituted lower alkoxy, cyano, halogen, lower perhaloalkyl, NH₂, and a moiety which taken together with R₄ and R₅ forms a 1 or 2 carbon bridge, provided that R₇ and R₈ are not hydroxy or NH₂ when attached to a ring carbon atom adjacent to a ring nitrogen atom;

p is 1, 2 or 3, provided that the G₂ moiety comprises a 5, 6 or 7-membered ring;

G₃ is selected from the group consisting of a bond, a double bond, —(CR₉R₁₀)ₘ—; carbonyl, and —(CR₉R₁₀)ₘCR₉═CR₁₀—, wherein m is 0, 1, or 2, and wherein each R₉ and each R₁₀ is independently hydrogen, optionally substituted lower alkyl, optionally substituted lower alkoxy, optionally substituted aryl, lower perhaloalkyl, cyano, and nitro; and

G₄ is selected from the group consisting of hydrogen, optionally substituted aryl, optionally substituted heteroaryl, optionally substituted cycloalkyl, optionally substituted cycloheteroalkyl, optionally substituted cycloalkenyl, optionally substituted fused aryl, optionally substituted fused heteroaryl, and optionally substituted fused cycloalkyl; provided that when G₃ is a bond, G₄ may be covalently linked to G₂. In certain embodiments of the invention, it is further provided that when G₄ is said optionally substituted cycloheteroalkyl, said optional substituents are non-cyclic.

A preferred embodiment of the invention is a compound having structural formula (I ) wherein G₁ is —(CR₁R₂)ₙ—.

Another preferred embodiment of the invention is a compound having structural formula (I) wherein each R₁ and each R₂ are each independently selected from the group consisting of hydrogen, methyl, ethyl, and propyl, or together may form a cyclopropyl, cyclobutyl, cyclopentyl, or cyclohexyl.

Another preferred embodiment of the invention is a compound having structural formula (I) wherein each R₁ and each R₂ are each hydrogen.

Another embodiment of the invention is a compound having structural formula (I) wherein n=1.

A preferred embodiment of the invention is a-compound having structural formula (I), wherein G₁ is —CH₂— and A is selected from the group consisting of lower alkyl, optionally substituted cycloalkyl, optionally substituted cycloheteroalkyl, hydroxy, NH₂, and optionally substituted heteroalkyl wherein said heteroalkyl is attached to the phenyl ring at a carbon atom and said heteroalkyl contains at least one heteroatom selected from the group consisting of O, N, and S.

Another embodiment of the invention is a compound of having a structural formula selected from the group consisting of:

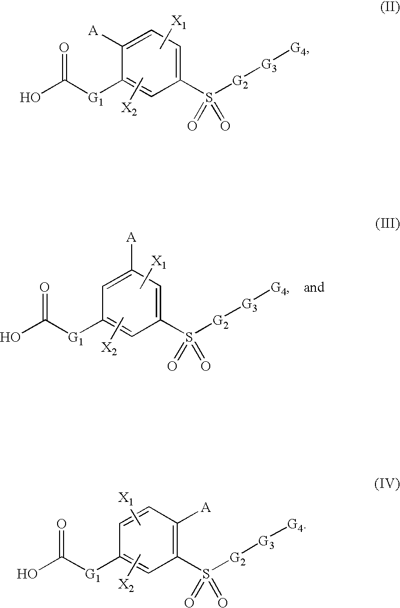

Other preferred embodiment of the invention are compounds of structure (II)-(IV) wherein A is selected from the group consisting of optionally substituted lower alkyl, optionally substituted cycloalkyl, halogen, optionally substituted heteroalkyl, optionally substituted cycloheteroalkyl, lower perhaloalkyl, hydroxy, and NH₂.

Another preferred embodiment of the invention is a compound of structure (II)-(IV) wherein A is selected from the group consisting of lower alkyl, optionally substituted cycloalkyl, optionally substituted cycloheteroalkyl, hydroxy, NH₂, and optionally substituted heteroalkyl wherein said heteroalkyl is attached to the phenyl ring at a carbon atom and said heteroalkyl contains at least one heteroatom selected from the group consisting of O, N, and S.

Another preferred embodiment of the invention is a compound of structure (II)-(IV) wherein A is selected from the group consisting of lower alkyl and an optionally substituted heteroalkyl.

Another preferred embodiment of the invention is a compound of structure (II)-(IV) wherein A, X₁, and X₂ are each independently selected from the group consisting of hydrogen, optionally substituted lower alkyl, lower perhaloalkyl, and halogen.

Another preferred embodiment of the invention is a compound of structure (II)-(IV) wherein at least one of A, X₁, and X₂ is methyl.

Another embodiment of the invention is a compound wherein G₂ is selected from the group consisting of:

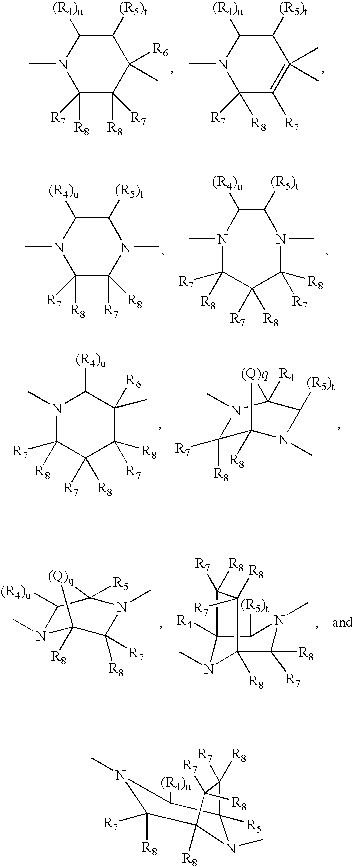

wherein each R₄, each R₅, each R₇ and each R₈ are each independently selected from the group consisting of hydrogen, optionally substituted lower alkyl, halogen, lower perhaloalkyl, hydroxy, optionally substituted lower alkoxy, nitro, cyano, carboxy, and NH₂, or together may form an optionally substituted cycloalkyl;

each Q is each independently —CR₇R₈—, provided that R₄, R₅, R₇ and R₈ are not hydroxy or NH₂ when attached to a ring carbon atom adjacent to a ring nitrogen atom;

q is 1 or 2.

Another embodiment of the invention is a compound wherein A is selected from the group consisting of lower alkyl, optionally substituted cycloalkyl, optionally substituted cycloheteroalkyl, hydroxy, NH₂, and optionally substituted heteroalkyl wherein said heteroalkyl is attached to the phenyl ring at a carbon atom and said heteroalkyl contains at least one heteroatom selected from the group consisting of O, N, and S.

Another embodiment of the invention is a compound of structural formula (I), wherein p is 2; each W is CR₇R₈ or is a moiety —CR₇— joined to Y₂ by a double bond; and Y₁ is N.

Another embodiment of the invention is a compound of structural formula (I), wherein each W is —CR₇R₈—, and Y₂ is N. This embodiment is further preferred where, additionally, Y₁ is N.

Another embodiment of the invention is a compound of structural formula (1), wherein G₂ comprises at least one chiral center.

Another embodiment of the invention is a compound having a structural formula selected from the group consisting of:

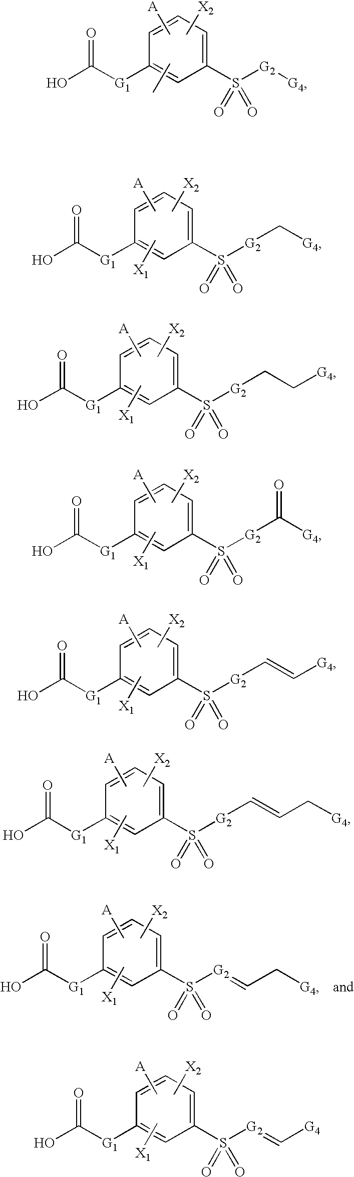

Another embodiment of the invention is a compound of structural formula (I), wherein G₃ is a bond.

Another embodiment of the invention is a compound of structural formula (I), wherein G₄ is optionally substituted aryl, optionally substituted heteroaryl, optionally substituted fused aryl or optionally substituted fused heteroaryl.

Another embodiment of the invention is a compound of structural formula (I), wherein G₄ has a structural formula selected from the group consisting of:

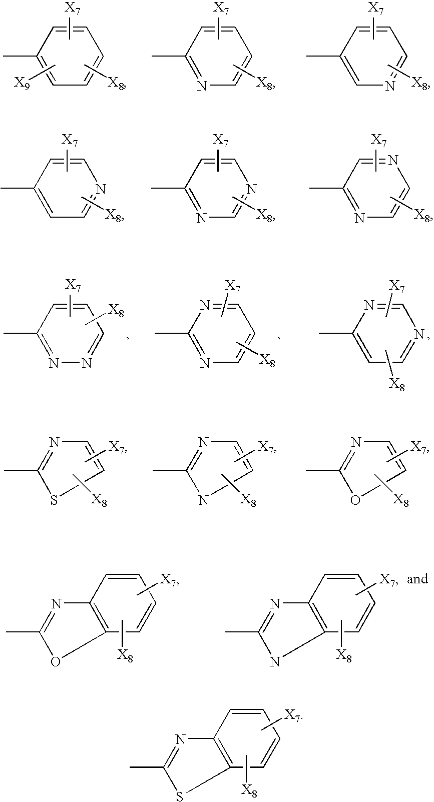

wherein each X₇, each X₈, and each X₉ are each independently selected from the group consisting of hydrogen, optionally substituted lower alkyl, optionally substituted lower alkynyl, halogen, optionally substituted lower heteroalkyl, lower perhaloalkyl, hydroxy, optionally substituted lower alkoxy, lower perhaloalkoxy, nitro, cyano, NH₂, and —CO₂R₁₂, where R₁₂ is selected from the group consisting of optionally substituted lower alkyl and H; further provided that when X₇ and X₈ are present at adjacent ring positions of G₄, X₇ and X₈ may together form an optionally substituted aryl, heteroaryl, aliphatic or heteroaliphatic ring.

Another embodiment of the invention is a compound wherein X₇ is selected from the group consisting of halogen, lower perhaloalkyl or lower perhaloalkoxy and X₈ is selected from the group consisting of hydrogen, halogen, optionally substituted lower alkyl, lower perhaloalkyl and lower perhaloalkoxy.

Another embodiment of the invention is a compound wherein the compound is an hPPAR-delta modulator.

Another embodiment of the invention is a compound wherein the compound is a selective hPPAR-delta modulator.

Another embodiment of the invention is a compound, wherein the compound modulates hPPAR-delta having an EC₅₀ value less than 5 μM as measured by a functional cell assay.

Another embodiment of the invention is a compound having a structural formula selected from the group consisting of:

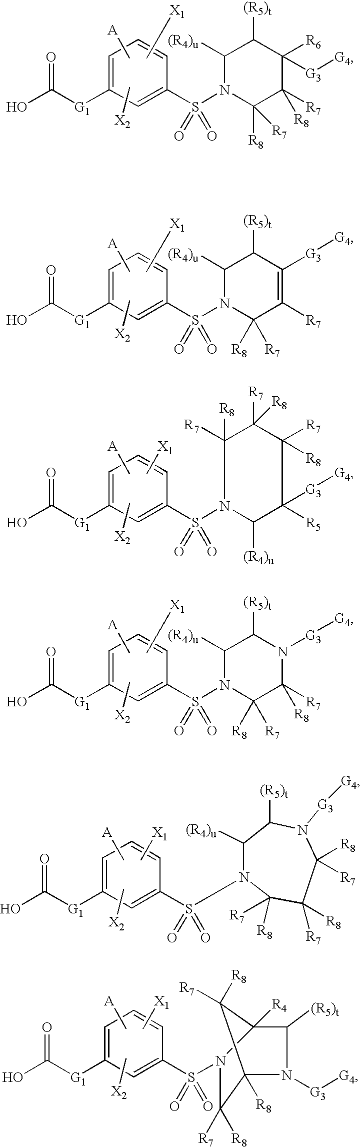

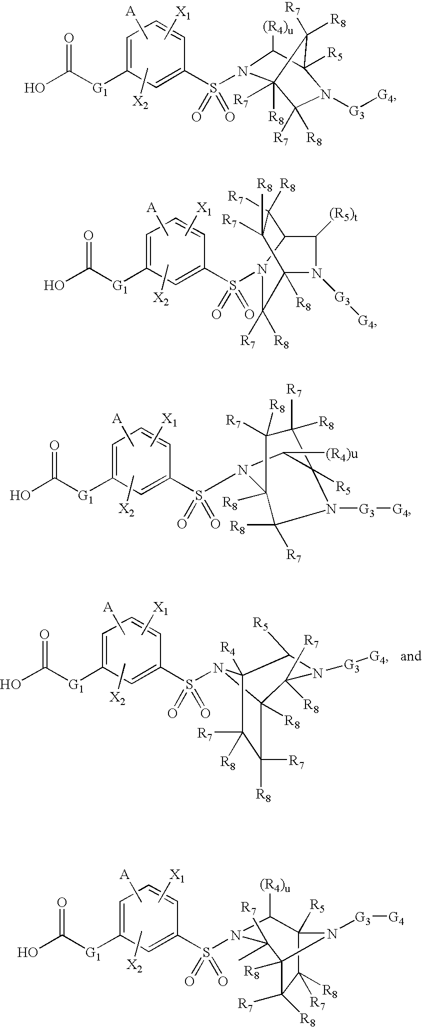

or a pharmaceutically acceptable N-oxide, pharmaceutically acceptable prodrug, pharmaceutically acceptable metabolite, pharmaceutically acceptable salt, pharmaceutically acceptable ester, pharmaceutically acceptable amide, or pharmaceutically acceptable solvate thereof;

wherein:

G₁ is —CCR₁R₂)ₙ— wherein n is 1 to 5 and each R₁ and each R₂ are each independently hydrogen, fluoro, optionally substituted lower alkyl, optionally substituted lower heteroalkyl, optionally substituted lower alkoxy, and lower perhaloalkyl or together may form an optionally substituted cycloalkyl;

A, X₁ and X₂ are each independently selected from the group consisting of hydrogen, optionally substituted lower alkyl, optionally substituted cycloalkyl, halogen, optionally substituted heteroalkyl, optionally substituted cycloheteroalkyl, optionally substituted lower alkynyl, perhaloalkyl, perhaloalkoxy, hydroxy, optionally substituted lower alkoxy, nitro, cyano, and NH₂;

each R₄, each R₅, each R₇, and each R₈ are each independently selected from the group consisting of hydrogen, optionally substituted lower alkyl, halogen, lower perhaloalkyl, hydroxy, optionally substituted heteroalkyl, optionally substituted cycloalkyl, optionally substituted lower alkoxy, nitro, cyano, lower perhaloalkoxy, NH₂, and —C(O)—O—R₁₁, wherein R₁₁ is hydrogen or optionally substituted lower alkyl;

R₆ is selected from the group consisting of hydrogen, optionally substituted lower alkyl, hydroxy, and C₁₋₄ perhaloalkyl;

u is 1 or 2; t is 1 or 2;

G₃ is selected from the group consisting of a bond, a double bond, —(CR₉R₁₀)ₘ—, carbonyl, and —(CR₉R₁₀)ₘCR₉═CR₁₀—, wherein m is 0, 1, or 2, and wherein each R₉ and each R₁₀ is independently hydrogen, optionally substituted lower alkyl, optionally substituted lower alkoxy, optionally substituted aryl, lower perhaloalkyl, cyano, and nitro; and

G₄ is selected from the group consisting of optionally substituted aryl, optionally substituted heteroaryl, optionally substituted cycloalkyl, optionally substituted cycloheteroalkyl, optionally substituted cycloalkenyl, optionally substituted fused aryl, optionally substituted fused heteroaryl, and optionally substituted fused cycloalkyl; provided that when G₄ is said optionally substituted cycloheteroalkyl, said optional substitutents are non-cyclic; and further provided that when G₃ is a bond, G₄ may be covalently linked to G₂.

Another embodiment of the invention is a compound wherein A is selected from the group consisting of optionally substituted lower alkyl, optionally substituted cycloalkyl, halogen, optionally substituted heteroalkyl, optionally substituted cycloheteroalkyl, lower perhaloalkyl, hydroxy, and NH₂.

Another embodiment of the invention is a compound wherein A is selected from the group consisting of lower alkyl, optionally substituted cycloalkyl, optionally substituted cycloheteroalkyl, hydroxy, NH₂, and optionally substituted heteroalkyl wherein said heteroalkyl is attached to the phenyl ring at a carbon atom and said heteroalkyl contains at least one heteroatom selected from the group consisting of O, N, and S.

Another embodiment of the invention is a compound wherein A is selected from the group consisting of lower alkyl and an optionally substituted heteroalkyl.

Another embodiment of the invention is a compound wherein A, X₁ and X₂ are each independently selected from the group consisting of hydrogen, optionally substituted lower alkyl, halogen, optionally substituted lower heteroalkyl, perhaloalkyl, perhaloalkoxy, and optionally substituted lower alkoxy.

Another embodiment of the invention is a compound wherein A, X₁ and X₂ are each independently selected from the group consisting of hydrogen and methyl and at least one of A, X₁ and X₂ is methyl.

Another embodiment of the invention is a compound wherein n=1.

Another embodiment of the invention is a compound wherein R₁ and R₂ are each independently selected from the group consisting of hydrogen, lower alkyl, or together may form an optionally substituted cycloalkyl.

Another embodiment of the invention is a compound wherein R₁ and R₂ are each hydrogen.

Another embodiment of the invention is a compound having the structure

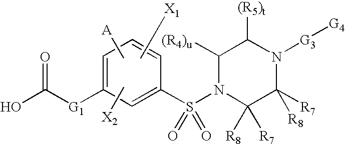

Another embodiment of the invention is a compound wherein at least one of R₄, R₅, R₇, and R₈ is not hydrogen.

Another embodiment of the invention is a compound wherein said at least one of R₄, R₅, R₇, and R₈ is lower alkyl.

Another embodiment of the invention is a compound wherein said at least one of R₄, R₅, R₇, and R₈ is methyl. Yet another embodiment of the invention is a compound wherein at least two of R₄, R₅, R₇, and R₈ are methyl.

Another embodiment of the invention is a compound wherein the at least two of R₄, R₅, R₇, and R₈ which are methyl are oriented cis to each other.

Another embodiment of the invention is a compound wherein R₄ and R₇ are methyl and are attached to the piperazine ring at the 2 and 6 positions.

Another embodiment of the invention is a compound wherein the R₄ and R₇ methyl groups are oriented cis to each other.

Another embodiment of the invention is a compound wherein R₄ and R₅ are methyl.

Another embodiment of the invention is a compound wherein the R₄ and R₅ methyl groups are oriented cis to each other.

Another embodiment of the invention is a compound wherein at least two of R₄, R₅, R₇, and R₈ are methyls oriented cis to each other.

Another embodiment of the invention is a compound wherein G₃ is a bond.

Another embodiment of the invention is a compound wherein G₄ has a structural formula selected from the group consisting of:

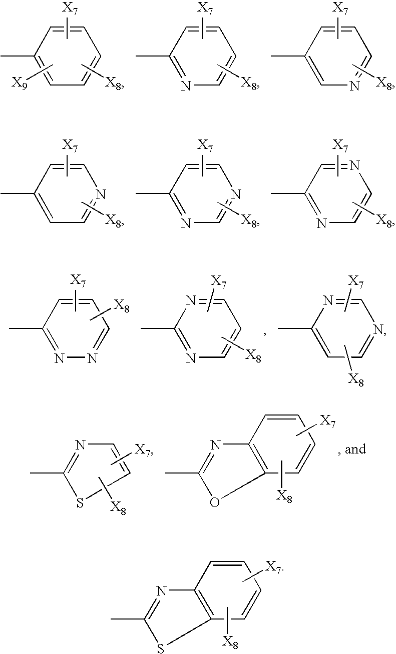

wherein each X₇, X₈ and X₉ are each independently selected from the group consisting of hydrogen, optionally substituted lower alkyl, halogen, lower perhaloalkyl, hydroxy, optionally substituted lower alkoxy, lower perhaloalkoxy, nitro, cyano, NH₂, and CO₂R₁₂ where R₁₂ is optionally substituted lower alkyl and H;

X₇ and X₈, if present on adjacent sites of G₄, may together form an aryl, heteroaryl, aliphatic or heteroaliphatic ring.

Another embodiment of the invention is a compound wherein G₃ is a bond.

Another embodiment of the invention is a compound wherein the compound is an hPPAR-delta modulator.

Another embodiment of the invention is a compound wherein the compound is a selective hPPAR-delta modulator.

Another embodiment of the invention is a compound wherein the compound modulates hPPAR-delta having an EC₅₀ value less than 5 μM as measured by a functional cell assay.

Another embodiment of the invention is a compound having the structure 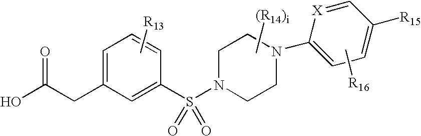
or a pharmaceutically acceptable N-oxide, pharmaceutically acceptable prodrug, pharmaceutically acceptable metabolite, pharmaceutically acceptable salt, pharmaceutically acceptable ester, pharmaceutically acceptable amide, or pharmaceutically acceptable solvate thereof;

wherein:

X is C or N;

R₁₃ is selected from the group consisting of hydrogen, C₁-C₄ alkyl, and singly or multiply fluoro substituted C₁-C₄ alkyl;

each R₁₄ is selected from the group consisting of hydrogen, C₁-C₃ alkyl;

i is 0, 1, or 2;

R₁₅ is selected from the group consisting of halogen, perhalomethyl, and perhalomethoxy; and

R₁₆ is selected from the group consisting of hydrogen, halogen, lower alkyl and lower alkoxy.

Another embodiment of the invention is a compound wherein R₁₃ is selected from the group consisting of hydrogen, methyl, perfluoromethyl, difluoromethyl and —CH₂—CF₃.

Another embodiment of the invention is a compound wherein R₁₄ is selected from the group consisting of hydrogen, methyl, ethyl, and isopropyl.

Another embodiment of the invention is a compound wherein i=2 and R₁₄ is selected from the group consisting of methyl.

Another embodiment of the invention is a compound wherein the two R₁₄ moieties are oriented cis to each other.

Another embodiment of the invention is a compound wherein the two R₁₄ moieties are attached to the piperazine ring at the 2 and 6 positions.

Another embodiment of the invention is a compound wherein the two R₁₄ moieties are attached to the piperazine ring at the 2 and 3 positions.

Another embodiment of the invention is a compound wherein R₁₃ is selected from the group consisting of hydrogen, methyl, perfluoromethyl, difluoromethyl and —CH₂—CF₃.

Another embodiment of the invention is a compound wherein R₁₅ is selected from the group consisting of halogen, perfluoromethyl, and perfluoromethoxy.

Another embodiment of the invention is a compound wherein R₁₃ is selected from the group consisting of hydrogen, methyl, perfluoromethyl, difluoromethyl and —CH₂—CF₃.

Another embodiment of the invention is a compound wherein the compound is an hPPAR-delta modulator.

Another embodiment of the invention is a compound wherein the compound is a selective hPPAR-delta modulator.

Another embodiment of the invention is a compound wherein the compound modulates hPPAR-delta having an EC₅₀ value less than 5 μM as measured by a functional cell assay.

Another embodiment of the invention is a compound having a structure, or a pharmaceutically acceptable N-oxide, pharmaceutically acceptable prodrug, pharmaceutically acceptable metabolite, pharmaceutically acceptable salt, pharmaceutically acceptable ester, pharmaceutically acceptable amide, or pharmaceutically acceptable solvate thereof, wherein the structure is selected from the group consisting of the structures disclosed as Examples 1-233 herein.

Another embodiment of the invention is a compound, or a pharmaceutically acceptable N-oxide, pharmaceutically acceptable prodrug, pharmaceutically acceptable metabolite, pharmaceutically acceptable salt, pharmaceutically acceptable ester, pharmaceutically acceptable amide, or pharmaceutically acceptable solvate thereof, selected from the group consisting of:

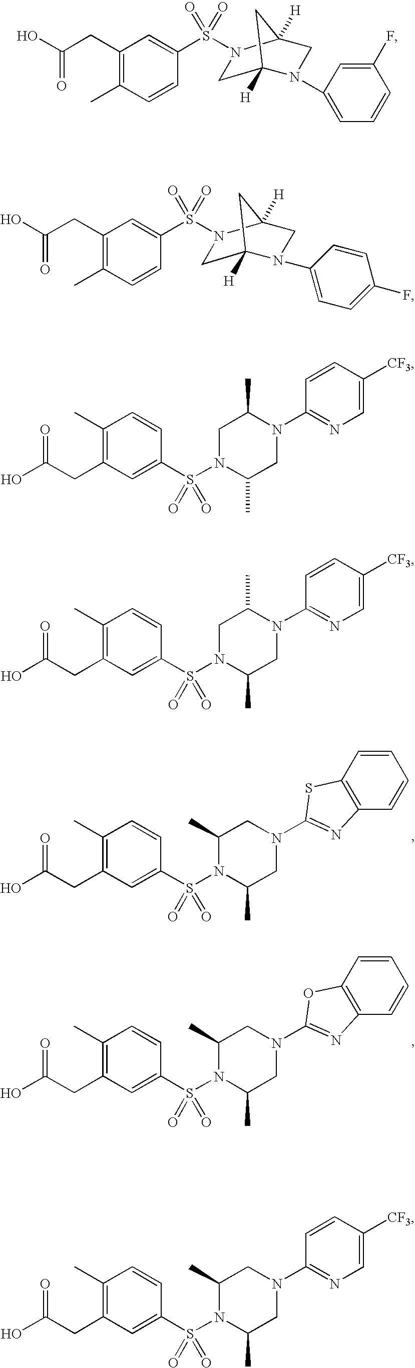

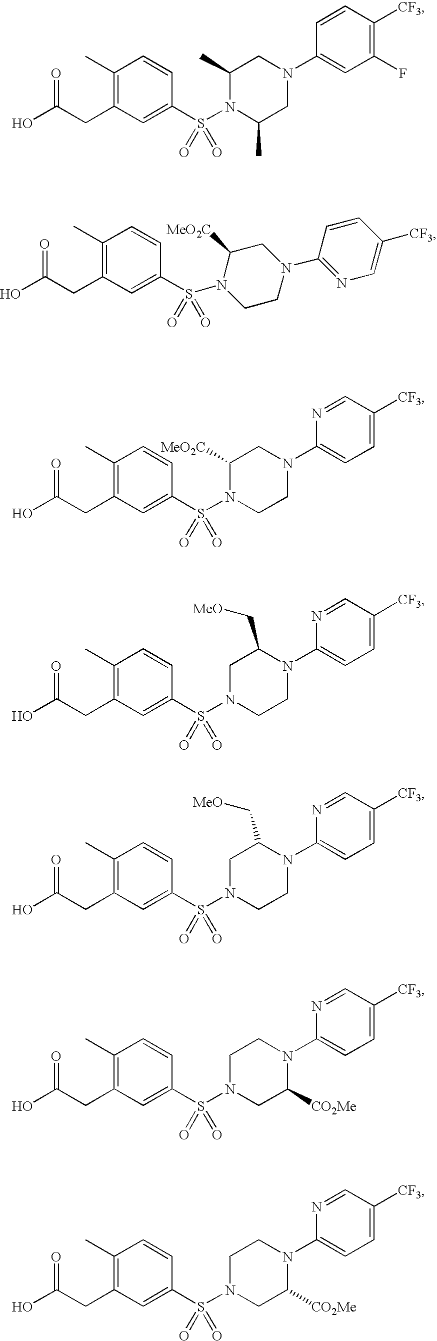

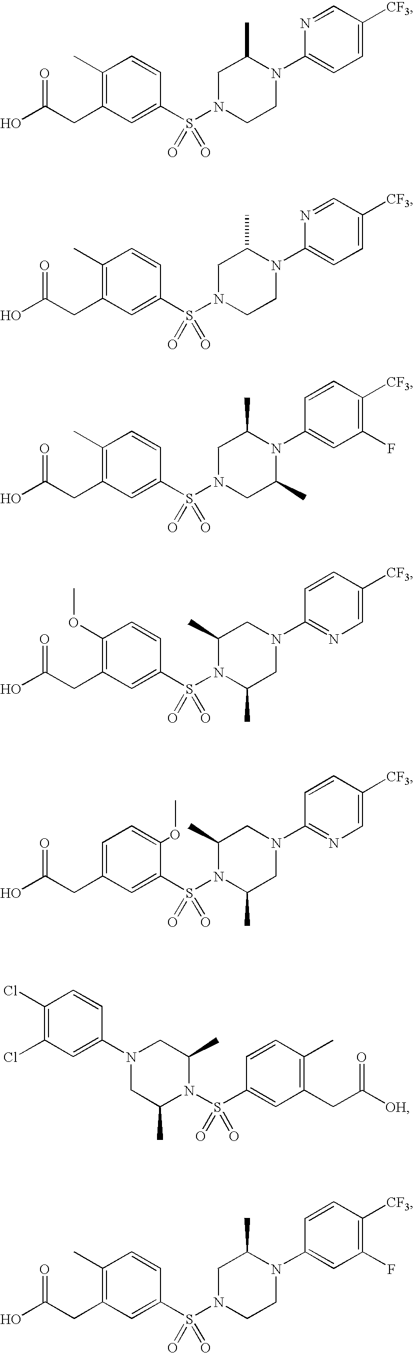

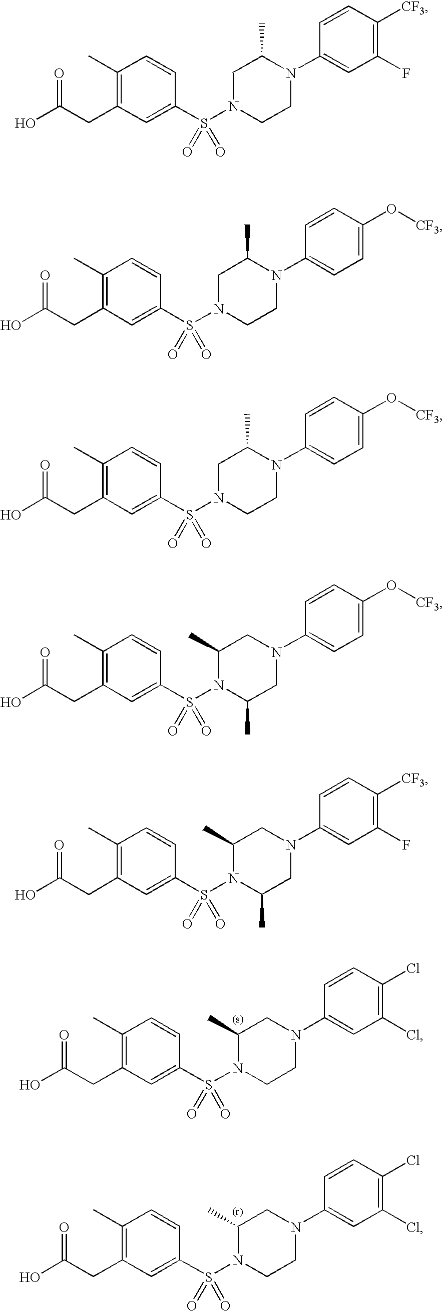

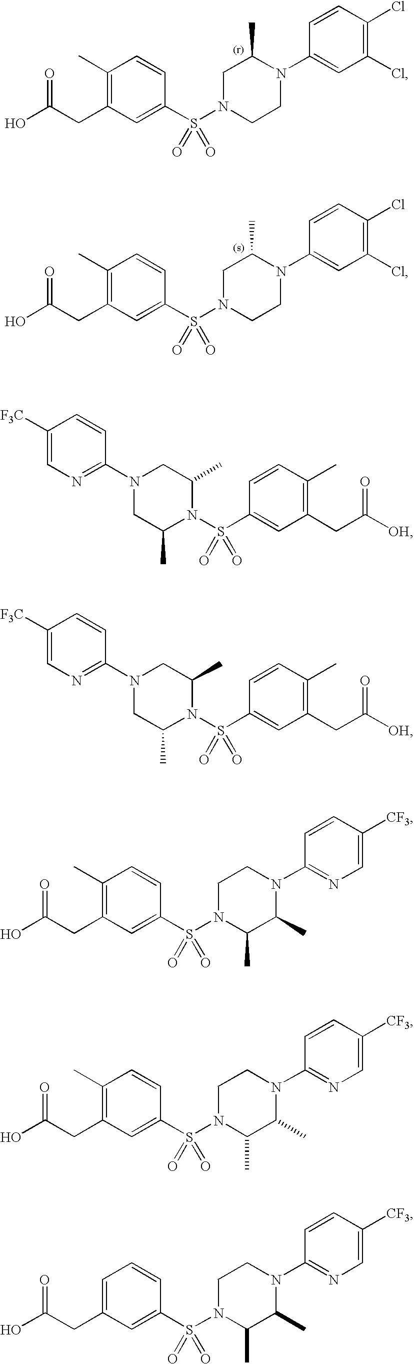

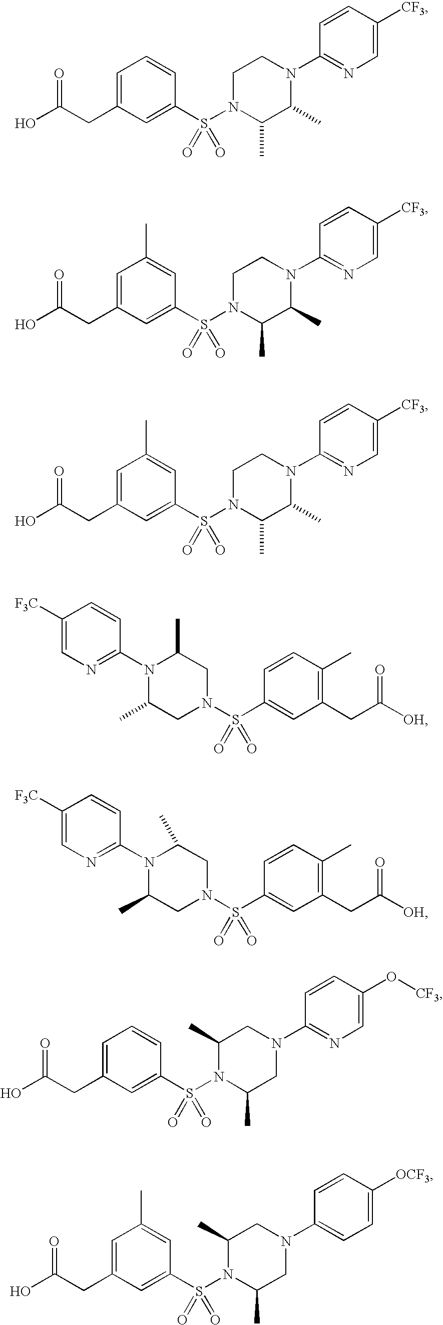

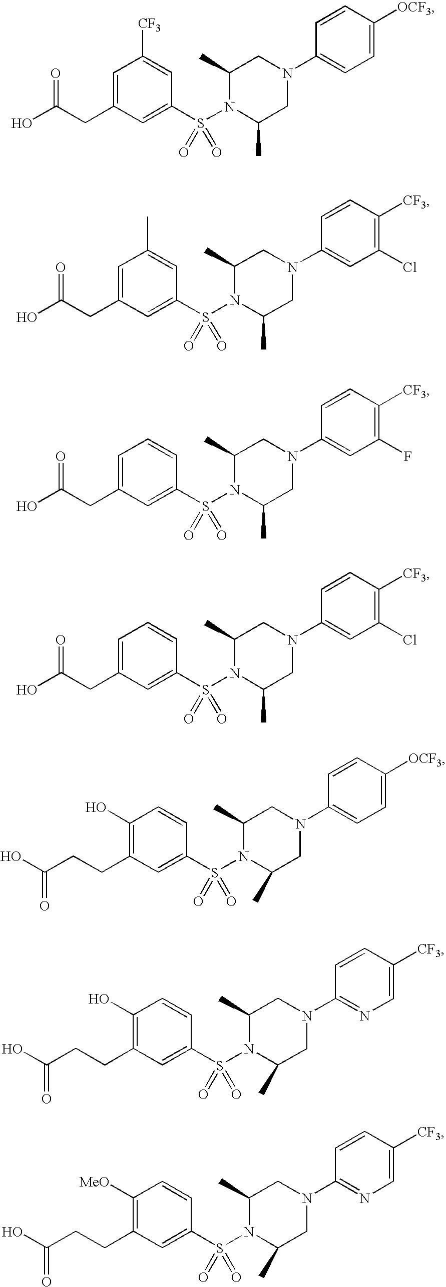

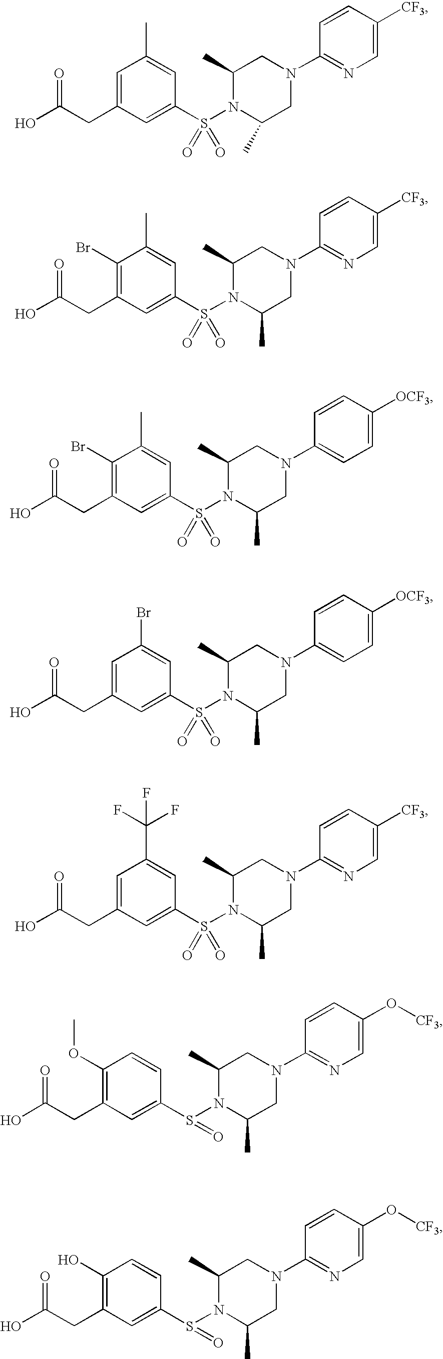

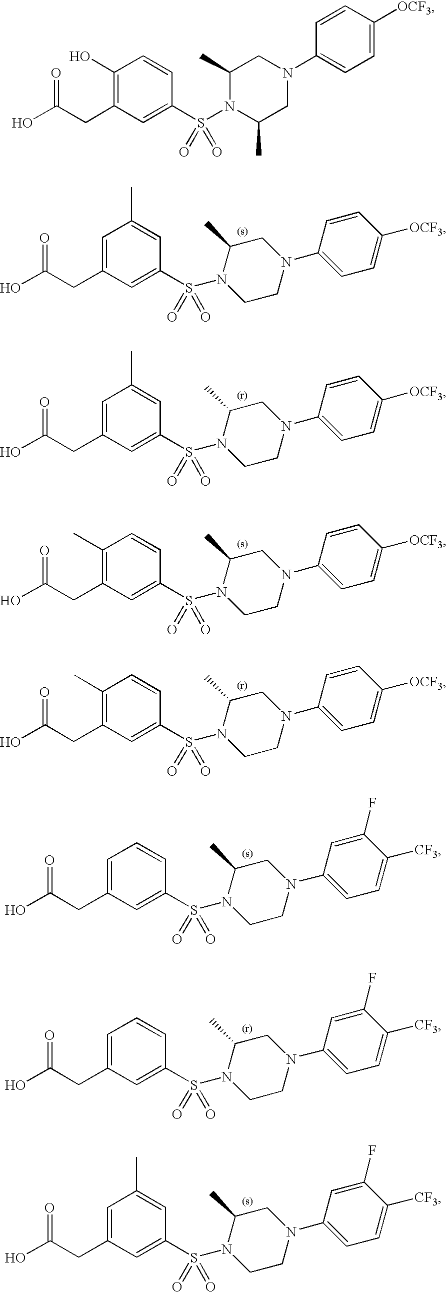

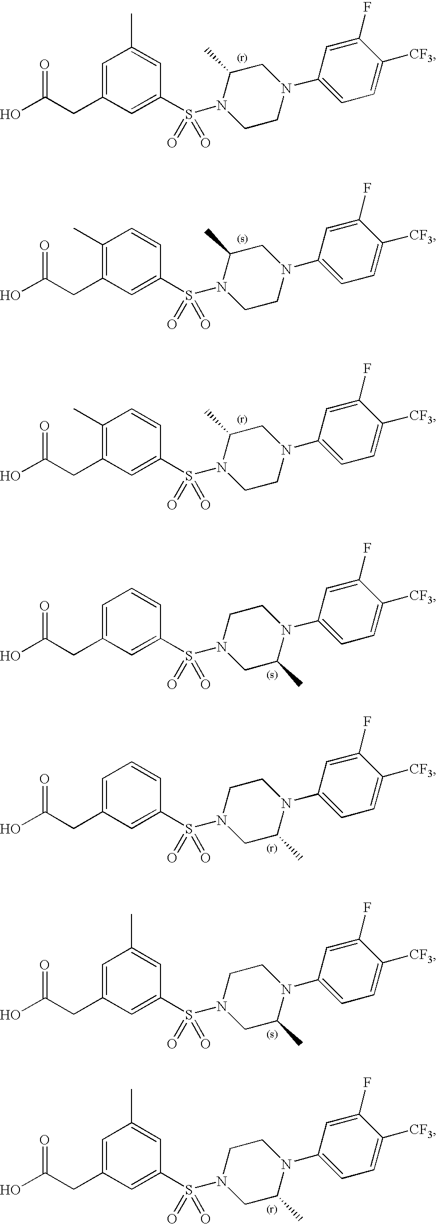

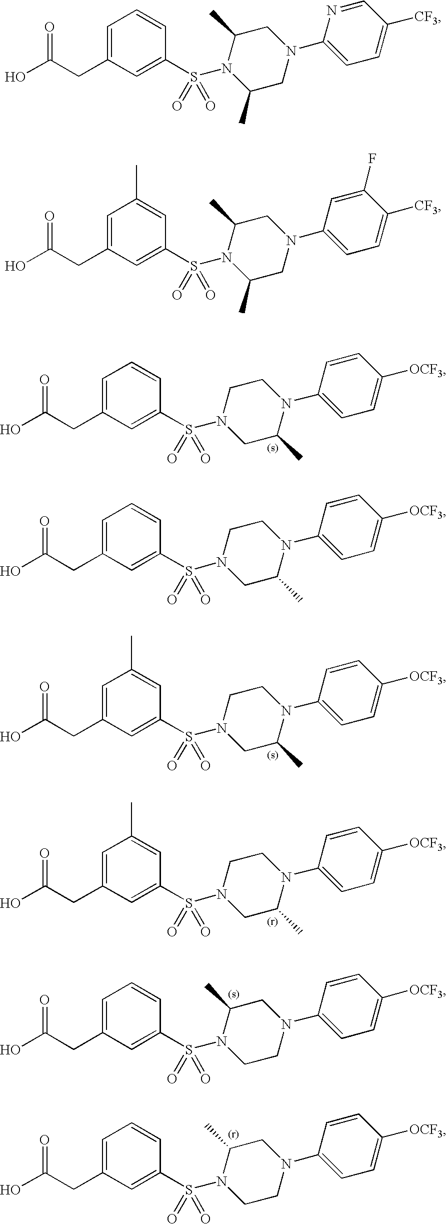

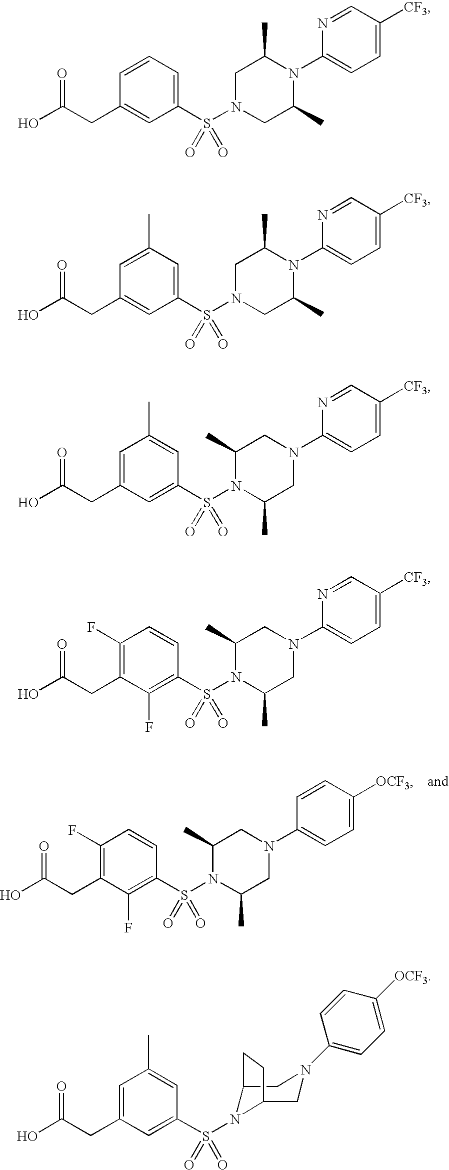

Another embodiment of the invention is a compound, or a pharmaceutically acceptable N-oxide, pharmaceutically acceptable prodrug, pharmaceutically acceptable metabolite, pharmaceutically acceptable salt, pharmaceutically acceptable ester, pharmaceutically acceptable amide, or pharmaceutically acceptable solvate thereof, wherein the compound is of the structure A-B-C wherein the A, B and C moieties are independently selected from the respective columns in Table 1. The compounds of this embodiment are predicted to have PPAR-delta modulating activity.

TABLE 1

| | A | B | C |
|---|---|---|---|
| 1 | 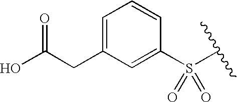 | 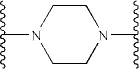 | 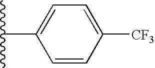 |
| 2 | 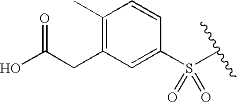 | 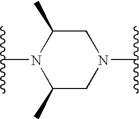 | 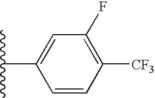 |
| 3 | 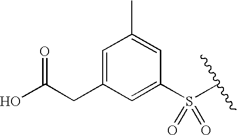 | 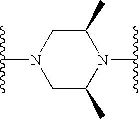 | 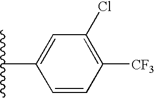 |
| 4 | 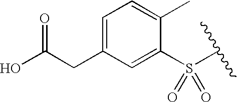 | 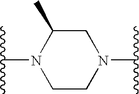 | 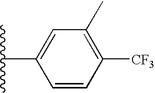 |
| 5 | 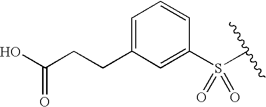 | 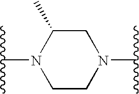 | 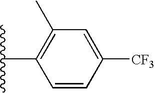 |
| 6 | 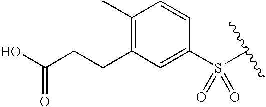 | 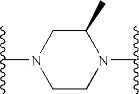 | 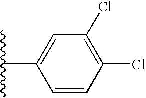 |
| 7 | 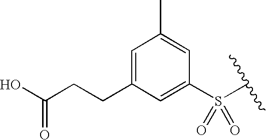 | 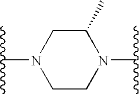 | 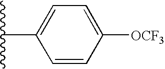 |
| 8 | 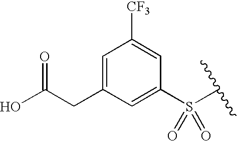 | 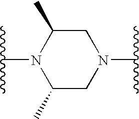 | 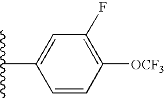 |
| 9 | 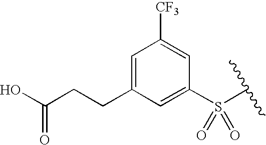 | 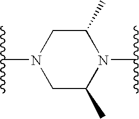 | 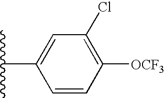 |
| 10 | 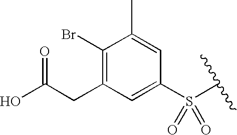 | 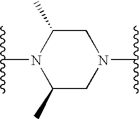 | 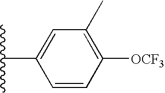 |
| 11 | 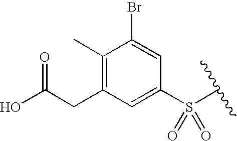 | 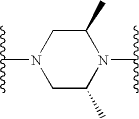 | 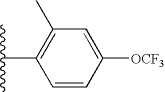 |
| 12 | 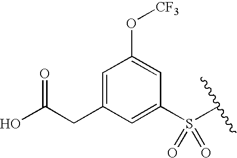 | 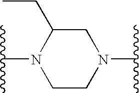 | 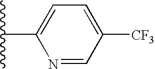 |
| 13 | 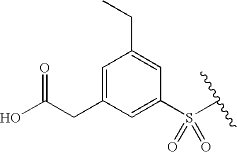 | 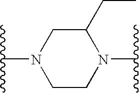 | 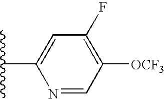 |
| 14 | 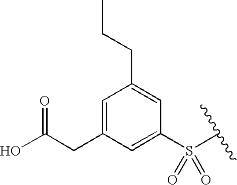 | 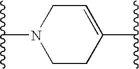 | 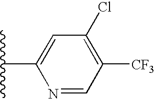 |
| 15 | 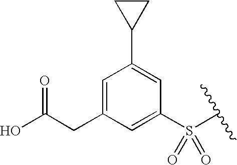 | 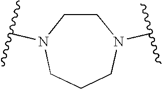 | 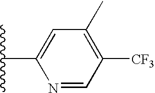 |
| 16 | 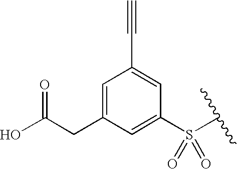 | 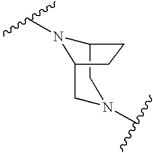 | 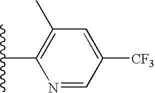 |
| 17 | 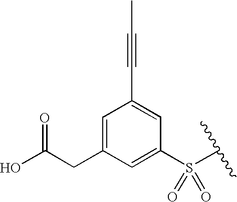 | 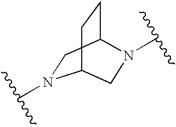 | 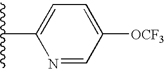 |
| 18 | 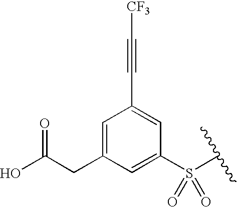 | 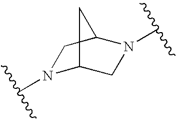 | 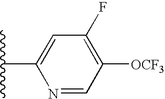 |
| 19 | 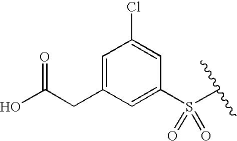 | | 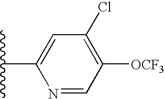 |
| 20 | 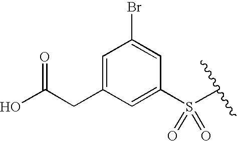 | | 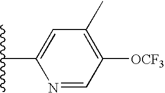 |
| 21 | 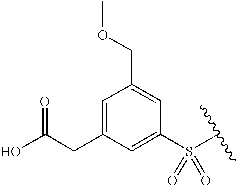 | | 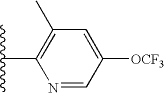 |
| 22 | 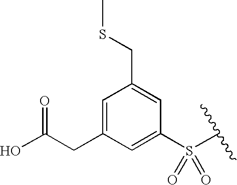 | | 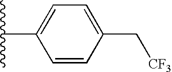 |
| 23 | 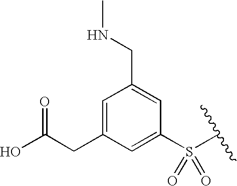 | | 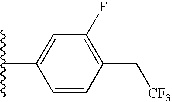 |
| 24 | 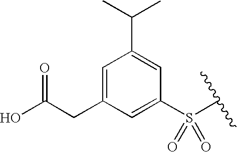 | | 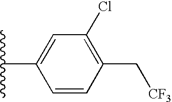 |
| 25 | 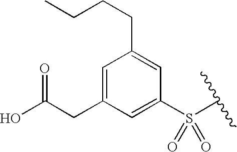 | | 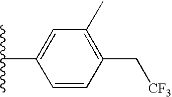 |
| 26 | 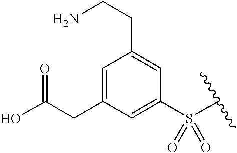 | | 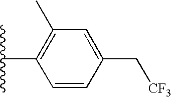 |
| 27 | 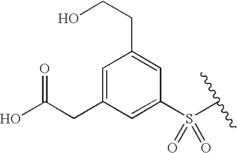 | | 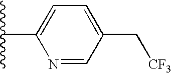 |
| 28 | 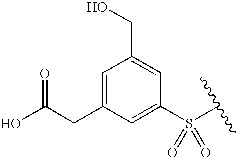 | | 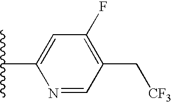 |
| 29 | 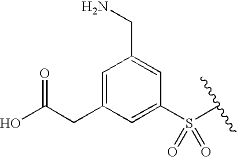 | | 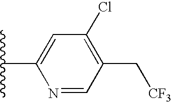 |
| 30 | 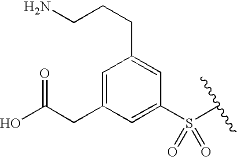 | | 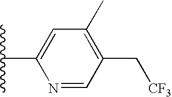 |
| 31 | 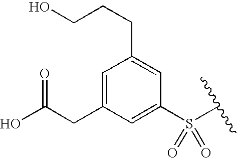 | | 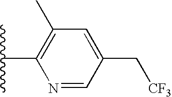 |
| 32 | 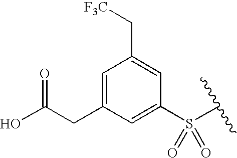 | | 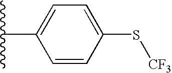 |
| 33 | 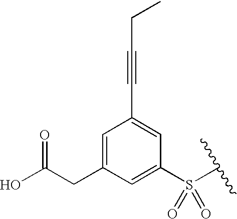 | | 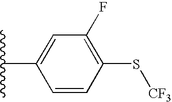 |
| 34 | | | 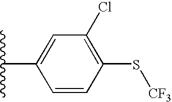 |
| 35 | | | 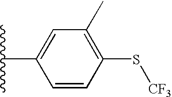 |
| 36 | | | 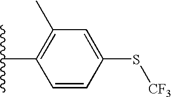 |
| 37 | | | 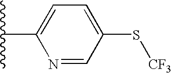 |
| 38 | | | 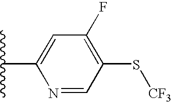 |
| 39 | | | 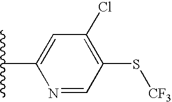 |
| 40 | | | 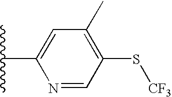 |
| 41 | | | 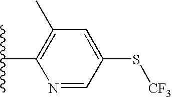 |
| 42 | | | 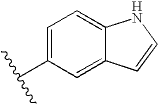 |
| 43 | | | 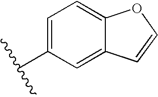 |
| 44 | | | 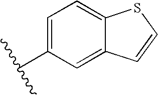 |
| 45 | | | 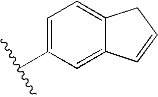 |
| 46 | | | 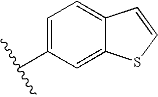 |
| 47 | | | 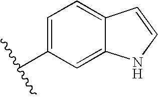 |
| 48 | | | 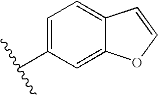 |
| 49 | | | 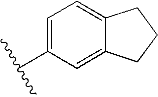 |
| 50 | | | 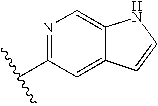 |
| 51 | | | 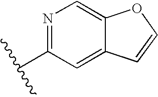 |
| 52 | | | 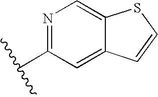 |
| 53 | | | 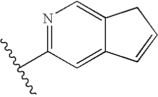 |
| 54 | | | 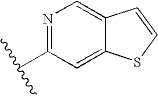 |
| 55 | | | 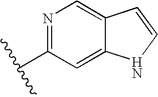 |
| 56 | | | 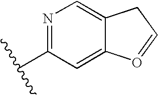 |
| 57 | | | 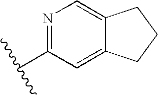 |
| 58 | | | 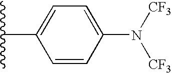 |
| 59 | | | 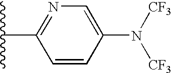 |
| 60 | | | 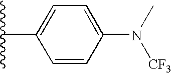 |
| 61 | | | 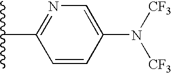 |
| 62 | | | 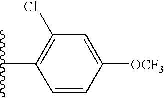 |
| 63 | | | 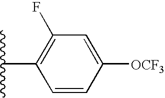 |
| 64 | | | 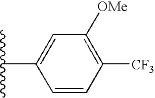 |
| 65 | | | 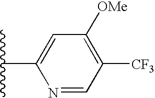 |
| 66 | | | 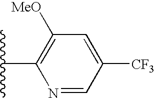 |
| 67 | | | 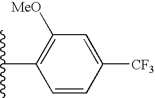 |

Those skilled in the art will recognize that Table 1 discloses individual compounds as if all combinations of moieties A, B, and C were individually drawn out. By way of illustration, specific examples of the compounds of this embodiment as disclosed above in Table 1 are as follows:

The A moiety drawn from row 2, the B moiety drawn from row 4, and the C moiety drawn from row 9 together combine to form the following specific example:

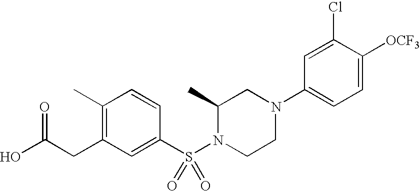

The A moiety drawn from row 19, the B moiety drawn from row 2, and the C moiety drawn from row 7 together combine to form the following specific example:

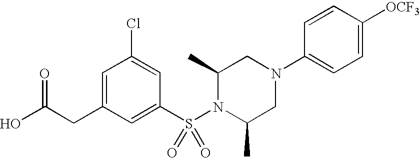

The A moiety drawn from row 6, the B moiety drawn from row 9, and the C moiety drawn from row 43 together combine to form the following specific example:

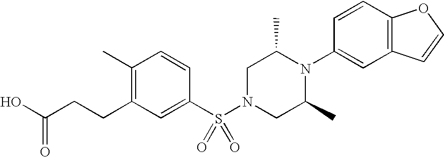

Another embodiment of the invention is a compound for use in the treatment of disease or condition ameliorated by the modulation of a hPPAR-delta.

Another embodiment of the invention is a compound pharmaceutical composition comprising a compound of structural formula (I).

Another embodiment of the invention is a pharmaceutical composition further comprising a pharmaceutical acceptable diluent or carrier.

Another embodiment of the invention is a composition for use in the treatment of disease or condition ameliorated by the modulation of a hPPAR-delta. Specific diseases or conditions include but are not limited to dyslipidemia, metabolic syndrome X, heart failure, hypercholesteremia, cardiovascular disease, type II diabetes mellitus, type 1 diabetes, insulin resistance hyperlipidemia, obesity, anorexia bulimia, inflammation, a wound, and anorexia nervosa.

Another embodiment of the invention is a compound for use in the manufacture of a medicament for the prevention or treatment of a disease or condition ameliorated by the modulation of a hPPAR-delta.

Another embodiment of the invention is a compound, a pharmaceutically acceptable prodrug, pharmaceutically active metabolite, or pharmaceutically acceptable salt having an EC₅₀ value less than 5 μM as measured by a functional cell assay.

Another embodiment of the invention is a method for raising HDL in a subject comprising the administration of a therapeutic amount of a compounds of the invention.

Another embodiment of the invention is the use of a hPPAR-delta modulator compound of the invention for the manufacture of a medicament for the raising of HDL in a patient in need thereof.

Another embodiment of the invention is a method for treating Type 2 diabetes, decreasing insulin resistance or lowering blood pressure in a subject comprising the administration of a therapeutic amount of a compound of the invention.

Another embodiment of the invention is the use of a hPPAR-delta modulator compound of the invention for the manufacture of a medicament for the treatment of Type 2 diabetes, decreasing insulin resistance or lowering blood pressure in a patient in need thereof.

Another embodiment of the invention is a method for decreasing LDLc in a subject comprising the administration of a therapeutic amount of a compound of the invention.

Another embodiment of the invention is the use of a hPPAR-delta modulator compound of the invention for the manufacture of a medicament for decreasing LDLc in a patient in need thereof.

Another embodiment of the invention is a method for shifting LDL particle size from small dense to normal dense LDL in a subject comprising the administration of a therapeutic amount of a hPPAR-delta modulator compound of the invention.

Another embodiment of the invention is the use of a hPPAR-delta modulator compound of the invention for the manufacture of a medicament for shifting LDL particle size from small dense to normal LDL in a patient in need thereof.

Another embodiment of the invention is a method for treating atherosclerotic diseases including vascular disease, coronary heart disease, cerebrovascular disease and peripheral vessel disease in a subject comprising the administration of a therapeutic amount of a hPPAR-delta modulator compound of the invention.

Another embodiment of the invention is the use of a hPPAR-delta modulator compound of the invention for the manufacture of a medicament for the treatment of atherosclerotic diseases including vascular disease, coronary heart disease, cerebrovascular disease and peripheral vessel disease in a patient in need thereof.

Another embodiment of the invention is a method for treating inflammatory diseases, including rheumatoid arthritis, asthma, osteoarthritis and autoimmune disease in a subject comprising the administration of a therapeutic amount of a hPPAR-delta modulator compound of the invention.

Another embodiment of the invention is the use of a hPPAR-delta modulator compound of the invention for the manufacture of a medicament for the treatment of inflammatory diseases, including rheumatoid arthritis, asthma, osteoarthritis and autoimmune disease in a patient in need thereof.

Another embodiment of the invention is a method of treatment of a hPPAR-delta mediated disease or condition comprising administering a therapeutically effective amount of a compound of the invention or a pharmaceutically acceptable salt, ester, amide, or prodrug thereof.

Another embodiment of the invention is a method of modulating a peroxisome proliferator-activated receptor (PPAR) function comprising contacting said PPAR with a compound of claim 1 and monitoring a change in cell phenotype, cell proliferation, activity of said PPAR, or binding of said PPAR with a natural binding partner.

Another embodiment of the invention is a method of modulating a peroxisome proliferator-activated receptor (PPAR) function, wherein the PPAR is selected from the group consisting of PPAR-alpha, PPAR-delta, and PPAR-gamma.

Another embodiment of the invention is a method of treating a disease comprising identifying a patient in need thereof, and administering a therapeutically effective amount of a compound of the invention to said patient wherein the disease is selected from the group consisting of obesity, diabetes, hyperinsulinemia, metabolic syndrome X, polycystic ovary syndrome, climacteric, disorders associated with oxidative stress, inflammatory response to tissue injury, pathogenesis of emphysema, ischemia-associated organ injury, doxorubicin-induced cardiac injury, drug-induced hepatotoxicity, atherosclerosis, and hypertoxic lung injury.

Another embodiment of the invention is a compound having structural formula (I) which modulates a peroxisome proliferator-activated receptor (PPAR) function.

Another embodiment of the invention is a compound of the invention which modulates a peroxisome proliferator-activated receptor (PPAR) function, wherein the PPAR is selected from the group consisting of PPARα, PPARδ, and PPARγ.

Another embodiment of the invention is a compound of the invention for use in the treatment of a disease or condition ameliorated by the modulation of PPARα, PPARδ, or PPARγ. Specific diseases or conditions include but are not limited to dyslipidemia, metabolic syndrome X, heart failure, hypercholesteremia, cardiovascular disease, type II diabetes mellitus, type 1 diabetes, insulin resistance hyperlipidemia, obesity, anorexia bulimia, inflammation and anorexia nervosa.

Another embodiment of the invention is a compound or composition for use in the manufacture of a medicament for the prevention or treatment of disease or condition ameliorated by the modulation of a PPARα, PPARδ, and PPARγ.

### DETAILED DESCRIPTION OF THE INVENTION

The present invention discloses that alkyl-substituted phenyl sulfonamide compounds also substituted with an acid or ester moiety can modulate at least one peroxisome proliferator-activated receptor (PPAR) function. Compounds described herein may be activating both PPAR-delta and PPAR-gamma or PPAR-alpha and PPAR-delta, or all three PPAR subtypes, or selectively activating predominantly hPPAR-gamma, hPPAR-alpha or hPPAR-delta.

The present invention relates to a method of modulating at least one peroxisome proliferator-activated receptor (PPAR) function comprising the step of contacting the PPAR with a compound of Formula I, as described herein. The change in cell phenotype, cell proliferation, activity of the PPAR, expression of the PPAR or binding of the PPAR with a natural binding partner may be monitored. Such methods may be modes of treatment of disease, biological assays, cellular assays, biochemical assays, or the like.

The present invention describes methods of treating a disease comprising identifying a patient in need thereof, and administering a therapeutically effective amount of a compound of Formula I, as described herein, to a patient. Thus, in certain embodiments, the disease to be treated by the methods of the present invention is selected from the group consisting of obesity, diabetes, hyperinsulinemia, metabolic syndrome X, polycystic ovary syndrome, climacteric, disorders associated with oxidative stress, inflammatory response to tissue injury, pathogenesis of emphysema, ischemia-associated organ injury, doxorubicin-induced cardiac injury, drug-induced hepatotoxicity, atherosclerosis, and hypertoxic lung injury.

### CHEMICAL TERMINOLOGY

An “acetyl” group refers to a —C(═O)CH₃, group.

The term “acyl” includes alkyl, aryl, or heteroaryl substituents attached to a compound via a carbonyl functionality (e.g., —C(O)-alkyl, —C(O)-aryl, etc.).

An “alkoxy” group refers to a RO— group, where R is as defined herein. The alkoxy group could also be a “lower alkoxy” having 1 to 5 carbon atoms. The alkoxy group of the compounds of the invention may be designated as “C₁-C₄ alkoxy” or similar designations. An alkoxy group may be optionally substituted at a carbon with one or more groups or substituents replacing a hydrogen atom. Groups and substituents which may replace hydrogen atoms include but are not limited to halogen, perhaloalkyl, hydroxy, alkoxy, perhaloalkoxy, aryloxy, mercapto, alkylthio, arylthio, perfluoroalkyl, cyano, carbonyl, carboxy, carboxyester, ether, amine, thiocarbonyl, O-thiocarbamyl, N-thiocarbamyl, C-amido, N-amido, S-sulfonamido, N-sulfonamido, C-carboxy, O-carboxy, isocyanato, thiocyanato, isothiocyanato and nitro.

An “alkoxyalkoxy” group refers to a ROR′O— group, where R is as defined herein.

An “alkoxyalkyl” group refers to a R′OR— group, where R and R′ are as defined herein.

As used herein, the term “alkyl” refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The “alkyl” moiety may have 1 to 40 carbon atoms (whenever it appears herein, a numerical range such as “1 to 40” refers to each integer in the given range; e.g., “1 to 40 carbon atoms” means that the alkyl group may consist of 1 carbon atom, 2 carbon atoms, 3 carbon atoms, etc., up to and including 40 carbon atoms, although the present definition also covers the occurrence of the term “alkyl” where no numerical range is designated). The alkyl group may be a “medium alkyl” having 1 to 20 carbon atoms. The alkyl group could also be a “lower alkyl” having 1 to 5 carbon atoms. The alkyl group of the compounds of the invention may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. An alkyl group may be optionally substituted with one or more groups or substituents replacing a hydrogen atom. Groups and substituents which may replace hydrogen atoms include but are not limited to halogen, perhaloalkyl, hydroxy, alkoxy, perhaloalkoxy, aryloxy, mercapto, alkylthio, arylthio, perfluoroalkyl, cyano, carbonyl, carboxy, carboxyester, ether, amine, thiocarbonyl, O-thiocarbamyl, N-thiocarbamyl, C-amido, N-amido, S-sulfonamido, N-sulfonamido, C-carboxy, O-carboxy, isocyanato, thiocyanato, isothiocyanato and nitro.

The term “alkylamino” refers to the —NRR′ group, where R and R′ are as defined herein. R and R′, taken together, can optionally form a cyclic ring system.

The term “alkylene” refers to an alkyl group that is substituted at two ends (i.e., a diradical). Thus, methylene (—CH₂—) ethylene (—CH₂CH₂—), and propylene (—CH₂CH₂CH₂—) are examples of alkylene groups. Similarly, “alkenylene” and “alkynylene” groups refer to diradical alkene and alkyne moieties, respectively. An alkylene group may be optionally substituted.

An “amide” is a chemical moiety with formula —C(O)NHR or —NHC(O)R, where R is optionally substituted and is selected from the group-consisting of alkyl, cycloalkyl, aryl, heteroaryl (bonded through a ring carbon) and heteroalicyclic (bonded through a ring carbon). An amide may be an amino acid or a peptide molecule attached to a molecule of the present invention, thereby forming a prodrug. Any amine, hydroxy, or carboxyl side chain on the compounds of the present invention can be amidified. The procedures and specific groups to be used to achieve makes such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

A “C-amido” group refers to a —C(═O)—NR₂ group with R as defined herein.

An “N-amido” group refers to a RC(═O)NH— group, with R as defined herein.

The term “aromatic” or “aryl” refers to an aromatic group which has at least one ring having a conjugated pi electron system and includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. An aromatic or aryl group may be optionally substituted with one or more groups or substituents replacing a hydrogen atom. Groups and substituents which may replace hydrogen atoms include but are not limited to halogen, perhaloalkyl, heteroalkyl, hydroxy, alkoxy, perhaloalkoxy, aryloxy, mercapto, alkylthio, arylthio, perfluoroalkyl, cyano, carbonyl, carboxy, carboxyester, ether, amine, thiocarbonyl, O-thiocarbamyl, N-thiocarbamyl, C-amido, N-amido, S-sulfonamido, N-sulfonamido, C-carboxy, O-carboxy, isocyanato, thiocyanato, isothiocyanato and nitro.

An “O-carbamyl” group refers to a —OC(═O)—NR, group-with R as defined herein.

An “N-carbamyl” group refers to a ROC(═O)NH— group, with R as defined herein.

An “O-carboxy” group refers to a RC(═O)O— group, where R is as defined herein.

A “C-carboxy” group refers to a —C(═O)OR groups where R is as defined herein.

A “cyano” group refers to a —CN group.

The term “cycloalkyl” refers to a monocyclic or polycyciic radical which contains only carbon and hydrogen, and may be saturated, partially unsaturated, or fully unsaturated. A cycloalkyl group may be optionally substituted. Preferred cycloalkyl groups include groups having from three to twelve ring atoms, more preferably from 5 to 10 ring atoms. Illustrative examples of cycloalkyl groups include the following moieties: 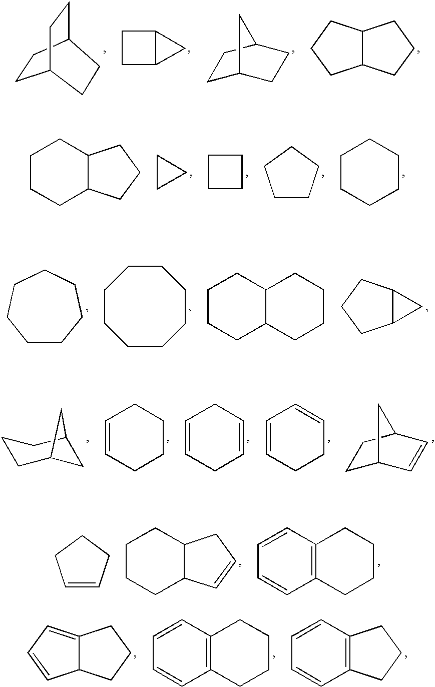
and the like. A cycloalkyl group may be optionally substituted with one or more groups or substituents replacing a hydrogen atom. Groups and substituents which may replace hydrogen atoms include but are not limited to halogen, perhaloalkyl, hydroxy, alkoxy, perhaloalkoxy, aryloxy, mercapto, alkylthio, arylthio, perfluoroalkyl, cyano, carbonyl, carboxy, carboxyester, ether, amine, thiocarbonyl, O-thiocarbamyl, N-thiocarbamyl, C-amido, N-amido, S-sulfonamido, N-sulfonamido, C-carboxy, O-carboxy, isocyanato, thiocyanato, isothiocyanato and nitro.

The term “ester” refers to a chemical moiety with formula —COORₑ, where Rₑ is optionally substituted and is selected from the group consisting of alkyl, cycloalkyl, aryl, heteroaryl (bonded through a ring carbon) and heteroalicyclic (bonded through a ring carbon). Any amine, hydroxy, or carboxyl side chain on the compounds of the present invention can be esterified. The procedures and specific groups to be used to achieve makes such esters are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “halo” or, alternatively, “halogen” means fluoro, chlord, bromo or iodo. Preferred halo groups are fluoro, chloro and bromo.

The terms “haloalkyl,” “haloalkenyl,” “haloalkynyl” and “haloalkoxy” include alkyl, alkenyl, alkynyl and alkoxy structures, that are substituted with one or more halo groups or with combinations thereof. The terms “fluoroalkyl” and “fluoroalkoxy” include haloalkyl and haloalkoxy groups, respectively, in which the halo is fluorine.

The terms “heteroalkyl” “heteroalkenyl” and “heteroalkynyl” include optionally substituted alkyl, alkenyl and alkynyl radicals and which have one or more skeletal chain atoms selected from an atom other that carbon, e.g., oxygen, nitrogen, sulfur, phosphorus or combinations thereof. The heteroatom in a heteroalkyl group may be within the skeletal chain or at an end of the skeletal chain (e.g., both —CH₂—O—CH₃ and —CH₂—CH₂—OH are heteroalkyl groups). A heteroalkyl group may be optionally substituted with one or more groups or substituents replacing a hydrogen atom. Groups and substituents which may replace hydrogen atoms include but are not limited to halogen, perhaloalkyl, hydroxy, alkoxy, perhaloalkoxy, aryloxy, mercapto, alkyltbio, arylthio, perfluoroalkyl, cyano, carbonyl, carboxy, carboxyester, ether, amine, thiocarbonyl, 0-thiocarbamyl, N-thiocarbamyl, C-amido, N-amido, S-sulfonamido, N-sulfonamido, C-carboxy, O-carboxy, isocyanato, thiocyanato, isothiocyanato and nitro.

The terms “heteroaryl” or, alternatively, “heteroaromatic” refers to an aryl group that includes one or more ring heteroatoms selected from nitrogen, oxygen and sulfur. A heteroaryl group may be optionally substituted. An N-containing “heteroaromatic” or “heteroaryl” moiety refers to an aromatic group in which at least one of the skeletal atoms of the ring is a nitrogen atom. The polycyclic heteroaryl group may be fused or non-fused. Illustrative examples of heteroaryl groups include the following moieties: 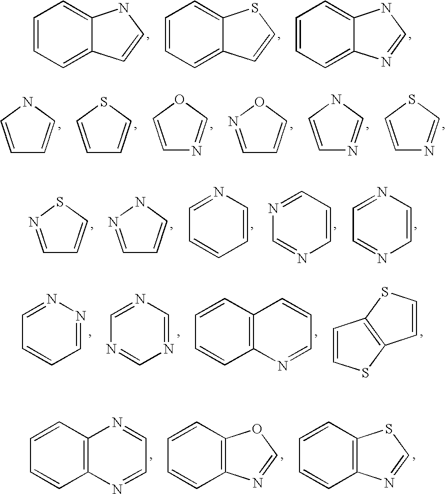
and the like. A heteroaryl group may be optionally substituted with one or more groups or substituents replacing a hydrogen atom. Groups and substituents which may replace hydrogen atoms include but are not limited to halogen, perhaloalkyl, hydroxy, alkoxy, perhaloalkoxy, aryloxy, mercapto, alkylthio, arylthio, perfluoroalkyl, cyano, carbonyl, carboxy, carboxyester, ether, amine, thiocarbonyl, O-thiocarbamyl, N-thiocarbamyl, C-amido, N-amido, S-sulfonamido, N-sulfonamido, C-carboxy, O-carboxy, isocyanato, thiocyanato, isothiocyanato and nitro.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. A heterocycle group may be optionally substituted with one or more groups or substituents replacing a hydrogen atom. Groups and substituents which may replace hydrogen atoms include but are not limited to halogen, perhaloalkyl, hydroxy, alkoxy, perhaloalkoxy, aryloxy, mercapto, alkylthio; arylthio, perfluoroalkyl, cyano, carbonyl, carboxy, carboxyester, ether, amine, thiocarbonyl, O-thiocarbamyl, N-thiocarbamyl, C-amido, N-amido, S-sulfonamido, N-sulfonamido, C-carboxy, O-carboxy, isocyanato, thiocyanato, isothiocyanato and nitro.

A cycloheteroalkyl group refers to a cycloalkyl group that includes at least one heteroatom selected from nitrogen, oxygen and sulfur. The radicals may be fused with an aryl or heteroaryl. Illustrative examples of cycloheteroalkyl groups include: 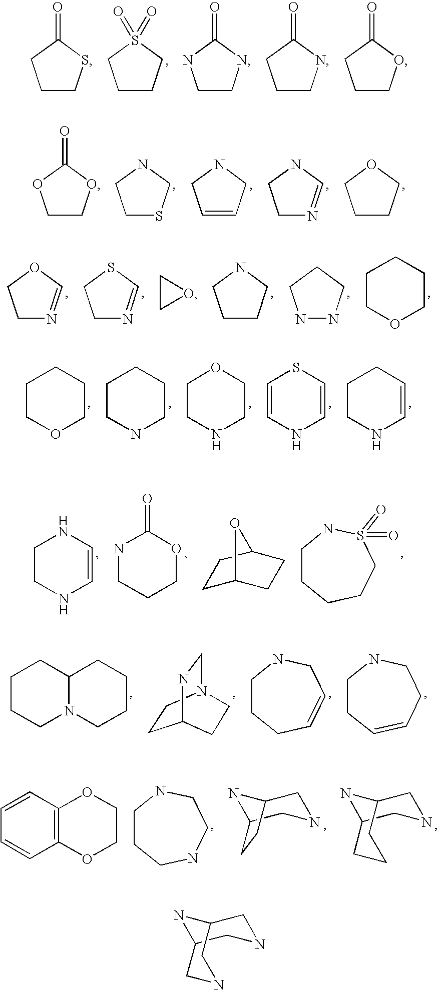
and the like. A cycloheteroalkyl group may be optionally substituted with one or more groups or substituents replacing a hydrogen atom. Groups and substituents which may replace hydrogen atoms include but are not limited to halogen, perhaloalkyl, hydroxy, alkoxy, perhaloalkoxy, aryloxy, mercapto, alkylthio, arylthio, perfluoroalkyl, cyano, carbonyl, carboxy, carboxyester, ether, amine, thiocarbonyl, O-thiocarbamyl, N-thiocarbamyl, C-amido, N-amido, S-sulfonamido, N-sulfonamido, C-carboxy, O-carboxy, isocyanato, thiocyanato, isothiocyanato and nitro.

The term “hydrocarbon chain” refers to a series of covalently linked carbon atoms. A hydrocarbon chain may saturated or unsaturated having sp³, sp², and sp hybridized carbons. A hydrocarbon chain may be part of linear or cyclic moiety. Hydrocarbon chains may be found within bicyclic ring structures.

The term “heteroatom-comprising hydrocarbon chain” refers to a hydrocarbon chain substituted atoms other than carbon within the chain.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

An “isocyanato” group refers to a —NCO group.

An “isothiocyanato” group refers to a —NCS group.

A “mercaptoalkyl” group refers to a R′SR— group, where R and R′ are as defined herein.

A “mercaptomercaptyl” group refers to a RSR′S— group, where R is as defined herein.

A “mercaptyl” group refers to a RS— group, where R is as defined herein.

The terms “nucleophile” and “electrophile” as used herein have their usual meanings familiar to synthetic and/or physical organic chemistry. Carbon electrophiles typically comprise one or more alkyl, alkenyl, alkynyl or aromatic (sp³, sp², or sp hybridized) carbon atoms substituted with any atom or group having a Pauling electronegativity greater than that of carbon itself. Examples of carbon electrophiles include but are not limited to carbonyls (aldehydes, ketones, esters, amides), oximes, hydrazones, epoxides, aziridines, alkyl-, alkenyl-, and aryl halides, acyls, sulfonates (aryl, alkyl and the like). Other examples of carbon electrophiles include unsaturated carbon atoms electronically conjugated with electron withdrawing groups, examples being the 6-carbon in a alpha-unsaturated ketones or carbon atoms in fluorine substituted aryl groups. Methods of generating carbon electrophiles, especially in ways which yield precisely controlled products, are known to those skilled in the art of organic synthesis. Other electrophiles which find broad uses herein include by way of example only include sulfonyl halides.

The term “moiety” refers to a specific segment or functional group of a molecule. Chemical moieties are often recognized chemical entities embedded in or appended to a molecule.

The term “null” refers to a lone electron pair.

The term “perhaloalkoxy” refers to an alkoxy group where all of the hydrogen atoms are replaced by halogen atoms.

The term “perhaloalkyl” refers to an alkyl group where all of the hydrogen atoms are replaced by halogen atoms. The term perfluoralkyl refers to a perhaloalkyl wherein said halogen is fluorine.

The substituent R or R′ appearing by itself and without a number designation refers to an optionally substituted substituent selected from the group consisting of alkyl, cycloalkyl, aryl, heteroalkyl, heteroaryl (bonded through a ring carbon) and cycloheteroalkyl (bonded through a ring carbon).

The term “single bond” refers to a chemical bond between two atoms, or two moieties when the atoms joined by the bond are considered to be part of larger substructure.

In the event that G₃ is designated to be “a bond”, the structure shown below (right side) is intended: the entity designated G₃ collapses to a single bond connecting G₂ and G₄:

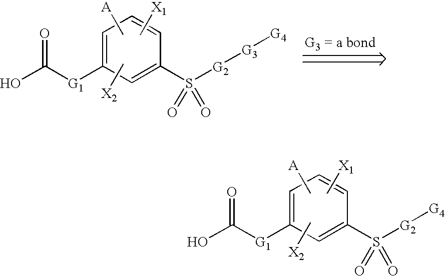

A “sulfinyl” group refers to a —S(═O)—R group, with R as defined herein.

A “sulfonyl” group refers to a —S(═O)₂—R group, with R as defined herein.

A “N-sulfonamido” group refers to a RS(═O)₂NH— group with R as defined herein.

A “S-sulfonamido” group refers to a —S(═O)₂NR₂, group, with R as defined herein.

An “N-thiocarbamyl” group refers to an ROC(═S)NH— group, with R as defined herein.

An “O-thiocarbamyl” group refers to a —OC(═S)—NR, group with R as defined herein.

A “thiocyanato” group refers to a —CNS group.

A “trihalomethanesulfonamido” group refers to a X₃CS(═O)₂NR— group with X is a halogen and R as defined herein.

A “trihalomethanesulfonyl” group refers to a X₃CS(═O)₂— group where X is a halogen.

A “trihalomethoxy” group refers to a X₃CO— group where X is a halogen.

Unless otherwise indicated, when a substituent is deemed to be “optionally substituted,” it is meant that the substituent is a group that may be substituted with one or more group(s) individually and independently selected from alkyl, cycloalkyl, aryl, heteroaryl, heteroalicyclic, heteroalkyl, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, perhaloalkyl, perfluoroalkyl, perhaloalkoxy, silyl, trihalomethanesulfonyl, and amino, including mono- and di-substituted amino groups, and the protected derivatives thereof. The protecting groups that may form the protective derivatives of the above substituents are known to those of skill in the art and may be found in references such as Greene and Wuts, above.

Many embodiments of the present invention are named using a conventional ring-numbering system. For example, a piperazine ring embedded within the structure of a preferred molecular embodiment of the invention uses the following atom numbering scheme:

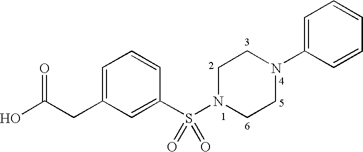

Many embodiments of the present invention possess one or more chiral centers and each center may exist in the R or S configuration, giving rise to numerous enantiomeric and diastereomeric forms of the same molecular formula. The present invention includes all diastereomeric, enantiomeric, and epimeric forms as well as the appropriate mixtures thereof. By way of illustration only, a G₂ moiety may comprise any of the following configurations: 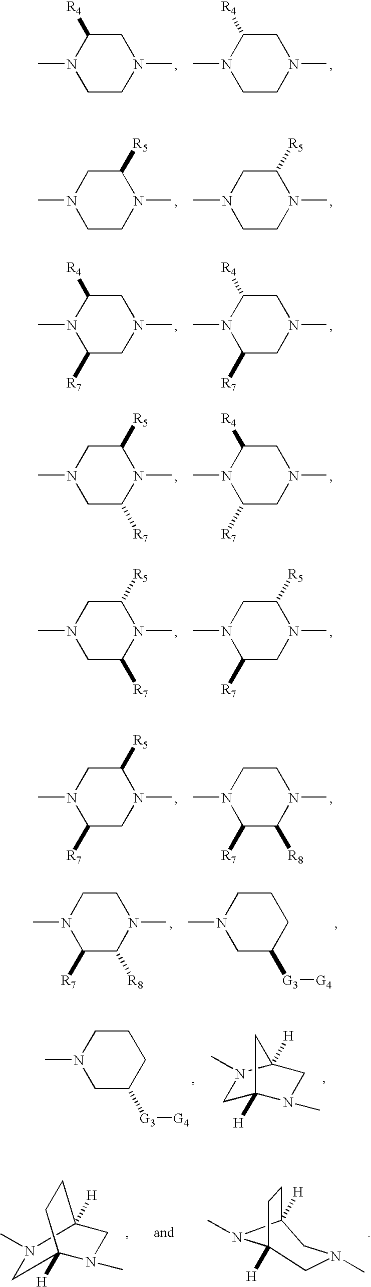
wherein substituents R₄, R₅, R₇, R₈ are defined herein.

Stereoisomers may be obtained, if desired, by methods known in the art as, for example, the separation of stereoisomers by chiral chromatographic columns. Additionally, the compounds of the present invention may exist as geometric isomers. The present invention includes all cis, trans, syn, anti, entgegen (E), and zusammen (Z) isomers as well as the appropriate mixtures thereof.

In some situations, compounds may exist as tautomers. All tautomers are included within Formula I and are provided by this invention.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

### METHODS OF MODULATING PROTEIN FUNCTION

In another aspect, the present invention relates to a method of modulating at least one peroxisome proliferator-activated receptor (PPAR) function comprising the step of contacting the PPAR with a compound of Formula I, as described herein. The change in cell phenotype, cell proliferation, activity of the PPAR, or binding of the PPAR with a natural binding partner may be monitored. Such methods may be modes of treatment of disease, biological assays, cellular assays, biochemical assays, or the like. In certain embodiments, the PPAR may be selected from the group consisting of PPARα, PPARδ, and PPARγ.

The term “activate” refers to increasing the cellular function of a PPAR. The term “inhibit” refers to decreasing the cellular function of a PPAR. The PPAR function may be the interaction with a natural binding partner or catalytic activity.

The term “cell phenotype” refers to the outward appearance of a cell or tissue or the function of the cell or tissue. Examples of cell or tissue phenotype are cell size (reduction or enlargement), cell proliferation (increased or decreased numbers of cells), cell differentiation, (a change or absence of a change in cell shape), cell survival, apoptosis (cell death), or the utilization of a metabolic nutrient (e.g., glucose uptake). Changes or the absence of changes in cell phenotype are readily measured by techniques known in the art.

The term “cell proliferation” refers to the rate at which a group of cells divides. The number of cells growing in a vessel can be quantified by a person skilled in the art when that person visually counts the number of cells in a defined area using a common light microscope. Alternatively, cell proliferation rates can be quantified by laboratory apparatus that optically measure the density of cells in an appropriate medium.

The term “contacting” as used herein refers to bringing a compound of this invention and a target PPAR together in such a manner that the compound can affect the activity of the PPAR, either directly; i.e., by interacting with the PPAR itself, or indirectly; i.e., by interacting with another molecule on which the activity of the PPAR is dependent. Such “contacting” can be accomplished in a test tube, a petri dish, a test organism (e.g., murine, hamster or primate), or the like. In a test tube, contacting may involve only a compound and a PPAR of interest or it may involve whole cells. Cells may also be maintained or grown in cell culture dishes and contacted with a compound in that environment. In this context, the ability of a particular compound to affect a PPAR related disorder; i.e., the EC₅₀ of the compound can be determined before use of the compounds in vivo with more complex living organisms is attempted. For cells outside the organism, multiple methods exist, and are well-known to those skilled in the art, to get the PPARs in contact with the compounds including, but not limited to, direct cell microinjection and numerous transmembrane carrier techniques.

The term “modulate” refers to the ability of a compound of the invention to alter the function of a PPAR. A modulator may activate the activity of a PPAR. The term “modulate” also refers to altering the function of a PPAR by increasing or decreasing the probability that a complex forms between a PPAR and a natural binding partner. A modulator may increase the probability that such a complex forms between the PPAR and the natural binding partner, may increase or decrease the probability that a complex forms between the PPAR and the natural binding partner depending on the concentration of the compound exposed to the PPAR, and or may decrease the probability that a complex forms between the PPAR and the natural binding partner.

The term “monitoring” refers to observing the effect of adding the compound of the invention to the cells of the method. The effect can be manifested in a change in cell phenotype, cell proliferation, PPAR activity, or in the interaction between a PPAR and a natural binding partner. Of course, the term “monitoring” includes detecting whether a change has in fact occurred or not.

### BIOLOGICAL ASSAYS

### TRANSFECTION ASSAYS

Compounds may be screened for functional potency in transient transfection assays in CV-1 cells or other cell types for their ability to activate the PPAR subtypes (transactivation assay). A previously established chimeric receptor system was utilized to allow comparison of the relative transcriptional activity of the receptor subtypes on the same synthetic response element and to prevent endogenous receptor activation from complicating the interpretation of results. See, for example; Lehmann, J. M.; Moore, L. B.; Smith-Oliver, T. A; Wilkinson, W. O.; Willson, T. M.; Kliewer, S. A., An antidiabetic thiazolidinedione is a high affinity ligand for peroxisome proliferatur-activated receptor γ (PPARγ), J. Biol. Chem., 1995, 270, 12953-6. The ligand binding domains for murine and human PPAR-alpha, PPAR-gamma, and PPAR-delta are each fused to the yeast transcription factor GAL4 DNA binding domain. CV-1 cells were transiently transfected with expression vectors for the respective PPAR chimera along with a reporter construct containing four or five copies of the GAL4 DNA binding site driving expression of luciferase. After 8-16 h, the cells are replated into multi-well assay plates and the media is exchanged to phenol-red free DME medium supplemented with 5% delipidated calf serum. 4 hours after replating, cells were treated with either compounds or 1% DMSO for 20-24 hours. Luciferase activity was then assayed with Britelite (Perkin Elmer) following the manufacturer's protocol and measured with either the Perkin Elmer Viewlux or Molecular Devices Acquest Xsee, for example, Kliewer, S. A., et. al. Cell 1995, 83, 813-819). Rosiglitazone is used as a positive control in the hPPAR-γ assay. Wy-14643 and GW7647 is used as a positive control in the hPPAR-α assay. GW501516 is used as the positive control in the hPPAR-δ assay.

### TARGET DISEASES TO BE TREATED

In another aspect, the present invention relates to a method of treating a disease comprising identifying a patient in need thereof, and administering a therapeutically effective amount of a compound of Formula I, as described herein, to the patient.

The third subtype of PPARs, PPARδ (PPARβ, NUCl), is broadly expressed in the body and has been shown to be a valuable molecular target for treatment of dyslipedimia and other diseases. For example, in a recent study in insulin-resistant obese rhesus monkeys, a potent and selective PPARδ compound was shown to decrease VLDL and increase HDL in a dose response manner (Oliver et al., Proc. Natl. Acad. Sci. U.S.A. 98: 5305, 2001).

The compounds of the invention are useful in the treatment of a disease or condition ameliorated by the modulation, activation, or inhibition of an hPPAR-delta. Specific diseases and conditions modulated by PPAR-delta and for which the compounds and compositions are useful include but are not limited to dyslipidemia, syndrome X, heart failure, hypercholesteremia, cardiovascular disease, type II diabetes mellitus, type 1 diabetes, insulin resistance hyperlipidemia, obesity, anorexia bulimia, inflammation, anorexia nervosa, and modulation of wound healing.

The compounds of the invention may also be used (a) for raising HDL in a subject; (b) for treating Type 2 diabetes, decreasing insulin resistance or lowering blood pressure in a subject; (c) for decreasing LDLc in a subject; (d) for shifting LDL particle size from small dense to normal LDL in a subject; (e) for treating atherosclerotic diseases including vascular disease, coronary heart disease, cerebrovascular disease and peripheral vessel disease in a subject; and (f) for treating inflammatory diseases, including rheumatoid arthritis, asthma, osteoarthritis and autoimmune disease in a subject.

The compounds of the invention may also be used for treating, ameliorating, or preventing a disease or condition selected from the group consisting of obesity, diabetes, hyperinsulinemia, metabolic syndrome X, polycystic ovary syndrome, climacteric, disorders associated with oxidative stress, inflammatory response to tissue injury, pathogenesis of emphysema, ischemia-associated organ injury, doxorubicin-induced cardiac injury, drug-induced hepatotoxicity, atherosclerosis, and hypertoxic lung injury.

### TREATMENT METHODS, DOSAGES AND COMBINATION THERAPIES

The term “patient” means all mammals including humans. Examples of patients include humans, cows, dogs, cats, goats, sheep, pigs, and rabbits.

The term “therapeutically effective amount” as used herein refers to that amount of the compound being administered which will relieve to some extent one or more of the symptoms of the disease, condition or disorder being treated. In reference to the treatment of diabetes or dyslipidemia a therapeutically effective amount refers to that amount which has the effect of (1) reducing the blood glucose levels; (2) normalizing lipids, e.g. triglycerides, low-density lipoprotein; (3) relieving to some extent (or, preferably, eliminating) one or more symptoms associated with the disease, condition or disorder to be treated; and/or (4) raising HDL.

The compositions containing the compound(s) described herein can be administered for prophylactic and/or therapeutic treatments. In therapeutic applications, the compositions are administered to a patient already suffering from a disease, condition or disorder mediated, modulated or involving the PPARs, including but not limited to metabolic diseases, conditions, or disorders, as described above, in an amount sufficient to cure or at least partially arrest the symptoms of the disease, disorder or condition. Amounts effective for this use will depend on the severity and course of the disease, disorder or condition, previous therapy, the patient's health status and response to the drugs, and the judgment of the treating physician. It is considered well within the skill of the art for one to determine such therapeutically effective amounts by routine experimentation (e.g., a dose escalation clinical trial).

In prophylactic applications, compositions containing the compounds described herein are administered to a patient susceptible to or otherwise at risk of a particular disease, disorder or condition mediated, modulated or involving the PPARs, including but not limited to metabolic diseases, conditions, or disorders, as described above. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial).

The terms “enhance” or “enhancing” means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system. When used in a patient, amounts effective for this use will depend on the severity and course of the disease, disorder or condition (including, but not limited to, metabolic disorders), previous therapy, the patient's health status and response to the drugs, and the judgment of the treating physician. It is considered well within the skill of the art for one to determine such enhancing-effective amounts by routine experimentation.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. When the symptoms have been alleviated to the desired level, treatment can cease. Patients can, however, require intermittent treatment on a long-term basis upon any recurrence of symptoms.

The amount of a given agent that will correspond to such an amount will vary depending upon factors such as the particular compound, disease condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, preferably 1-1500 mg per day. The desired dose may conveniently be presented in a single dose or as divided doses administered at appropriate intervals, for example as two, three, four or more sub-doses per day.

In certain instances, it may be appropriate to administer at least one of the compounds described herein (or a pharmaceutically acceptable salt, ester, amide, prodrug, or solvate) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compound of formula (I) with: (a) statin and/or other lipid lowering drugs for example MTP inhibitors and LDLR upregulators; (b) antidiabetic agents, e.g. metformin, sulfonylureas, or PPAR-gamma, PPAR-alpha and PPAR-alpha/gamma modulators (for example thiazolidinediones such as e.g. Pioglitazone and Rosiglitazone); and (c) antihypertensive agents such as angiotensin antagonists, e.g., telmisartan, calcium channel antagonists, e.g. lacidipine and ACE inhibitors, e.g., enalapril.

In any case, the multiple therapeutic agents (one of which is one of the compounds described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks.

### ROUTES OF ADMINISTRATION

Suitable routes of administration may, for example, include oral, rectal, transmucosal, pulmonary, ophthalmic or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into an organ, often in a depot or sustained release formulation or topically in the form of a cream or transdermal patch. Furthermore, one may administer the drug in a targeted drug delivery system, for example, in a liposome coated with organ-specific antibody. The liposomes will be targeted to and taken up selectively by the organ.

### COMPOSITION/FORMULATION

The pharmaceutical compositions of the present invention may be manufactured in a manner that is itself known, e.g., by means of conventional mixing, dissolving, granulating, dragee-making, levigating, emulsifying, encapsulating, entrapping or compression processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art, e.g., in Remington's Pharmaceutical Sciences, above.

For intravenous injections, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art. For other parenteral injections, the agents of the invention may be formulated in aqueous or nonaqueous solutions, preferably with physiologically compatible buffers or excipients. Such excipients are generally known in the art.

For oral administration, the compounds can be formulated readily by combining the active compounds with pharmaceutically acceptable carriers or excipients well known in the art. Such carriers enable the compounds of the invention to be formulated as tablets, powders, pills, dragees, capsules, liquids, gels, syrups, elixirs, slurries, suspensions and the like, for oral ingestion by a patient to be treated. Pharmaceutical preparations for oral use can be obtained by mixing one or more solid excipient with one or more compound of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets, lozenges, or gels formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system may be a 10% ethanol, 10% polyethylene glycol 300, 10% polyethylene glycol 40 castor oil (PEG-40 castor oil) with 70% aqueous solution. This cosolvent system dissolves hydrophobic compounds well, and itself produces low toxicity upon systemic administration. Naturally, the proportions of a cosolvent system may be varied considerably without destroying its solubility and toxicity characteristics. Furthermore, the identity of the cosolvent components may be varied: for example, other low-toxicity nonpolar surfactants may be used instead of PEG-40 castor oil, the fraction size of polyethylene glycol 300 may be varied; other biocompatible polymers may replace polyethylene glycol, e.g., polyvinyl pyrrolidone; and other sugars or polysaccharides maybe included in the aqueous solution.

Alternatively, other delivery systems for hydrophobic pharmaceutical compounds may be employed. Liposomes and emulsions are well known examples of delivery vehicles or carriers for hydrophobic drugs. Certain organic solvents such as N-methylpyrrolidone also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

### SYNTHESIS OF THE COMPOUNDS OF THE INVENTION

Compounds of the present invention may be synthesized using standard synthetic techniques known to those of skill in the art or using methods known in the art in combination with methods described herein. As a guide the following synthetic methods may be utilized.

### FORMATION OF COVALENT LINKAGES BY REACTION OF AN ELECTROPHILE WITH A NUCLEOPHILE

Selected examples of covalent linkages and precursor functional groups which yield them are given in the Table entitled “Examples of Covalent Linkages and Precursors Thereof.” Precursor functional groups are shown as electrophilic groups and nucleophilic groups. The functional group on the organic substance may be attached directly, or attached via any useful spacer or linker as defined below.

TABLE 2
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Suitable carbon nucleophiles include, but are not limited to alkyl, alkenyl, aryl and alkynyl Grignard, organolithium, organozinc, alkyl-, alkenyl , aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other carbon nucleophiles include phosphorus ylids, enol and enolate reagents; these carbon nucleophiles have the advantage of being relatively easy to generate from precursors well known to those skilled in the art of synthetic organic chemistry. Carbon nucleophiles, when used in conjunction with carbon electrophiles, engender new carbon-carbon bonds between the carbon nucleophile and carbon electrophile.

Non-carbon nucleophiles suitable for coupling to carbon electrophiles include but are not limited to primary and secondary amines, thiols, thiolates, and thioethers, alcohols, alkoxides, azides, semicarbazides, and the like. These non-carbon nucleophiles, when used in conjunction with carbon electrophiles, typically generate heteroatom linkages (C—X—C), wherein X is a hetereoatom, e. g, oxygen or nitrogen.

### USE OF PROTECTING GROUPS

The term “protecting group” refers to chemical moieties that block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester derivatives as exemplified herein, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd⁰-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

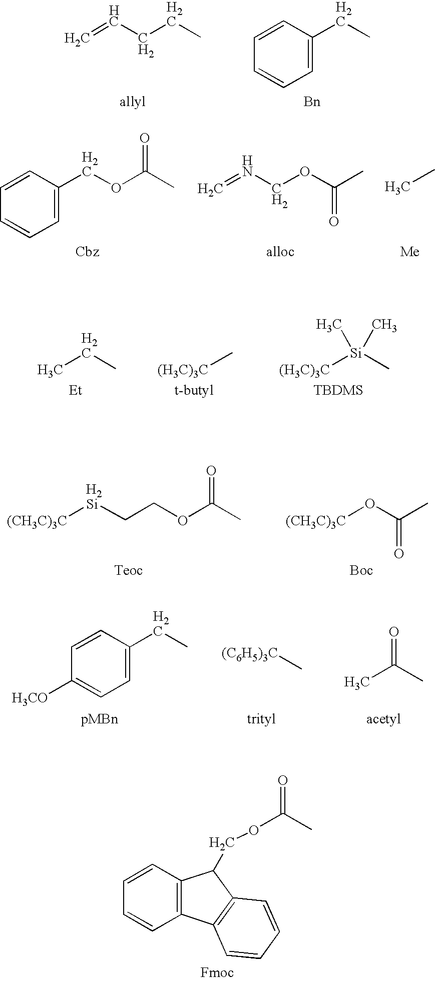

Other protecting groups are described in Greene and Wuts, Protective Groups in Organic Synthesis, 3^{rd} Ed., John Wiley & Sons, New York, N.Y., 1999, which is incorporated herein by reference in its entirety.

### GENERAL SYNTHETIC METHODS FOR PREPARING COMPOUNDS

Molecular embodiments of the present invention can be synthesized using standard synthetic techniques known to those of skill in the art. Compounds of the present invention can be synthesized using the general synthetic procedures set forth in Schemes I-XXII. Specific synthetic procedures are set forth in subsequent schemes.

### PREPARATION OF EXAMPLES 1-233

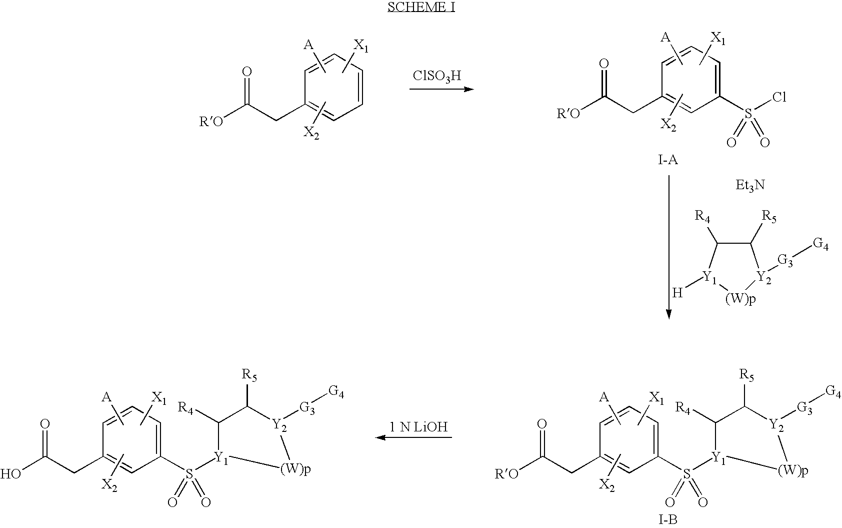

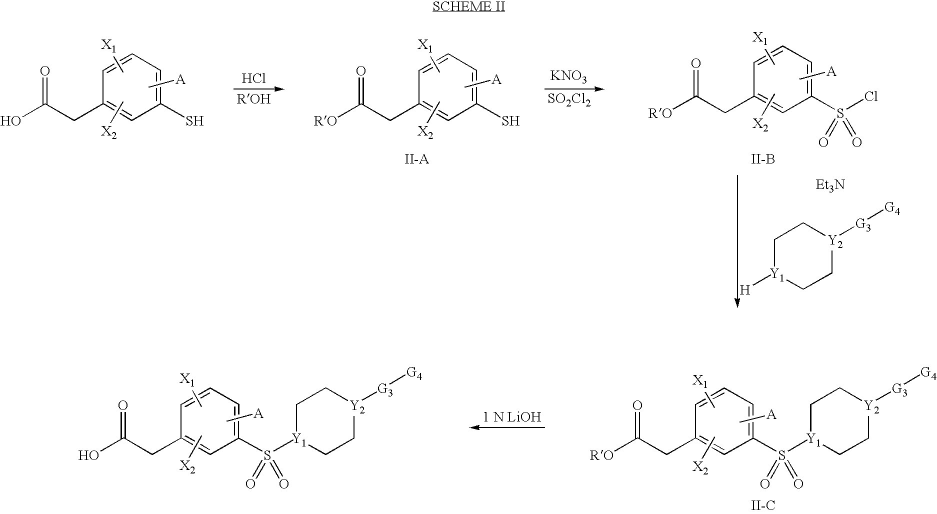

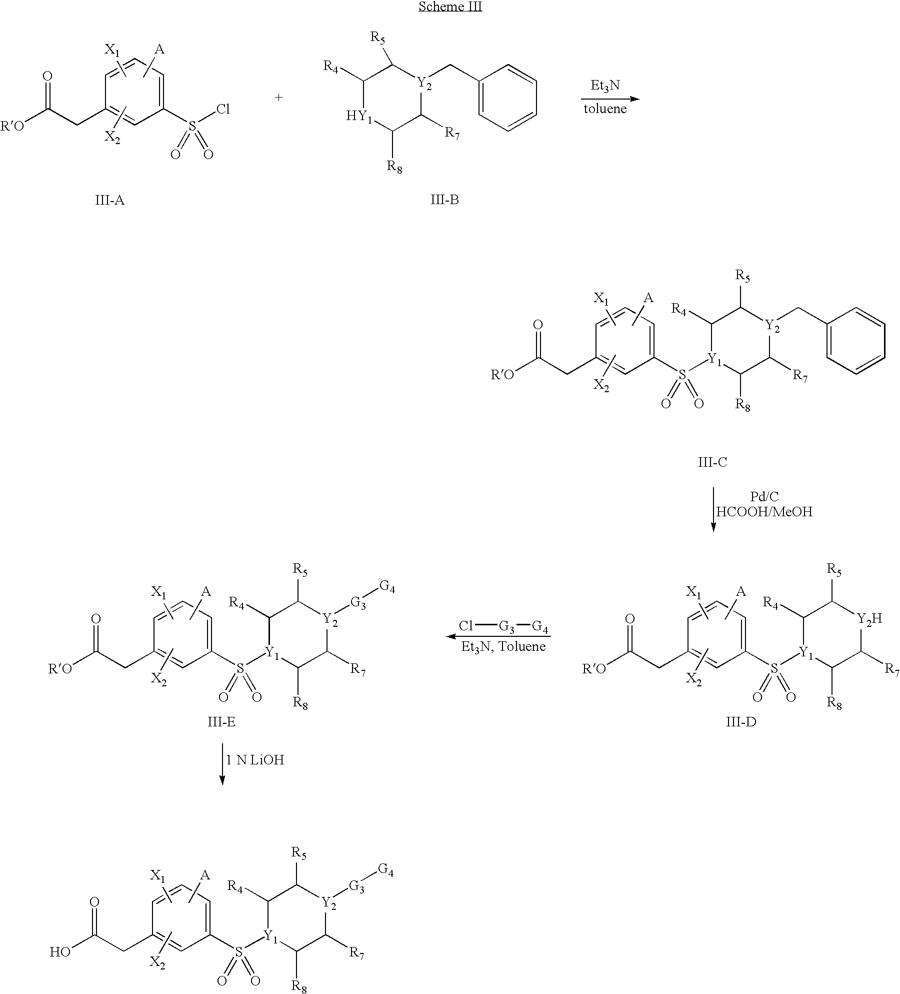

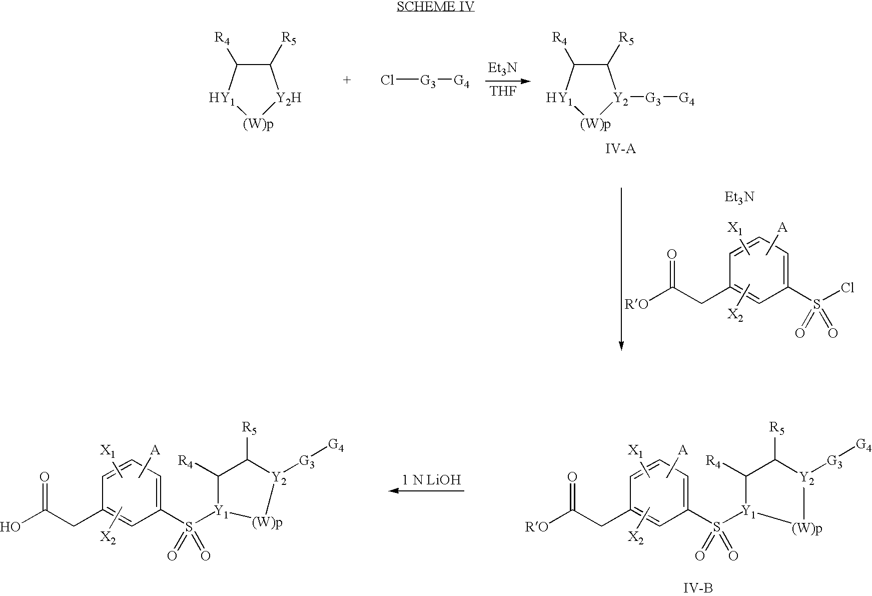

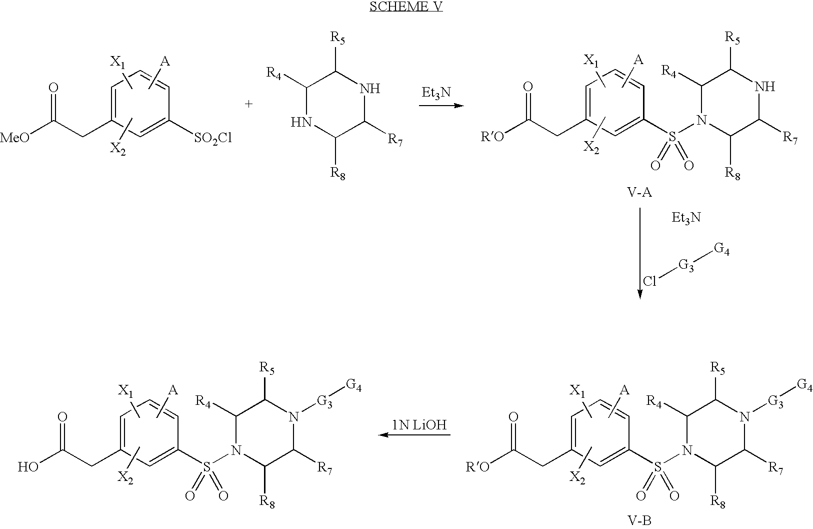

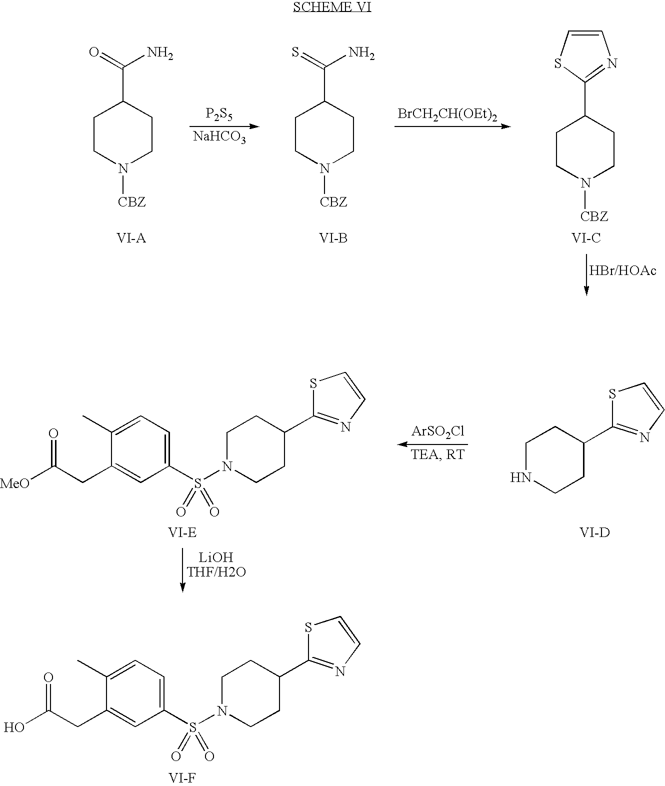

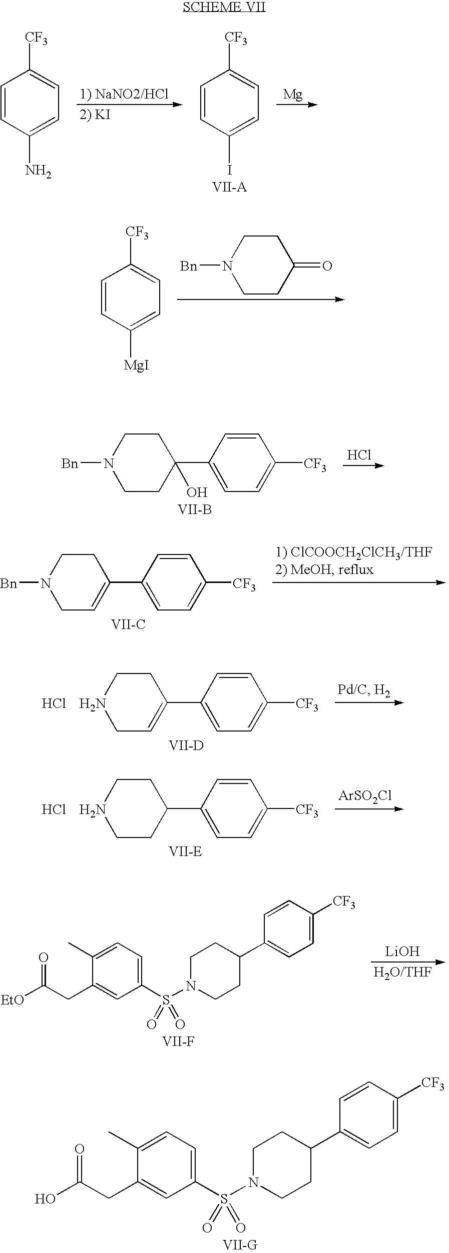

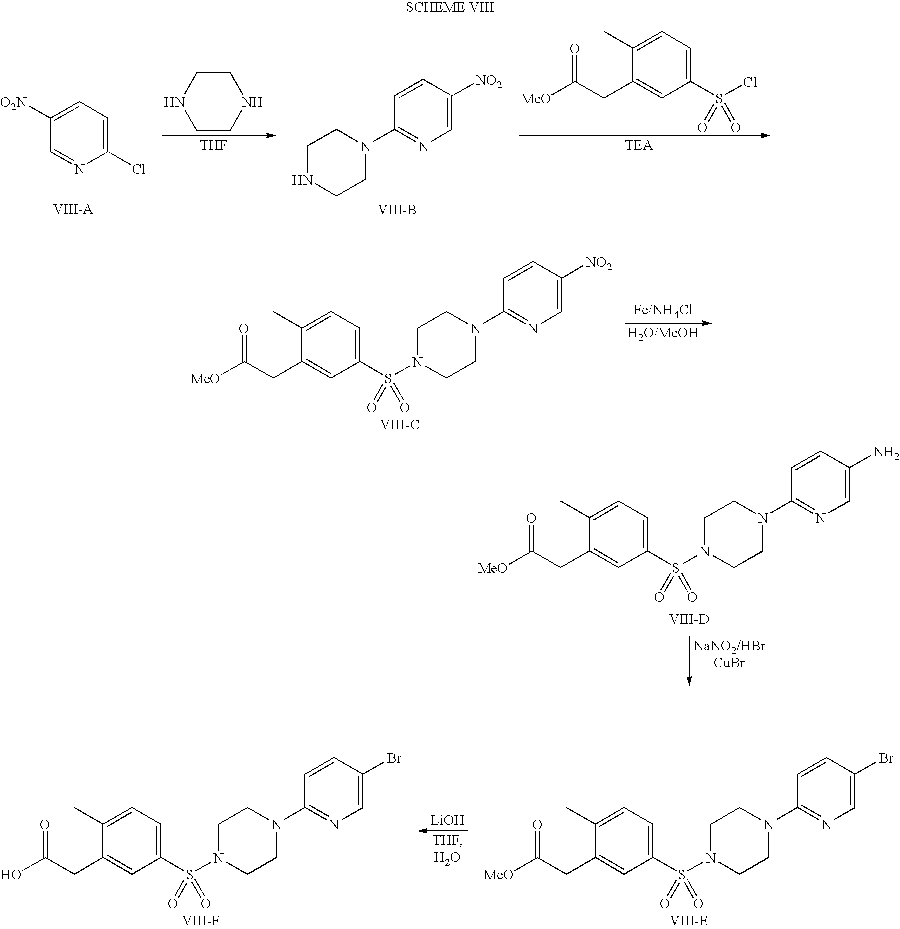

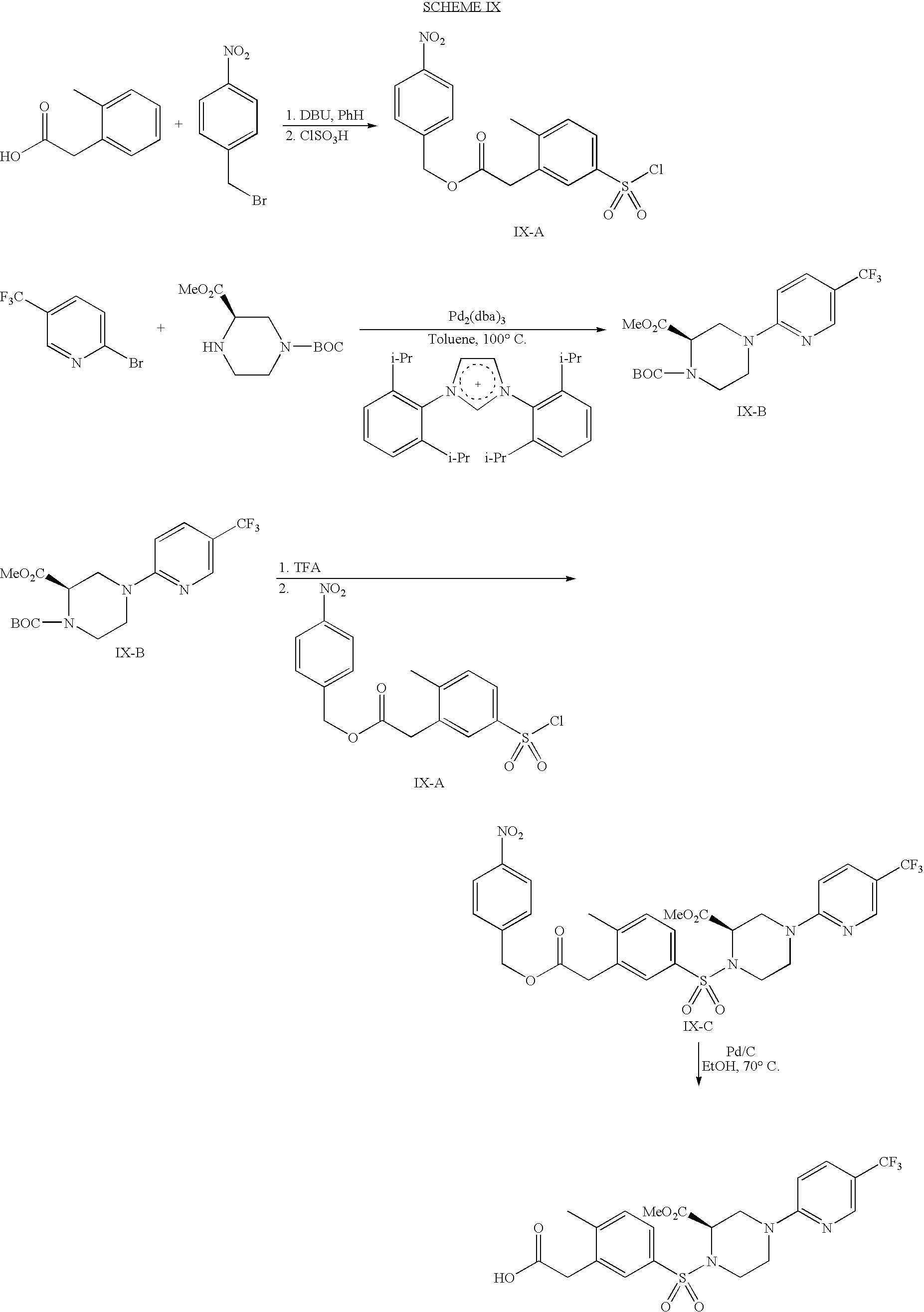

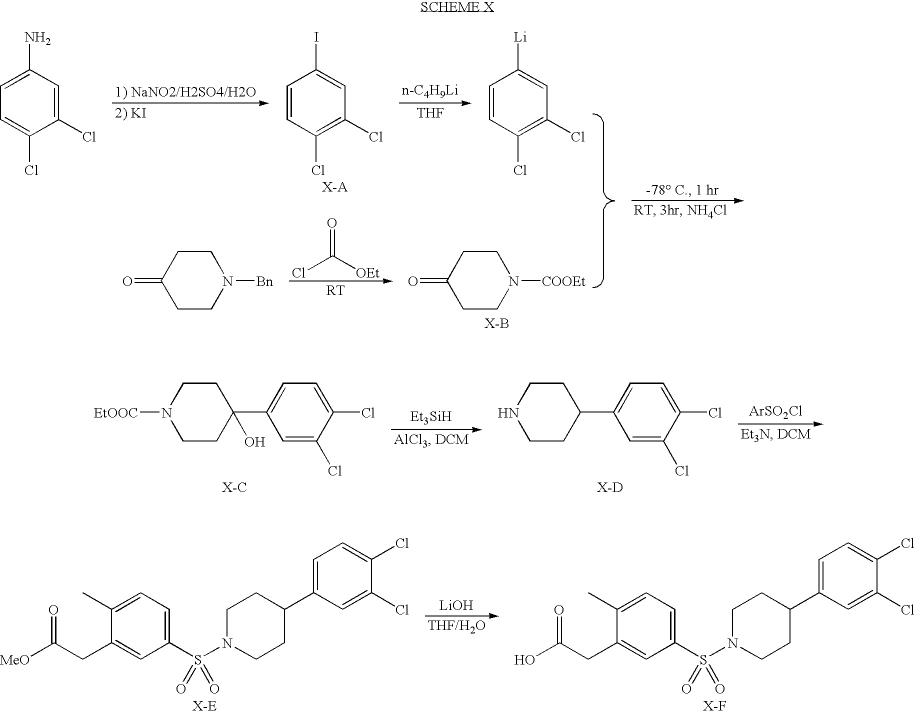

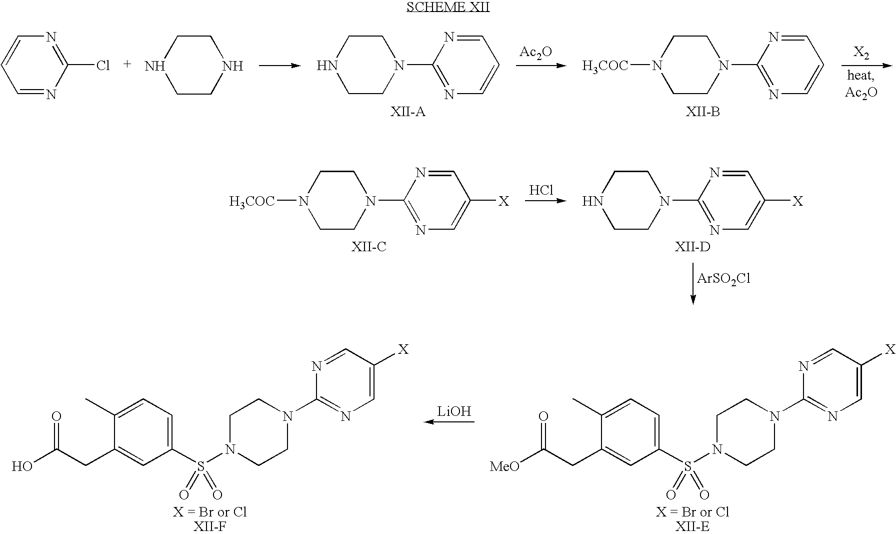

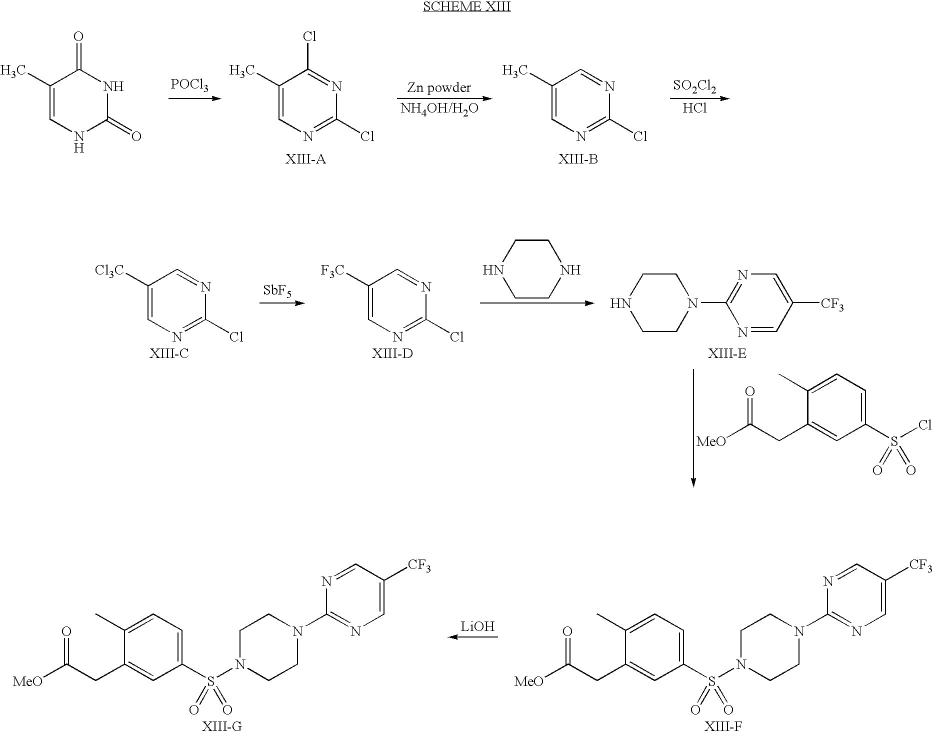

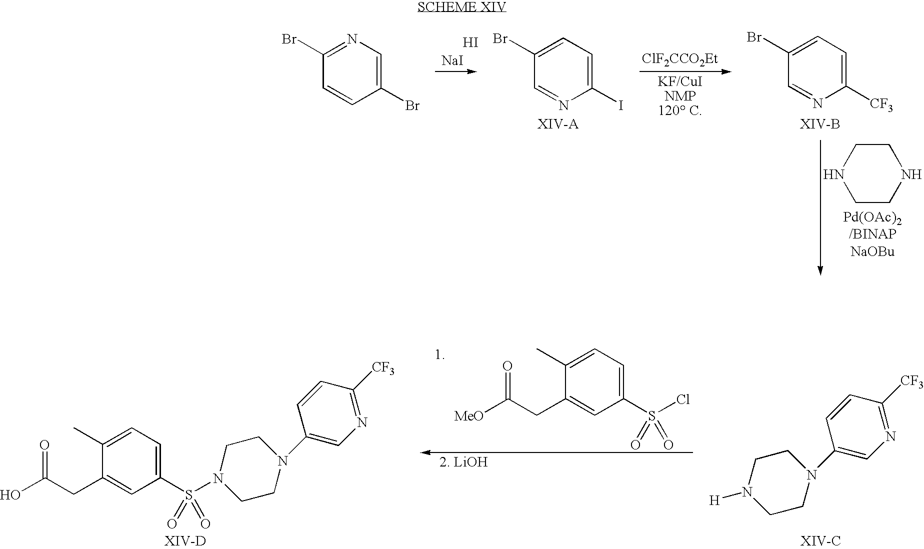

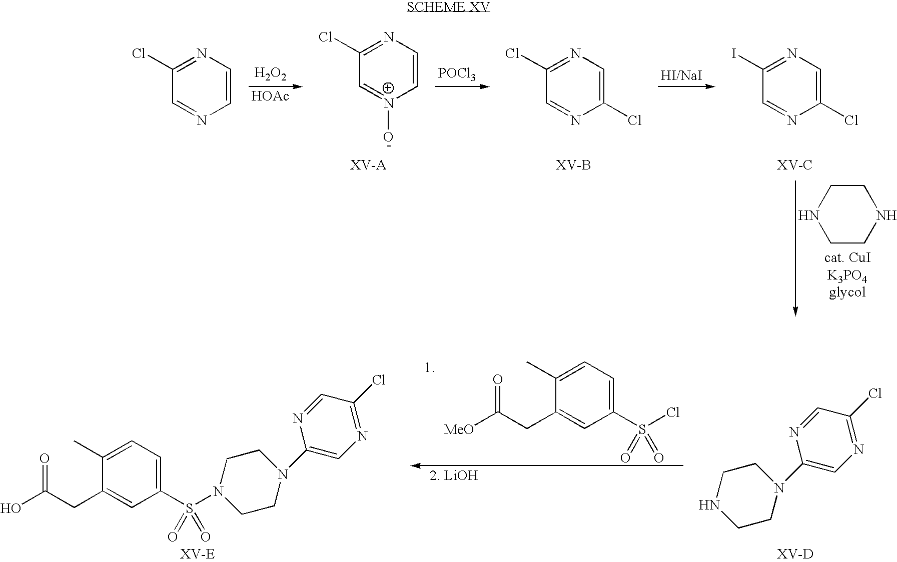

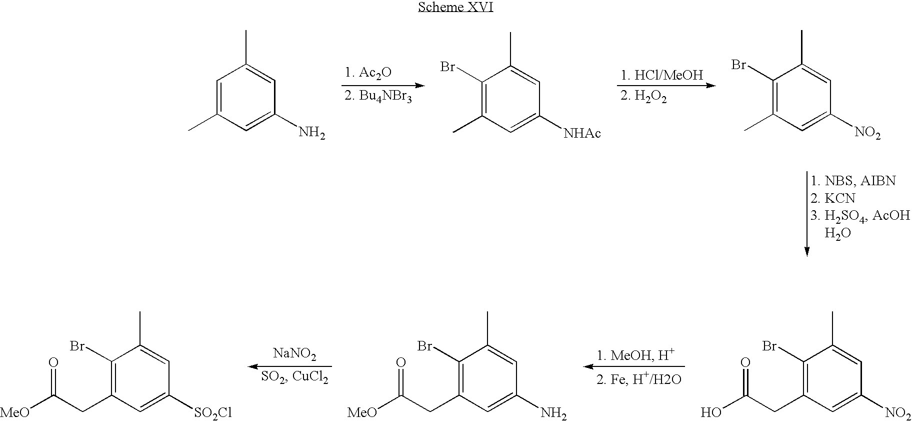

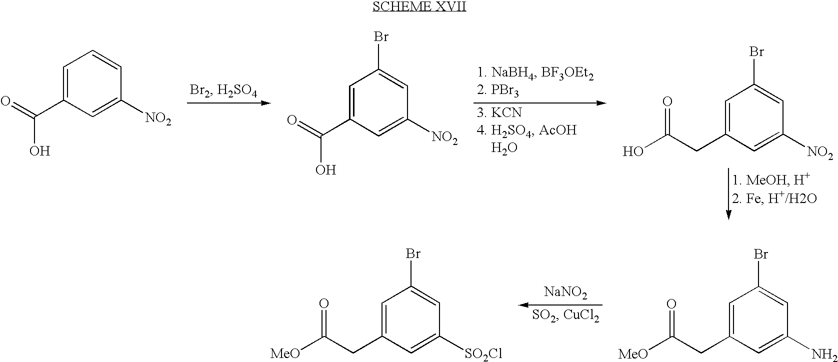

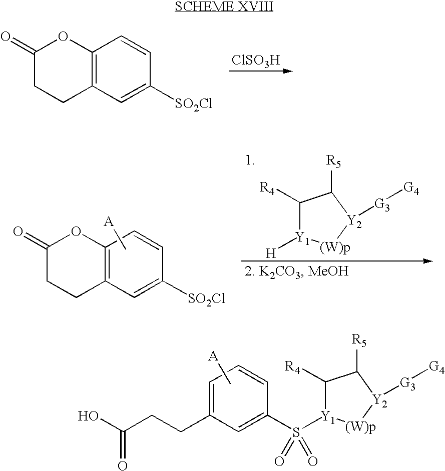

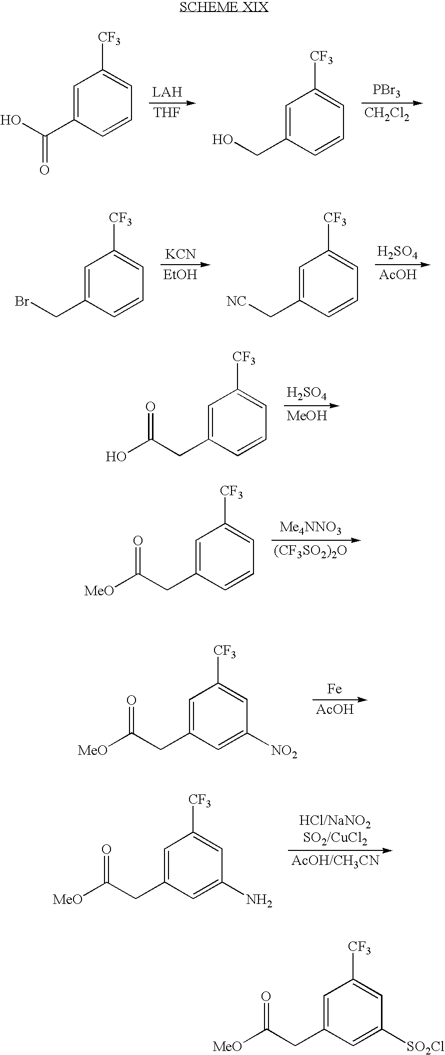

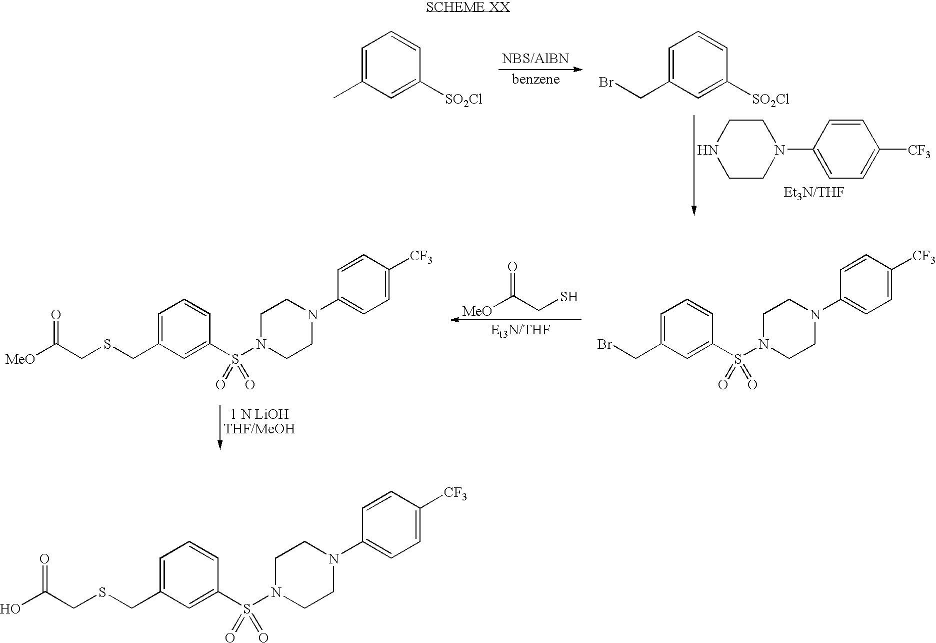

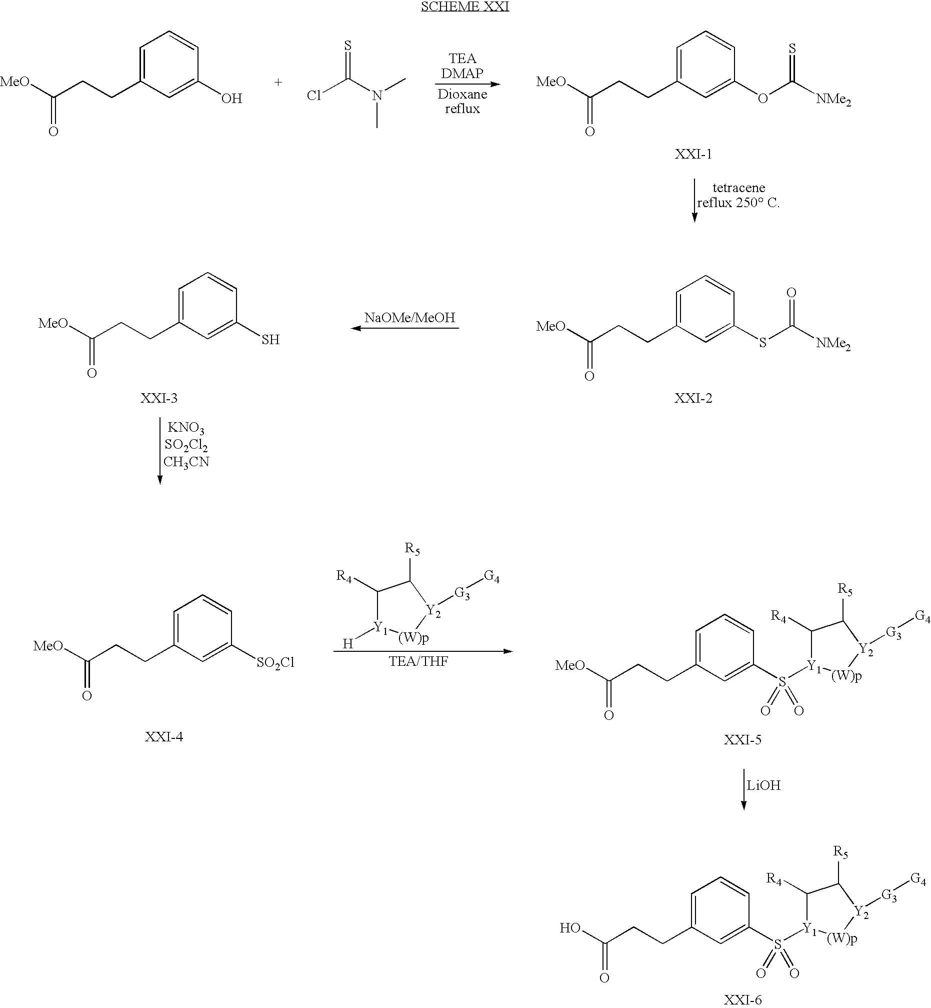

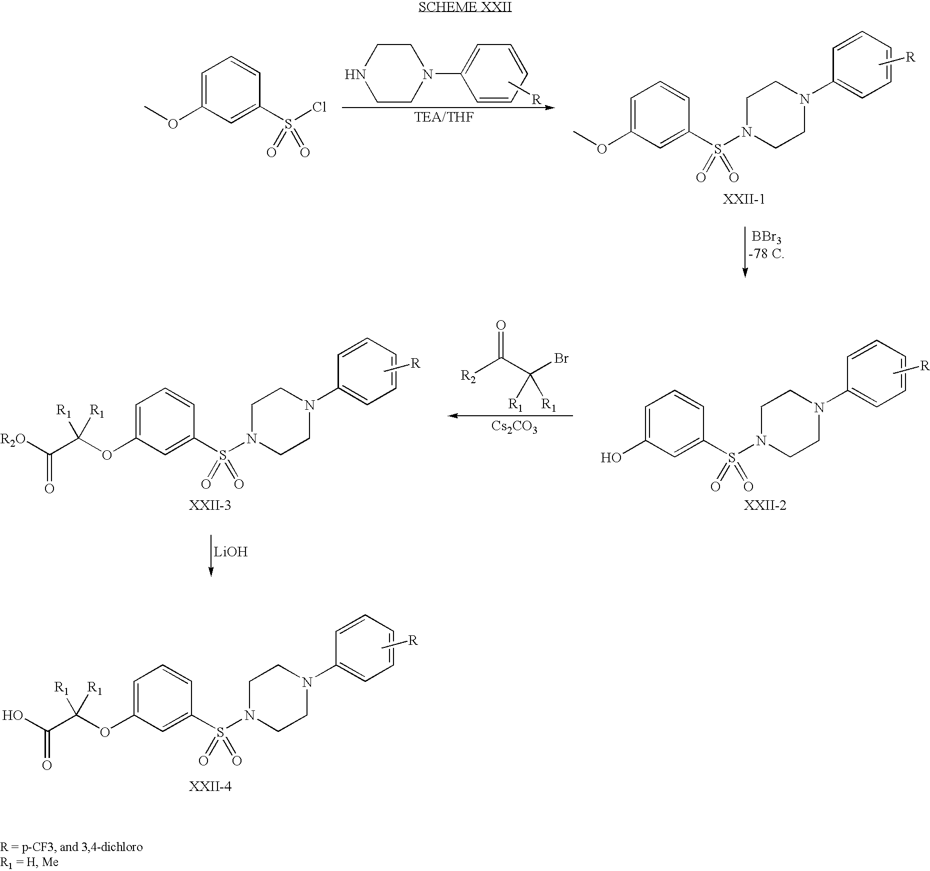

Numerous additional molecular embodiments of the invention may be envisioned. The following examples resemble the molecular embodiments already described, but feature several additional characteristics. Among them, by way of example only, are a quaternary or sp² hybridized carbon at position Y₂: 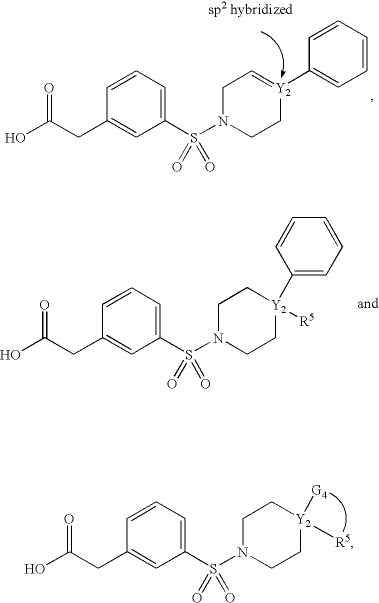
in which the connection between G₄ and R⁵ can occur between any atom present on R⁵ and any atom present on G₄.
The molecular embodiments of the present invention which incorporate quaternary and sp²-hybridized carbons may be synthesized using methods cited in J. Med Chem. 999, 42, 4778 and references cited therein.

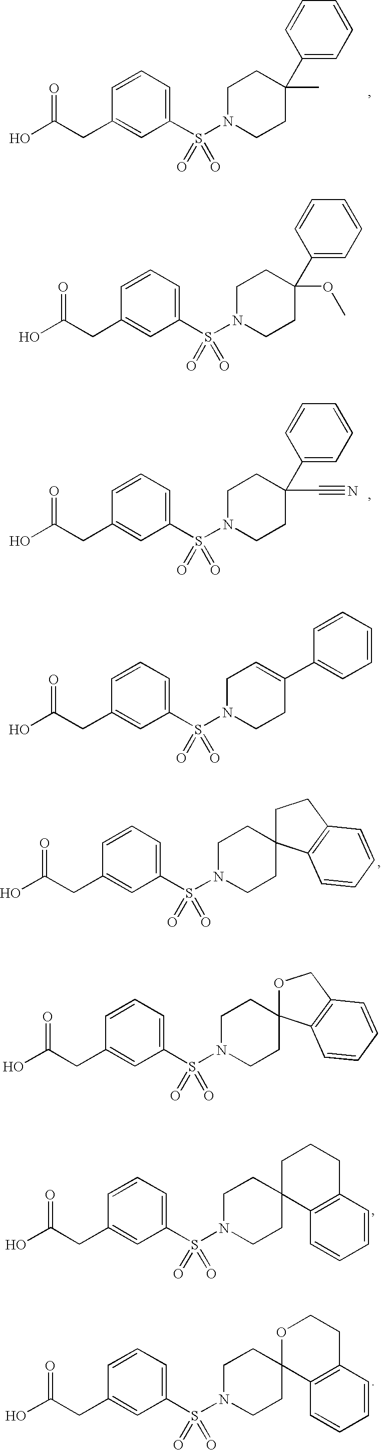

### ARYL SULFONE COMPOUNDS

Additional molecular embodiments of the invention feature a carbon-to-sulfur bond linking G₂ to an aryl sulfonyl moiety. The following synthetic schemes may be employed to synthesize a wide range of such sulfone compounds

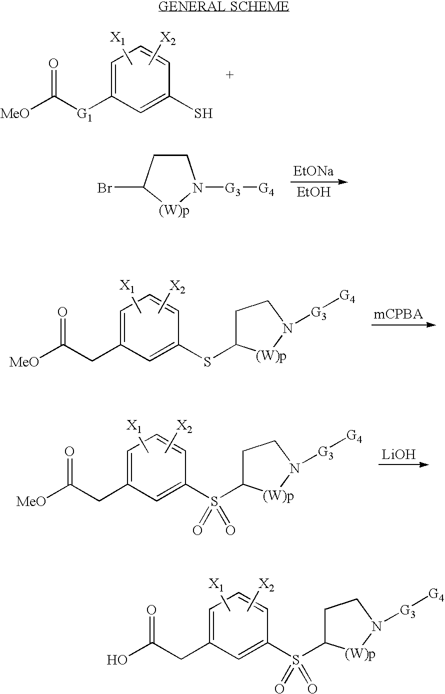

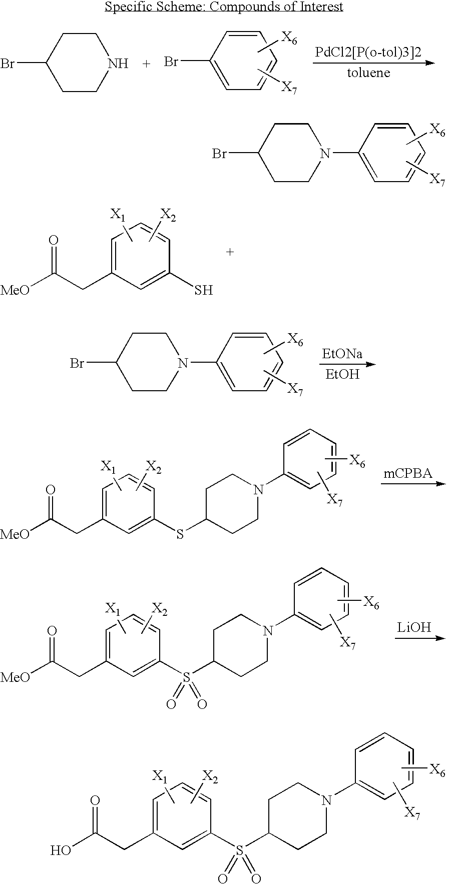

### SYNTHESES OF MOLECULAR EMBODIMENTS

### EXAMPLE 1

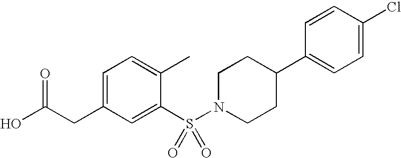

### {3-[4-(4-Chloro-phenyl-piperidine-1-sulfonyl]-4-methyl-phenyl)-acetic acid

Step 1

(3-Chlorosulfonyl-4-methyl-phenyl)-acetic acid ethyl ester I-A-1. Ethyl p-tolylacetate (25.0 g, 0.14 mmol) was slowly added to chilled chlorosulfonic acid (30 mL) at 0° C. After completion of the addition, the mixture was removed from the ice bath and continually stirred overnight. The reaction solution was added dropwise into 250 mL of ice and extracted with chloroform (2×100 mL). The combined organic extracts were washed with brine, and dried over Na₂SO₄. After removal of solvent, the crude product was purified by chromatography to afford 19 g of intermediate. ¹H NMR (400 MHz, CDCl₃) δ ppm: 7.95 (s, 1H), 7.54 (d, 1H), 7.37 (d, 1H), 4.17 (q, 2H), 3.69 (s, 2H), 2.76 (s, 3H), 1.25 (t, 3H).

Step 2

(3-[4-(4-Chlorophenyl)-piperidine-1-sulfonyl]-4-methyl-phenyl}-acetic acid ethyl ester 1-B-1. To a solution of intermediate I-A-1 (260 mg, 0.93 mmol, 1.0 equiv.) in THF (2 mL), was added 4-(4-chlorophenyl)-piperidine (181 mg, 0.93 mmol, 1.0 equiv.), followed by Et₃N (1.86 mmol, 2.0 equiv.). The reaction mixture was stirred at room temperature overnight. The solvent was evaporated and the residue, identified as intermediate I-B-1 was purified by chromatography. ¹H NMR (400 MHz, CDCl₃) δ ppm: 7.81 (s, 1H), 7.40 (d, 1H), 7.25 (m, 3H), 7.1 1 (d, 2H), 4.16 (q, 2H), 3.83 (d, 2H), 3.65 (s, 2H), 2.72 (t, 2H), 2.63 (s, 3H), 2.55 (m, 1H), 1.83 (d, 2H), 1.74 (m, 2H), 1.25 (t, 2H).

Step 3

{3-[4-(4-Chloro-phenyl-piperidine-1-sulfonyl]-4-methyl-phenyl}-acetic acid. Ethyl ester I-B-1 (1.0 equiv.) was dissolved in 3 mL of THF/MeOH (3:1), followed by addition of IN LiOH (5.0 equiv.). The resulting mixture was stirred at 40° C. for 2 hours. The organic solvent was evaporated under N₂. To the residue was added 1N HCl (5.0 equiv.) and then extracted with EtOAc (5 mL). The organic layers were washed with water, brine, and dried over Na₂SO₄. Solvent was evaporated to afford the compound of Example 1. ¹H NMR (400 MHz, CDCl₃) δ ppm. 7.81 (s, 1H), 7.40 (d, 1H), 7.29 (d, 1H), 7.22 (d, 2H), 7.08 (d, 2H), 3.84 (d, 2H), 3.69 (s, 2H), 2.72 (t, 2H), 2.62 (s, 3H), 2.53 (t, 1H), 1.82 (d, 2H), 1.70 (m, 2H).

### EXAMPLE 2

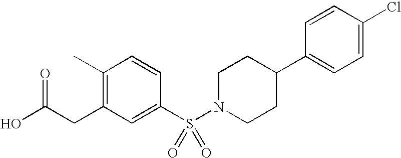

### {5-[4-(4-Chlorophenyl)-piperidine-1-sulfonyl]-2-methyl-phenyl}-acetic acid

The compound of Example 2 was prepared according to the method described for preparing Example 1. ¹H NMR (400 MHz, CD₃OH-d₃) δ ppm: 7.64 (s, 1H), 7.60 (d, 1H), 7.43 (d, 1H), 7.30 (d, 2H), 7.20 (d, 2H), 3.90 (d, 2H), 3.81 (s, 2H), 2.45 (m, 3H), 2.41 (s, 3H), 1.84 (d, 2H), 1.79 (t, 2H).

### EXAMPLE 3

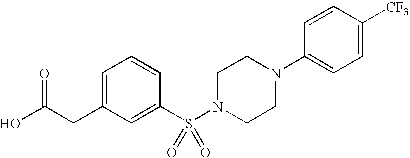

### {3-[4-(4-Trifluoromethyl-phenyl)-piperazine-1-sulfonyl]-phenyl}-acetic acid

The compound of Example 3 was synthesized according to Scheme II

Step 1

(3-Mercapto-phenyl)-acetic acid methyl ester II-A-3. To a solution of 3-mercaptophenyl acetic acid (10 g) in 100 mL of methanol was added concentrated HCl (catalytic amount). The reaction solution was heated under reflux for 5 hours. The solution was evaporated to dryness under reduced pressure. The residue was dissolved in EtOAc and the solution was washed with H₂O (2 times), dried over Na₂SO₄, and evaporated to dryness to yield 6.13 g of the intermediate II-A-3.

Step 2

(3-Chlorosulfonyl-phenyl)-acetic acid methyl ester II-B-3. A solution of II-A-3 from Step 1 (6.13 g) in 30 mL of CH₃CN was cooled to 0° C. To the chilled solution was added in KNO₃, followed by careful addition of SO₂Cl₂ with stirring. The resulting mixture was stirred at 0° C. for 15 minutes, the reaction solution was removed from the ice bath and stirred for an additional of 4 hours. The mixture was then diluted with ether (100 mL), and neutralized with saturated Na₂CO₃ to pH 8. After separation, the aqueous layer was extracted with ether. The combined organic layer was washed with brine and dried over Na₂SO₄ to afford intermediate II-B-3. ¹H NMR (400 MHz, CDCl₃) δ ppm: 8.01 (m, 2H), 7.74 d, 1H), 7.62 (t, 1H), 3.78 (s, 3H), 3.71 (s, 2H).

Step 3

{3-[4-(4-Trifluoromethyl-phenyl)-piperazine-1-sulfonyl]9-phenyl}-acetic acid methyl ester II-C-3. To a solution of II-B-3 from Step 2 in THF (2 mL), was added N-(α,α,α-trifluoro-p-tolyl)piperazine (187 mg, 0.81 mmol, 1.0 equiv.), followed by Et₃N (1.61 mmol, 2.0 equiv.). The reaction mixture was stirred at room temperature overnight. The solvent was evaporated and the residue was purified by chromatography. ¹H NMR (400 MHz, CDCl₃) δ ppm: 7.75 (m, 2H), 7.58 (m, 2H), 7.48 (d, 2H), 6.90(d, 2H), 3.76 (s, 3H), 3.74 (s, 2H), 3.65 (m, 4H), 3.22 (m, 4H).

Step 4

{3-[4-(4-Trifluoromethyl-phenyl)-piperazine-1-sulfonyl]-phenyl}-acetic acid. The compound of Example 3 was prepared from II-C-3 according to the method described for preparing Example 1, Step 3. ¹H NMR (400 MHz, CDCl₃) δ ppm: 7.75 (m, 2H), 7.58 (m, 2H), 7.48 (d, 2H), 6.90 (d, 2H), 3.74 (s, 2H), 3.65 (m, 4H), 3.22 (m, 4H).

### EXAMPLE 4

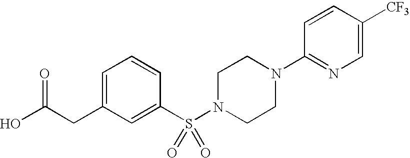

### {3-[4-(5-Trifluoromethyl-pyridin-2-yl)-piperazine-1-sulfonyl]-phenyl}-acetic acid

The compound of Example 4 was prepared according to the method described for preparing Example 3. ¹H NMR (400 MHz, CDCl₃) δ ppm: 8.19 (s, 1H), 7.73 (m, 2H), 7.63 (d, 1H), 7.58 (m, 2H), 6.61 (d, 1H), 3.79 (t, 4H), 3.73 (s, 4H), 3.76 (s, 2H).

### EXAMPLE 5

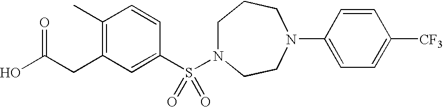

### {2-Methyl-5-[4-(4-trifluoromethyl-phenyl)-[1,4]diazepane-1-sulfonyl]-phenyl}-acetic acid

The compound of Example 5 was prepared according to the method described for preparing Example 3. ¹H NMR (400 MHz, CDCl₃) δ ppm: ¹H NMR (400 MHz, CDCl₃) δ ppm. 7.63 (s, 1H), 7.58 (1H), 7.40 (d, 2H), 7.30 (d, 1H), 6.65 (d, 2H), 3.71 (m, 4H), 3.68 (s, 2H), 3.47 (t, 2H), 3.19 (t, 2H), 2.38 (s, 3H), 2.09 (t, 2H).

### EXAMPLE 6

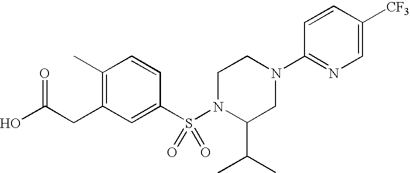

### {5-[2-Isopropyl-4-(5-trifluoromethyl-pyridin-2-yl)-piperazine-1-sulfonyl]-2-methyl-phenyl}-acetic acid

The compound of Example 6 was synthesized according to Scheme III.

Step 1

[5-(4-Benzyl-2-isopropyl-piperazine-1-sulfonyl)-2-methyl-phenyl]-acetic acid methyl ester III-C-6. To a solution of (5-chlorosulfonyl-2-methyl-phenyl)-acetic acid methyl ester intermediate (Example 2, Step 1) (176 mg, 0.67 mmol, 1.0 eqv.) in 2 mL of THF was added intermediate 1-benzyl-3-isopropyl-piperazine III-B-6 (145 mg, 0.67 mmol, 1.0 eqv.), followed by Et₃N (93 μL, 2 eqv). The reaction mixture was stirred at room temperature overnight. The solvent was removed. The residue was dissolved in a minimum amount of CHCl₃ and purified by chromatography with a solvent system of MeOH/CH₂Cl₂ (2:98) to yield III-C-6 (233 mg). ¹H NMR (400 MHz, CDCl₃) δ ppm: 7.63 (m, 2H), 7.30 (m, 6H), 3.74 (s, 3H), 3.73 (s, 2H), 3.43 (d, 1H), 3.41 (d, 1H), 3.30 (t, 1H), 3.21 (d, 1H), 2.71 (d, 1H), 2.59 (d, 1H), 2.42 (s, 3H), 1.79 (m, 2H), 0.98 (d, 3H), 0.80 (d, 3H).

Step 2

[5-(2-Isopropyl-piperazine-1-sulfonyl)-2-methyl-phenyl]-acetic acid methyl ester III-D-6. To a solution of III-C-6 (233 mg) in 4.4% of formic acid /MeOH (10 mL) was added Pd-C (160 mg). The resulting mixture was stirred at room temperature overnight. The reaction sample was filtered through a plug of celite, and the solvent was evaporated to dryness. The residue was dissolved in CH₂Cl₂, and the solution was washed with saturated Na₂CO₃, H₂O, and brine. The solution was dried (Na₂SO₄) and the solvent was removed in vacuo to yield III-D-6 (92 mg).

Step 3

{5-[2-Isopropyl-4-(5-trifluoromethyl-pyridin-2-yl)-piperazine-1-sulfonyl]-2-methyl-phenyl}-acetic acid methyl ester III-E-6. To a solution of III-D-6 (90 mg, 0.25 mmol) in toluene (10 mL) was 2-chloro-5-(trifluoromethyl)pyridine, followed by Et₃N (35 μL, 0.50 mmol). The reaction mixture was stirred at 150° C. in a sealed high pressure flask overnight. The mixture was cooled to room temperature and the solvent was removed under reduced pressure. The residue was dissolved in a small amount of dichloromethane and purified by chromatography. ¹H NMR (400 MHz, CDCl₃) δ ppm: 8.36(s, 1H), 7.75 (s, 1H), 7.70 (d, 1H), 7.60 (d, 1H), 7.28 (d, 1H), 6.47 (d, 1H), 4.39 d, 1H), 3.95 (bt, 2H), 3.71 (s, 2H), 3.71 (m, 1H), 3.30 (m, 1H), 2.93 (dd, 1H), 2.80 (dt, 1H), 2.38 (s, 3H), 2.01 (m, 1H), 1.06 (d, 3H), 0.98 (d, 3H).

Step 4

{5-[2-isopropyl-4-(5-trifluoromethyl-pyridin-2-yl)-piperazine-1-sulfonyl]-2-methyl-phenyl}-acetic acid. The compound of Example 6 was prepared from III-E-6 according to the method described for preparing Example 1, Step 3. ¹H NMR (400 MHz, CDCl₃) δ ppm: 8.35 (s, 1H), 7.75 (s, 1H), 7.70 (d, 1H), 7.60 (d, 1H), 7.28 (d, 1H), 6.47 (d, 1H), 4.39 (d, 1H), 3.95 (bt, 2H), 3.71 (m, 1H), 3.71 (s, 2H), 3.30 (m, 1H), 2.93 (dd, 1H), 2.80 (dt, 1H), 2.38 (s, 2H), 2.01 (m, 1H), 1.06 (d, 2H), 0.98 (d, 3H).

### EXAMPLE 7

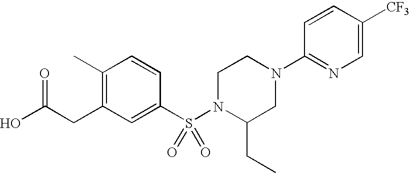

### {5-[2-Ethyl-4-(5-trifluoromethyl-pyridin-2-yl)-piperazine-1-sulfonyl]-2-methyl-phenyl)-acetic acid

The compound of Example 7 was prepared according to the method described for preparing Example 6. ¹H NMR (400 MHz, CDCl₃) δ ppm: 8.39 (s, 1H), 7.75 (s, 1H), 7.70 (d, 1H), 7.61 (d, 1H), 7.32 (d, 1H), 6.51 (d, 1H), 4.20 (d, 1H), 4.15 (d, 1H), 4.00 (bt, 1H), 3.82 (d, 1H), 3.72 As, 3H), 3.30 (m, 1H), 3.09 (dd, 1H), 2.91 (dt, 1H), 2.39 (s, 2H), 1.59 (q, 2H), 0.98 (t, 3H).

### EXAMPLE 8

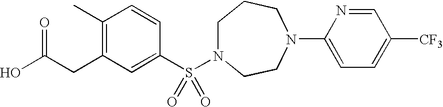

### {2-Methyl-5-[4-(5-trifluoromethyl-pyridin-2-yl)-[1,4]diazepane-1-sulfonyl]-phenyl}-acetic acid

The compound of Example 8 was prepared according to the method described for preparing Example 3. ¹H NMR (400 MHz, CDCl₃) δ ppm: 8.35 (s, 1H), 7.67 (s, 1H), 7.58 (t, 1H), 7.28 (d, 2H), 6.50 (d, 1H), 3.96 (t, 4H), 3.79 (t, 2H), 3.73 (s, 2H), 3.47 (t, 2H), 3.23 (t, 2H), 2.39 (s, 3H).

### EXAMPLE 9

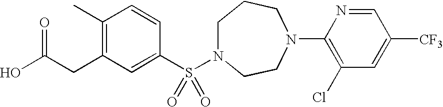

### {5-[4-(3-Chloro-5-trifluoromethyl-pyridin-2-yl)-[1,4]diazepane-1-sulfonyl]-phenyl}-2-methyl-phenyl}acetic acid

The compound of Example 9 was prepared according to the method described for preparing Example 3. ¹H NMR (400 MHz, CDCl₃) δ ppm: 8.28 (s, 1H), 7.72 (s, 1H), 7.70 (t, 1H), 7.35 (d, 2H), 3.95 (t, 4H), 3.78 (s, 2H), 3.69 (t, 2H), 3.35 (1, 2H), 2.42 (s, 3H), 2.08 (m, 2H).

### EXAMPLE 10

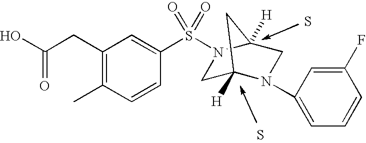

### S,S-{5-[4-(3-Fluorophenyl)-2,5-diaza-bicyclo[2.2.1]heptane-1-sulfonyl]-2-methyl-phenyl}acetic acid

The compound of Example 10 was prepared according to the method described for preparing Example 3. The compound has the absolute stereochemistry indicated. ¹H NMR (400 MHz, CDCl₃) δ ppm: 7.68 (s, 1H), 7.62 (d, 1H), 7.27 (d, 1H), 7.11 (q, 1H), 6.41 (t, 1H), 6.20 (d, 1H), 6.08 (d, 1H), 4.56(s, 1H), 4.29 (s, 1H), 3.72 (d, 2H), 3.53 (d, 1H), 3.41 (d, 1H), 3.35 (d, 1H), 3.17 (d, 1H), 2.41 (s, 3H), 1.90 (d. 1H), 1.59 (d, 1H).

### EXAMPLE 11

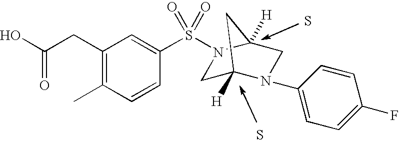

### S,S-15-[4-(4-Fluorophenyl)-2,5-diaza-bicyclo[2.2.1]heptane-1-sulfonyl]-2-methyl-phenyl) acetic acid

The compound of Example 11 was prepared according to the method described for preparing Example 3. ¹H NMR (400 MHz, CDCl₃) δ ppm: 7.68 (s, 1H), 7.62 (d, 1H), 7.30 (d, 1H), 6.91 (t, 2H), 6.40 (m, 2H), 4.56 (s, 1H), 4.29 (s, 1H), 3.72 (d, 2H), 3.53 (d, 1H), 3.47 (d, 1H), 3.31 (d, 1H), 3.18 (d, 1H), 2,42 (s, 3H), 1.90 (d. 1H), 1.59 (d, 1H).

### EXAMPLE 12

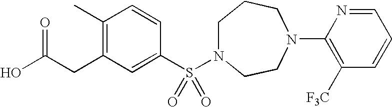

### {2-Methyl-5-[4-(3-trifluoromethyl-pyridin-2-yl)-[1,4]diazepane-1-sulfonyl]-phenyl}-acetic acid

The compound of Example 12 was prepared according to the method described for preparing Example 3. ¹H NMR (400 MHz, CDCl₃) δ ppm: 8.33 (d, 1H), 7.84 (d, 1H), 7.64 (s, 1H), 7.61 (d, 1H), 7.33 (d, 1H), 6.87 (m, 1H), 3.72 (s, 2H), 3.67 (t, 2H), 3.61 (t, 2H), 3.58 (t, 2H), 3.48 (t, 2H), 2.40 (s, 3H), 2.08 (m, 2H).

### EXAMPLE 13

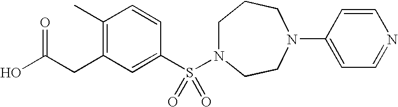

### [2-Methyl-5-(4-pyridin-4-yl)-[1,4]diazepane-1-sulfonyl)-phenyl]-acetic acid

The compound of Example 12 was prepared according to the method described for preparing Example 3. ¹H NMR (400 MHz, CDCl₃) δ ppm: 8.38 (s, 1H), 7.60 (m, 3H), 7.25 (m, 2H), 6.43 (d, 1H), 3.95 (bt, 2H), 3.79 (bt, 2H), 3.74 (s, 1H), 3.70 (s, 1H), 3.42 (t, 2H), 3.22 (t, 2H), 2.38 (s, 3H), 2.10 (m, 2H).

### EXAMPLE 14

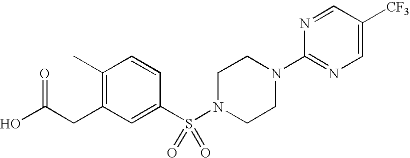

### {2-Methyl-5-[4-(4-trifluoromethyl-pyrimidine-2-yl)-piperazine-1-sulfonyl]-phenyl)-acetic acid

The compound of Example 14 was prepared according to the method described for preparing Example 3. ¹H NMR (400 MHz, CDCl₃) δ ppm: 8.48 (d, 1H), 7.61 (s, 1H), 7.60 (d, 1H), 7.38 (d, 1H), 6.80 (d, 1H), 4.00 (t, 4H), 3.73 (s, 2H), 3.11 (t, 4H), 2.39 (s, 3H).

### EXAMPLE 15

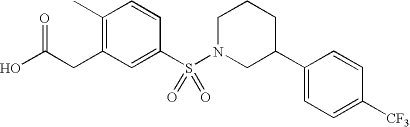

### {2-Methyl-5-[3-(4-trifluoromethyl-phenyl)-piperidine-1-sulfonyl]-phenyl}-acetic acid

The compound of Example 15 was prepared according to the method described for preparing Example 3. (400 MHz, CDCl₃) δ ppm: 7.62 (t, 4H), 7.40 (d, 1H), 7.32 (s, 1H), 7.29 (d, 1H), 3.83 (m, 2H), 3.78 (s, 2H), 3.00 (m, 1H), 2.44 (s, 3H), 2.35 (m, 2H), 2.00 (d, 1H), 1.82 (m, 2H), 1.42 (m, 1H).

### EXAMPLE 16

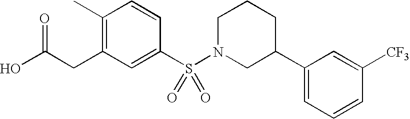

### {2-Methyl-5-[3-(3-trifluoromethyl-phenyl)-piperidine-1-sulfonyl]-phenyl}-acetic acid

The compound of Example 16 was prepared according to the method described for preparing Example 3. ¹H NMR (400 MHz, CDCl₃) δ ppm: 7.61 (s, 1H), 7.60 (s, 1H), 7.51 (d, 1H), 7.41 (m, 3H), 7.40 (t, 1H), 3.84 (bt, 2H), 3.79 (s, 2H), 2.99 (bt, 1H), 2.44 (s, 3H), 2.32 (m, 2H), 2.00 (d, 1H), 1.82 (m, 2H), 1.43 (m, 1H).

### EXAMPLE 17

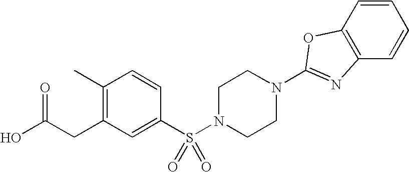

### [5-(4-Benzoxazol-2-yl-piperazine-1-sulfonyl)-2-methyl-phenyl]-acetic acid

The compound of Example 17 was synthesized according to Scheme IV.

Step 1

2-Piperazin-1-yl-benzoxazole IV-A-17. To a solution of piperazine (2.24 g, 26 mmol, 1 equiv.) in toluene was added 2-chlorobenzoxazole (1.0 g, 6.51 mmol, 1 equiv.), followed by Et₃N (3.62 mL, 4 equiv.). The resulting mixture was stirred at 40° C. for 5 hours. The solvent was removed under reduced pressure, and the residue was dissolved in EtOAc. The solution was washed with H₂O (×4), brine and dried over Na₂SO₄. The solvent was evaporated under reduced pressure to afford 0.87 g of intermediate IV-A-17.

Step 2

[5-(4-Benzoxazol-2-yl-piperazine-1-sulfonyl)-2-methyl-phenyl]-acetic acid methyl ester IV-B-17. [5-(4-Benzooxazol-2-yl-piperazine-1-sulfonyl)-2-methyl-phenyl]-acetic acid methyl ester was prepared according to the procedure outlined for Example 3 step 3.

Step 3

[5-(4-Benzoxaol-2-yl-piperazine-1-sulfonyl)-2-methyl-phenyl]-acetic acid. The compound of Example 17 was prepared according to the method described for preparing Example 1 in step 3. ¹H NMR (400 MHz, CDCl₃) δ ppm: 7.61 (m, 2H), 7.47 (d, 1H), 7.40 (d, 1H), 7.30 (m, 2H), 7.08 (m, 1H), 3.74 (bm, 6H), 3.20 (bm, 4H), 2.40 (s, 3H).

### EXAMPLE 18

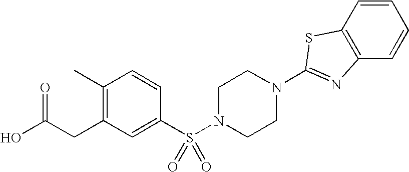

### [5-(4-Benzothiazol-2-yl-piperazine-1-sulfonyl)-2-methyl-phenyl]-acetic acid

The compound of Example 18 was prepared according to the method described for preparing Example 17. ¹H NMR (400 MHz, CDCl₃) δ ppm: 7.60 (m, 4H), 7.38 (d, 1H), 7.35 (t, 1H), 7.15 (t, 1H), 3.73 (s, 2H), 3.74 (t, 4H), 3.20 (t, 4H), 2.40 (s, 3H).

### EXAMPLE 19

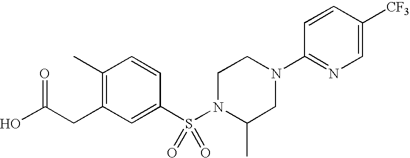

### {2-Methyl-5-[2-methyl-4-(5-trifluoromethyl-pyridin-2-yl)-piperazine-1-sulfonyl-phenyl}-acetic acid

Step 1

3-Methyl-1-(5-trifluoromethyl-pyridin-2-yl)-piperazine. A solution of 2-chloro-5-trifluoromethylpyridine (2.34 g, 12.9 mmol, 1.0 equiv.), 2-methypiperazine (2.59 g, 25.8 mmol, 2.0 equiv.) and triethylamine (5.4 mL, 38.7 mmol, 3.0 equiv.) in toluene (20 mL) was sealed in a 50 mL of high pressure reaction tube. The reaction mixture was heated to 150° C. with stirring. After stirring at 150° C. for 20 hours, the reaction mixture was cooled to room temperature and then diluted with CH₂Cl₂ (200 mL). The organic mixture was washed with water (100 mL×2), brine and then dried over Na₂SO₄. After filtration and removal of solvent, 3.05 g (96% yield) of the desired intermediate was obtained as a bright yellow solid, which was used without purification. ¹H NMR (400 MHz, CDCl₃), δ (ppm): 8.42 (m, 1H), 7.65 (dd, 1H), 6.66 (d, 1H), 4.26 (m, 2H), 3.14 (m, 1H), 2.94 (m, 3H), 2.60 (dd, 1H), 1. 18 (d, 3H).

Step 2

{2-Methyl-5-[2-methyl-4-(5-trifluoromethyl-pyridin-2-yl)-piperazine-1-sulfonyl]-phenyl}-acetic acid methyl ester. To a solution of (5-Chlorosulfonyl-2-methyl-phenyl)-acetic acid methyl ester (316 mg, 1.2 mmol, 1.0 equiv.) and the product from step 1 (295 mg, 1.2 mmol, 1.0 equiv.) in THF (10 mL) was added Et₃N (334.5 μL, 2.4 mmol, 2.0 equiv.) and catalytic amount of DMAP. The resulting mixture was warmed to 55° C. and stirred at same temperature for 6 hours. The reaction mixture was concentrated under nitrogen. The residue was diluted with ethyl acetate (20 mL) and then washed with water, saturated NaHCO₃, brine and dried over Na₂SO₄. After removal of solvent, the crude product was purified by chromatography to give desired intermediate methyl ester (417 mg, 89% yield). ¹H NMR (400 MHz, CDCl₃), δ (ppm):): 8.33 (d, 1H), 7.67 (d, 1H), 7.63 (dd, 1H), 7.59 (dd, 1H), 7.28 (d, 1H), 6.51 (d, 1H), 4.22 (m, 2H), 4.02 (d, 1H), 3.75 (dt, 1H), 3.69 (s, 5H), 3.26 (m, 2H), 3.01 (td, 1H), 2.35 (s, 3H), 1.08 (d, 3H).

Step 3

{2-Methyl-5-[2-methyl-4-(5-trifluoromethyl-pyridin-2-yl)-piperazine-1-sulfonyl]-phenyl}-acetic acid. To a solution of the product from step 2 (417 mg, 0.88 mmol, 1.0 equiv.) in THF/MeOH (3:1) (5 mL) was added 1N LiOH aqueous solution (1.8 mL, 1.8 mmol, 2.0 eqiv.). The resulting mixture was stirred at room temperature for 4.5 hours and then concentrated under nitrogen. The residue was diluted with water (5 mL) and then partitioned with diethyl ether (5 mL). After separation, the aqueous solution was neutralized with 1N HCl (1.8 mL, 1.8 mmol, 2.0 equiv) and extracted with ethyl acetate (10 mL). The organic layer was washed with brine and dried over Na₂SO₄. After removal of solvent, 407 mg (99% yield) of the desired compound was obtained ¹H NMR (400 MHz, CDCl₃), δ (ppm): 8.36 (d, 1H), 7.73 (s, 1H), 7.67 (d, 1H), 7.62 (d, 1H), 7.33 (d, 1H), 6.55 (d, 1H), 4.20 (m, 2H), 4.03 (d, 1H), 3.78 (d, 1H), 3.75 (s, 2H), 3.27 (m, 2H), 3.03 (td, 1H), 2.41 (s, 3H), 1.13 (d, 3H).

### EXAMPLE 20

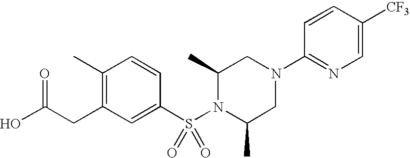

### {5-[2,6-Dimethyl-4-(5-trifluoromethyl-pyridin-2-yl)-piperazine-1-sulfonyl]-2-methyl-phenyl}-acetic acid

The compound of Example 20 was prepared following the procedure for the compound of Example 19. ¹H NMR (400 MHz, CDCl₃), δ (ppm): 8.35 (d, 1H), 7.73 (s, 1H), 7.67 (d, 1H), 7.60 (d, 1H), 7.30 (d, 1H), 6.52 (d, 1H), 4.24 (m, 2H), 4.00 (d, 2H), 3.73 (s, 2H), 3.05 (dd, 2H), 2.38(s, 3H), 1.40(d, 6H).

### EXAMPLE 21

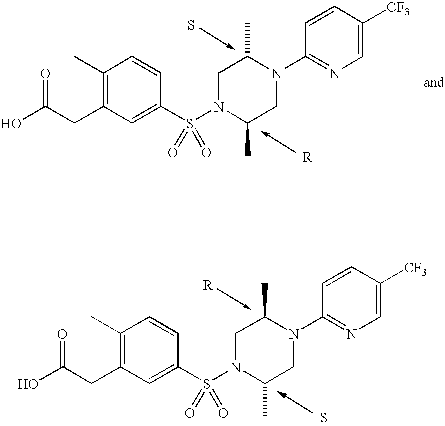

### SR and RS-{5-[2,5-Dimethyl-4-(5-trifluoromethyl-pyridin-2-yl)-piperazine-1-sulfonyl]-2-methyl-phenyl}-acetic acid

The compound of Example 21 was prepared following the procedure for the compound of Example 19. ¹H NMR (400 MHz, CDCl₃), δ (ppm): 8.40 (s, 1H), 7.72 (s, 1H), 7.68 (d, 1H), 7.65 (d, 1H), 7.36 (d, 1H), 6.60 (d, 1H), 4.64 (m, 1H), 4.29 (m, 1H), 4.07 (d, 1H), 3.77 (s, 2H), 3.58(d, 1H), 3.37 (td, 2H), 2.41 (s, 3H), 1.22 (d, 3H), 1.00 (d, 3H).

### EXAMPLE 22

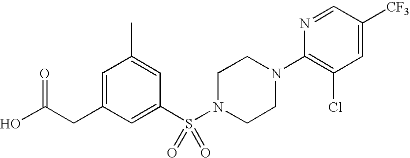

### {5-Methyl-3-[4-(3-chloro-5-trifluoromethyl-pyridin-2-yl)-piperazine-1-sulfonyl]-phenyl)-acetic acid

The compound of Example 22 was prepared following the procedure for compound of Example 19 by using (3-chlorosulfonyl-5-methyl-phenyl)-acetic acid methyl ester. ¹H NMR (400 MHz, CDCl₃), δ (ppm): 8.37 (s, 1H), 7.75 (s, 1H), 7.51 (s, 2H), 7.35 (s, 1H), 3.71 (s, 2H), 3.59-3.56 (m, 2H), 3.20-3.17 (m, 2H), 2.44 (s, 3H). ESMS (M+H): 477.9

### EXAMPLE 23

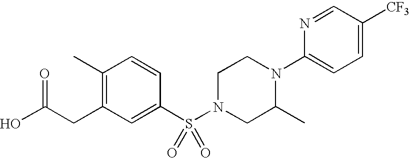

### [2-Methyl-5-[3-methyl-4-(5-trifluoromethyl-pyridin-2-yl)-piperazine-1-sulfonyl]-phenyl}-acetic acid

Step 1

2-Methyl-5-(3-methyl-piperazine-1-sulfonyl)-phenyl]-acetic acid methyl ester was synthesized following the procedure for preparation of intermediate in Example 19, Step 2 by using 2-equivalents of 2-methyl-piperazine in 95% yield. ¹H NMR (400 MHz, CDCl₃), δ (ppm): 7.60 (s, 1H), 7.58 (d, 1H), 7.37 (d, 1H), 3.73 (s, 5H), 3.64 (m, 2H), 2.99 (m, 3H), 2.33 (s, 3H), 2.30 (td, 1H), 1.95 (t, 1H), 1.06 (d, 3H).

Step 2

[2-methyl-5-[3-methyl-4-(5-trifluoromethyl-pyridin-2-yl)-piperazine-1-sulfonyl]-phenyl}-acetic acid methyl ester was synthesized following the procedure for the preparation of intermediate in Example 19, Step 1 in 2% yield. ¹H NMR (400 MHz, CDCl₃), δ (ppm): 8.35 (d, 1H), 7:59 (m, 3H), 7.33 (d, 1H), 6.54 (d, 1H), 4.63 (m, 1H), 4.22 (d, 1H), 3.81 (d, 1H), 3.70 (s, 3H), 3.69 (s, 2H), 3.62 (d, 1H), 3.29 (td, 1H), 2.54 (dd, 1H), 2.37 (s, 3H), 2.35 (m, 1H), 1.31 (d, 3H).

Step 3

{2-Methyl-5-[3-methyl-4-(5-trifluoromethyl-pyridin-2-yl)-piperazine-1-sulfonyl]-phenyl}-acetic acid. The compound of Example 23 was synthesized following the procedure for the preparation of intermediate in Example 19 Step 3 with 96% yield. ¹H NMR (400 MHz, CDCl₃), δ (ppm): 8.36 (d, 1H), 7.61 (m, 3H), 7.34 (d, 1H), 6.55 (d, 1H), 4.62 (m, 1H), 4.21 (d, 1H), 3.81 (m, 1H), 3.73 (s, 2H), 3.62 (m, 1H), 3.29 (td, 1H), 2.53 (dd, 1H), 2.38 (s, 3H), 2.37 (m, 1H), 1.31 (d, 3H).

### EXAMPLE 24

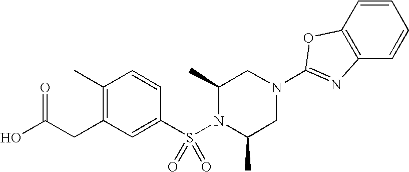

### [5-(4-Benzooxazol-2-yl-2,6-dimethyl-piperazine-1-sulfonyl)-2-methyl-phenyl]-acetic acid

The compound of Example 24 was prepared according to the method described for the preparation of Example 17. ¹H NMR (400 MHz, CDCl₃), δ (ppm): 7.67 (s, 1H), 7.62 (d, 1H), 7.25 (d, 1H), 7.24 (d, 1H), 7.11 (m, 2H), 6.98 (t, 1H), 4.20 (m, 2H), 3.82 (d, 2H), 3.64 (s, 2H), 2.99 (dd, 2H), 2.31 (s, 3H), 1.36(d, 6H).

### EXAMPLE 25

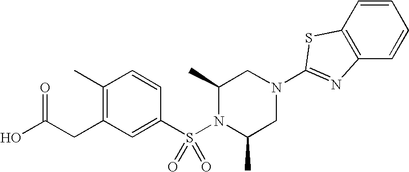

### [5-(4-Benzothiazol-2-yl-2,6-dimethyl-piperazine-1-sulfonyl)-2-methyl-phenyl]-acetic acid

The compound of Example 25 was prepared according to the method described for the preparation of Example 17. ¹H NMR (400 MHz, CDCl₃), δ (ppm): 7.65 (s, 1H), 7.60 (d, 1H), 7.49 (d, 1H), 7.42 (d, 1H), 7.24 (t, 1H), 7.22 (d, 1H), 7.03 (t, 1H), 4.20 (m, 2H), 3.68 (d, 2H), 3.61 (s, 2H), 3.05 (dd, 2H), 2.28 (s, 3H), 1.36 (d, 6H).

### EXAMPLE 26

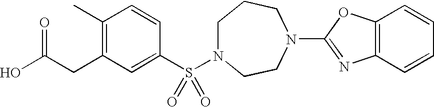

### [5-(4-Benzooxazol-2-yl-[1,4]diazepane-1-sulfonyl)-2-methyl-phenyl]-acetic acid

The compound of Example 26 was prepared according to the method described for the preparation of Example 17. ¹H NMR (400 MHz, CDCl₃), δ (ppm): 7.59 (s, 1H), 7.52 (d, 1H), 7.28 (d, 1H), 7.16 (d, 1H), 7.14 (d, 1H), 7.11 (t, 1H), 6.97 (t, 1H), 3.79 (t, 2H), 3.72 (t, 2H), 3.60 (s, 2H), 3.47 (t, 2H), 3.30(t, 2H), 2.22 (s, 3H), 2.00 (q, 2H).

### EXAMPLE 27

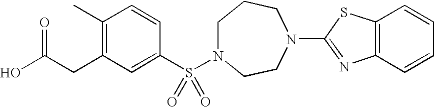

### [5-(4-Benzothiazol-2-yl-[1,4]diazepane-1-sulfonyl)-2-methyl-phenyl]-acetic acid

The compound of Example 27 was prepared according to the method described for the preparation of Example 17. ¹H NMR (400 MHz, CDCl₃), δ (ppm): 7.58 (s, 1H), 7.52 (m, 2H), 7.45 (d, 1H), 7.23 (t, 1H), 7.15 (d, 1H), 7.02 (t, 1H), 3.81 (t, 2H), 3.68 (t, 2H), 3.59 (s, 2H), 3.48 (t, 2H), 3.27 (t, 2H), 2.22 (s, 3H), 2.04 (q, 2H).

### EXAMPLE 28

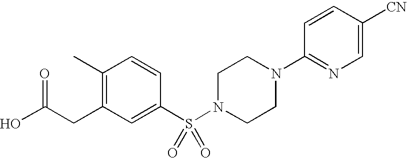

### {5-[4-(5-Cyano-pyridin-2-yl)-piperazine-1-sulfonyl]-2-methyl-phenyl}-acetic acid

The compound of Example 28 was prepared according to the method described for the preparation of Example 17. ¹H NMR (400 MHz, MeOH-D₄) δ 8.36 (d, 1H), 7.69 (dd, 1H), 7.62 (s, 1H), 7.59 (dd, 1H), 7.42 d, 1H), 6.82 (d, 1H), 3.80-3.78 (m, 4H), 3.76 (s, 2H), 3.07-3.04 (m, 4H), 2.38 (s, 3H); LCMS: 401.0 (m+1)⁺.

### EXAMPLE 29

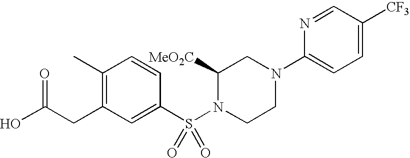

### (R)-1-(3-Carboxymethyl-4-methyl-benzenesulfonyl)-4-(5-trifluoromethyl-pyridin-2-yl)-piperazine-2-carboxylic acid methyl ester

Step 1

o-Tolylacetic acid (2.0 g, 13.3 mmol) was combined with p-nitrobenzyl bromide (5.8 g, 26.8 mmoles) and 1,8-diazabicyclo[5.4.0]undec-7-ene (2.4 mL, 16.0 mmol) in 65 mL of benzene, and was stirred at 50° C. for 20 hours. After this period the heterogeneous mixture was gravity filtered and the filtrate was evaporated in vacuo. The residue was combined with CH₂Cl₂ and was washed with 1N HCl (2×25 mL) and sat'd NaHCO₃ (2×25 mL), and the resulting CH₂Cl₂ solution was dried over anhydrous Na₂SO₄. The crude solid was purified using flash silica chromatography (0-10% EtOAc/Hexane) to yield 3.61 g (95%) of the intermediate as a white solid. ¹H NMR (400 MHz, CDCl₃) δ 8.16 (d, 2H), 7.39 (d, 2H), 7.22-7.16 (m, 4H), 5.21 (s, 2H), 3.72 (s, 2H), 2.30 (s, 3H).

Step 2

o-Tolylacetic acid 4-nitro-benzyl ester (2.3 g, 8.1 mmol) was dissolved into 13 mL of anhydrous CHCl₃. To this stirring solution at −20° C. was added chlorosulfonic acid (2.8 g, 24.0 mmol) over a period of 10 minutes. The mixture was then allowed to warm to ambient temperature and was allowed to stir for 16 hours. After this period the reaction mixture was combined with ice-water and the resulting layer was extracted with copious CH₂Cl₂. The CH₂Cl₂ layer was washed with brine and was dried over anhydrous Na₂SO₄. The crude product was purified using flash silica chromatography (0-30% EtOAc/Hex) to yield 0.84 g (27%) of (5-Chlorosulfonyl-2-methyl-phenyl)-acetic acid 4-nitro-benzyl ester, intermediate IX-A as a white, crystalline solid. ¹H NMR (400 MHz, CDCl₃) δ 8.22 (d, 2H), 7.88 (d, 2H), 7.49-7.44 (m, 3H), 5.26 (s, 2H), 3.84 (s, 2H), 2.42 (s, 3H).

Step 3

(R)-Piperazine-1,2-dicarboxylic acid 1-tert-butyl ester 2-methyl ester (120 mg, 0.49 mmol) and 2-Bromo-5-trifluoromethyl-pyridine (133 mg, 0.59 mmol) were dissolved into 2.0 mL of anhydrous toluene (degassed). In a separate, septum-equipped vial were placed tri(dibenzylideneacetone)dipalladium (0) (22 mg, 0.024 mmol), 1,3-bis(2,6-di-i-propylphenyl)imidazolium chloride (42 mg, 0.1 mmol) and sodium t-butoxide (57 mg, 0.59 mmol). This “catalytic” vial was equipped with a magnetic stir bar and flushed with dry nitrogen. The reactant solution was next transferred to the “catalytic” vial and the mixture was stirred at 100° C. for 5 h. After this period the mixture was combined with 20 mL of hexane/EtOAc (2:1) and was passed through a pad of Celite. The resulting filtrate was evaporated in vacuo and purified using flash silica chromatography (0-20% EtOAc/Hexane) to yield 110 mg (58%) of (R)-4-(5-Trifluoromethyl-pyridin-2-yl)-piperazine-1,2-dicarboxylic acid 1-tert-butyl ester 2-methyl ester, intermediate IX-B, as a yellow residue. ¹H NMR (400 MHz, CDCl₃) δ 8.39-8.38 (m, 1H), 7.65 (d, 1H), 6.68 (m, 1H), 4.89-4.68 (m, 2H), 4.29 (dd, 1H), 3.95 (dd, 1H), 3.69 (s, 3H), 3.43-3.26 (m, 2H), 3.12-2.97 (m, 1H), 1.51-1.46 (m, 9H).

Step 4

(R)-4-(5-Trifluoromethyl-pyridin-2-yl)-piperazine-1,2-dicarboxylic acid 1-tert-butyl ester 2-methyl ester, IX-B (110 mg, 0.28 mmol) was combined with 2.0 mL of 25% TFA/CH₂Cl₂ and was stirred at room temperature for 30 min. After this period the reaction mixture was combined with 25 mL of CH₂Cl₂ and was washed with sat'd NaHCO₃ (2×10 mL) and brine. The resulting CH₂Cl₂ layer was dried over anhydrous Na₂SO₄ and was evaporated in vacuo to yield crude amine. The crude amine was purified using flash silica chromatography (0-10% MeOH/CH₂Cl₂) to yield 77 mg (94%) of (R)-4-(5-Trifluoromethyl-pyridin-2-yl)-piperazine-2-carboxylic acid methyl ester as a yellow residue. This material was combined with (5-Chlorosulfonyl-2-methyl-phenyl)-acetic acid 4-nitro-benzyl ester, IX-A (102 mg, 0.27 mmoles) and triethylamine (46 μL, 0.33 mmol) in 2.0 mL of anhydrous THF, and was stirred at 60° C. for 5 hours. After this period the reaction mixture was evaporated in vacuo and the resulting residue was combined with 30 mL of benzene. The resulting heterogeneous mixture was filtered with benzene washings. The filtrate was then evaporated in vacuo and purified using flash silica chromatography (0-30% EtOAc/Hexane) to yield 87 mg (51%) of (R)-1-[4-Methyl-3-(4-nitro-benzyloxycarbonylmethyl)-benzenesulfonyl]-4-(5-trifluoromethyl-pyridin-2-yl)-piperazine-2-carboxylic acid methyl ester, intermediate IX-C as a yellow residue. ¹H NMR (400 MHz, CDCl₃) δ 8.33 (s, 1H), 8.20 (d, 2H), 7.67-7.60 (m, 3H), 7.45 (d, 2H), 7.32 (d, 1H), 6.62 (d, 1H), 5.22 (s, 2H), 4.82 (d, 1H), 4.76-4.75 (m, 1H), 4.37 (d, 1H), 3.80-3.77 (m, 3H), 3.46-3.39 (m, 4H), 3.38-3.27 (m, 1H), 3.07-3.00 (m, 1H), 2.35 (s, 3H).

Step 5

(R)-1-[4-Methyl-3-(4-nitro-benzyloxycarbonylmethyl)-benzenesulfonyl]-4-(5-trifluoromethyl-pyridin-2-yl)-piperazine-2-carboxylic acid methyl ester (87 mg, 0.14 mmol) was combined with 10% Pd/C (75 mg), cyclohexadiene (260 μL, 2.8 mmol) and 2.0 mL of ethanol within an 8 mL Teflon-capped vial. This mixture was stirred at 70° C. for 6 h and then passed through a Celite plug (with MeOH washings). The resulting filtrate was evaporated in vacuo, and the crude residue was purified using flash silica chromatography (0-10% MeOH/CH₂Cl₂) to yield 39 mg (56%) of (R)-1-(3-Carboxymethyl-4-methyl-benzenesulfonyl)-4-(5-trifluoromethyl-pyridin-2-yl)-piperazine-2-carboxylic acid methyl ester as a yellow residue. ¹H NMR (400 MHz, d6-DMSO) δ 12.4 (bs, 1H), 8.37 (s, 1H), 7.81-7.78 (m, 1H), 7.67 (s, 1H), 7.60-7.58 (m, 1H), 7.38 (d, 1H), 6.88 (d, 1H), 4.78-4.72 (m, 2H), 4.28-4.25 (m, 1H), 3.72-3.65 (m, 3H), 3.38-3.23 (m, 6H), 2.97-2.90 (m, 1H), 2.29 (s, 3H). ESMS (M+H): 501.9.

### EXAMPLE 30

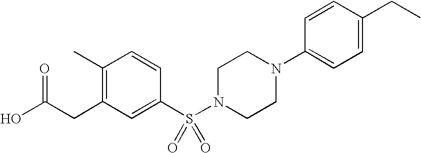

### {5-[4-(4-Ethyl-phenyl)-piperazine-1-sulfonyl]-2-methyl-phenyl}-acetic acid

The compound of Example 30 was synthesized according to the procedure outlined for Example 17. ¹H NMR (400 MHz, d6-DMSO) δ 7.61 (s, 1H), 7.55 (s, 1H), 7.47 (d, 1H), 7.03 (d, 2H), 6.81 (d, 2H), 3.76 (s, 2H), 3.14-3.12 (m, 4H), 2.99-2.97 (m, 4H), 2.45 (q, 2H), 1.10 (t, 3H). ESMS (M+H): 403.04

### EXAMPLE 31

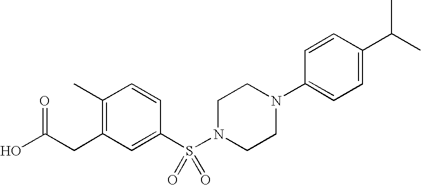

### {5-14-(4-Isopropyl-phenyl)-piperazine-1-sulfonyl]-2-methyl-phenyl}-acetic acid

The compound of Example 31 was synthesized according to the procedure outlined for Example 17. ¹H NMR (400 MHz, d6-DMSO) δ 7.60 (s, 1H), 7.54 (m, 1H), 7.45 (d, 1H), 7.06 (d, 2H), 6.82 (d, 2H), 3.73 (s, 2H), 3.14-3.11 (m, 4H), 2.99-2.96 (m, 4H), 2.78-2.75 (m, 1H), 2.32 (s, 3H), 1.13 (d, 6H). ESMS (M+H): 417.01

### EXAMPLE 32

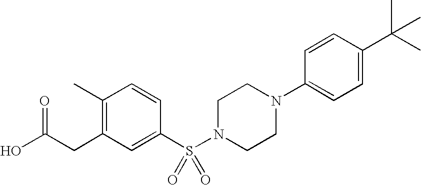

### {5-[4-(4-tert-Butyl-phenyl)-piperazine-1-sulfonyl]-2-methyl-phenyl}-acetic acid

The compound of Example 32 was synthesized according to the procedure outlined for Example 17. ¹H NMR (400 MHz, d6-DMSO) δ 7.63 (m, 1H), 7.58-7.56 (m, 1H), 7.49-7.47 (m, 1H), 7.22 (d, 2H), 6.84 (d, 2H), 3.76 (s, 2H), 3.16-3.14 (m, 4H), 3.01-3.00 (m, 4H), 2.34 (s, 3H), 1.23 (s, 9H). ESMS (M+H): 431.04

### EXAMPLE 33

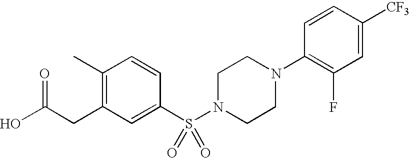

### (5-[4-(2-Fluoro-4-trifluoromethyl-phenyl)-piperazine-1-sulfonyl-2-methyl-phenyl)-acetic acid

The compound of Example 33 was synthesized according to the procedure outlined for Example 17. ¹H NMR (400 MHz, d6-DMSO) δ 7.59-7.53 (m, 3H), 7.46 (t, 2H), 7.18 (t, 1H), 3.68 (bs, 2H), 3.20-3.17 (m, 4H), 3.02 (m, 4H), 2.33 (s, 3H) ESMS (M+H): 460.93.

### EXAMPLE 34

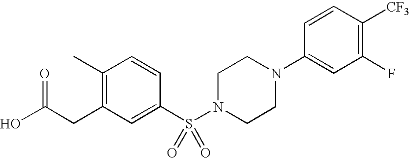

### {5-[4-(3-Fluoro-4-trifluoromethyl-phenyl)-piperazine-1-sulfonyl]-2-methyl-phenyl}-acetic acid

The compound of Example 34 was synthesized according to the procedure outlined for Example 17. ¹H NMR (400 MHz, d6-DMSO) δ 7.60 (s, 1H), 7.56-7.53 (m, 1H), 7.49-7.44 (m, 2H), 6.94 (d, 1H), 6.81 (d, 1H), 4.10 (bs, 1H), 3.73 (s, 2H), 3.43-3.41 (m, 4H), 3.17-3.16 (m, 2H), 2.69 (m, 4H), 2.31 (s, 3H). ESMS (M+H): 460.93.

### EXAMPLE 35

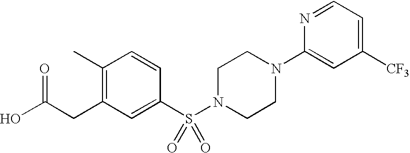

### {2-Methyl-5-[4-(4-trifluoromethyl-pyridin-2-yl)-piperazine-1-sulfonyl]-phenyl}-acetic acid

The compound of Example 35 was synthesized according to the procedure outlined for Example 17. ¹H NMR (400 MHz, d6-DMSO) δ 8.29 (d, 1H), 7.60 (s, 1H), 7.56-7.53 (m, 1H), 7.44 (d, 1H), 7.09 (s, 1H), 6.89 (d, 1H), 3.74 (s, 2H), 3.71-3.68 (m, 4H), 2.96-2.93 (m, 4H), 2.30 (s, 3H). ESMS (M+H): 443.95

### EXAMPLE 36

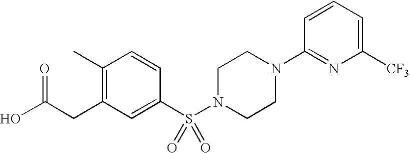

### {2-Methyl-5-[4-(6-trifluoromethyl-pyridin-2-yl)-piperazine-1-sulfonyl]-phenyl}-acetic acid

The compound of Example 36 was synthesized according to the procedure outlined for Example 17. ¹H NMR (400 MHz, d6-DMSO) δ 7.73 (t, 1H), 7.60 (s, 1H), 7.56-7.53 (m, 1H), 7.44 (d, 1H), 7.09 (d, 1H), 7.05 (d, 1H), 3.74 (s, 2H), 3.67-3.64 (m, 4R), 2.97-2.96 (m, 4H), 2.30 (s, 3H). ESMS (M+H): 443.94

### EXAMPLE 37

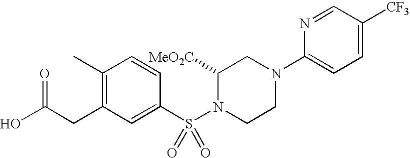

### (S)-1-(3-Carboxymethyl-4-methyl-benzenesulfonyl)-4-(S-trifluoromethyl-pyridin-2-yl)-piperazine-2-carboxylic acid methyl ester

The compound of Example 37 was synthesized according to the procedure outlined for Example 29. ¹H NMR (400 MHz, CDCl₃) δ 8.30 (s, 1H), 7.71-7.68 (m, 2H), 7.63-7.61 (m, 1H), 7.37 (d, 1H), 6.82 (d, 1H), 4.88-4.85 (m, 1H), 4.75 (m, 1H), 4.35-4.32 (m, 1H), 3.80-3.77 (m, 1H), 3.74 (s, 2H), 3.51-3.44 (m, 1H), 3.42 (s, 3H), 3.31-3.27 (m, 1H), 3.04-2.98 (m, 1H), 2.37 (s, 3H). ESMS (M+H): 501.92

### EXAMPLE 38

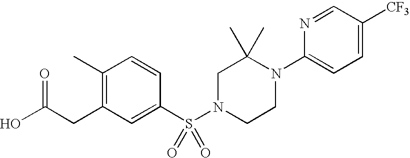

### {5-[3,3-Dimethyl-4-(5-trifluoromethyl-pyridin-2-yl)-piperazine-1-sulfonyl]-2-methyl-phenyl}-acetic acid

The compound of Example 38 was synthesized according to the procedure outlined for Example 23. ¹H NMR (400 MHz, CD₃OD) δ 8.46 (m, 1H), 7.80-7.77 (m, 1H), 7.70 (m, 1H), 7.67-7.64 (m, 1H), 7.49 (d, 1H), 7.05 (d, 1H), 3.82 (s, 2H), 3.67-3.65 (m, 2H), 3.26-3.23 (m, 2H), 2.97 (s, 2H), 2.45 (s, 3H), 1.51 (s, 6H). ESMS (M+H): 472.0

### EXAMPLE 39

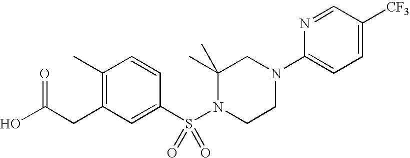

### {5-[2,2-Dimethyl-4-(5-trifluoromethyl-pyridin-2-yl)-piperazine-1-sulfonyl]-2-methyl-phenyl}-acetic acid

The compound of Example 39 was synthesized according to the procedure outlined for Example 17. ¹H NMR (400 MHz, CDCl₃) δ 8.35 (m, 1H), 7.69-7.61 (m, 3H), 7.31 (d, 1H), 6.51 (d, 1H), 3.72-3.63 (m, 8H), 2.38 (s, 3H), 1.38 (s, 6H). ESMS (M+H): 472.0

### EXAMPLE 40

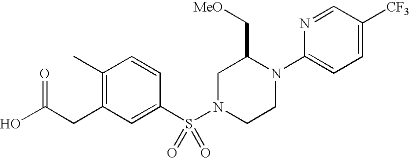

### (S)-15-[3-Methoxymethyl-4-(5-trifluoromethyl-pyridin-2-yl)-piperazine-1-sulfonyl]-2-methyl-phenyl)-acetic acid

The compound of Example 40 was synthesized according to the procedure outlined for Example 23. ¹H NMR (400 MHz, CD₃OD) δ 8.51 (s, 1H), 7.99-7.96 (m, 1H), 7.66 (s, 1H), 7.60 (d, 1H), 7.44 (d, 1H), 6.99 (d, 1H), 4.60-4.56 (m, 1H), 4.51-4.47 (m,1H), 3.76 (s, 2H), 3.71-3.68 (m, 1H), 3.57-3.53 (m, 1H), 2.98-2.93 (m, 1H), 2.73-2.48 (m, 5H), 2.43 (s, 6H). ESMS (M+H): 488.0

### EXAMPLE 41

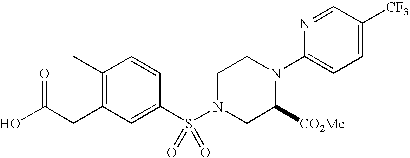

### (R)-4-(3-Carboxymethyl-4-methyl-benzenesulfonyl)- -(5-trifluoromethyl-pyridin-2-yl)-piperazine-2-carboxylic acid methyl ester

The compound of Example 41 was synthesized according to the procedure outlined for Example 29. ¹H NMR (400 MHz, CDCl₃) δ 8.35 (m, 1H), 7.70-7.67 (m, 1H), 7.62-7.59 (m, 2H), 6.66 (d, 1H), 5.52 (m, 1H), 4.35-4.32 (m, 1H), 3.89-3.81 (m, 2H), 3.74 (m, 5H), 3.58-3.51 (m, 1H), 2.64-2.60 (m, 1H), 2.50-2.44 (m, 1H), 2.39 (s, 3H). ESMS (M+H): 502.0

### EXAMPLE 42

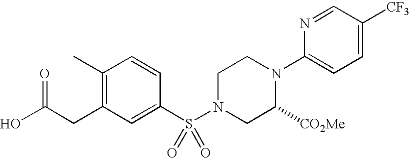

### (S)-4-(3-Carboxymethyl-4-methyl-benzenesulfonyl)-1-(5-trifluoromethyl-pyridin-2-yl)-piperazine-2-carboxylic acid methyl ester

The compound of Example 42 was synthesized according to the procedure outlined for Example 29. ¹H NMR (400 MHz, CD₃OD) δ 8.37 (m, 1H), 7.81-7.79 (m, 1H), 7.68 (m, 1H), 7.65-7.63 (m, 1H), 7.48 (d, 1H), 6.94 (d, 1H), 5.55 (m, 1H), 4.33-4.30 (m, 1H), 4.15-4.12 (m, 1H), 3.85-3.84 (m, 1H), 3.81 (s, 2H), 3.75 (s, 3H), 3.47-3.41 (m, 1H), 2.67-2.63 (m, 1H), 2.50-2.44 (m, 1H), 2.42 (s, 3H). ESMS (M+H): 501.98

### EXAMPLE 43

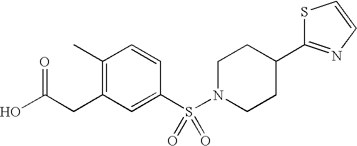

### [2-Methyl-5-(4-thiazol-2-yl-piperidine-1-sulfonyl)-phenyl]-acetic acid

Step 1

A mixture of compound VI-A-43 (13.8 g), P₂S₅ (15.4 g) and anhydrous NaHCO₃ (17.9 g) in ethylene glycol dimethyl ether (207 μL) was stirred at 60° C. overnight. After cooling to room temperature, the solution was filtered and concentrated to about ⅓ of original volume, then poured into ice/water. The precipitated light yellow solid was collected by filtration and dried to give 13.5 g of intermediate X-B-43.

Step 2

A mixture of compound VI-B-43 (0.51 g) and 2-bromoacetaldehyde diethyl acetal (0.43 g) in anhydrous EtOH (30 mL) was refluxed overnight. After cooling to room temperature, the reaction mixture was concentrated. The residue was purified by column chromatography to give 0.3 g of intermediate VI-C-43 as yellow oil.

Step 3

Compound VI-C-43 (0.3 g) was stirred in a solution of HBr in HOAc (33%, 10 mL) at 10° C. for an hour, then concentrated to give 0.3 g of intermediate VI-D-43 as light yellow solid.

Steps 4 and 5

[2-Methyl-5-(4-thiazol-2-yl-piperidine-1-sulfonyl)-phenyl]-acetic acid. The compound of Example 43 was synthesized from intermediate VI-D-43 according to the method described for the preparation of Example 17, Steps 2 and 3.

¹H NMR (400 MHz, CDCl₃) δ ppm. 7.84 (m, 1H), 7.70 (s, 1H), 7.67 (d, 1H), 7.64 (s, 1H), 7.36 (s, 1H), 7.34 (d, 1H), 3.94 (d, 2H), 3.74 (s, 2H), 2.62 (t, 2H), 2.43 (s, 3H), 2.15 (m, 2H), 1.94 (t, 2H), 1.26 (m, 1H).

### EXAMPLE 44

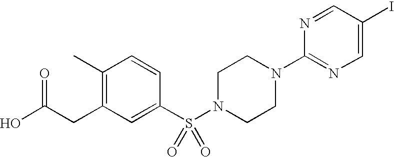

### {5-[4-(5-Iodo-pyrimidin-2-yl)-piperazine-1-sulfonyl]-2-methyl-phenyl}-acetic acid

Step 1

2-Piperazin-1-yl-pyrimidine. A mixture of 2-chloropyrimidine (10 g) and piperazine (25 g) in DMF (100 mL) was stirred at 75° C. for 30 min. After cooling to room temperature, the reaction mixture was diluted with CH₂Cl₂ and washed with water. The CH₂Cl₂ solution was dried and concentrated. The residue was purified by column chromatography eluting with CH₂Cl₂/MeOH (40: 1) to afford 6.4 g of 2-Piperazin-1-yl-pyrimidine.

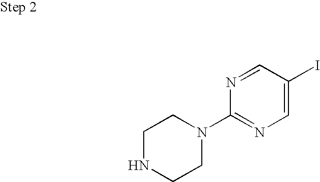

5-Iodo-2-piperazin-1-yl-pyrimidine. 2-Piperazin-1-yl-pyrimidine from Step 1 (0.5 g) was placed in the reaction vessel, followed by adding 12 (0.21 g), HIO₄.H₂O (0.095 g), HOAc (1.25 mL), H₂O (0.25 mL), and H₂SO₄ (0.0375 mL). The mixture was then heated at 100° C. for 6 hours. After cooling to room temperature, it was diluted with CH₂Cl₂ and washed with water. The CH₂Cl₂ solution was dried and concentrated. The residue was purified by column chromatography to afford 0.5 g of 5-Iodo-2-piperazin-1-yl-pyrimidine.

Step 3 and 4

{5-[4-(5-Iodo-pyrimidin-2-yl)-piperazine-1-sulfonyl]-2-methyl-phenyl}-acetic acid. The compound of Example 44 was synthesized from 5-Iodo-2-piperazin-1-yl-pyrimidine according to the method described for the preparation of Example 17, Steps 2 and 3. LCMS: 503.0 (M+1)⁺.

### EXAMPLE 45

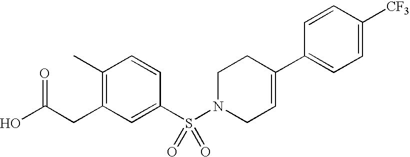

### {2-Methyl-5-[4-(4-trifluoromethyl-phenyl)-3,6-dihydro-2H-pyridine-1-sulfonyl]-phenyl}-acetic acid

Step 1

4-(4-Trifluoromethyl-phenyl)-1,2,3,6-tetrahydro-pyridine. The compound 4-(4-Trifluoromethyl-phenyl)-1,2,3,6-tetrahydro-pyridine was synthesized according to the procedures described for Example 48 Steps 1-4.

Step 2

{2-Methyl-5-[4-(4-trifluoromethyl-phenyl)-3,6-dihydro-2H-pyridine-1-sulfonyl]-phenyl}-acetic acid ethyl ester. Methyl 2-(5-chlorosulfonyl-2-methyl)phenyl acetate (0.2 g) and K₂CO₃ (0.5 g) were added to a solution of 4-(4-Trifluoromethyl-phenyl)-1,2,3,6-tetrahydro-pyridine (0.2 g) in 5-Iodo-2-piperazin-1-yl-pyrimidine (10 mL). The resulting suspension was stirred at room temperature overnight. The reaction mixture was then filtered and concentrated to give {2-methyl-5-14-(4-trifluoromethyl-phenyl)-3,6-dihydro-2H-pyridine-1-sulfonyl]-phenyl}-acetic acid ethyl ester, which was used directly in the next step.

Step 3

{2-Methyl-5-[4-(4-trifluoromethyl-phenyl)-3,6-dihydro-2H-pyridine-1-sulfonyl]-phenyl}-acetic acid. The compound of Example 45 was synthesized from the compound of Step 2 according to the procedure described for the preparation of Example 1, Step 3. ¹H NMR (400 MHz, CDCl₃) δ ppm. 7.67 (m, 2H), 7.55 (d, 2H), 7.26 (m, 3H), 6.04 (s, 1H), 3.79 (d, 2H), 3.75 (s, 2H), 3.49 t, 2H), 2.61 (bt, 2H), 2.39 (s, 3H).

### EXAMPLE 46

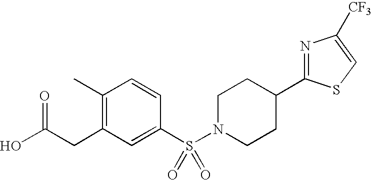

### {2-Methyl-5-[4-(4-trifluoromethyl-thiazol-2-yl)-piperidine-1-sulfonyl]-phenyl}-acetic acid

The compound of Example 46 was prepared following the procedure described for the compound of Example 43. ¹H NMR (400 MHz, CDCl₃) δ ppm. 7.64 (d, 1H) 7.63 (s, 1H), 7.52 (d, 1H), 7.14 (s, 1H), 3.91 (d, 2H), 3.76 (s, 2H), 2.79 (t, 1H), 2.47 (t, 2H), 2.41 (s, 3H), 2.13 (d, 2H), 1.86 (t, 2H). LCMS: 449.0 (M+1)⁺.

### EXAMPLE 47

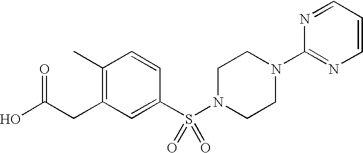

### {2-Methyl-5-[4-(pyrimid-2-yl)-piperidine-1-sulfonyl]-phenyl}-acetic acid

The compound of Example 47 was prepared following the procedure described for the preparation of Example 17. ¹H NMR (400 MHz, CDCl₃) δ ppm. 8.39 (bs, 2H), 7.68 (s, 1H), 7.63 (d, 1H), 7.40 (d, 1H), 7.22 (s, 1H), 6.60 (s, 1H), 4.19 (bs, 4H), 4.01 (s, 2H), 3.14 (sb, 4H), 2.44 (s, 3H). LCMS: 377.0 (M+1)⁺.

### EXAMPLE 48

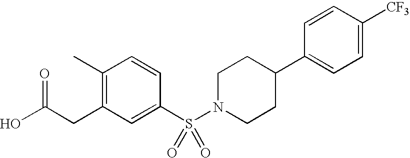

### {2-Methyl-5-[4-(4-trifluoromethyl-phenyl)-piperidine-1-sulfonyl]-phenyl}-acetic acid

Step 1

1-Methyl-4-trifluoromethyl-benzene VII-A-48: To a solution of p-trifluoromethylaniline (80.6 g) in concentrated HCl (152.1 g) and water (200 mL) cooled at 0° C. was added drop wise a solution of NaNO₂ (39.7 g) in water (90 mL) over a 30-minute period. The temperature was kept at 0-5° C. during the addition of NaNO₂ solution. After stirring at 0˜5° C. for an hour, the cold reaction mixture was filtered to remove an insoluble yellow solid. The filtrate was then treated with urea until KI-starch paper not turning blue, followed by adding an aqueous KI (124.5 g) solution over a 1˜1.5 hour period. The reaction mixture was stirred for an additional hour, decolorized by adding saturated NaHSO₃ solution, then extracted 3 times with petroleum ether. The combined petroleum ether solution was dried and concentrated. The residue was purified by column chromatography to give 75.7 g of intermediate VII-A48 as a red oil.

Step 2

Freshly activated Mg (prepared by washing successively with dilute HCl, acetone and ether, then dried at room temperature) (6 g) in THF (10 mL) was purged with nitrogen for 30 minutes, then added a small crystalline of iodine. To the mixture was added dropwise a solution of compound VII-A-48 (32.6 g) in anhydrous THF (100 mL) over a 1-hour period. The temperature was kept around 35˜38° C. during the addition. After stirring for an additional hour, added drop wise a solution of 1-benzyl-4-piperidone (25 g) in anhydrous THF (50 mL) over a 1-hour period. The temperature was kept around 35˜38° C. After stirring for an additional hour, the reaction was cooled in an ice-water bath and added drop wise aqueous saturated solution of NH₄Cl, followed by extraction with THF. The combined THF solution was dried and concentrated. The residue was purified by column chromatography to give 2.7 g of compound VII-B-48 as yellow solid.

Step 3

Concentrated HCl (40 mL) was added to a solution of compound VII-B-48 (7 g) in p-dioxane (10 mL). The mixture was then refluxed until the starting material was all consumed, about 4 hours. After cooling to room temperature, the mixture was treated with saturated Na₂CO₃ till pH 9, followed by extraction with EtOAc. The combined EtOAc solution was dried and concentrated. The residue was purified by column chromatography to give 3.8 g of compound VII-C-48 as a dark yellow solid.

Step 4

A solution of ethyl chloroformate (6.2 g) in THF was added dropwise to a cooled solution of compound VII-C-48 (9.2 g) in anhydrous THF (50 mL). The temperature was kept around −15˜−7° C. during the addition of ethyl chloroformate solution. After stirring at −7° C. for 3 h, the reaction mixture was concentrated in vacuo. The residue was dissolved in MeOH (100 mL) and refluxed for 2 hours. Removal of MeOH gave crude compound VII-D-48 as dark yellow solid that was used directly in next step reaction.

Step 5

A solution of crude compound VII-D-48 from above reaction in MeOH (50 mL) was added to a suspension of Pd/C (2.8 g) in MeOH (30 mL). The mixture was then treated with hydrogen at room temperature overnight. After filtering out the catalyst, the MeOH solution was concentrated to give crude compound VII-E-48 that was used directly in the next step reaction.

Steps 6 and 7

{2-Methyl-5-[4-(4-trifluoromethyl-phenyl)-piperidine-1-sulfonyl]-phenyl}-acetic acid. The compound of Example 48 was synthesized from VII-E-48 according to the method described for the preparation of Example 17, Steps 2 and 3. ¹H NMR (400 MHz, CDCl₃) δ ppm. 7.69 (s, 1H), 7.67 (d, 1H), 7.66 (d, 2H), 7.44 (d, 1H), 7.24 (s, 1H), 4.01 (d, 2H), 3.85 (s, 2H), 2.57 (m, 1H), 2.50(s, 3H), 2,41 (m, 2H), 1.94 (m, 4H). LCMS: 442.0 (M+1)⁺.

### EXAMPLE 49

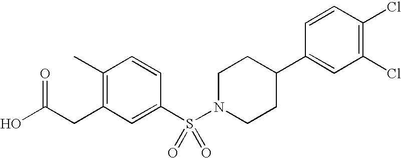

### {5-[4-(3,4-Dichloro-phenyl)-piperidine-1-sulfonyl]-2-methyl-phenyl)-acetic acid

Step 1

3,4-Dichloroaniline (15 g) was added to a stirred solution of concentrated H₂SO₄ (27.2 g) in water (350 mL). The mixture was heated to 80° C. and stirred at 80° C. for 10 minutes. The mixture was cooled to below 5° C., added drop wise a solution of NaNO₂ (6.4 g) in water (40 mL). It was stirred for an hour after the addition of NaNO₂, followed by addition drop wise a solution of KI (15.4 g) in water (40 mL). The mixture was stirred for an additional 30 minutes, then heated in a 40° C. water bath for another 30 minutes. The mixture was finally extracted with CH₂Cl₂. The combined CH₂Cl₂ solution was dried over CaCl₂ and concentrated. The residue was purified by column chromatography eluting with petroleum ether to give 20 g of compound X-A-49 in 79% yield.

Step 2

Ethyl chloroformate (7 g) was added drop wise to a stirred solution of 1-benzyl-4-piperidone (10 g) in benzene (60 mL) at 0° C. The mixture was allowed to warm up to room temperature and stirred overnight. The solution was filtered to remove insoluble solid. The filtrate was concentrated and purified by column chromatography. The column was first eluted with petroleum ether to remove benzene, followed with petroleum ether/5-Iodo-2-piperazin-1-yl-pyrimidine (9:2) to remove benzylchloride, and finally with diethyl ether to obtain 7 g of compound X-B-49.

Step 3

A 3M solution of n-BuLi in hexane (24 mL) was added to anhydrous THF (60 mL) at −78° C., followed by the addition of a solution of compound X-A-49 (15 g) in anhydrous THF (10 mL) dropwise. The mixture was stirred for an hour, then compound X-B-49 was added dropwise. The resulting mixture was stirred at −78° C. for an additional hour then allowed to warm up gradually to room temperature. After stirring at room temperature for 3 hours, the reaction was quenched by adding a saturated aqueous NH₄Cl solution dropwise. The separated organic layer was set aside. The aqueous was concentrated to remove most of the THF, then extracted with EtOAc (3×30 mL). The combined organic solution was dried over Na2SO4 and concentrated. The residue was purified by column chromatography eluting with petroleum ether/EtOAc (5:1) to give 9.5 g of compound X-C-49 in 54% yield.

Step 4

AlCl₃ (19.5 g) was added to a solution of Et₃SiH (25 g) in DCM (46 mL) at 0° C. The mixture was stirred at 0° C. for 10 minutes, followed by the drop wise addition of a solution of compound X-C-49 (9.2 g) in 5-Iodo-2-piperazin-1-yl-pyrimidine (184 mL). After stirring at 0° C. for an additional hour, the cooling bath was removed and stirring was continued at room temperature overnight. The reaction mixture was poured into saturated aqueous Na₂CO₃, then filtered through Celite. The filtrate was extracted with 5-Iodo-2-piperazin-1-yl-pyrimidine. The combined DCM solution was dried over anhydrous K₂CO₃ and concentrated. The residue was purified by column chromatography eluting with 5-Iodo-2-piperazin-1-yl-pyrimidine/MeOH/NH₄OH (250:32:2) to give compound X-D-49.

Step 5

{5-[4-(3,4-Dichloro-phenyl)-piperidine-1-sulfonyl]-2-methyl-phenyl}-acetic acid. The compound of Example 49 was synthesized from X-D-49 according to the method described for the preparation of Example 17, Steps 2 and 3. ¹H NMR (400 MHz, CDCl₃) δ ppm. 7.63 (s, 1H), 7.60 (d, 1H), 7.38 (d, 2H), 7.20 (s, 1H), 6.95 (d, 1H), 3.94 (d, 2H), 3.76 (s, 2H), 2.41 (s, 3H), 2.34 (m, 2H), 1.86 (t, 2H), 1.73 (t, 2H). LCMS: 442.0 (M+1)⁺.

### EXAMPLE 50

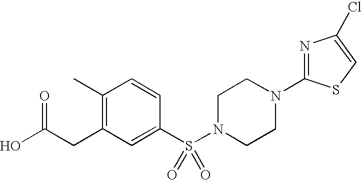

### {5-[4-(4-Chloro-thiazol-2-yl)-piperazine-1-sulfonyl]-2-methyl-phenyl}-acetic acid

Step 1

Synthesis of 2,4-Dichloro-thiazole: A mixture of thiazole-2,4-dione (25 g), POCl₃ (130 mL) and freshly distilled pyridine (17 mL) were heated at 120° C. for 3 hours. After cooling to room temperature, excess POCl₃ was removed under reduced pressure. The residue was poured into ice/water, and extracted with ether. The combined ether solution was washed with aqueous 5% NaOH, water, then dried. Removal of solvent gave the desired intermediate in 70% yield.

Step 2

Synthesis of (5-[4-(4-Chloro-thiazol-2-yl)-piperazine-1-sulfonyl]-2-methyl-phenyl}-acetic acid. The compound of Example 50 was prepared from the intermediate from Step I following the procedure outlined for the preparation of Example 17. ¹H NMR (400 MHz, CDCl₃) δ ppm. 7.59 (s, 1H), 7.56 (d, 1H), 7.42 (d, 1H), 6.77 (s, 1H), 3.73 (s, 2H), 3.47 (t, 4H), 2.97 (t, 4H), 2.29 (s, 3H). LCMS: 416.0 (M+1)⁺.

### EXAMPLE 51

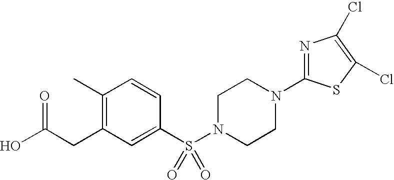

### {5-[4-(4,5-Dichloro-thiazol-2-yl)-piperazine-1-sulfonyl]-2-methyl-phenyl}-acetic acid

The compound of Example 51 was prepared following the procedure for the compound of Example 50. ¹H NMR (400 MHz, CDCl₃) δ ppm. 7.59 (s, 1H), 7.58 (s, 1H), 7.53 (d, 1H), 7.43 (d, 1H), 4.04 (s, 2H), 3.44 (t, 4H), 2.98 (t, 4H), 2.48 (s, 3H). LCMS: 450.0 (M+1)⁺.

### EXAMPLE 52

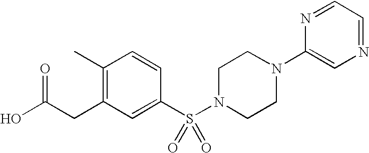

### [2-Methyl-5-(4-pyrimidin-2-yl-piperazine-1-sulfonyl)-phenyl]-acetic acid

The compound of Example 52 was prepared following the procedure for the compound of Example 17. ¹H NMR (400 MHz, CDCl₃) δ ppm. 8.18 (bs, 2H), 7.82 (s, 1H), 7.59 (s, 1H), 7.57 (d, 1H), 7.36 (d, 1H), 3.80 (s, 2H), 3.72 (t, 4H), 3.10 (t, 4H), 2.33 (s, 3H). LCMS: 377.0 (M+1)⁺.

### EXAMPLE 53

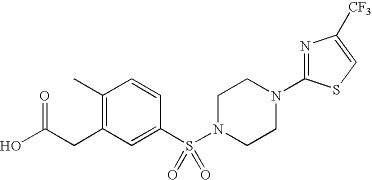

### {2-Methyl-5-[4-(4-trifluoromethyl-thiazol-2-yl)-piperazine-1-sulfonyl]-phenyl}-acetic acid

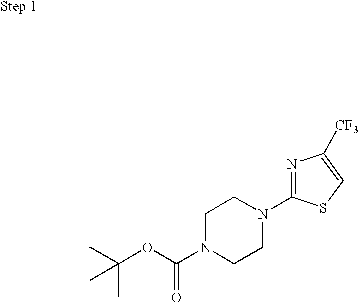

4-(4-Trifluoromethyl-thiazol-2-yl)-piperazine-1-carboxylic acid tert-butyl ester: A mixture of 4-thiocarbamoyl-piperazine-1-carboxylic acid tert-butyl ester (0.2 g), 1,1,1-trifluoro-3-bromo-acetone (0.19 g) and triethylamine (0.33 g) in xylene (20 mL) were refluxed overnight. After cooling to room temperature, the solution was concentrated and purified by column chromatography to give 0.3 g of the desired intermediate as yellow oil.

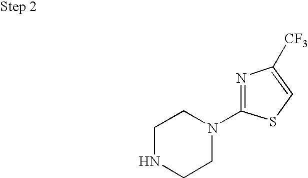

1-(4-Trifluoromethyl-thiazol-2-yl)-piperazine: The intermediate from step 1 (0.5 g) was stirred in a mixture of TFA (10 mL) and CH₂Cl₂ (40 mL) at room temperature for 2 hours, then concentrated. To remove remaining TFA, the residue was re-dissolved in CH₂Cl₂ (50 mL) and concentrated again to give 0.3 g of intermediate 1-(4-Trifluoromethyl-thiazol-2-yl)-piperazine as a light yellow oil.

Step 3

{2-Methyl-5-[4-(4-trifluoromethyl-thiazol-2-yl)-piperazine-1-sulfonyl]-phenyl}-acetic acid. The compound of Example 53 was synthesized from 1-(4-Trifluoromethyl-thiazol-2-yl)-piperazine according to the method described for the preparation of Example 17. ¹H NMR (400 MHz, CDCl₃) δ ppm. 7.59 (s, 1H), 7.45 (m, 1H), 7.43 (m, 1H), 7.30 (m, 1H), 3.55 (t, 4H), 3.04 (t, 4H), 2.68 (s, 2H), 2.34 (s, 3H).

### EXAMPLE 54

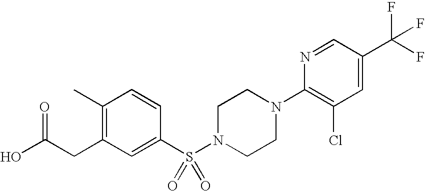

### {5-[4-(3-Chloro-5-trifluoromethyl-pyridin-2-yl)-piperazine-1-sulfonyl]-2-methyl-phenyl}-acetic acid

The compound of Example 54 was prepared following the method described for the preparation of the compound of Example 17. ¹H NMR (400 MHz, CDCl₃) δ ppm. 8.50 (s, 1H), 8.13 (s, 1H), 7.59 (s, 1H), 7.54 (d, 1H), 7.43 (d, 1H), 3.73 (s, 2H), 3.48 (t, 4H), 3.02 (t, 4H), 2.31 (s, 3H). LCMS: 480.0 (M+1)⁺.

### EXAMPLE 55

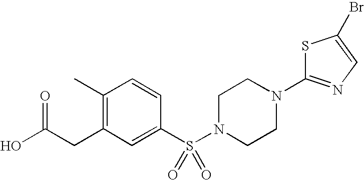

### {5-[4-(5-Bromo-thiazol-2-yl)-piperazine-1-sulfonyl]-2-methyl-phenyl}-acetic acid

The compound of Example 55 was synthesized according to the method described for the preparation of Example 53. ¹H NMR (400 MHz, CDCl₃) δ ppm. 7.61 (s, 1H), 7.58 (d, 1H), 7.42 (d, 1H), 7.20(s, 1H), 3.76 (s, 2H), 3.43 (t, 4H), 3.00 (t, 4H), 2.31 (s, 3H). LCMS: 460.0 (M+1)⁺.

### EXAMPLE 56

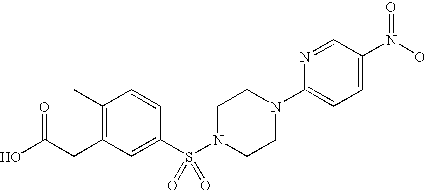

### {2-Methyl-5-[4-(5-nitro-pyridin-2-yl)-piperazine-1-sulfonyl]-phenyl}-acetic acid

The compound of Example 56 was synthesized following the procedure described for the preparation of Example 17. ¹H NMR (400 MHz, CDCl₃) δ ppm. 9.01 (s, 1H), 8.23 (d, 1H), 7.62 (s, 1H), 7.61 (d, 1H), 7.39 (d, 1H), 6.54 (d, 1H), 3.90 (t, 4H), 3.76 (s, 2H), 3.15 (t, 4H), 2.41 (s, 3H). LCMS: 421.0 (M+1 )⁺.

### EXAMPLE 57

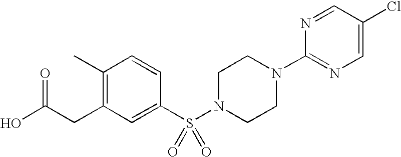

### {5-[4-(5-Chloro-pyrimidin-2-yl)-piperazine-1-sulfonyl]-2-methyl-phenyl}-acetic acid

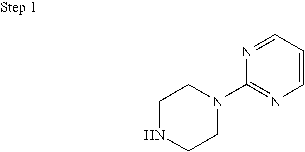

2-Piperazin-1-yl-pyrimidine was synthesized according to the method described for the preparation of Intermediate IV-A-17 in Example 17, Step 1.

Step 2

A solution of acetic anhydride (28.5 g) in CH₂Cl₂ (80 mL) was added drop wise to a solution of compound XII-A-57 (30 g) in CH₂Cl₂ (150 mL). The resulting mixture was stirred for 1 hour, followed by addition of a solution of triethylamine (28 g) in CH₂Cl₂ (80 mL). The mixture was stirred for an additional hour before washing three times with brine. The organic layer was dried and concentrated to give 36.1 g of yellow solid XII-B-57.

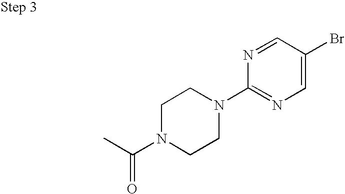

1-[4-(5-Bromo-pyrimidin-2-yl)-piperazin-1-yl]-ethanone XII-C-57: A solution of ]-(4-Pyrimidin-2-yl-piperazin-1-yl)-ethanone XII-B-57 (4.1 g) in acetic acid (10 mL) was heated to 90° C. for 30 minutes. To the reaction solution was added to a solution of bromine (3.4 g) in acetic anhydride (5 mL). The reaction flask was covered to avoid light and temperature was kept at 85-90° C. during the addition of bromine. The reaction mixture was stirred at 85-90° C. for an additional 3 hours. After cooling to room temperature, the separated solid was filtered and washed with petroleum ether. 3.8 g of yellowish brown solid XII-C-57 was obtained.

Step 4

A mixture of compound XII-C-57 (1.2 g), concentrated hydrochloric acid (15 mL) and water (15 mL) was heated to reflux overnight. After cooling to room temperature, the solution was neutralized with aqueous sodium hydroxide and extracted with ethyl acetate. The combined ethyl acetate solution was dried and concentrated to give 0.7 g of light yellow solid XII-D-57.

Step 5, 6

The compound of Example 57 was synthesized from XII-D-57 according to the method described for preparing Example 1, Step 2, 3. ¹H NMR (400 MHz, CDCl₃) δ ppm. 8.21 (s, 1H), 7.60 (d, 1H), 7.37 (.d, 1H), 7.00 (s, 1H), 3.93 (t, 4H), 3.75 (s, 2H), 3.08 (t, 4H), 2.41 (s, 3H). LCMS: 411.0 (M+1)⁺.

### EXAMPLE 58

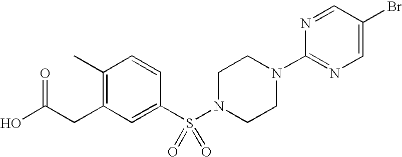

### {5-[4-(5-Bromo-pyrimidin-2-yl)-piperazine-1-sulfonyl]-2-methyl-phenyl}-acetic acid

The compound Example 58 was synthesized according the method described for the preparation of Example 57. The requisite intermediate XII-C-58 was prepared follows:

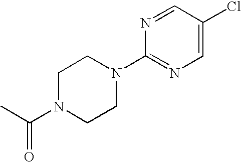

1-[4-(5-Chloro-pyrimidin-2-yl)-piperazin-1-yl]-ethanone XII-C-58: A mixture of 1-(4-Pyrimidin-2-yl-piperazin-1-yl)-ethanone XII-B-58 (2.0 g) and NCS (1.3 g) in CCl₄ (50 mL) was heated to reflux overnight. The flask was shielded from light to minimize free radical side reactions. After cooling to room temperature, the solution was washed with saturated brine and dried. Removal of solvent gave 2.0 g of compound XII-C-58.

### {5-[4-(5-Bromo-pyrimidin-2-yl)-piperazine-1-sulfonyl]-2-methyl-phenyl}-acetic Acid

¹H NMR (400 MHz, CDCl₃) δ ppm. 8.42 (s, 2H), 7.71 (s, 1H), 7.52 (d, 1H), 7.40 (d, 1H), 4.00 (t, 4H), 3.71 (s, 2H), 2.90 (t, 4H), 2.28 (s, 3H).

### EXAMPLE 59

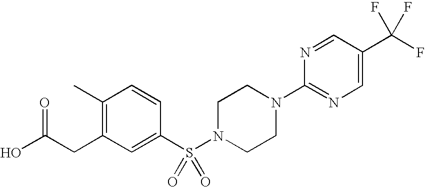

### {2-Methyl-5-[4-(5-trifluoromethyl-pyrimidin-2-yl)-piperazine-1-sulfonyl]-phenyl}-acetic acid

The compound of Example 59 was synthesized according to Scheme XIII.

Step 1

5-Methyl (1H,3H)-pyrimidine-2,4-dione (3 g) was refluxed in POCl₃ (20 mL) for 3 hours. After cooling to room temperature, it was poured into ice/water and extracted with CH₂Cl₂. The combined CH₂Cl₂ was dried and concentrated to give 2.3 g of crude product that was further purified by column chromatography, eluting with petroleum ether/EtOAc (10:1) to afford 2 g of compound XIII-A-59.

Step 2

A solution of concentrated NH₄OH (4.4 mL) in water (20 mL) was added to a suspension of compound XIII-A-59 (2 g) and Zn (2.4 g) in benzene (8 mL). The mixture was heated to reflux overnight. After cooling to room temperature, the solution was filtered and extracted twice with ether. The combined ether solution was dried and concentrated to give 1.0 g of crude product that was more than 90% pure thus used directly in the next step.

Step 3

HCl gas was bubbled through a solution of compound XIII-B-59 (2.0 g) in CCl₄ (250 mL) until there was solid precipitated out of the solution, followed by addition of SO₂Cl₂ (20 mL). The mixture was then refluxed for 72 hours under radiation of a 250 W high-pressure mercury lamp. After cooling to room temperature, the solution was filtered and concentrated. The residue was purified by column chromatography, eluting with petroleum ether/EtOAc (20-10:1) to give 0.6 g of compound XII-C-59.

Step 4

Under a nitrogen atmosphere, compound XIII-C-59 (1.0 g) and SbF₅ were mixed in a sealed tube and then heated slowly to 150° C. for 15 minutes. After cooling to room temperature, the reaction mixture was poured into ice followed by extraction with ether. The combined ether solution was washed with water and aqueous NaHCO₃. Removal of solvent gave 0.3 g of crude compound XIII-D-59.

Step 5

{2-Methyl-5-[4-(5-trifluoromethyl-pyrimidin-2-yl)-piperazine-1-sulfonyl]-phenyl}-acetic acid. The compound of Example 59 was synthesized from XIII-D-59 according to the preparation of Example 17, Steps 2 and 3. ¹H NMR (400 MHz, DMSO-d6) δ ppm. 8.69 (s, 2H), 7.59 (s, 1H), 7.52 (d, 1H), 7.41 (d, 1H), 3.93 (t, 4H), 3.73 (s, 2H), 2.97 (t, 4H), 2.29 (s, 3H). LCMS: 445.0(M+1)⁺.

### EXAMPLE 61

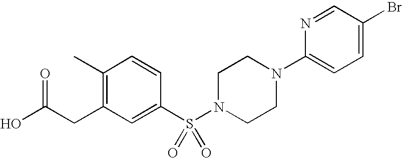

### {5-[4-(5-Bromo-pyridin-2-yl)-piperazine-1-sulfonyl]-2-methyl-phenyl}-acetic acid

The compound of Example 61 was synthesized according to Scheme VIII.

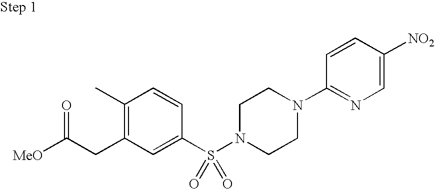

{2-Methyl-5-[4-(5-nitro-pyridin-2-yl)-piperazine-1-sulfonyl]-phenyl}-acetic acid methyl ester VIII-C-61 was synthesized following the procedure for Example 17.

Step 2

A mixture of compound VIII-C-61 (5.0 g), Fe (2.3 g) and NH₄Cl (3.1 g) in water (40 mL) and MeOH (110 mL) was heated to reflux. The hot reaction mixture was filtered. The insoluble solid residue was washed with hot MeOH. The combined MeOH solution was evaporated. The resulting black residue was dissolved in chloroform and refluxed with activated charcoal for 15 minutes. Removing the charcoal gave a red solution that was concentrated and purified by column chromatography to afford 2.2 g of compound VIII-D-61.

Step 3

A solution of NaNO₂ (0.5 g) in water (2 mL) was added dropwise to a suspension of compound VIII-D-61 (3.0 g) dilute H₂SO₄ (2 mL) at −3° C. The mixture was stirred for 20 min. The diazonium solution was then added dropwise to a solution of CuBr (1.27 g) in HBr (3 mL) preheated at 60° C. The mixture was stirred at 50˜60° C. for 40 min. After cooling to room temperature, the reaction mixture was extracted with CH₂Cl₂. The combined CH₂Cl₂ solution was dried and concentrated to give 0.3 g of VIII-E-61.

Step 4

{5-[4-(5-Bromo-pyridin-2-yl)-piperazine-1-sulfonyl]-2-methyl-phenyl}-acetic acid. The compound of Example 61 was synthesized from VIII-E-61 according to the procedure described for Example 1, Step 3. LCMS: 456.0 (M+1)⁺.

### EXAMPLE 62

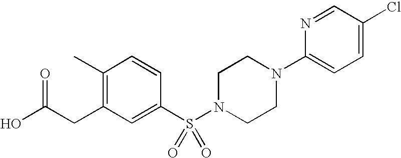

### {5-[4-(5-Chloro-pyridin-2-yl)-piperazine-1-sulfonyl]-2-methyl-phenyl}-acetic acid

The compound of Example 62 was synthesized according to the procedure described for Example 61. ¹H NMR (400 MHz, CDCl₃) δ ppm. 8.05 (s, 1H), 7.57 (s, 1H), 7.53 (d, 2H), 7.41 (d, 1H), 6.81 (d, 1H), 4.26 (s, 2H), 3.72 (t, 4H), 2.92 (t, 4H), 2.30 (s, 3H). LCMS: 410.0 (M+1)⁺.

### EXAMPLE 63

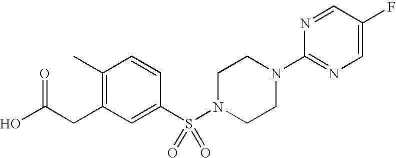

### {5-[4-(5-Fluoro-pyrimidin-2-yl)-piperazine-1-sulfonyl]-2-methyl-phenyl}-acetic acid

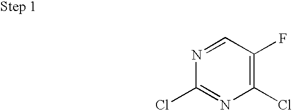

Dichloro-5-fluoro-pyrimidine: A mixture of 5-fluoro-pyrimidine-2,4-diol (5.2 g, 0.04 mol), Et₃N.HCl (1.65 g, 0.012 mol) and POCl₃ (21.5 g, 0.14 mol) was heated to reflux for 3 hours. After cooling down to about 30˜40° C., a solution of PCl₅ (20.85 g, 0.1 mol) in POCl₃ (8 mL) was added drop wise to the reaction mixture over a 1-hour period. The temperature was kept around 50° C. during the addition of PCl₅/POCl₃. The mixture was stirred for an additional hour at 50-60° C. POCl₃ was then removed under reduced pressure. The residue was diluted with EtOAc (25 mL) and heated to reflux for 15 minutes, filtered to remove insoluble solid. The filtrate was evaporated and purified by column chromatography to give 3.1 g of 2,4-dichloro-5-fluoro-pyrimidine as colorless oil that become colorless crystalline upon standing at below 25° C.

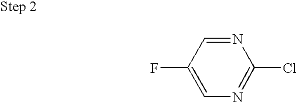

2-Chloro-5-fluoro-pyrimidine: A solution of HOAc (2.4 g, 0.04 mol) in THF (15 mL) was added drop wise to a refluxing mixture of dichloro-5-fluoro-pyrimidine (3.34 g, 0.02 mol) and Zn (7.8 g, 0.02 mol) in THF (40 mL) over a 1-hour period. The mixture was refluxed for another 9 h. After cooling to room temperature, the solution was filtered to remove an insoluble solid. The solution containing 2-chloro-5-fluoro-pyrimidine was used directly in the next step reaction.

Step 3

{5-[4-5-Fluoro-pyrimidin-2-yl)-piperazine-1-sulfonyl]-2-methyl-phenyl}-acetic acid. The compound of Example 63 was synthesized from the intermediate of Step 2 according to the procedure described for Example 17, Steps 2 and 3. ¹H NMR (400 MHz, CDCl₃) δ ppm. 8.17 (s, 2H), 7.61 (s, 1H), 7.56 (d, 1H), 7.34 (d, 1H), 3.89 (t, 4H), 3.72 (s, 2H), 3.08 (t, 4H), 2.38 (s, 3H).

### EXAMPLE 64

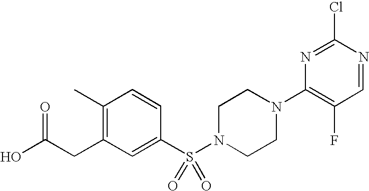

### {5-[4-(2-Chloro-5-fluoro-pyrimidin-4-yl)-piperazine-1-sulfonyl]-2-methyl-phenyl}-acetic acid

The compound of Example 64 was synthesized following the procedure for Example 63. ¹H NMR (400 MHz, CDCl₃) δ ppm. 7.96 (s, 1H), 7.62 (s, 1H), 7.60 (d, 1H), 7.40 (d, 1H), 3.94(t, 4H), 3.77 (s, 2H), 3.15 (t, 4H), 2.42 (s, 3H).

### EXAMPLE 66

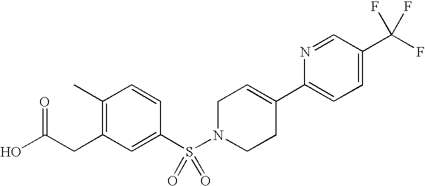

### [2-Methyl-5-(5-trifluoromethyl-3′,6′-dihydro-2¹H-[2,4′]bipyridinyl-1′-sulfonyl)-phenyl]-acetic acid

The compound of Example 66 was synthesized according to the method described for the preparation of Example 45. ¹H NMR (400 MHz, CDCl₃) δ ppm. 8.82 (s, 1H), 7.93 (d, 1H), 7.68 (s, 1H), 7.66 (d, 1H), 7.48 (d, 1H), 7.38 (d, 1H), 6.68 (s, 1H), 3.90 (s, 2H), 3.76 (s, 2H), 3.40 (t, 2H), 2.74 (s, 2H), 2.41 (s, 3H). LCMS: 441.0 (M+1)⁺.

### EXAMPLE 67

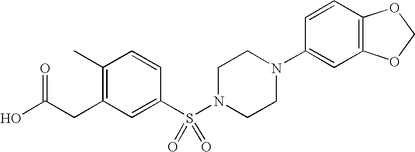

### [5-(4-(Benzo[1,3]dioxol-4-yl)-piperazine-1-sulfonyl)-2-methyl-phenyl]-acetic acid

The compound of Example 67 was synthesized according to the procedure outlined for Example 17. ¹H NMR, (400 MHz, MeOH-D₄) δ 7.60 (s, 1H), 7.55 (dd, 1H), 7.42 (d, 1H), 6.78 (s, 1H), 6.73 (s, 2H), 5.91 (s, 2H), 3.51 (s, 2H), 3.05-3.00 (m, 4H), 2.59-2.57 (m, 4H), 2.40 (s, 3H).

### EXAMPLE 68

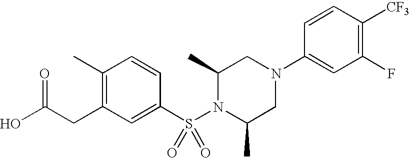

### {5-[4-(3-Fluoro-4-trifluoromethyl-phenyl)-2,6-dimethyl-piperazine-1-sulfonyl]-2-methyl-phenyl}-acetic acid

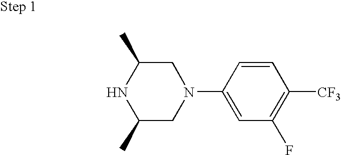

1-(3-Fluoro-4-trifluoromethyl-phenyl)-3,5-dimethyl-piperazine. The compound 1-(3-Fluoro-4-trifluoromethyl-phenyl)-3,5-dimethyl-piperazine was synthesized according to the procedure in Example 29, Step 3 starting with cis-2,6-dimethyl piperazine. ¹H NMR (400 MHz, CDCl₃) δ 7.38 (m, 1H), 6.64-6.57 (m, 2H), 3.57 (dd, 2H), 3.02-2.94 (m, 2H), 2.42-2.36 (m, 2H), 1.14 (d, 6H); LCMS 277.4 (M+1)⁺.

Step 2

{5-[4-(3-Fluoro-4-trifluoromethyl-phenyl)-2,6-dimethyl-piperazine-1-sulfonyl]-2-methyl-phenyl}-acetic acid. The compound of Example 68 was synthesized from the product of Step 1 according to the procedure outlined for Example 19 (Steps 2 and 3). ¹H NMR (400 MHz, MeOH-D₄) δ 7.71 (s, 1H), 7.64-7.62 (m, 1H), 7.38-7.30 (m, 2H), 6.62 (s, 1H), 6.59 (d, 1H), 4.25-4.15 (m, 2H), 3.71 (s, 2H), 3.46 (d, 2H), 3.90 (dd, 2H), 2.33 (s, 3H), 1.40 (d, 6H).

### EXAMPLE 69

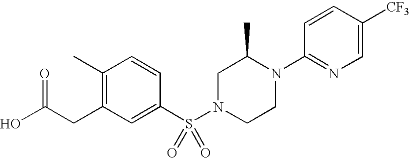

### {2-Methyl-5-[(R)-3-methyl-4-(5-trifluoromethyl-pyridin-2-yl)-piperazine-1-sulfonyl]-phenyl}-acetic acid

Example 69 is a single enantiomer of Example 23. It was synthesized from (R)-2-Methylpiperazine followed the same procedure and showed identical ¹H NMR data.

### EXAMPLE 70

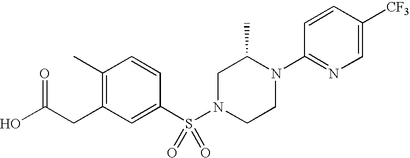

### {2-Methyl-5-[(S)-3-methyl-4-(5-trifluoromethyl-pyridin-2-yl)-piperazine-1-sulfonyl]-phenyl}-acetic acid

Example 70 is the enantiomer of Example 69. It was synthesized from (S)-2-Methylpiperazine followed the same procedure and showed identical ¹H NMR data.

### EXAMPLE 72

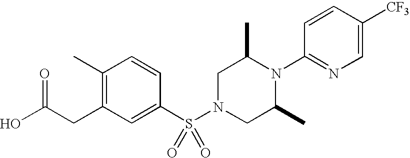

### {2-Methyl-5-[3,5-dimethyl-4-(5-trifluoromethyl-pyridin-2-yl)-piperazine-1-sulfonyl]-phenyl}-acetic acid

The compound of Example 72 was synthesized following the procedure for Example 23. ¹H NMR (400 MHz, CDCl₃), δ (ppm): 8.39 (s, 1H), 7.59 (m, 2H), 7.33 (d, 1H), 7.24 (m, 1H), 6.51 (d, 1H), 4.54 (b, 2H), 3.66 (d, 2H), 3.60 (s, 2H), 2.50 (dd, 2H), 2.37 (s, 3H), 1.37 (d, 6H).

### EXAMPLE 73

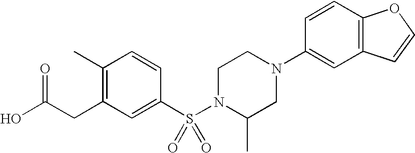

### [5-(4-Benzofuran-5-yl-2-methyl-piperazine-1-sulfonyl)-2-methyl-phenyl]-acetic acid

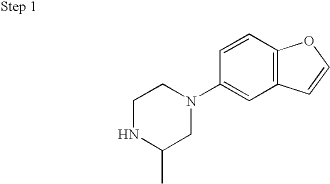

1-Benzofuran-5-yl-3-methyl-piperazine. To a solution of 5-bromobenzofuran (250 mg, 1.27 mmol, 1.0 equiv.) and 2-methylpiperazine (508.4 mg, 5.08 mmol, 4.0 equiv.) in toluene (7 mL) was added PdCl₂[P(o-Tol)₃]₂ (30 mg, 0.04 mmol, 0.04 equiv.) followed by sodium tert-butoxide (183 mg, 1.91 mmol, 1.5 equiv.). The resulting mixture was heated to 100° C. with stirring under nitrogen. After stirred at same temperature for 16 hours, the reaction mixture was cooled to room temperature and then diluted with ethyl acetate (100 mL). The resulting solution was washed with water, brine and then dried over Na₂SO₄. After removal of solvent, the crude product was purified by chromatography to give 132 mg (48% yield) of 1-Benzofuran-5-yl-3-methyl-piperazine. ¹H NMR (400 MHz, CDCl₃), δ (ppm): 7.56 (d, 1H), 7.39(d, 1H), 7.10 (d, 1H), 7.00 (dd, 1H), 6.69 (m, 1H), 3.44 (d, 2H), 306 (m, 3H), 2.72 (dt, 1H), 2.38 (d, 1H), 1.14 (d, 3H).

Step 2

[5-(4-Benzofuran-5-yl-2-methyl-piperazine-1-sulfonyl)-2-methyl-phenyl]-acetic acid. The compound of Example 73 was synthesized from 1-Benzofuran-5-yl-3-methyl-piperazine according to the method described for the preparation of Example 17 in Steps 2 and 3. ¹H NMR (400 MHz, CDCl₃), δ (ppm): 7.69 (s, 1H), 7.66 (dd, 1H), 7.57 (d, 1H), 7.36 (d, 1H), 7.31 (d, 1H), 7.01 (d, 1H), 6.87 (dd, 1H), 6.67 (dd, 1H), 4.21 (m, 1H), 3.74 (d, 1H), 3.71 (s, 2H), 3.34 (m, 2H), 3.18 (d, 1H), 2.87 (dd, 1H), 2.74 (dt, 1H), 2.37 (s, 3H), 1.25 (d, 3H).

### EXAMPLE 74

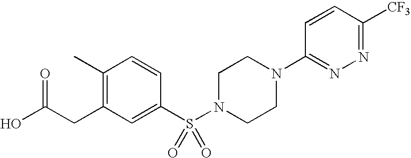 Step 1

A mixture of 3,6-dichloropyridazine (10 g, 67 mmol), sodium iodide (13.5 g, 90 mmol), and 45% aq. H1 (60 mmol) was stirred at 40° C. for 4 h. The reaction mixture was cooled to room temperature and poured into cold NaOH solution. The mixture (pH>9) was stirred for 10 min and extracted with (100 mL×3). The combined organic solution was washed with brine, dried and concentrated in vacuo to give 6-chloro-3-iodopyridazine 13.6 g, 85%.

Step 2

A mixture of 6-chloro-3-iodopyridazine (12.0 g, 50 mmol), ethyl chlorodifluoromethyl acetate (45 g, 280 mmol), KF (168 g, 290 mmol), CuI (14.4 g, 76 mmol) in DMF (600 mL) was stirred at 120° C. for 5 h. The mixture was cooled to room temperature and concentrated in vacuo. The residue was dissolved in CH₂Cl₂ (500 mL) and washed with brine. The solution was concentrated in vacuo and the residue was purified by column chromatography to afford 3-chloro-6-trifluoromethylpyridazine, 2.9 g.

Step 3

The compound of Step 3 was prepared from 3-chloro-6-trifluoromethylpyridazine according to the method described for Example 44, Step 1 to afford 3-piperazin-1-yl-6-trifluoromethylpyridazine.

Step 4

The compound of Example 74 was prepared from 3-piperazin-1-yl-6-trifluoromethylpyridazine according to the method described for Example 1, Steps 2 and 3. ¹H NMR (4.00 MHz, DMSO-₆), δ (ppm): 7.79 (d, 1H), 7.60 (s, 1H), 7.54 (d, 1H), 7.43 (d, 1H), 7.36 (d, 1H), 3.81 (t, 4H), 3.72 (s, 2H), 3.00 (t, 4H), 2.3 (s, 3H).

### EXAMPLE 75

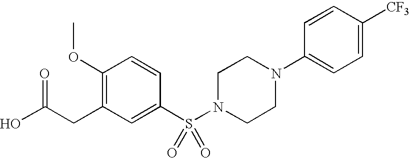

### {2-Methoxy-5-[4-(4-trifluoromethyl-phenyl)-piperazine-1-sulfonyl]-phenyl}-acetic acid

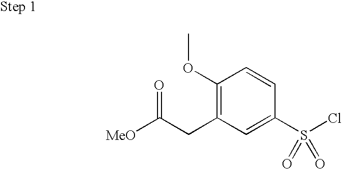

(5-Chlorosulfonyl-2-methoxy-phenyl)-acetic acid methyl ester: ClSO₃H (10 mL, 150 mmol, 10 equiv.) was cooled to 0° C. To this cold chlorosulfonic acid was added (2-Methoxy-phenyl)-acetic acid methyl ester (2.7 g, 15 mmol, 1.0 equiv.) drop wise with stirring at same temperature. After removal of cooling-bath, the reaction mixture was stirred at room temperature for 1 hour. The reaction mixture was slowly poured into ice water and then extracted with ethyl acetate (125 mL×2). The combined organic layers were washed with brine and dried over Na₂SO₄. After removal of solvent, 3.93 g (94% yield) of the desired intermediate was obtained, which was used without purification in next step. ¹H NMR (400 MHz, CDCl₃), δ (ppm): 7.97 (dd, 1H), 7.86 (d, 1H), 7.02 (d, 1H), 3.93 (s, 3H), 3.72 (s, 3H), 3.70 (s, 2H).

Step 2

{2-Methoxy-5-[4-(4-trifluoromethyl-phenyl)-piperazine-1-sulfonyl]-phenyl}-acetic acid: The compound of Example 75 was synthesized from the intermediate of Step 1 according to the method described for the preparation of Example 3 (Steps 3 and 4). ¹H NMR (400 MHz, CDCl₃), δ (ppm): 7.72 (dd, 1H), 7.62 (d, 1H), 7.46 (d, 2H), 6.99 (d, 1H), 6.86 (d, 2H), 3.90 (s, 3H), 3.71 (s, 2H), 3.33 (m, 4H), 3.15 (m, 4H).

### EXAMPLE 76

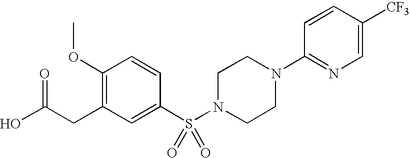

### {2-Methoxy-5-[4-(5-trifluoromethyl-pyridin-2-yl)-piperazine-1-sulfonyl]-phenyl}-acetic acid

The compound of Example 76 was synthesized according to the method described for the preparation of Example 75. ¹H NMR (400 MHz, CDCl₃), δ (ppm): 8.34 (d, 1H), 7.70 (dd, 1H), 7.60 (m, 2H), 6.97 (d, 1H), 6.59 (d, 1H), 3.89 (s, 3H), 3.74 (m, 4H), 3.69 (s, 2H), 3.09 (m, 4H).

### EXAMPLE 77

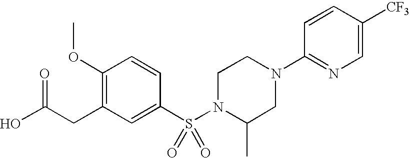

### {2-Methoxy-5-[2-methyl-4-(5-trifluoromethyl-pyridin-2-yl)-piperazine-1-sulfonyl]-phenyl}-acetic acid

The compound of Example 77 was synthesized according to the method described for the preparation of Example 19 using (5-Chlorosulfonyl-2-methoxy-phenyl)-acetic acid methyl ester. ¹H NMR (400 MHz, CDCl₃), δ (ppm): 8.33 (d, 1H), 7.75 (dd, 1H), 7.67 (d, 1H), 7.58 (dd, 1H), 6.91 (d, 1H), 6.51 (d, 1H), 4.21 (m, 1H), 4.16 (m, 1H), 3.98 (m, 1H), 3.87 (s, 3H), 3.71 (m, 1H), 3.68 (s, 2H), 3.27 (m, 2H), 3.01 (dt, 1H), 1.09 (d, 3H).

### EXAMPLE 78

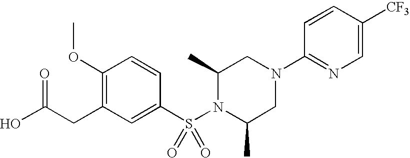

### {5-[2,6-Dimethyl-4-(5-trifluoromethyl-pyridin-2-yl)-piperazine-1-sulfonyl]-2-methoxy-phenyl}-acetic acid

The compound of Example 78 was synthesized according to the method described for the preparation of Example 20 using (5-Chlorosulfonyl-2-methoxy-phenyl)-acetic acid methyl ester. ¹H NMR (400 MHz, CDCl₃), δ (ppm): 8.31 (m, 1H), 7.75 (dd, 1H), 7.68 (d, 1H), 7.56 (dd, 1H), 6.88 (d, 1H), 6.48 (d, 1H), 4.19 (m, 2H), 3.95 (md, 2H), 3.86 (s, 3H), 3.67 s, 2H), 3.05 (dd, 2H), 1.36 (d, 6H).

### EXAMPLE 79

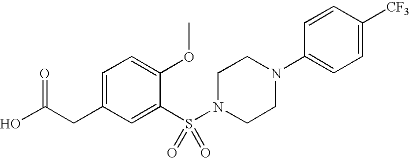

### {4-Methoxy-3-[4-(4-trifluoromethyl-phenyl)-piperazine-1-sulfonyl]-phenyl}-acetic acid

The compound of Example 79 was synthesized according to the method described for the preparation of Example 75 using (3-Chlorosulfonyl4-methoxy-phenyl)-acetic acid methyl ester. ¹H NMR (400 MHz, CDCl₃), δ (ppm): 7.83 (d, 1H), 7.48 (m, 3H), 7.00 (d, 1H), 6.91 (d, 2H), 3.93 (s, 3H), 3.66 (s, 2H), 3.39 (m, 4H), 3.32 (m, 4H).

### EXAMPLE 80

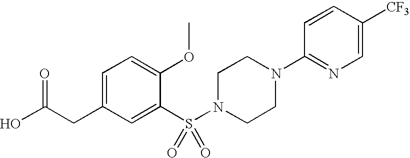

### {(4-Methoxy-3-[4-(5-trifluoromethyl-pyridin-2-yl)-piperazine-1-sulfonyl]-phenyl}-acetic acid

The compound of Example 80 was synthesized according to the method described for the preparation of Example 76 using (3-Chlorosulfonyl-4-methoxy-phenyl)-acetic acid methyl ester. ¹H NMR (400 MHz, CDCl₃), δ (ppm): 8.37 (d, 1H), 7.81 (d, 1H), 7.63 (dd, 1H), 7.46 (dd, 1H), 6.98 (d, 1H), 6.64 (d, 1H), 3.90 (s, 3H), 3.72 (m, 4H), 3.64 (s, 2H), 3.34 (m, 4H).

### EXAMPLE 81

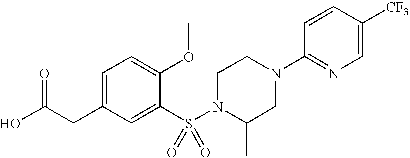

### {4-Methoxy-3-[2-methyl-4-(5-trifluoromethyl-pyridin-2-yl)-piperazine-1-sulfonyl]-phenyl}-acetic acid

The compound of Example 81 was synthesized according to the method described for the preparation of Example 77 using (3-Chlorosulfonyl-4-methoxy-phenyl)-acetic acid methyl ester. ¹H NMR (400 MHz, CDCl₃), δ (ppm): 8.36 (d, 1H), 7.86 (d, 1H), 7.61 (dd, 1H), 7.44 (dd, 1H), 6.95 (d, 1H), 6.58 (d, 1H), 4.26 (m, 2H), 4.08 (d, 1H), 3.91 (s, 3H), 3.87 (d, 1H), 3.65 (s, 2H), 3.39 (dt, 1H), 3.20 (dd, 1H), 2.97 (dt, 1H), 1.10 (d, 3H).

### EXAMPLE 82

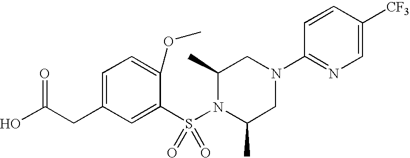

### {3-[2,6-Dimethyl-4-(5-trifluoromethyl-pyridin-2-yl)-piperazine-1-sulfonyl]-4-methoxy-phenyl}-acetic acid

The compound of Example 82 was synthesized according to the method described for the preparation of Example 78 using (3-Chlorosulfonyl-4-methoxy-phenyl)-acetic acid methyl ester. ¹H NMR (400 MHz, CDCl₃), δ (ppm): 8.35 (s, 1H), 7.89 (m, 1H), 7.61 (dd, 1H), 7.44 (dd, 1H), 6.97 (d, 1H), 6.59 (d, 1H), 4.16(m, 4H), 3.93 (s, 3H), 3.66 (s, 2H), 2.98 (dd, 2H), 1.42 (d, 6H).

### EXAMPLE 83

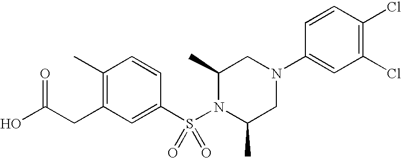

### {5-[4-(3,4-Dichloro-phenyl)-2,6-dimethyl-piperazine-1-sulfonyl]-2-methyl-phenyl}-acetic acid

The compound of Example 83 was synthesized according to the procedure outlined for Example 92. ¹H NMR (400 MHz, MeOH-D₄) δ 7.74 (s, 1H), 7.67 (d, 1H), 7.36 (d, 1H), 7.28 (d, 1H), 6.93 (d, 1H), 6.76 (dd, 1H), 4.25-4.15 (m, 2H), 3.75 (s, 2H), 3.32 (d, 2H), 2.72 (dd, 2H), 2.39 (s, 3H), 1.47 (d, 6H); LCMS 470.9 (M+1)⁺.

### EXAMPLE 84

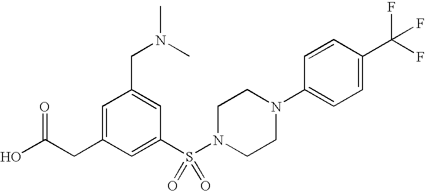

### {3-Dimethylaminomethyl-5-[4-(4-trifluorometbyl-phenyl)-piperazine-1-sulfonyl]-phenyl}-acetic acid

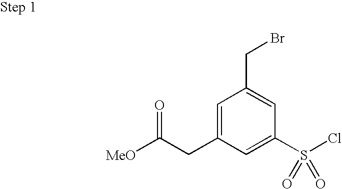

(3-Bromomethyl-5-chlorosulfonyl-phenyl)-acetic acid methyl ester. A mixture of (3-Chlorosulfonyl-5-methyl-phenyl)-acetic acid methyl ester (5.64 g, 21.5 mmol, 1.0 equiv.), NBS (4.2 g, 23.6 mmol, 1.1 equiv.) and AIBN (106 mg, 0.64 mmol, 0.03 equiv.) in benzene (100 mL) were heated to reflux for 30 h. The reaction mixture was cooled to room temperature and, then diluted with ethyl acetate (500 mL). The organic mixture was washed with water, brine and dried over Na₂SO₄. After removal of solvent, the crude product was purified by chromatography to give 3.24 g (44% yield) of (3-Bromomethyl-5-chlorosulfonyl-phenyl)-acetic acid methyl ester. ¹H NMR (400 MHz, CDCl₃), δ (ppm): 8.01 (s, 1H), 7.93 (s, 1H), 7.74 (s, 1H), 4.56 (s, 2H), 3.80 (s, 2H), 3.79 (s, 3H).

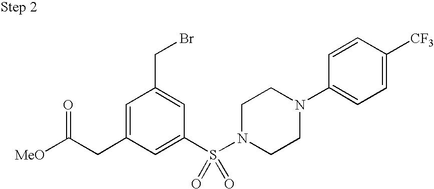

{3-Bromomethyl-5-[4-(4-trifluoromethyl-phenyl)-piperazine-1-sulfonyl]-phenyl}-acetic acid methyl ester. The compound was synthesized according to the method described for the preparation of II-C-3 in Example 3, Step 3 using 4-(4-trifluoromethylphenyl)-piperazine. ¹H NMR (400 MHz, CDCl₃), δ (ppm): 7.76 (s, 1H), 7.68 (s, 1H), 7.60 (s, 1H), 7.51 (d, 2H), 6.92 (d,-2H), 4.54 (s, 2H), 3.76 (s, 5H), 3.39 (m, 4H), 3.23 (m, 4H).

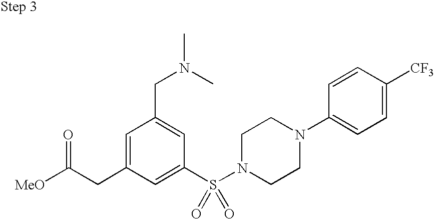

{3-Dimethylaminomethyl-5-[4-(4-trifluoromethyl-phenyl)-piperazine-1-sulfonyl]-phenyl}-acetic acid methyl ester. A mixture of the intermediate from Step 2 (209.7 mg, 0.39 mmol, 1.0 equiv.) and dimethylamine (0.39 mL of 2.0 M in THF, 0.78 mmol, 2.0 equiv.) in THF (5 mL) was stirred at room temperature for 2 hours. The reaction mixture was concentrated under reduced pressure and the residue was diluted with ethyl acetate (20 mL). The organic mixture was washed with water, brine and dried over Na,SO₄. After removal of solvent, the crude product was purified by chromatography to give 143 mg (73% yield) of {3-Dimethylaminomethyl-5-[4-(4-trifluoromethyl-phenyl)-piperazine-1-sulfonyl]-phenyl}-acetic acid methyl ester. ¹H NMR (400 MHz, CDCl₃), δ (ppm): 7.67 (s, 1H), 7.63 (s, 1H), 7.54 (s, 1H), 7.49 (d, 2H), 6.90 (d, 2H), 3.74 (s, 5H), 3.51 (s, 2H), 3.36 (m, 4H), 3.21 (m, 4H), 2.27 (s, 6H).

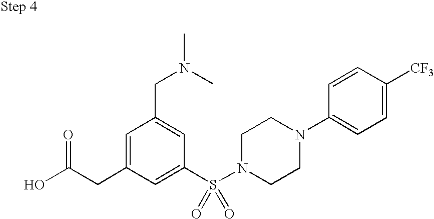

{3-Dimethylaminomethyl-5-[4-(4-trifluoromethyl-phenyl)-piperazine-1-sulfonyl]-phenyl}-acetic acid. The compound {3-Dimethylaminomethyl-5-[4-(4-trifluoromethyl-phenyl)-piperazine-1-sulfonyl]-phenyl}-acetic acid was synthesized according to the method described for the preparation of Example 1 in Step 3. ¹H NMR (400 MHz, CDCl₃), δ (ppm): 7.95 (s, 1H), 7.68 (s, 1H), 7.55 (s, 1H), 7.50 (d, 2H), 6.90 (d, 2H), 3.96 (s, 2H), 3.75 (s, 2H), 3.36 (m, 4H), 3.21 (m, 4H), 2.54 (s, 6H).

### EXAMPLE 85

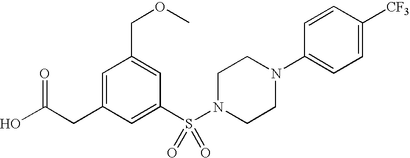

### {3-Methoxymethyl-5-[4-(4-trifluoromethyl-phenyl)-piperazine-1-sulfonyl]-phenyl}-acetic acid

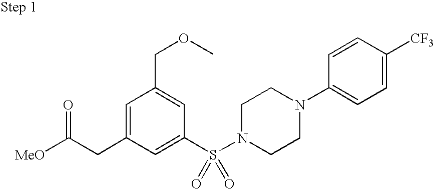

{3-Methoxymethyl-5-[4-(4-trifluoromethyl-phenyl)-piperazine-1-sulfonyl]-phenyl}-acetic acid methyl ester. A mixture of the product from Example 84, Step 2 (176 mg, 0.33 mmol, 1.0 equiv.) and sodium methoxide (1.0 mL of 0.5 M solution in MeOH, 1.0 mmol, 3 equiv.) in MeOH/THF (⅔) (5 mL) was stirred at room temperature for 2 hours. The reaction mixture was concentrated under reduced pressure and the residue was diluted with ethyl acetate (30 mL). The organic mixture was washed with water, brine and dried over Na₂SO₄. After removal of solvent, the crude product was purified by chromatography to give 20 mg (12% yield) of {3-Methoxymethyl-5-[4-(4-trifluoromethyl-phenyl)-piperazine-1-sulfonyl]-phenyl}-acetic acid methyl ester. ¹H NMR (400 MHz, CDCl₃), δ (ppm): 7.70 (s, 1H), 7.67 (s, 1H), 7.55 (s, 1H), 7.50 (d, 2H), 6.91 (d, 2H), 4.55 (s, 2H), 3.75 (s, 2H), 3.74 (s, 3H), 3.47 (s, 3H), 3.38 (m, 4H), 3.22 (m, 4H).

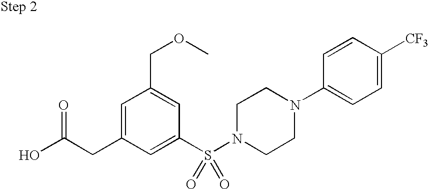

{3-Methoxymethyl-5-[4-(4-trifluoromethyl-phenyl)-piperazine-1-sulfonyl]-phenyl)}-acetic acid. The compound of Example 85 was synthesized from the product of Step 1 according to the method described for the preparation of Example 1 in Step 3. ¹H NMR (400 MHz, CDCl₃), δ (ppm): 7.71 (s, 1H), 7.67 (s, 1H), 7.55 (s, 1H), 7.50 (d, 2H), 6.91 (d, 2H), 4.55 (s, 2H), 3.77 (s, 2H), 3.47 (s, 3H), 3.37 (m, 4H), 3.21 (m, 4H).

### EXAMPLE 86

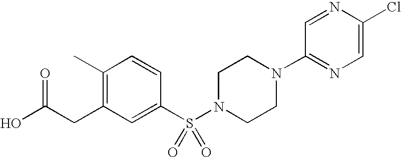

### {2-Methyl-5-[4-(5-trifluoromethyl-pyridin-2-yl)-piperazine-1-sulfonyl]-phenyl}-acetic acid

The compound of Example 86 was synthesized according to Scheme XV.

Step 1

3-Chloro-pyrazin-1-ol XV-A-86. Into a 1000 ml 3-necked round bottom flask, was placed acetic acid (300 ml). To this was added 2-chloropyrazine (142 g, 1.24 mol). To the mixture was added 30% oxydol (250 ml). The resulting solution was stirred for 22 h while the temperature was maintained at 65-75° C. The solution was cooled and concentrated to one-third volume, diluted with an equal quantity of water and concentrated. The residue was extracted four times with CH₂Cl₂ and the organic layers combined, dried and concentrated by evaporation under vacuum using a rotary evaporator. This resulted in 74.4 g (46%) of compound XV-A-86 as a white solid.

Step 2

2,5-Dichloro-pyrazine XV-B-86. Into a 250 ml 3-necked round bottom flask, was placed phosphoryl chloride (115 g, 0.75 mol). To the mixture was added XV-A-86(39 g, 0.30 mol), while warming to a temperature of 60-70° C. The resulting solution was heated to reflux, with stirring, for an additional 1 h. After cooling to room temperature, the resulting solution was poured cautiously onto 3000 g of chopped ice with stirring and extracted four times with 800 ml CH₂Cl₂ and the organic layers combined and concentrated by evaporation under vacuum using a rotary evaporator. The residue was purified by eluting through a column with a 1:10 EtOAc/PE solvent system. The collected fractions were combined and concentrated by evaporation under vacuum using a rotary evaporator. This resulted in 16.5 g (37%) of compound XV-B-86 as a colorless liquid.

Step 3

2-Chloro-5-iodo-pyrazine XV-C-86. Into a 250 ml 3-necked round bottom flask, was placed 45% hydriodic acid (60 ml). To this was added sodium iodide (25 g, 0.17 mol). To the mixture was added XV-B-86 (10.5 g, 0.07 mol). The resulting solution was allowed to react at room temperature. Adjustment of the pH to >8 was accomplished by the addition of 20 g NaOH in 50 g ice to afford XV-C-86.

Step 4

5′-Chloro-3,4,5,6-tetrahydro-2H-[1,2′]bipyrazinyl XV-D-86. Into a 250 ml round bottom flask, was placed isopropanol (150 ml). To the mixture was added XV-C-86 (5 g, 0.02 mol). To the mixture was added CuI (0.2 g, 1 mmol). To the mixture were added ethylene glycol (2.0 g, 0.03 mol), anhydrous potassium phosphate (6.5 g)and piperazine (1.3 g, 0.02 mol). The resulting solution was stirred, for 14 h while the temperature was maintained at 80-85° C. The resulting solution was concentrated in vacuo. To the residue was added 40 mL water and then extracted four times with 200 mL CH₂Cl₂. The organic layers were combined and dried with anhydrous sodium sulfate and concentrated in vacuo. The residue was purified by silica gel column chromatography. The collected fractions were combined and concentrated in vacuo to afford XV-D-86.

Step 5 & 6

{2-Methyl-5-[4-(5-trifluoromethyl-pyridin-2-yl)-piperazine-1-sulfonyl]-phenyl}-acetic acid. The compound of Example 86 was prepared from the compound of Step.4 following the procedures described for Example 1, Steps 2 and 3. ¹H NMR (400 MHz, CDCl₃) 8:8.31 (s, 1H), 8.19 (d 1H), 7.62 (d 1H), 7.64(s, 1H), 7.38 (d, 1H), 3.77(s, 2H), 3.48(t, 4H), 3.16(t, 4H), 2.42 (s, 3H).

### EXAMPLE 87

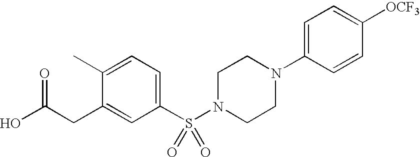

### {2-Methyl-5-[4-(4-trifluoromethoxy-phenyl)-piperazine-1-sulfonyl]-phenyl}-acetic acid

The compound {2-methyl-5-[4-(4-trifluoromethoxy-phenyl)-piperazine-1-sulfonyl]-phenyl}-acetic acid was synthesized according to the procedure outlined for Example 92. ¹H NMR (400 MHz, MeOH-D₄) δ 7.64 (d, 1H), 7.58 (dd, 1H), 7.42 (d, 1H), 7.10(d, 2H), 6.96 (d, 2H), 3.70 (s, 2H), 3.24-3.22 (m, 4R), 3.14-3.11 (m, 4H), 2.41 (s, 3H); LCMS 458.9 (M+1)⁺.

### EXAMPLE 88

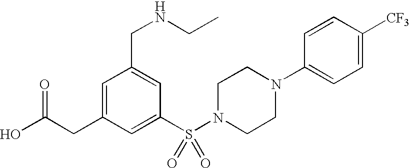

### {3-Ethylaminomethyl-5-[4-(4-trifluoromethyl-phenyl)-piperazine-1-sulfonyl]-phenyl}-acetic acid

The compound of Example 88 was synthesized according to the method described for the preparation of Example 84. ¹H NMR (400 MHz, DMSO), δ (ppm): 7.76 (s, 1H), 7.67 (s, 1H), 7.66 (s, 1H), 7.38 (d, 2H), 6.83 (d, 2H), 4.10 (s, 2H), 3.66 (s, 2H), 3.28 (m, 4H), 3.13 (m, 4H), 2.97 (q, 2H), 1.33 (t, 3H).

### EXAMPLE 89

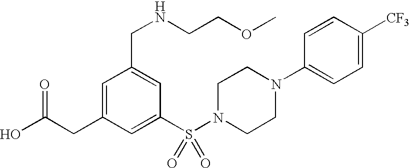

### {3-[(2-Methoxy-ethylamino)-methyl]-5-[4-(4-trifluoromethyl-phenyl)-piperazine-1-sulfonyl]-phenyl}-acetic acid

The compound of Example 89 was synthesized according to the method described for the preparation of Example 84. ¹H NMR (400 MHz, DMSO), δ (ppm): 7.41 (s, 1H), 7.38 (s, 1H), 7.36 (s, 1H), 7.19 (d, 2H), 6.65 (d, 2H), 3.80 (s, 2H), 3.40 (s, 2H), 3.34 (t, 2H), 3.13 (s, 3H), 3.07 (m, 4H), 2.92 (m, 4H), 2.77 (t, 2H).

### EXAMPLE 90

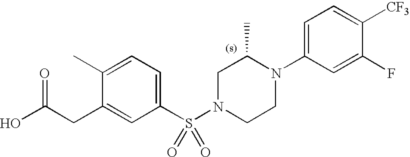

### {5-[4-(3-Fluoro-4-trifluoromethyl-phenyl)-3-(S)-methyl-piperazine-1-sulfonyl]-2-methyl-phenyl}-acetic acid

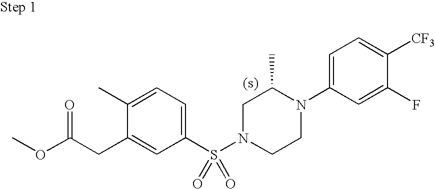

### {5-[4-(3-Fluoro-4-trifluoromethyl-phenyl)-3-(S)-methyl-piperazine-1-sulfonyl]-2-methyl-phenyl}-methyl ester

The compound {5-[4-(3-fluoro-4-trifluoromethyl-phenyl)-3-(S)-methyl-piperazine-1-sulfonyl]-2-methyl-phenyl}-methyl ester was synthesized according to the procedure outlined for Example 29 steps 3 and 4 in Scheme IX using 4-bromo-2-fluoro-1-trifluoromethyl-benzene and 3-Methyl-piperazine-1-carboxylic acid tert-butyl ester.

Step 2

{5-[4-(3-Fluoro-4-trifluoromethyl-phenyl)-3-(S)-methyl-piperazine-1-sulfonyl]-2-methyl-phenyl}-acetic acid. The compound of Example 90 was synthesized from the compound of Step 1 according to the procedure described for Example 1, Step 3. ¹H NMR (400 MHz, MeOH-D₄) δ 7.64 (s, 1H), 7.59(dd, 1H), 7.45 (d, 1H), 7.39 (d, 1H), 7.33 (d, 1H), 7.17 (t, 1H), 3.85-3.80 (m, 1H), 3.76 (s, 2H), 3.46-3.07 (m, 4H), 2.98-2.94 (m, 1H), 2.84-2.79 (m, 1H), 2.42 (s, 3H), 1.08 (d, 3H); LCMS 474.9 (M+1)⁺.

### EXAMPLE 91

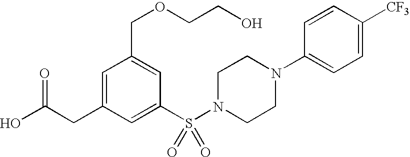

### {3-(2-Hydroxy-ethoxymethyl)-5-[4-(4-trifluoromethyl-phenyl)-piperazine-1-sulfonyl]-phenyl}-acetic acid

To a solution of ethylene glycol (0.2 mL, 3.6 mmol, 10 equiv.) in THF (5 mL) was added sodium hydride (68 mg of 60% in mineral oil, 1.7 mmol, 5 equiv) in three portions. After stirred for 5 min, the product from Example 84, Step 2 (196 mg, 0.37 mmol, 1.0 equiv.) was added with stirring. The resulting mixture was stirred at room temperature for 2 hours and then quenched with 1n HCl (1.7 mL). The mixture was diluted with ethyl acetate (50 mL) and washed with water, brine and dried over Na₂SO)₄. After removal of solvent, 17.3 mg (10% yield) of desired product was obtained. ¹H NMR (400 MHz, CDCl₃), δ (ppm): 7.68 (s, 1H), 7.65 (s, 1H), 7.57 (s, 1H), 7.50 (d, 2H), 6.90 (d, 2H), 4.63 (s, 2H), 3.81 (t, 2H), 3.74 (s, 2H), 3.65 (t, 2H), 3.36 (m, 4H), 3.20 (m, 4H).

### EXAMPLE 92

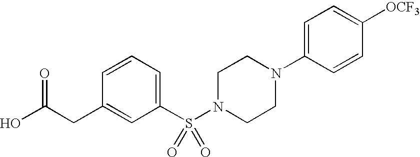

### {3-[4-(4-Trifluoromethoxy-phenyl)-piperazine-1-sulfonyl]-phenyl}-acetic acid

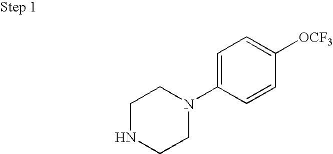

1-(4-Trifluoromethoxy-phenyl)-piperazine. The compound 1-(4-trifluoromethoxy-phenyl)-piperazine was synthesized according to the procedure outlined for Example 29, Step 3.

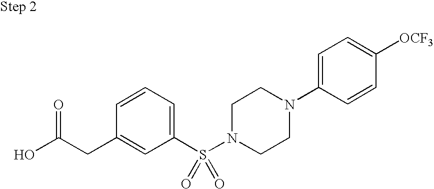

{3-[4-(4-Trifluoromethoxy-phenyl)-piperazine-1-sulfonyl]-phenyl}-acetic acid. The compound of Example 92 was synthesized from the product of Step 1 according to the method described for the preparation of Example 1, Steps 2 and 3.

¹H NMR (400 MHz, CDCl₃) δ 7.73-7.70 (m, 2H), 7.56-7.50 (m, 2H), 7.12 (d, 2H), 6.89(d, 2H), 3.75 (s, 2H), 3.26-3.16 (m, 8H); LCMS 444.8 (M+1)⁺.

### EXAMPLE 93

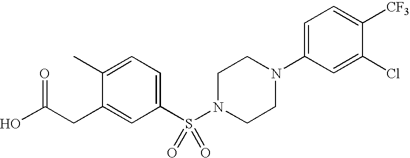

### {5-[4-(3-Chloro-4-trifluorometbyl-phenyl)-piperazine-1-sulfonyl]-2-methyl-phenyl}-acetic acid

The compound of Example 93 was synthesized according to the procedure outlined for example 92 using 4-bromo-2-chloro-1-trifluoromethyl-benzene and piperazine. ¹H NMR (400 MHz, CDCl₃) δ 7.62 (s, 1H), 7.60 (d, 1H), 7.48 (d, 1H), 7.37 (d, 1H), 6.86 (d, 1H), 6.70 (dd, 1H), 3.74 (s, 2H), 3.36-3.33 (m, 4H), 3.15-3.13 (m, 4H), 2.40 (s, 3H); LCMS 476.9 (M+1)⁺.

### EXAMPLE 94

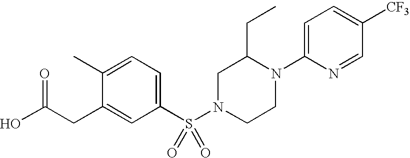

### {5-[3-Ethyl-4-(5-trifluoromethyl-pyridin-2-yl)-piperazine-1-sulfonyl]-2-methyl-phenyl}-acetic acid

The compound of Example 94 was synthesized according to the procedure outlined for Example 90. ¹H NMR (400 MHz, MeOH-D₄) δ 8.30 (s, 1H), 7.66 (d, 1H), 7.62 (s, 1H), 7.58 (d, 1H), 7.41 (d, 1H), 6.80 (d, 1H), 4.49 (s, 1H), 4.34 (d, 1H), 3.75 (s, 2H), 3.34-3.14(m, 3R), 2.44-2.30 (m, 2H), 2.39 (s, 3H), 1.90-1.82 (m, 1H), 1.73-1.66 (m, 1H), 0.92 (t, 3H); LCMS 471.9 (M+1)⁺.

### EXAMPLE 95

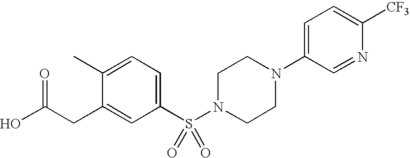

### {2-Methyl-5-[4-(6-trifluoromethyl-pyridin-3-yl)-piperazine-1-sulfonyl]-phenyl}-acetic acid

The compound of Example 95 was synthesized according to Scheme XIV.

Step 1

5-Bromo-2-iodo-pyridine XIV-A-95. Into a 250 ml 3-necked round bottom flask, was placed 45% HI (110 ml). To the above was added NaI (15 g, 0.10 mol) and 2,5-dibromopyridine (20 g, 0.08 mol). The resulting solution was stirred for 17 h while the temperature was maintained at 115-125° C. After cooling to room temperature, the pH was adjusted to >8 by addition of 20 g NaOH in 200 g ice. The resulting solution was extracted three times with CH₂Cl₂ (100 mL 4 times) and the organic layers combined was washed one time with 50 ml of saturated NaCl solution, and then dried with NaSO₄. The organic solution was concentrated in vacuo to give XIV-A-95, 23.2 g.

Step 2

5-Bromo-2-trifluoromethyl-pyridine XIV-B-95. Into a 250 ml 3-necked round bottom flask purged and maintained with an inert atmosphere of nitrogen, was placed NMP (80 ml). To the above was added KF (6.8 g, 0.12 mol) and CuI (15 g, 0.08 mol). After stirring 5-10 min, XIV-A-95 (11 g, 0.04 mol) and ClF₂CCO₂Et (18 g, 0.12 mol) were added. The resulting solution was stirred for 6 h while the temperature was maintained at 115-125° C. After cooling, 300 ml CH₂Cl₂ was added to the reaction system. The organic layer was washed with saturated NaCl solution (80 ml×5) and dried with Na₂SO₄. After evaporating the solvent, the residue was purified by column chromatography (eluant: PE:EtOAc=10:1) and compound XIV-B-95 was collected (4.65 g, 53.1%) as a yellow solid (m.p.: 38-40° C.)

Step 3

1-(6-Trifluoromethyl-pyridin-3-yl)-piperazine XIV-C-95. In a 50 ml 3-necked round bottom flask purged and maintained with an inert atmosphere of nitrogen was added toluene (1 5 mL), Pd(OAc)₂ (25 mg, 0.11 mmol) and BINAP (90 mg, 0.14 mmol). The reaction mixture was heated to 40-50° C. After stirring for 10 min, sodium tert-butoxide (1.5 g, 20 mmol), piperazine (1 g, 15 mmol), XIV-B-95 (2.2 g, 10 mmol) were added. The resulting solution was heated for 18 h while the temperature was maintained at 110° C. After cooling to room temperature, 50 mL CH₂Cl₂ was added to reaction system. The organic solution was washed with brine, dried with Na₂SO₄ and concentrated in vacuo. The residue was purified by silica gel column chromatography (eluant: first using PE:EtOAc=1:1, then using MeOH collect the product) to give 0.8 g (36%) of XIV-C-95 as a yellow liquid.

Step 4 & 5

{2-Methyl-5-[4-(6-trifluoromethyl-pyridin-3-yl)-piperazine-1-sulfonyl]-phenyl}-acetic acid XIV-D-95. The compound of Example 95 was prepared from the product of Step 3 following the procedures described for Example 1 steps 2 and 3. ¹H NMR (400 MHz, CDCl₃): 8.18 (s, 1H), 7.46 (d 1H), 7.28 (d 1H), 7.49(s, 1H), 7.42 (d, 1H), 7.13(d, 1H), 3.59(s, 2H), 3.31 (t, 4H), 3.06(t, 4H), 2.30 (s, 3H)

### EXAMPLE 96

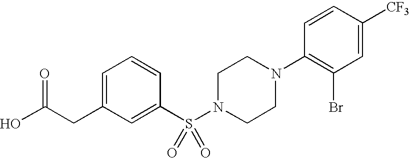

### {3-[4-(4-Trifluoromethyl-phenyl)-piperazine-1-sulfonyl]-phenyl)}-acetic acid

A mixture of {3-[4-(4-trifluoromethyl-phenyl)-piperazine-1-sulfonyl]-phenyl}-acetic acid methyl ester, II-C-3, (4.06 g, 9.48 mmol, 1.0 equiv.), NBS (2.5 g, 14.2 mmol, 1.5 equiv.) and AIBN (47 mg, 0.28 mmol, 0.03 equiv.) in benzene (80 mL) was heated to reflux for 12 h. The reaction mixture was cooled to room temperature and then diluted with ethyl acetate (500 mL). The organic mixture was washed with water, brine and dried over Na₂SO₄. After removal of solvent, the crude product was purified by chromatography to give 3.66 g (74% yield) of {3-[4-(4-Trifluoromethyl-phenyl)-piperazine-1-sulfonyl]-phenyl}-acetic acid methyl ester. The compound of Example 96 was synthesized from {3-[4-(4-Trifluoromethyl-phenyl)-piperazine-1-sulfonyl]-phenyl}-acetic acid methyl ester according to the method described for the preparation of Example 1, Step 3. ¹H NMR (400 MHz, CDCl₃): 7.78 (d, 1H), 7.73 (m, 2H), 7.55 (m, 3H), 7.06 (d, 1H), 3.77 (s, 2H), 3.23 (m, 4H), 3.16 (m, 4H).

### EXAMPLE 97

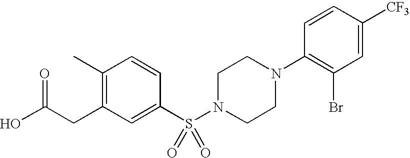

{5-[4-(2-Bromo-4-trifluoromethyl-phenyl)-piperazine-1-sulfonyl]-2-methyl-phenyl-}acetic acid

The compound of Example 97 was synthesized followed the procedure for Example 96. ¹H NMR (400 MHz, CDCl₃), δ (ppm): 7.77 (d, 1H), 7.62 (m, 2H), 7.52 (dd, 1H), 7.38 (d, 1H), 7.06 (d, 1H), 3.77 s, 2H), 3.22 (m, 4H), 3.15 (m, 4H), 2.42 (s, 3H).

### EXAMPLE 98

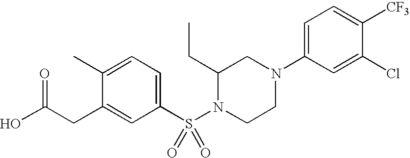

{5-[2-Ethyl-4-(3-chloro-4-trifluoromethyl-phenyl)-piperazine-1-sulfonyl]-2-methyl-phenyl}-acetic acid

The compound of Example 98 was synthesized according to the procedure outlined for Example 92. ¹H NMR (400 MHz, MeOH-D₄) δ 7.73 (d, 1H), 7.65 (dd, 1H), 7.45 (d, 1H), 7.33 (d, 1H), 6.86 (d, 1H), 6.73 (dd, 1H), 4.89-3.95 (m, 1H), 3.85-3.82 (m, 1H), 3.72 (s, 2H), 3.54-3.46 (m, 2H), 3.40-3.30 (m, 1H), 2.92 (dd, 1H), 2.74-2.68 (m, 1H), 2.34 (s, 3H), 1.73-1.57 (2H), 0.94 (t, 3H); LCMS 504.8 (M+1)⁺.

### EXAMPLE 99

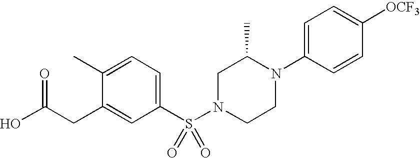

### {5-[4-(4-trifluoromethyl-phenyl)-3-(S)-methyl-piperazine-1-sulfonyl]-2-methyl-phenyl}-acetic acid

The compound of Example 99 was synthesized according to the procedure outlined for Example 92. ¹H NMR (400 MHz, MeOH-D₄) δ 7.64 (d, 1H), 7.60 (dd, 1H), 7.45 (d, 1H), 7.11 (d, 2H), 6.95 (d, 2H), 4.82-3.95 (m, 1H), 3.77 (s, 2H), 3.58-3.55 (m, 1H), 3.37-3.25 (m, 2H), 3.19-3.13 (m, 1H), 2.78 (dd, 1H), 2.67-2.60 (m, 1H), 2.41 (s, 3H), 1.06 (d, 3H); LCMS 472.9 (M+1)⁺.

### EXAMPLE 100

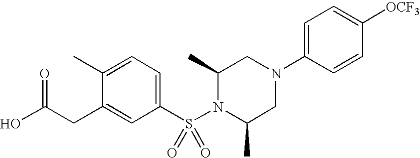

### {5-[2,6-Dimethyl-4-(4-trifluoromethoxy-phenyl)-piperazine-1-sulfonyl]-2-methyl-phenyl}-acetic acid

The compound of Example 100 was synthesized according to the procedure outlined for Example 92. ¹H NMR (400 MHz, MeOH-D₄) δ 7.72 (d, 1H), 7.66 (dd, 1H), 7.36 (d, 1H), 7.08 (d, 2H), 6.87 (d, 2H), 4.20-4.16 (m, 2H), 3.73 (s, 2H), 3.31-3.27 (m, 2H), 2.61 (dd, 2H), 2.37 (s, 3H), 1.47 (d, 6H); LCMS 487.0 (M+1)⁺.

### EXAMPLE 101

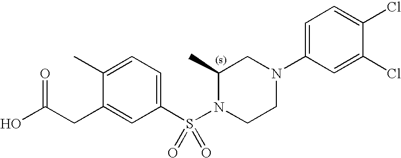

### {5-[4-(3,4-Dichloro-phenyl)-2-(S)-methyl-piperazine-1-sulfonyl]-2-methyl-phenyl}-acetic acid

The compound of Example 101 was synthesized according to the procedure outlined for Example 92. ¹H NMR (400 MHz, MeOH-D₄) δ 7.71 (d, 1H), 7.64 (dd, 1H), 7.37 (d, 1H), 7.26 (d, 1H), 6.94 (d, 1H), 6.76 (dd, 1H), 4.20-4.16 (m, 1H), 3.77-3.72 (m, 1H), 3.73 (s, 2H), 3.47-3.44 (m, 1H), 3.39-3.30 (m, 2H), 2.89-2.85 (dd, 1H), 2.74-2.68 (m, 1H), 2.37 (s, 3H), 1.18 (d, 3H); LCMS 456.9 (M+1)⁺.

### EXAMPLE 102

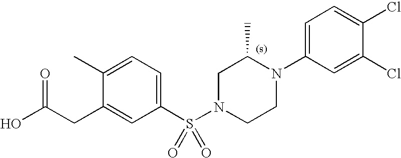

### {5-[4-(3,4-Dichloro-phenyl)-3-(S)-methyl-piperazine-1-sulfonyl]-2-methyl-phenyl}-acetic acid

The compound of Example 102 was synthesized according to the procedure outlined for Example 90. ¹H NMR (400 MHz, MeOH-D₄) δ 7.63 (d, 1H), 7.60 (dd, 1H), 7.44 (d, 1H), 7.29 (d, 1H), 7.01 kd, 1H), 6.82 (dd, 1H), 4.03-4.00 (m, 1H), 3.78 (s, 2H), 3.67-3.64 (m, 1H), 3.47-3.44 (m, 1H), 3.40-3.20 (m, 1H), 3.17-3.12 (m, 1H), 2.71-2.68 (dd, 1H), 2.56-2.51 (m, 1H), 2.40 (s, 3H), 1.10 (d, 3H); LCMS 456.9 (M+1)⁺.

### EXAMPLE 103

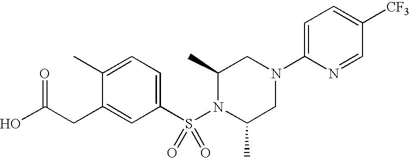

### {5-[2,6-(S,S)-Dimethyl-4-(5-trifluoromethyl-pyridin-2-yl)-piperazine-1-sulfonyl]-2-methyl-phenyl}-acetic acid

The compound of Example 103 was synthesized according to the procedure outlined for Example 90. ¹H NMR (400 MHz, MeOH-D₄) δ 8.29 (s, 1H), 7.71 (d, 1H), 7.64 (dd, 1H), 7.61 (dd, 1H), 7.22 (d, 1H), 6.59 (d, 1H), 4.22-4.17 (m, 2H), 3.78 (dd, 2H), 3.67 (s, 2H), 3.47 (dd, 2H), 2.31 (s, 3H), 1.30 (d, 6H); LCMS 471.8 (M+1)⁺.

### EXAMPLE 104

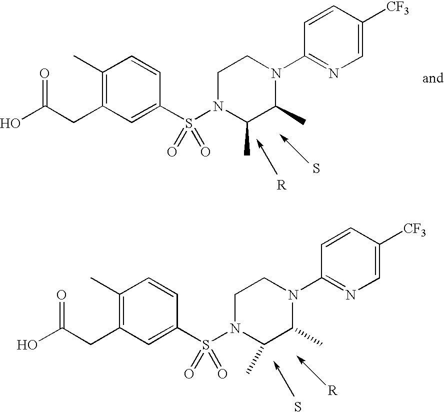

### RS and SR-{5-[2,3-Dimethyl-4-(5-trifluoromethyl-pyridin-2-yl)-piperazine-1-sulfonyl]-2-methyl-phenyl}-acetic acid

Synthesis of 2,3-Dimethyl-1-(5-trifluoromethyl-pyridin-2-yl)-piperazine. The compound 2,3-Dimethyl-1-(5-trifluoromethyl-pyridin-2-yl)-piperazine was synthesized according to the procedures outlined for Step 1 and 2 as follows.

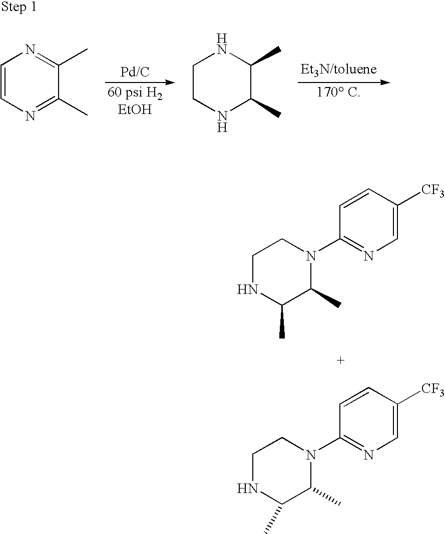

2,3-Dimethylpiperazine. 2.56 g of 2,3-dimethyl-pyrazine (23.67 mmol) was dissolved in 100 mL of ethanol with 2.1 g 10% palladium on active carbon. The reaction mixture was hydrogenated under pressure (55-60 psi) for 3 days. The solid was filtered and removed. The filtrate was concentrated to afford 3.0 g of 2,3-dimethylpiperazine, which was used without purification. ¹H NMR (400 MHz, CDCl₃), δ (ppm): 2.95 (m, 4H), 2.74 (m, 2H), 1.04 (d, 6H).

Step 2

2,3-Dimethyl-1-(5-trifluoromethyl-pyridin-2-yl)-piperazine. The compound was prepared from 2,3-dimethylpiperazine according to the procedure from Example 6, Step 3. ¹H NMR (400 MHz, CDCl₃), δ (ppm): 8.39 (d, 1H), 7.60 (dd, 1H), 6.58 (d, 1H), 4.36 (b, 1H), 4.06 (m, 1H), 3.13 (m, 1H), 3.07 (m, 2H), 2.90 (dt, 1H), 1.12 (dd, 6H).

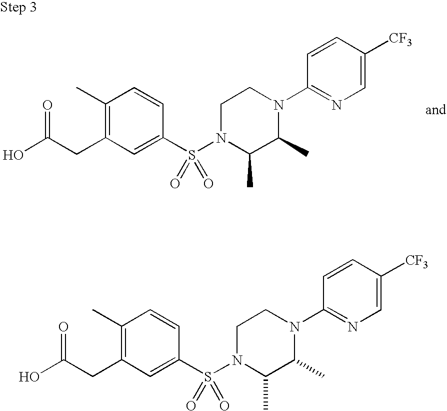

{5-[2,3-Dimethyl-4-(5-trifluoromethyl-pyridin-2-yl)-piperazine-1-sulfonyl]-2-methyl-phenyl}-acetic acid. The compound {5-[2,3-Dimethyl-4-(5-trifluoromethyl-pyridin-2-yl)-piperazine-1-sulfonyl]-2-methyl-phenyl}-acetic acid was synthesized according to the method described for Example 1 (Steps 2 and 3). ¹H NMR (400 MHz, CDCl₃), δ (ppm): 8.33 (s, 1H), 7.63 (s, 1H), 7.57 (m, 2H), 7.24 (d, 1H), 6.41 (d, 1H), 4.38 (m, 1H), 3.97 (m, 2H), 3.68 (s, 2H), 3.32 (m, 1H), 3.23 (m, 1H), 3.08 (m, 1H), 2.33 (s, 3H), 1.41 (d, 3H), 1.18 (d, 3H).

### EXAMPLE 105

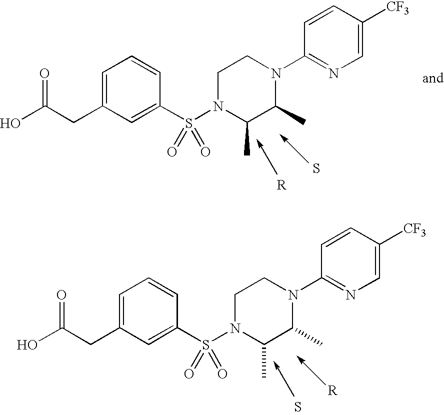

### RS and SR-{3-[2,3-Dimethyl-4-(S-trifluoromethyl-pyridin-2-yl)-piperazine-1-sulfonyl]-phenyl}-acetic acid

The compound {3-[2,3-Dimethyl4-(5-trifluoromethyl-pyridin-2-yl)-piperazine-1-sulfonyl]-phenyl}-acetic acid was synthesized according to the procedure outlined for Example 104. ¹H NMR (400 MHz, CDCl₃), δ (ppm): 8.34 (s, 1H), 7.74 (s, 1H), 7.69 (m, 1H), 7.57 (dd, 1H), 7.43 (m, 2H), 6.43 (d, 1H), 4.37 (m, 2H), 3.99 (m, 2H), 3.69 (s, 2H), 3.27 (m, 2H), 3.08 (m, 1H), 1.42 (d, 3H), 1.18 (d, 3H).

### EXAMPLE 106

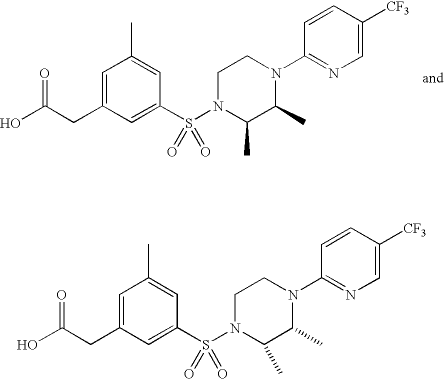

### RS and SR-{3-[2,3-Dimethyl-4-(5-trifluoromethyl-pyridin-2-yl)-piperazine-1-sulfonyl]-5-methyl-phenyl}-acetic acid

The compound {3-[2,3-Dimethyl-4-(5-trifluoromethyl-pyridin-2-yl)-piperazine-1-sulfonyl]-5-methyl-phenyl}-acetic acid was synthesized according to the procedure outlined for Example 104. ¹H NMR (400 MHz, CDCl₃), δ (ppm): 8.32(s, 1H),7.57(dd, 1H),7.53(s, 1H), 7.48 (s, 1H), 7.21 (s, 1H), 6.41 (d, 1H), 4.39 (m, 1H), 3.97 (m, 2H), 3.64 (s, 2H), 3.34 (m, 1H), 3.24 (m, 1H), 3.11 (m, 1H), 2.34 (s, 3H), 1.42 (d, 3H), 1.18 (d, 3H).

### EXAMPLE 107

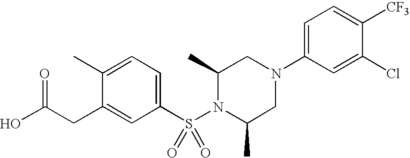

### {5-[4-(3-Chloro-4-trifluoromethyl-phenyl)-2,6-dimethyl-piperazine-1-sulfonyl]-2-methyl-phenyl}-acetic acid

The compound {5-[4-(3-chloro-4-trifluoromethyl-phenyl)-2,6-dimethyl-piperazine-1-sulfonyl]-2-methyl-phenyl}-acetic acid was synthesized according to the procedure outlined for Example 92. ¹H NMR (400 MHz, MeOH-D₄) δ 7.70 (d, 1H), 7.62 (dd, 1H), 7.45 (d, 1H), 7.29 (d, 1H), 6.86 (d, 1H), 6.73 (dd, 1H), 4.22-4.17 (m, 2H), 3.69 (s, 2H), 3.44 (dd, 2H), 2.91 (dd, 2H), 2.33 (s, 3H), 1.41 (d, 6H); LCMS 504.9 (M+1)⁺.

### EXAMPLE 108

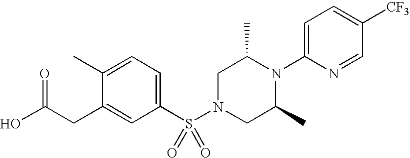

### {5-[3,5-(S,S)-Dimethyl-4-(5-trifluoromethyl-pyridin-2-yl)-piperazine-1-sulfonyl]-2-methyl-phenyl}-acetic acid

The compound {5-[3,5-(S,S)-dimethyl-4-(5-trifluoromethyl-pyridin-2-yl)-piperazine-1-sulfonyl]-2-methyl-phenyl}-acetic acid was synthesized according to the procedure outlined for example 90. ¹H NMR (400 MHz, MeOH-D₄) δ 8.33 (s, 1H), 7.75-7.65 (m, 3H), 7.44 (d, 1H), 6.67 (d, 1H), 4.37-4.32 (m, 2H), 3.77 (s, 2H), 3.60-3.59 (m, 4H), 2.39 (s, 3H), 1.03 (d, 6H); LCMS 472.2 (M+1)⁺.

### EXAMPLE 109

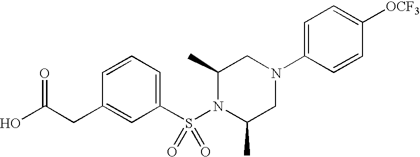

### {3-[2,6-Dimethyl-4-(4-trifluoromethoxy-phenyl)-piperazine-1-sulfonyl]-phenyl}-acetic acid

The compound {3-[2,6-dimethyl-4-(4-trifluoromethoxy-phenyl)-piperazine-1-sulfonyl)-phenyl}-acetic acid was synthesized according to the procedure outlined for Example 92. ¹H NMR (400 MHz, MeOH-D₄) δ 7.84 (s, 1H), 7.79-7.76 (m, 1H), 7.54-7.50 (m, 2H), 7.09 (d, 2H), 6.88 (d, 2H), 4.21-4.17 (m, 2H), 3.72 (s, 2H), 3.32-3.28 (m, 2H), 2.60 (dd, 2H), 1.47 (d, 6H); LCMS 472.9 (M+1)⁺.

### EXAMPLE 110

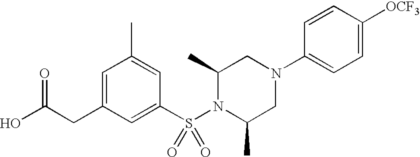

### {3-[2,6-Dimethyl-4-(4-trifluoromethoxy-phenyl)-piperazine-1-sulfonyl]-5-methyl-phenyl}-acetic acid

The compound {3-[2,6-dimethyl-4-(4-trifluoromethoxy-phenyl)-piperazine-1-sulfonyl]-5-methyl-phenyl}-acetic acid was synthesized according to the procedure in example 68. ¹H NMR (400 MHz, CD₃OD) δ 7.60 (d, 2H), 7.36 (s, 1H), 7.08 (d, 2H), 6.89-6.85 (m, 2H), 4.20-4.17 (m, 2H), 3.66 (s, 2H), 3.29 (d, 2H), 2.62 (dd, 2H), 2.40 (s, 3H), 1.47 (d, 6H); LCMS 486.9 (M+1)⁺.

### EXAMPLE 111

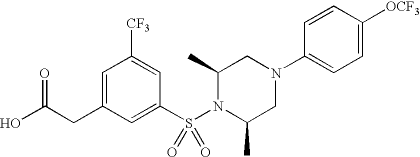

### {3-[2,6-Dimethyl-4-(4-trifluoromethoxy-phenyl)-piperazine-1-sulfonyl]-5-trifluoromethyl-phenyl}-acetic acid

¹H NMR (400 MHz, CDCl₃), δ (ppm): 8.03 (s, 1H), 7.98 (s, 1H), 7.70 (s, 1H), 7.10 (d, 2H), 6.80 (d, 2H), 4.20 (m, 2H), 3.78 (s, 2H), 3.24 (d, 2H), 2.67 (dd, 2H), 1.49 (d, 6H).

### EXAMPLE 112

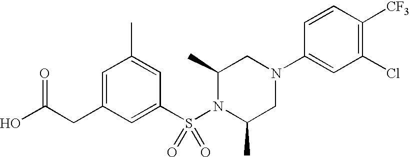

### {3-[4-(3-Chloro-4-trifluoromethyl-phenyl)-2,6-dimethyl-piperazine-1-sulfonyl]-5-methyl-phenyl}-acetic acid

The compound {3-[4-(3-chloro-4-trifluoromethyl-phenyl)-2,6-dimethyl-piperazine-1-sulfonyl]-5-methyl-phenyl]-acetic acid was synthesized according to the procedure in example 68. ¹H NMR (400 MHz, CD₃OD) δ 7.60 (s, 1H), 7.54 (s, 1H), 7.45 (d, 1H), 7.27 (s, 1H), 6.85 (d, 1H), 6.72 (dd, 1H), 4.22-4.18 (m, 2H), 3.65 (s, 2H), 3.44 (dd, 2H), 2.95 (dd, 2H), 2.35 (s, 3H), 1.42 (d, 6H); LCMS 504.9 (M+1)⁺.

### EXAMPLE 113

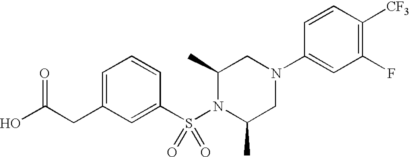

### {3-[4-(3-Fluoro-4-trifluoromethyl-phenyl)-2,6-dimethyl-piperazine-1-sulfonyl]-phenyl}-acetic acid

The compound {3-[4-(3-fluoro-4-trifluoromethyl-phenyl)-2,6-dimethyl-piperazine-1-sulfonyl]-phenyl}-acetic acid was synthesized according to the procedure in example 68. ¹H NMR (400 MHz, CD₃OD) δ 7.83 (s, 1H), 7.77-7.74 (m, 1H), 7.52-7.45 (m, 2H), 7.37 (t, 1H), 6.65 (s, 1H), 6:65-6.62 (m, 1H), 4.22-4.18 (m, 2H), 3.71 (s, 2H), 3.52 (d, 2H), 2.86 (dd, 2H), 1.42 (d, 6H); LCMS 474.8 (M+1)⁺.

### EXAMPLE 114

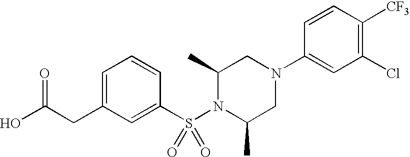

### {3-[4-(3-Chloro-4-trifluoromethyl-phenyl)-2,6-dimethyl-piperazine-1-sulfonyl]-phenyl}-acetic acid

The compound 13-[4-(3-chloro-4-trifluoromethyl-phenyl)-2,6-dimethyl-piperazine-1-sulfonyl]-phenyl}-acetic acid was synthesized according to the procedure in example 68. ¹H NMR (400 MHz, CD₃OD) δ 7.82 (s, 1H), 7.77-7.74 (m, 1H), 7.48-7.44 (m, 3H), 6.90 (d, 1H), 6.77 (dd, 1H), 4.22-4.18 (m, 2H), 3.71 (s, 2H), 3.50 (d, 2H), 2.89 (dd, 2H), 1.42 (d, 6H); LCMS 490.8 (M+1)⁺.

### PREPARATION OF EXAMPLES 115-146

Examples 115-146 were prepared from (3-Chlorosulfonyl-phenyl)-acetic acid methyl ester according to the general procedure below.

A) Parallel Syntheses of Piperazine Sulfonamide Intermediates.

(3-Chlorosulfonyl-phenyl)-acetic acid methyl ester (11.73 g, 47.17 mmol) was dissolved in THF (75 mL) and this resulting solution was allotted to 32 vials charged with piperazines substituted with various groups, G₃ and G₄ (1.47 mmol, 1.0 equiv) (each with 2.5 mL of solution). To each of the above 32 reaction mixtures was added NEt₃ (411 μL, 2.95 mmol, 2.0 equiv) followed by catalytic amount of DMAP and 5 mL of THF. The resulting suspensions were heated to 55° C. and stirred at same temperature for 18 hours. The reaction mixtures were concentrated under a stream of N₂. The residues were diluted with ethyl acetate (15 mL) and then washed with water, saturated NaHCO₃, brine and dried over Na₂SO₄. After removal of solvent, the crude products were purified by chromatography to give the desired coupled intermediates with 20-75% yield.

B) Parallel Syntheses of Examples 115-146.

The above Intermediates were charged in 32 vials, respectively. To each of the vials was added THF/MeOH (3:1) (5 mL) and then corresponding amount of 1N LiOH (2.0 equiv) to each of the resulting solutions. The resulting mixtures were stirred at room temperature for 6 hours and then concentrated under a stream of N₂. The residues were partitioned with diethyl ether (5 mL) and H₂O (5 mL). After separation, the aqueous solutions were neutralized with corresponding amounts of 1N HCl (2.0 equiv) and extracted with ethyl acetate (10 mL). The organic layers were washed with brine and dried over Na₂SO₄. After removal of solvent, products 115-146 were obtained with 50-85% yields. Their ¹H NMR data are described below.

### EXAMPLE 115

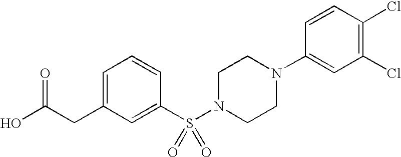

### {3-[4-(3,4-Dichloro-phenyl)-piperazine-1-sulfonyl]-phenyl}-acetic acid

¹H NMR (400 MHz, CDCl₃), δ (ppm): 7.72 (m, 2H), 7.54 (m, 2H), 7.26 (dd, 1H), 6.90 (d, 1H), 6.68 (dd, 1H), 3.76 (s, 2H), 3.21 (d, 4H), 3.15 (d, 4H).

### EXAMPLE 116

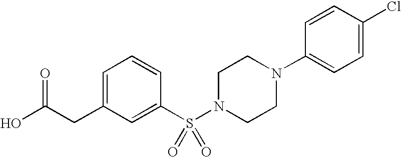

### {3-[4-(4-Chloro-phenyl)-piperazine-1-sulfonyl]-phenyl}-acetic acid

¹H NMR (400 MHz, CDCl₃), δ (ppm): 7.72 (m, 2H), 7.54 (m, 2H), 7.19 (d, 2H), 6.78 (d, 2H), 3.74 (s, 2H), 3.19 (m, 8H).

### EXAMPLE 117

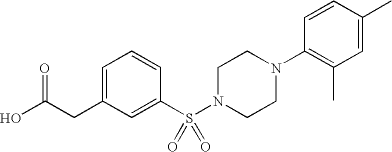

### {3-[4-(2,4-Dimethyl-phenyl)-piperazine-1-sulfonyl]-phenyl}-acetic acid

¹H NMR (400 MHz, CDCl₃), δ (ppm): 7.73 (m, 2H), 7.55 (m, 2H), 6.97 (m, 2H), 6.90 (d, 1H), 3.77 (s, 2H), 3.17 (b, 4H), 2.94(m, 4H), 2.26 (s, 3H), 2.14 (s, 3H).

### EXAMPLE 118

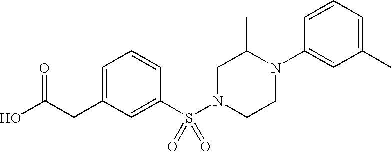

### [3-(3-methyl-4-m-tolyl-piperazine-1-sulfonyl)-phenyl]-acetic acid

¹H NMR (400 MHz, CDCl₃), δ (ppm): 7.69 (m, 2H), 7.51 (m, 2H), 7.13 (t, 1H), 6.73 (d, 1H), 6.68 (m, 2H), 3.80 (m, 1H), 3.73 (s, 2H), 3.47 (m, 1H), 3.22 (m, 3H), 2.95 (m, 1H), 2.79(m, 1H), 2.29 (s, 3H), 1.09 (s, 3H).

### EXAMPLE 119

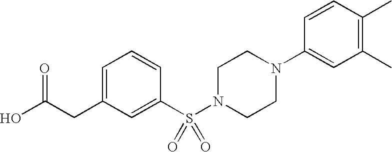

### {3-[4-(3,4-Dimethyl-phenyl)-piperazine-1-sulfonyl]-phenyl}-acetic acid

¹H NMR (400 MHz, CDCl₃), δ (ppm): 7.72 (m, 2H), 7.52 (m, 2H), 7.00 (d, 1H), 6.70 (s, 1H), 6.61 (d, 2H), 3.74 (s, 2H), 3.18 (s, 8H), 2.21 (s, 3H), 2.17 (s, 3H).

### EXAMPLE 120

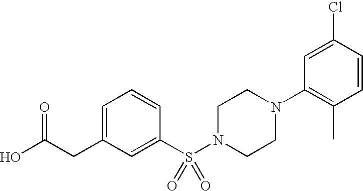

### {3-[4-(5-Chloro-2-methyl-phenyl)-piperazine-1-sulfonyl]-phenyl}-acetic acid

¹H NMR (400 MHz, CDCl₃), δ (ppm): 7.72 (m, 2H), 7.54 (m, 2H), 7.05 (d, 1H), 6.98 (d, 1H), 6.93 (s, 1H), 3.77 (s, 2H), 3.18 (s, 4H), 2.95 (m, 4H), 2.13 (s, 3H).

### EXAMPLE 121

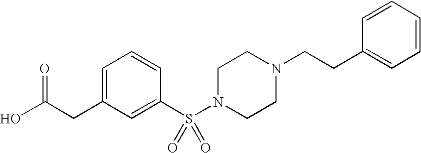

### [3-(4-phenethyl-piperazine-1-sulfonyl)-phenyl]-acetic acid

¹H NMR (400 MHz, CDCl₃), δ (ppm): 7.70 (m, 2H), 7.52 (m, 2H), 7.28 (m, 5H), 3.70 (s, 2H), 3.32 (s, 4H), 2.94 (m, 6H).

### EXAMPLE 122

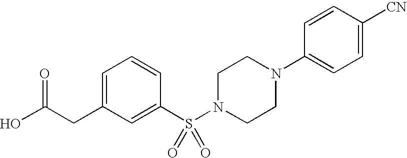

### {3-[4-(4-Cyano-phenyl)-piperazine-1-sulfonyl]-phenyl}-acetic acid

¹H NMR (400 MHz, CDCl₃), δ (ppm): 7.72 (m, 2H), 7.54 (m, 2H), 7.47 (d, 2H), 6.81 (d, 2H), 3.75 (s, 2H), 3.39 (m, 4H), 3.16 (m, 4H.

### EXAMPLE 123

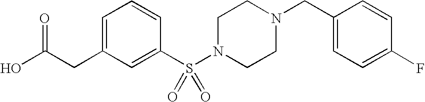

### {3-[4-(4-Fluoro-benzyl)-piperazine-1-sulfonyl]-phenyl}-acetic acid

¹H NMR (400 MHz, CDCl₃), δ (ppm): 7.67 (m, 2H), 7.52 (m, 2H), 7.23 (m, 2H), 6.98 (m, 2H), 3.741 (s, 2H), 3.51 (s, 2H), 3.06 (s, 4H), 2.57 (s, 4H).

### EXAMPLE 124

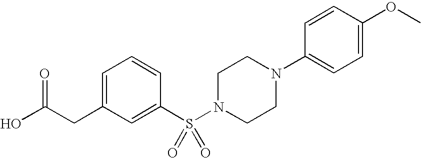

### {3-[4-(4-Methoxy-phenyl)-piperazine-1-sulfonyl]-phenyl}-acetic acid

¹H NMR (400 MHz, CDCl₃), δ (ppm): 7.72 (m, 2H), 7.54 (m, 2H), 6.82 (m, 5H), 3.76 (s, 3H), 3.72 (s, 2H), 3.17 (m, 4H), 3.11 (m, 4H).

### EXAMPLE 125

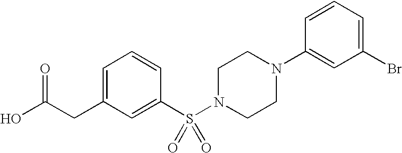

### {3-[4-(3-Bromo-phenyl)-piperazine-1-sulfonyl]-phenyl}-acetic acid

¹H NMR (400 MHz, CDCl₃), δ (ppm): 7.72 (m, 2H), 7.54 (m, 2H), 7.09 (m, 1H), 6.98 (m, 2H), 6.76 (m, 1H), 3.76 (s, 2H), 3.23 (m, 4H), 3.16 (m, 4H).

### EXAMPLE 126

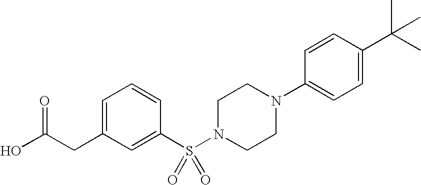

### {3-[4-(4-tert-butyl-phenyl)-piperazine-1-sulfonyl]-phenyl}-acetic acid

¹H NMR (400 MHz, CDCl₃), δ (ppm): 7.72 (m, 2H), 7.54 (m, 2H), 7.27 (d, 2H), 6.82 (d, 2H), 3.73 (s, 2H), 3.19 (m, 8H), 1.29 (s, 9H).

### EXAMPLE 127

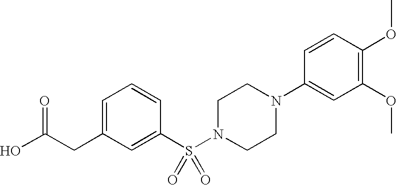

### {3-[4-(3,4-Dimethoxy-phenyl)-piperazine-1-sulfonyl]-phenyl}-acetic acid

¹H NMR (400 MHz, CDCl₃), δ (ppm): 7.72 (m, 2H), 7.52 (m, 2H), 6.76 (d, 1H), 6.49 (s, 1H), 6.42 (d, 1H), 3.82 (s, 3H), 3.80 (s, 3H), 3.70 (s, 2H), 3.15 (m, 8H).

### EXAMPLE 128

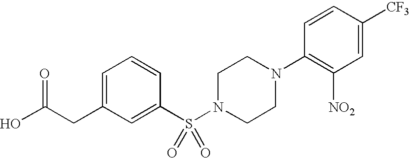

### {3-[4-(2-Nitro-4-trifluoromethyl-phenyl)-piperazine-1-sulfonyl]-phenyl}-acetic acid

¹H NMR (400 MHz, CDCl₃), δ (ppm): 8.06 (s, 1H), 7.72 (m, 3H), 7.56 (m, 2H), 7.18 (d, 1H), 3.77 (s, 2H), 3.20 (m, 8H).

### EXAMPLE 129

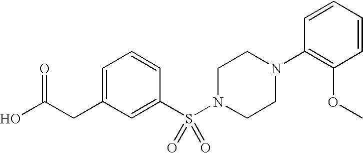

### {3-[4-(2-Methoxy-phenyl)-piperazine-1-sulfonyl]-phenyl}-acetic acid

¹H NMR (400 MHz, CDCl₃), δ (ppm): 7.71 (m, 2H), 7.52 (m, 2H), 7.02 (m, 1H), 6.90 (m, 2H), 6.83 (d, 1H), 3.79 (s, 3H), 3.71 (s, 2H), 3.19 (m, 4H), 3.11 (m, 4H).

### EXAMPLE 130

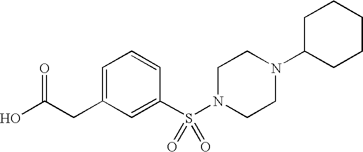

### [3-(4-Cyclohexyl-piperazine-1-sulfonyl)-phenyl]-acetic acid

¹H NMR (400 MHz, CDCl₃), δ (ppm): 7.62 (m, 2H), 7.46 (m, 2H), 3.51 (s, 2H), 3.18 (m, 4H), 2.92 (m, 4H), 2.62 (m, 1H), 1.88 (m, 2H), 1.80 (m, 2H), 1.63 (m, 1H), 1.25 (m, 4H), 1.08 (m, 1H).

### EXAMPLE 131

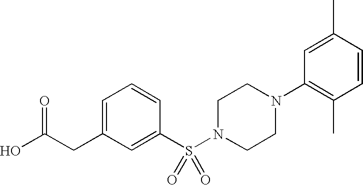

### {3-[4-(2,5-Dimethyl-phenyl)-piperazine-1-sulfonyl]-phenyl}-acetic acid

¹H NMR (400 MHz, CDCl₃), δ (ppm): 7.74 (m, 2H), 7.54 (m, 2H), 7.03 (d, 1H), 6.81 (m, 3H), 3.77 (s, 2H), 3.17 (m, 4H), 2.96 (m, 4H), 2.30 (s, 1H), 2.13 (S, 3H).

### EXAMPLE 132

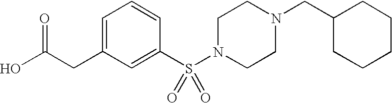

### [3-(4-Cyclohexylmethyl-piperazine-1-sulfonyl)-phenyl]-acetic acid

¹H NMR (400 MHz, CDCl₃), δ (ppm): 7.64 (m, 2H), 7.49 (m, 2H), 3.59 (s, 2H), 3.12 (m, 4H), 2.67 (m, 4H), 2.29 (d, 2H), 1.66 (m, 5H), 1.48 (m, 1H), 1.13 (m, 3H), 0.88 (m, 2H).

### EXAMPLE 133

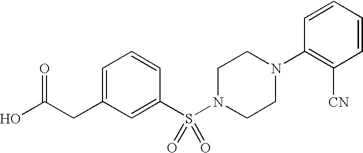

### {3-[4-(2-Cyano-phenyl)-piperazine-1-sulfonyl]-phenyl}-acetic acid

¹H NMR (400 MHz, CDCl₃), δ (ppm): 7.74 (m, 2H), 7.54 (m, 4H), 7.06 (t, 1H), 7.00 (d, 1H), 3.75 (s, 2H), 3.23 (m, 8H).

### EXAMPLE 134

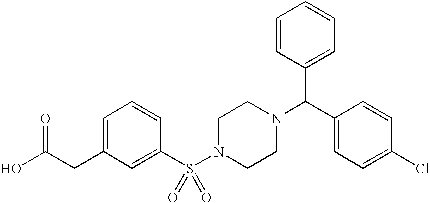

### (3-{4-[(4-Chloro-phenyl)-phenyl-methyl]-piperazine-1-sulfonyl}-phenyl)-acetic, acid

¹H NMR (400 MHz, CDCl₃), δ (ppm): 7.67 (m, 2H), 7.56 (m, 2H), 7.25 (m, 9H), 4.21 (s, 1H), 3.78 (s, 2H), 3.04 (s, 4H), 2.46 (s, 4H).

### EXAMPLE 135

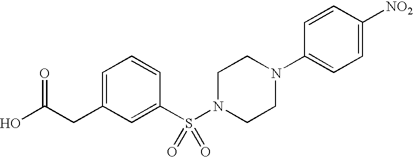

### {3-[4-(4-Nitro-phenyl)-piperazine-1-sulfonyl]-phenyl}-acetic acid

¹H NMR (400 MHz, CDCl₃), δ (ppm): 8.10 (d, 2H), 7.72 (m, 2H), 7.54 (m, 2H), 6.78 (d, 2H), 3.76 (s, 2H), 3.50 (m, 4H), 3.18 (m, 4H).

### EXAMPLE 136

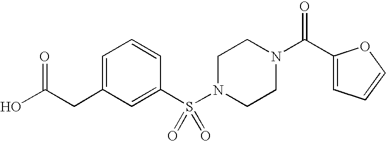

### {3-[4-(Furan-2-carbonyl)-piperazine-1-sulfonyl]-phenyl}-acetic acid

¹H NMR (400 MHz, CDCl₃), δ (ppm): 7.68 (m, 2H), 7.45 (m, 1H), 7.02 (m, 1H), 6.46 (m, 1H), 3.89 (b, 4H), 3.73 (s, 2H), 3.09 (m, 4H).

### EXAMPLE 137

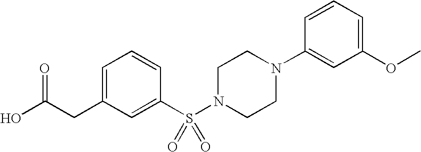

### {3-[4-(3-Methoxy-phenyl)-piperazine-1-sulfonyl]-phenyl}-acetic acid

¹H NMR (400 MHz, CDCl₃), δ (ppm): 7.72 (m, 2H), 7.53 (m, 2H), 7.16 (t, 1H), 6.49 (m, 2H), 6.40 (s, 1H), 3.77 (s, 3H), 3.74 (s, 2H), 3.23 (m, 4H), 3.16 (m, 4H).

### EXAMPLE 138

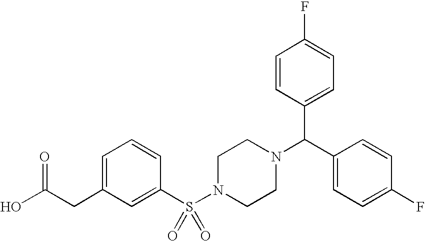

### (3-{4-[Bis-(4-fluoro-phenyl)-methyl]-piperazine-1-sulfonyl}-phenyl)-acetic acid

¹H NMR (400 MHz, CDCl₃), δ (ppm): 7.68 (m, 2H), 7.56 (m, 2H), 7.26 (t, 4H), 6.94 (t, 4H), 4.22 (s, 1H), 3.77 (s, 2H), 3.03 (s, 4H), 2.44 (m, 4H).

### EXAMPLE 139

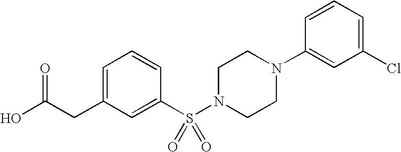

### {3-[4-(3-Chloro-phenyl)-piperazine-1-sulfonyl]-phenyl}-acetic acid

¹H NMR (400 MHz, CDCl₃), δ (ppm): 7.70 (m, 2H), 7.52 (m, 2H), 7.15 (t, 1H), 6.82 (m, 2H), 6.73 (d, 1H), 3.74 (s, 2H), 3.23 (m, 4H), 3.16 (m, 4H).

### EXAMPLE 140

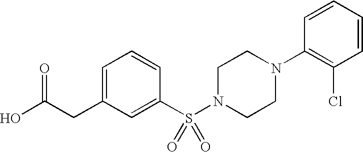

### {3-[4-(2-Chloro-phenyl)-piperazine-1-sulfonyl]-phenyl}-acetic acid

¹H NMR (400 MHz, CDCl₃), δ (ppm): 7.72 (m, 2H), 7.56 (m, 2H), 7.33 (m, 1H), 7.24 (m, 1H), 7.00 (m, 2H), 3.77 (s, 2H), 3.22 (s, 4H), 3.12 (m, 4H).

### EXAMPLE 141

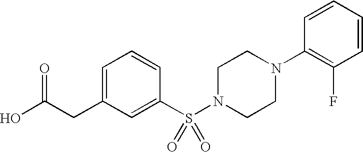

### {3-[4-(2-Fluoro-phenyl)-piperazine-1-sulfonyl]-phenyl}-acetic acid

¹H NMR (400 MHz, CDCl₃), δ (ppm): 7.72 (m, 2H), 7.56 (m, 2H), 7.02 (m, 4H), 3.76 (s, 2H), 3.20 (m, 4H), 3.15 (m, 4H).

### EXAMPLE 142

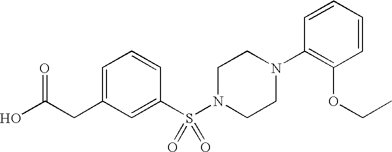

### {3-[4-(2-Ethoxy-phenyl)-piperazine-1-sulfonyl]-phenyl}-acetic acid

¹H NMR (400 MHz, CDCl₃), δ (ppm): 7.72 (m, 2H), 7.53 (m, 2H), 6.98 (m, 1H), 6.90 (m, 2H), 6.82 (d, 1H), 4.10 (q, 2H), 3.75 (s, 2H), 3.20 (m, 4H), 3.15 (m, 4H), 1.38 (t, 3H).

### EXAMPLE 143

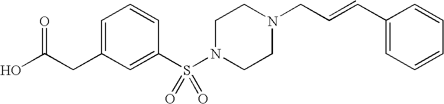

### {3-[4-(3-Phenyl-allyl)-piperazine-1-sulfonyl]-phenyl}-acetic acid

¹H NMR (400-MHz, CDCl₃), δ (ppm): 7.62 (m, 2H), 7.48 (m, 2H), 7.27 (m, 5H), 6.54 (d, 1H), 6.12 (m, 1H), 3.58 (s, 2H), 3.27 (d, 2H), 3.13 (s, 4H), 2.74 (s, 4H).

### EXAMPLE 144

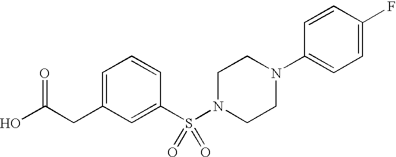

### {3-[4-(4-Fluoro-phenyl)-piperazine-1-sulfonyl]-phenyl}-acetic acid

¹H NMR (400 MHz, CDCl₃), δ (ppm): 7.72 (m, 2H), 7.54 (m, 2H), 6.95 (m, 2H), 6.83 (m, 2H), 3.75 (s, 2H), 3.15 (m, 8H).

### EXAMPLE 145

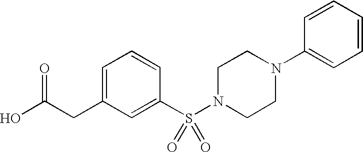

### [3-(4-Phenyl-piperazine-1-sulfonyl)-phenyl]-acetic acid

¹H NMR (400 MHz, CDCl₃), δ (ppm): 7.72 (m, 2H), 7.52 (m, 2H), 7.26 (m, 2H), 6.89 (m, 3H), 3.73 (s, 2H), 3.22 (m, 4H), 3.18 (m, 4H).

### EXAMPLE 146

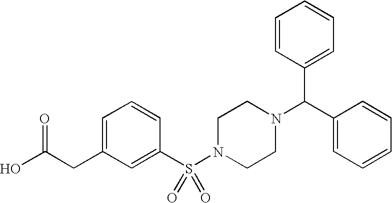

### [3-(4-Benzhydryl-piperazine-1-sulfonyl)-phenyl]-acetic acid

¹H NMR (400 MHz, CDCl₃), δ (ppm): 7.69 (m, 2H), 7.55 (m, 2H), 7.33 (m, 4H), 7.25 (m, 4H), 7.17 (m, 2H), 4.22 (s, 1H), 3.77 (s, 2H), 3.04 (s, 4H), 2.47 (s, 4H).

### PREPARATION OF EXAMPLES 147-165

Examples 147-165 were prepared from 3-Chlorosulfonyl-4-methyl-phenyl)-acetic acid ethyl ester as set forth in Example 1, step 1 according to the general procedure below:

A) Parallel Syntheses of Piperazine Sulfonamide Intermediates.

Nineteen separate solution vials were charged with the above intermediate (0.72 mmol, 1.0 eqv) in 3 mL of THF. To each vial was added the corresponding piperazine (0.72 mmol, 1.0 eqv), followed by triethylamine (1.45 mmol, 2.0 eqv) and a catalytic amount of DMAP. The reaction mixtures were stirred at 40° C. overnight. The solvent was evaporated and the residues were purified by chromatography.

B) Parallel Synthesis of Examples 147-165.

Ethyl esters (1.0 eqv) were dissolved in 2 mL of THF/MeOH (3:1), followed by addition of 1N LiOH (5.0 eqv). The resulting mixtures were stirred at 40° C. for 3 hours. The organic solvent was evaporated under N₂ and residues were diluted with water (2 mL). The aqueous layers were extracted with ether (2 mL). After removal of organic layers, the aqueous layers were neutralized by 1N HCl (5.0 eqv) and then extracted with ethyl acetate (5 mL). The organic layers were washed with water, brine, and dried over Na₂SO₄. Removal of solvent afforded compounds 147-165.

### EXAMPLE 147

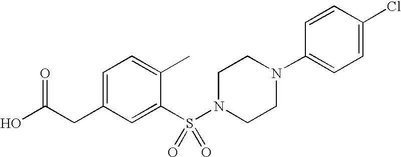

### {3-[4-(4-Chloro-phenyl)-piperazine-1-sulfonyl]-4-methyl-phenyl}-acetic acid

¹H NMR (400 MHz, CDCl₃) δ ppm. 2.62 (s, 3H), 3.16 (m, 4H), 3.31 (m, 4H), 3.68 (s, 2H), 6.79 (d, 2H), 7.19 (d, 2H), 7.30 (d, 1H), 7.38 (d, 1H), 7.84 (s, 1H).

### EXAMPLE 148

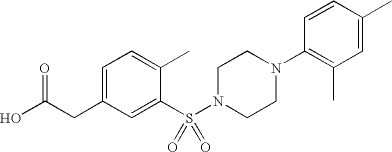

### {3-[4-(2,4-Dimethyl-phenyl)-piperazine-1-sulfonyl]-4-methyl-phenyl}-acetic acid

¹H NMR (400 MHz, CDCl₃) δ ppm. 2.21 (s, 3H), 2.26 (s, 3H), 2.65 (s, 3H), 2.90 (m, 4H), 3.31 (m, 4H), 3.69 (s, 2H), 6.89 (d, 1H), 6.97 (m, 2H), 7.30 (d, 1H), 7.39 (d, 1H), 7.86 (s, 1H).

### EXAMPLE 149

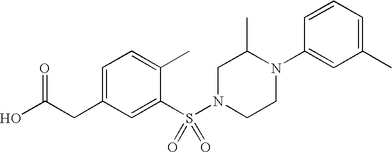

### [4-Methyl-3-(3-methyl-4-m-tolyl-piperazine-1-sulfonyl)-phenyl]-acetic acid

¹H NMR (400 MHz, CDCl₃) δ ppm. 1.00 (d, 3H), 2.28 (s, 3H), 2.64 (s, 3H), 3.02 (m, 1H), 3.19 (m, 3H), 3.30 (m, 1H), 3.53 (m, 1H), 3.64 (s, 2H), 3.80 (m, 1H), 6.72 (m, 3H), 7.13 (t, 1H), 7.27 (d, 1H), 7.28 (d, 1H), 7.37 (s, 1H).

### EXAMPLE 150

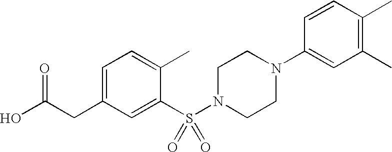

### {3-[4-(3,4-Dimethyl-phenyl)-piperazine-1-sulfonyl]-4-methyl-phenyl}-acetic acid

¹H NMR (400 MHz, CDCl₃) δ ppm. 2.20 (s, 3H), 2.24 (s, 3H), 2.65 (s, 3H), 3.17 (m, 4H), 3.34 (m, 4H), 3.70 (s, 2H), 6.67 (d, 1H), 6.74 (s, 1H), 7.04 (d, 1H), 7.31 (d, 1H), 7.41 (d, 1H), 7.86 (s, 1H).

### EXAMPLE 151

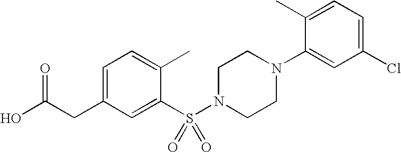

### {3-[4-(5-Chloro-2-methyl-phenyl)-piperazine-1-sulfonyl]-4-methyl-phenyl}-acetic acid

¹H NMR (400 MHz, CDCl₃) δ ppm. 2.22 (s, 3H), 2.66 (s, 3H), 2.82 (m, 4H), 3.34 (m, 4H), 3.73 (s, 2H), 6.95 (s, 1H), 6.78 (d, 1H), 7.09 (d, 1H), 7.34 (d, 1H), 7.43 (d, 1H), 7.88 (s, 1H).

### EXAMPLE 152

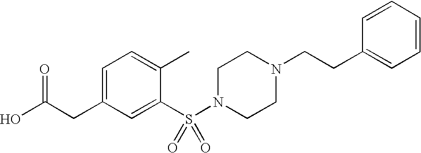

### [4-Methyl-3-(4-phenethyl-piperazine-1-sulfonyl)-phenyl]-acetic acid

¹H NMR (400 MHz, CDCl₃) δ ppm. 2.58 (s, 3H), 2.90 (m, 8H), 3.42 (m, 4H), 3.62 (s, 2H), 7.16 (d, 2H), 7.28 (m, 4H), 7.40 d, 1H), 7.81 (s, 1H).

### EXAMPLE 153

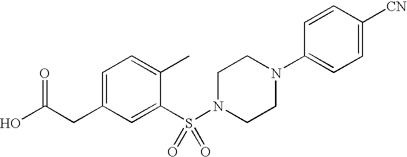

### {3-[4-(4-Cyano-phenyl)-piperazine-1-sulfonyl]-4-methyl-phenyl}-acetic acid

¹H NMR (400 MHz, CDCl₃) δ ppm. 2.64 (s, 3H), 3.33 (m, 4H), 3.38 (m, 4H), 3.71 (s, 2H), 6.85 (d, 2H), 7.32 (d, 1H), 7.41 (d, 1H), 7.49 (d, 2H), 7.86 (s, 1H).

### EXAMPLE 154

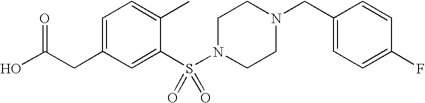

### {3-[4-(4-Fluoro-benzyl)-piperazine-1-sulfonyl]-4-methyl-phenyl}-acetic acid

¹H NMR (400 MHz, CDCl₃) δ ppm. 2.53 (s, 3H), 2.74 (m, 4H), 3.32 (m, 4H), 3.60 (s, 2H), 3.70 (s, 2H), 7.00 (t, 2H), 7.26 (m, 3H), 7.35 (d, 1H), 7.76 (s, 1H).

### EXAMPLE 155

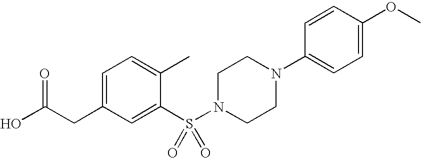

### {3-[4-(4-Methoxy-phenyl)-piperazine-1-sulfonyl]-4-methyl-phenyl}-acetic acid

¹H NMR (400 MHz, CDCl₃) δ ppm. 2.65 (s, 3H), 3.11 (m, 4H), 3.34 (m, 4H), 3.70 (s, 2H), 3.78 (s, 3H), 6.84 (d, 2H), 6.91 (d, 2H), 7.29 (d, 1H), 7.41 (d, 1H), 7.85 (s, 1H).

### EXAMPLE 156

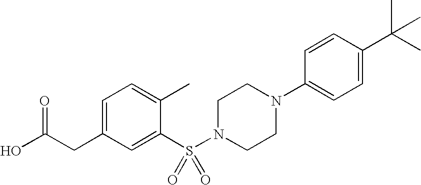

### {3-[4-(4-tert-Butyl-phenyl)-piperazine-1-sulfonyl]-4-methyl-phenyl}-acetic acid

¹H NMR (400 MHz, CDCl₃) δ ppm. 1.28 (s, 9H), 2.63 (s, 3H), 3.18 (m, 4H), 3.31 (m, 4H), 3.68 (s, 2H), 6.84 (d, 2H), 7.30 (d, 3H), 7.40 (d, 1H), 7.85 (s, 1H).

### EXAMPLE 157

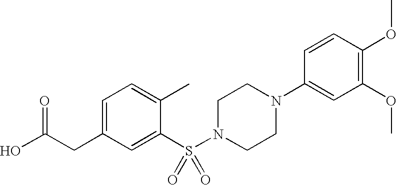

### {3-[4-(3,4-Dimethoxy-phenyl)-piperazine-1-sulfonyl]-4-methyl-phenyl}-acetic acid

¹H NMR (400 MHz, CDCl₃) δ ppm. 2.65 (s, 3H), 3.12 (m, 4H), 3.34 (m, 4H), 3.71 (s, 2H), 3.85 (s, 3H), 3.88 (s, 3H), 6.47 (d, 1H), 6.56 (s, 1H), 6.79 (d, 1H), 7.32 (d, 1H), 7.42 (d, 1H), 7.86 (s, 1H).

### EXAMPLE 158

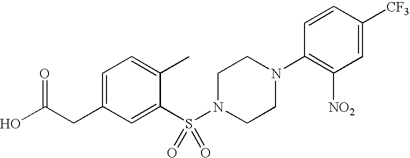

### {4-Methyl-3-[4-(2-nitro-4-trifluoromethyl-phenyl)-piperazine-1-sulfonyl]-phenyl}-acetic acid

¹H NMR (400 MHz, CDCl₃) δ ppm. 2.64 (s, 3H), 3.32 (m, 4H), 3.37 (m, 4H), 3.72 (s, 2H), 7.20 (d, 1H), 7.32 (d, 1H), 7.43 (d, 1H), 7.72 (d, 1H), 7.85 (s, 1H), 8.10 (s, 1H).

### EXAMPLE 159

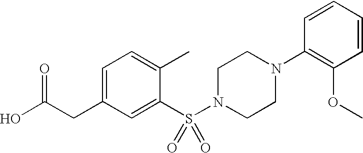

### {3-[4-(2-Methoxy-phenyl)-piperazine-1-sulfonyl]-4-methyl-phenyl}-acetic acid

¹H NMR (400 MHz, CDCl₃) δ ppm. 2.66 (s, 3H), 3.12 (m, 4H), 3.35 (m, 4H), 3.69 (s, 2H), 3.84(s, 3H), 6.86-6.93 (m, 3H), 7.04 (t, 1H), 7.31 (d, 1H), 7.41 (d, 1H), 7.83 (s, 1H).

### EXAMPLE 160

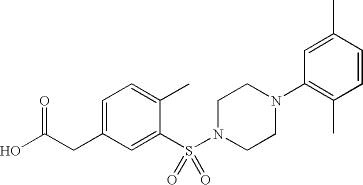

### {3-[4-(2,5-Dimethyl-phenyl)-piperazine-1-sulfonyl]-4-methyl-phenyl}-acetic acid

¹H NMR (400 MHz, CDCl₃) δ ppm. 2.05 (s, 3H), 2.19 (s, 3H), 2.65 (s, 3H), 2.93 (m, 4H), 3.32 (m, 4H), 3.70 (s, 2H), 6.79 (s, 1H), 6.82 (d, 1H), 7.04 (d, 1H), 7.32 (d, 1H), 7.41 (d, 1H), 7.86 (s, 1H).

### EXAMPLE 161

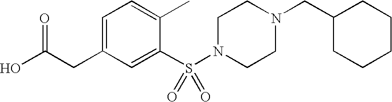

### [3-(4-Cyclohexylmethyl-piperazine-1-sulfonyl)-4-methyl-phenyl]-acetic acid

¹H NMR (400 MHz, CDCl₃) δ ppm. 0.89-0.92 (q, 2H), 1.1 5-1.18 (m, 4H), 1.62-1.74 (m, 4H), 2.47 (d, 2H), 2.56 (s, 3H), 2.85 (m, 4H), 3.39 (m, 4H), 3.39 (s, 2H), 7.25 (d, 1H), 7.38 (d, 1H), 7.77 (s, 1H).

### EXAMPLE 162

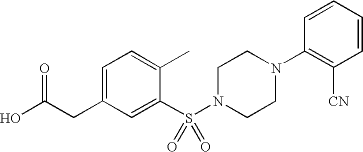

### {3-[4-(2-Cyano-phenyl)-piperazine-1-sulfonyl]-4-methyl-phenyl}-acetic acid

¹H NMR (400 MHz, CDCl₃), δ ppm. 2.63 (s, 3H), 3.24 (m, 4H), 3.37 (m, 4H), 3.69 (s, 2H), 6.99 (d, 1H), 7.05 (t, 1H), 7.29 (d, 1H), 7.40 (d, 1H), 7.50 (t, 1H), 7.56 (d, 1H), 7.83 (s, 1H).

### EXAMPLE 163

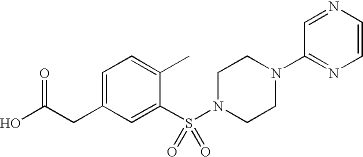

### [4-Methyl-3-(2,3,5,6-tetrahydro-[1,2]bipyrazinyl-4-sulfonyl)-phenyl]-acetic acid

¹H NMR (400 MHz, CDCl₃) δ ppm. 2.74 (s, 3H), 3.29 (m, 4H), 3.65 (m, 6H), 7.29 (d, 1H), 7.42 (d, 1H), 7.83 (d, 2H), 8.07 (s, 1H), 8.11 (s, 1H).

### EXAMPLE 164

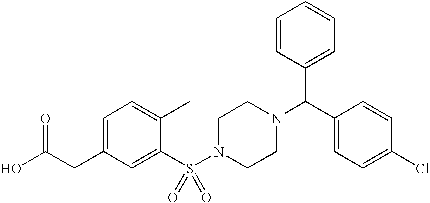

### {3-[4-(4-Chloro-phenyl)-phenyl-methyl]-piperazine-1-sulfonyl}-4-methyl-phenyl)-acetic acid

¹H NMR (400 MHz, CDCl₃) δ ppm. 2.44 (m, 4H), 2.59 (s, 3H), 3.18 (m, 4H), 3.66 (s, 2H), 7.19-7.32 (m, 10H), 7.38 (d, 1H), 7.78 (s, 1H).

### EXAMPLE 165

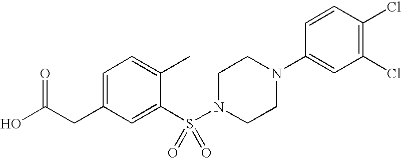

### {3-[4-(3,4-Dichloro-phenyl)-piperazine-1-sulfonyl]-4-methyl-phenyl}-acetic acid

¹H NMR (400 MHz, CDCl₃) δ ppm. 2.64 (s, 3H), 3.21 (m, 4H), 3.32 (m, 4H), 3.72 (s, 2H), 6.72 (d, 1H), 6.95 (s, 1H), 7.32 (m, 2H), 7.41 (d, 1H), 7.86 (s, 1H).

### PREPARATION OF EXAMPLES 166-174

A) 5-Chlorosulfonyl-3-methyl-phenyl)-acetic Acid Methyl Ester.

A solution of (3-mercapto-phenyl)-acetic acid methyl ester (10.45 g,57.3 mmol, 1.0 equiv.) in MeCN (200 mL) was cooled to 0° C. To this cold solution was added KNO₃ (14.5 g, 143.3 mmol, 2.5 equiv) followed by SO₂Cl₂ (11.7 mL, 143.3 mmol, 2.5 equiv) with stirring. The resulting suspension was vigorously-stirred at 0° C. for 3.0 hours and then diluted with ether (200 mL). The mixture was neutralized with saturated Na₂CO₃ to pH 7˜8. After separation, the aqueous solution was extracted with ether (200 mL×2) and the combined organic solution was washed with brine, dried over Na₂SO₄. After removal of solvent, 11.73 g (82% yield ) of desired intermediate was obtained as a brown oil, suitable for use without purification. ¹H NMR (400 MHz, CDCl₃), δ (ppm): 7.97 (m, 1H), 7.68 (d, 1H), 7.62 (t, 1H), 3.76 (s, 2H), 3.75 (s, 3H).

B) Parallel Synthesis of Examples 166-174.

The compounds of Examples 166-174 were prepared from 5-Chlorosulfonyl-3-methyl-phenyl)-acetic acid using the method of Examples 115-165.

### EXAMPLE 166

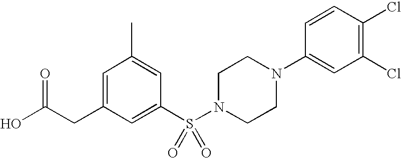

### {3-[4-(3,4-Dichloro-phenyl)-piperazine-1-sulfonyl]-5-methyl-phenyl}-acetic acid

¹H NMR (400 MHz, CDCl₃) δ ppm. 2.43 (s, 3H), 3.17 (m, 4H), 3.21 (m, 4H), 3.71 (s, 2H), 6.69 (d, 1H), 6.91 (s, 1H), 7.25 (d, 1H), 7.35 (s, 1H), 7.51 (s, 2H).

### EXAMPLE 167

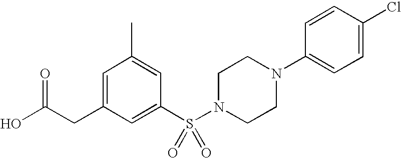

### {3-[4-(4-Chloro-phenyl)-piperazine-1-sulfonyl]-5-methyl-phenyl}-acetic acid

¹H NMR (400 MHz, CDCl₃) δ ppm. 2.41 (s, 3H), 3.22 (m, 8H), 6.88 (d, 2H), 7.22 (d, 2H), 7.38 (s, 1H), 7.51 (s, 2H).

### EXAMPLE 168

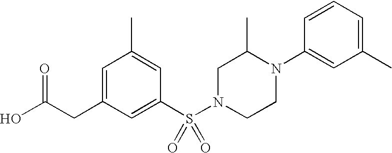

### [3-Methyl-5-(3-methyl-4-m-tolyl-piperazine-1-sulfonyl)-phenyl]-acetic acid

¹H NMR (400 MHz, CDCl₃) δ ppm. 1.12 (s, 3H), 2.22 (s, 3H), 2.44 (s, 3H), 3.28 (m, 4H), 3.51 (m, 1H), 3.70 (s, 2H), 3.82 (m, 2H), 7.25 (m, 4H), 7.35 (s, 1H), 7.52 (m, 2H).

### EXAMPLE 169

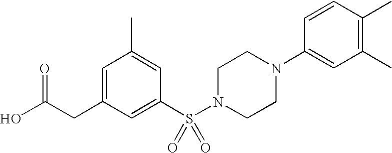

### {3-[4-(3,4-Dimethyl-phenyl)-piperazine-1-sulfonyl]-5-methyl-phenyl}-acetic acid

¹H NMR (400 MHz, CDCl₃) δ ppm. 2.19 (s, 3H), 2.21 (s, 3H), 2.41 (s, 3H), 3.22 (m, 8H), 3.71 (s, 2H), 7.03 (d, 1H), 7.24 (m, 3H), 7.35 (s, 1H), 7.52 (m, 2H).

### EXAMPLE 170

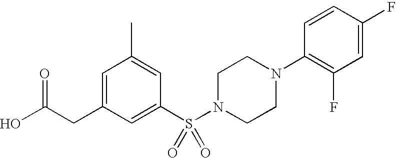

### {3-[4-(2,4-Difluoro-phenyl)-3-methyl-piperazine-a-sulfonyl]-5-methyl-phenyl}-acetic acid

¹H NMR (400 MHz, CDCl₃) δ ppm. 2.44 (s, 3H), 3.1 0 (m, 4H), 3.19 (m, 4H), 3.71 (s, 2H), 6.80 (m, 2H), 6.89 (m, 1H), 7.36 (s, 1H), 7.52 (s, 2H).

### EXAMPLE 171

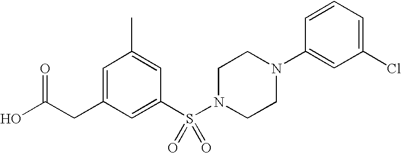

### {3-[4-(3-Chloro-phenyl)-piperazine-1-sulfonyl]-5-methyl-phenyl}-acetic acid

¹H NMR (400 MHz, CDCl₃) δ ppm. 2.44 (s, 3H), 3.20 (m, 4H), 3.28 (m, 4H), 3.71 (s, 2H), 6.77 (s, 1H), 6.87 (d, 2H), 7.17 (t, 1H), 7.37 (s, 1H), 7.52 (s, 2H).

### EXAMPLE 172

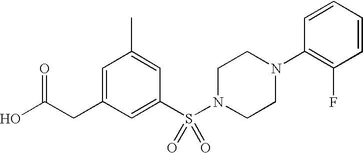

### {3-[4-(2-Fluoro-phenyl)-piperazine-1-sulfonyl]-5-methyl-phenyl}-acetic acid

¹H NMR (400 MHz, CDCl₃) δ ppm. 2.42 (s, 3H), 3.17 (m, 4H), 3.20 (m, 4H), 3.70 (s, 2H), 6.91 (t, 1H), 6.97 (d, 1H), 6.98 (d, 1H), 7.06 (t, 1H), 7.35 (s, 1H), 7.51 (s, 2H).

### EXAMPLE 173

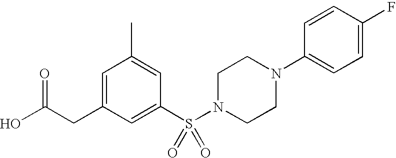

### {3-[4-(4-Fluoro-phenyl)-piperazine-1-sulfonyl]-5-methyl-phenyl}-acetic acid.

¹H NMR (400 MHz, CDCl₃) δ ppm. 2.42 (s, 3H), 3.21 (m, 4H), 3.24 (m, 4H), 3.71 (s, 2H), 6.99 (m, 4H), 7.36 (s, 1H), 7.51 (s, 2H).

### EXAMPLE 174

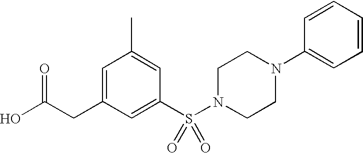

### [3-Methyl-5-(4-phenyl-piperazine-1-sulfonyl)-phenyl]-acetic acid

¹H NMR (400 MHz, CDCl₃) δ ppm. 2.41 (s, 3H), 3.19 (m, 4H), 3.23 (m, 4H), 3.70 (s, 2H), 6.90 (m, 3H), 7.23 (d, 2H), 7.31 (s, 1H), 7.51 (s, 2H).

### PREPARATION OF EXAMPLES 175-183

A) (5-Chlorosulfonyl-2-methyl-phenyl)-acetic Acid Methyl Ester.

Title compound was prepared according to Scheme 1 by chlorosulfonylating 2-methyl-phenyl-acetic acid methyl ester to give the product as a white solid. ¹H NMR (400 MHz, CDCl₃) δ ppm. 7.84 (d, 1H), 7.83 (s, 1H), 7.42 (d, 1H), 3.75 (s, 3H), 3.73 (s, 2H), 2.43 (s, 3H).

B) Parallel Synthesis of Piperazine Sulfonamide Intermediates

Solutions of intermediates (5-Chlorosulfonyl-2-methyl-phenyl)-acetic acid methyl ester (0.76 mmol, 1.0 eqv) in 4 mL of THF were charged in 9 reaction vials, respectively. To each vial was added the corresponding piperazine (0.76 mmol, 1.0 eqv), followed by triethylamine (1.52 mmol, 2.0 eqv) and a catalytic amount of DMAP. The reaction mixtures were stirred at room temperature overnight. The solvent was evaporated and the residues were purified by chromatography.

C) Parallel Synthesis of Examples 175-183

Compounds of Examples 175-183 were prepared from the above intermediates using the methods used to prepared Examples 115-174. NMR data of Compounds 175-183 are described as below.

### EXAMPLE 175

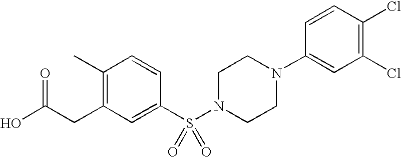

### {5-[4-(3,4-Dichloro-phenyl)-piperazine-1-sulfonyl]-2-methyl-phenyl}-acetic acid

¹H NMR (400 MHz, CDCl₃) δ ppm. 2.40 (s, 3H), 3.14 (m, 4H), 3.21 (m, 4H), 3.76 (s, 2H),6.66 (d, 1H), 6.89 (d, 1H), 7.25 (s, 1H), 7.38 (d, 1H), 7.64 (d, 1H), 7.66 (s, 1H).

### EXAMPLE 176

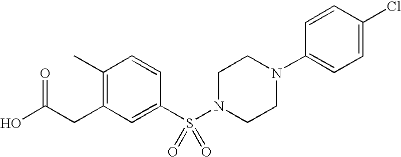

### {5-[4-(4-Chloro-phenyl)-piperazine-1-sulfonyl]-2-methyl-phenyl}-acetic acid

¹H NMR (400 MHz, CDCl₃) δ ppm. 2.40 (s, 3H), 3.17 (m, 4H), 3.19 (m, 4H), 3.76 (s, 2H), 6.79 (d, 2H), 7.20 (d, 2H), 7.38 (d, 1H), 7.60 (d, 1H), 7.61 (s, 1H).

### EXAMPLE 177

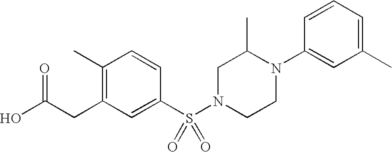

### [2-Methyl-5-(3-methyl-4-m-tolyl-piperazine-1-sulfonyl)-phenyl]-acetic acid

¹H NMR (400 MHz, CDCl₃) δ ppm. 1.08 (d, 3H), 2.30 (s, 3H), 2.39 (s, 3H), 2.75 (m, 1H), 2.90 (d, 1H), 3.18 (t, 2H), 3.21 (d, 1H), 3.48 (d, 1H), 3.75 (s, 2H), 3.80 (m, 1H).

### EXAMPLE 178

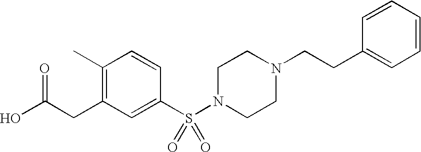

### [2-Methyl-5-(4-phenethyl-piperazine-1-sulfonyl)-phenyl]-acetic acid

¹H NMR (400 MHz, CDCl₃) δ ppm. 2.37 (s, 3H), 2.76-2.85 (m, 8H), 3.18 (m, 4H), 3.64 (s, 2H), 7.14(d, 2H), 7.21 (t, 1H), 7.28 (t, 2H), 7.32 (d, 1H), 7.53 (d, 1H), 7.59 (s, 1H).

### EXAMPLE 179

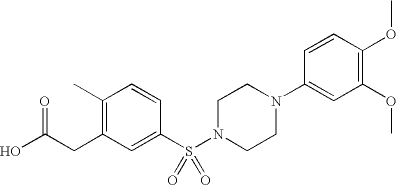

### {5-[4-(3,4-Dimethoxy-phenyl)-piperazine-1-sulfonyl]-2-methyl-phenyl}-acetic acid

¹H NMR (400 MHz, CDCl₃) δ ppm. 2.40 (s, 3H), 3.12-3.20 (m, 8H), 3.73 (s, 2H), 3.81 (s, 6H), 6.79 (d, 1H), 7.26 (s, 1H), 7.26 (d, 1H), 7.37 (d, 1H), 7.61 (d, 1H), 7.63 (s, 1H).

### EXAMPLE 180

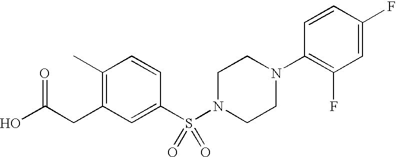

### {5-[4-(2,4-Difluoro-phenyl)-piperazine-1-sulfonyl]-2-methyl-phenyl}-acetic acid

¹H NMR (400 MHz, CDCl₃) δ ppm. 2.40 (s, 3H), 3.09 (m, 4H), 3.18 (m, 4H), 3.77 (s, 2H), 6.78 (m, 2H),6.89 (d, 1H), 7.38 (s, 1H), 7.62 (d, 1H), 7.64 (s, 1H).

### EXAMPLE 181

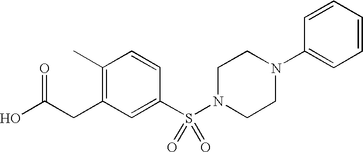

### [2-Methyl-5-(4-phenyl-piperazine-1-sulfonyl)-phenyl]-acetic acid

¹H NMR (400 MHz, CDCl₃) δ ppm. 2.40 (s, 3H), 3.15 (m, 4H), 3.21 (m, 4H), 3.75 (s, 2H), 6.89 (m, 3H), 7.23 (d, 1H), 7.25 (s, 1H), 7.37 (d, 1H), 7.61 (d, 1H), 7.62 (s, 1H).

### EXAMPLE 182

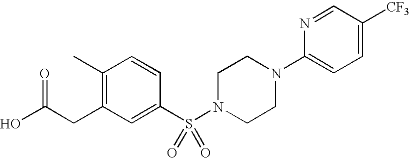

### {2-Methyl-S-[4-(5-trifluoromethyl-pyridin-2-yl)-piperazine-1-sulfonyl]-phenyl}-acetic acid

¹H NMR (400 MHz, CDCl₃) δ ppm. 2.39 (s, 3H), 3.09 (m, 4H), 3.72 (m, 4H), 3.72 (s, 2H), 6.59 (d, 1H), 7.32 (d, 1H), 7.59 (d, 1H), 7.60 (s, 1H), 8.34 (s, 1H).

### EXAMPLE 183

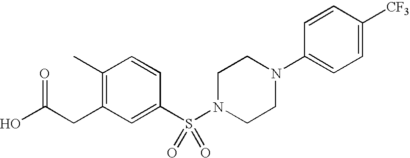

### {2-Methyl-5-[4-(4-trifluoromethyl-phenyl)-piperazine-1-sulfonyl]-phenyl}-acetic acid

¹H NMR (400 MHz, CDCl₃) δ ppm. 2.40 (s, 3H), 3.18 (m, 4H), 3.34 (m, 4H), 3.76 (s, 2H), 6.88 (d, 2H), 7.37 (d, 1H), 7.48 (d, 2H), 7.61 (d, 1H), 7.64 (s, 1H).

### SYNTHESES OF EXAMPLES 184 AND 185

Examples 183 and 184 were prepared from intermediate sulfonyl halide and the corresponding piperidine in place of a piperazine.

### EXAMPLE 184

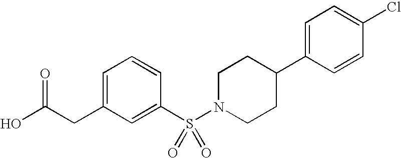

### {3-[4-(4-Chloro-phenyl)-piperidine-1-sulfonyl]-phenyl}-acetic acid

¹H NMR (400 MHz, CDCl₃) δ ppm. 7.72 (s, 2H), 7.64 (m, 2H), 7.25 (d, 2H), 7.05 (d, 2H), 3.95 (d, 2H), 3.76 (s, 2H), 2.39 (t, 3H), 1.82 (m, 4H).

### EXAMPLE 185

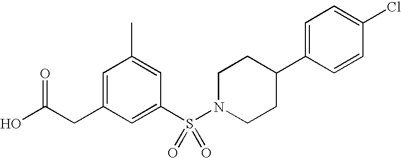

### {3-[4-(4-Chloro-phenyl)-piperidine-1-sulfonyl]-5-methyl-phenyl}-acetic acid

¹H NMR (400 MHz, CDCl₃) δ ppm. 7.51 (s, 2H), 7.36 (s, 1H), 7.24 (d, 2H), 7.07 (d, 2H), 3.95 (d, 2H), 3.71 (s, 2H), 2.43 (s, 3H), 2.40 (t, 3H), 1.82 (m, 4H).

### EXAMPLE 186

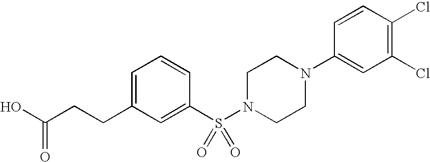

Example 186 was prepared according to Scheme XXI.

Step 1

3-(3-Dimethylthiocarbamoyloxy-phenyl)-propionic acid methyl ester. To a solution of methyl 3-(3-hydroxyphenyl) propionate (9.31 g, 51.7 mmol, 1.0 equiv.) in dioxane (100 mL), was added dimethylthiocarbamoyl chloride (7.66 g, 62.0 mmol, 1.2 equiv.), Et₃N (14.4 mL, 103.4 mmol, 2.0 equiv.), and DMAP (0.63 g, 5.2 mmol, 0.1 equiv.). The resulting mixture was stirred and heated to 100° C. overnight. The solution slowly changed color from yellow to brown over time. To the reaction mixture diluted with EtOAc (150 mL), and it was sequentially washed with water, brine, and dried over Na₂SO₄. After removal of solvent, the crude product was purified by chromatography to afford 9.6 g of yellow oil. ¹H NMR (400 MHz, CDCl₃) δ ppm. 7.29 (t, 1H), 7.19 (d, 1H), 6.90 (d, 1H), 6.89 (s, 1H), 3.65 (s, 3H), 3.41 (s, 3H), 3.32 (s, 3H), 2.94 (t, 2H), 2.64 (t, 2H).

Step 2

3-(3-Dimethylcarbamoylsulfanyl-phenyl)-propionic acid methyl ester. A high pressure reaction flask was charged with the product of Example 186, step 1 (4.40 g, 16.4 mmol) and tetradecane (30 mL). The reaction flask was sealed and the reaction mixture was heated to 250° C. in a sand bath with stirring overnight. The reaction flask was removed from the hot source and was cooled to room temperature. After tetradecane was decanted, the residue was washed with hexane (2×10 mL). The compound was dried under vacuum and purified by chromatography to afford 3.9 g of yellow oil. ¹H NMR (400 MHz, CDCl₃) δ ppm. 7.33 (t, 1H), 7.30 (d, 1H), 7.22 (s, 1H), 7.21 (d, 1H), 3.66 (s, 3H), 3.09 (s, 3H), 3.02 (s, 3H), 2.94 (t, 2H), 2.63 (t, 2H).

Step 3

3-(3-Mercapto-phenyl)-propionic acid methyl ester. To the solution of the product of Example 186, step 2 (2.25 g, 8.45 mmol) in dry MeOH (10 mL) was added 0.5N NaOMe solution in MeOH (18.6 mL, 9.30 mmol, equiv. 1.1). The reaction mixture was stirred and heated at 60° C. for 4 hours. The reaction mixture was cooled to room temperature and then neutralized with 1N HCl (9.3 mL). The reaction mixture was concentrated under reduced pressure. The residue was taken in by EtOAc, and then washed with water, brine, and dried over Na₂SO₄. The crude product was purified by chromatography to afford 1.59 g of colorless oil. ¹H NMR (400 MHz, CDCl₃) δ ppm. 7.19 (m, 3H), 7.02 (d, 1H), 3.66 (s, 3H), 2.93 (t, 2H), 2.62 (t, 2H).

Step 4

3-(3-Chlorosulfonyl-phenyl)-propionic acid methyl ester. A solution of the product of step 3 above (1.27 g, 6.50 mmol, equiv. 1.0) in CH₃CN (35 mL) was cooled to 0° C. To this cooled thiophenol solution was added KNO₃(1.64 g, 16.25 mmol, equiv. 2.5), followed by SO₂Cl₂ (1.32 mL, 16.25 mmol, equiv. 2.5). The resulting suspension was stirred vigorously at 0° C. for 2.5 hours. The reaction mixture was diluted with ethyl ether (50 mL), and then saturated Na₂CO₃ was added to the mixture to adjust the pH value to 8. After isolation of the organic layer, the aqueous layer was extracted with ethyl ether. The combined organic layers were washed with brine and then dried over Na₂SO₄. After removal of solvent, the crude product was purified by chromatography to afford 504 mg of the desired product.

Step 5

3-{3-[4-(3,4-Dichloro-phenyl)-piperazine-1-sulfonyl]-phenyl}-propionic acid methyl ester. To a solution of the product of step 4 above (0.87 mmol, 1.0 equiv.) in THF (2 mL), was added the corresponding piperazine (0.87 mmol, 1.0 equiv.), followed by Et₃N (1.74 mmol, 2.0 equiv.) and a catalytic amount of DMAP. The reaction mixtures were stirred at 40° C. overnight. The solvent was evaporated and the residue was purified by chromatography.

Step 6

3-{3-[4-(3,4-Dichloro-phenyl)-piperazine-1-sulfonyl]-phenyl}-propionic acid. The product of Example 186, step 5 above was dissolved in 3 mL of THF/MeOH (3: 1), followed by addition of 1 N LiOH (5.0 equiv.). The resulting mixture was stirred at 40° C. for 2 hours. The organic solvent was evaporated under N₂. To the residue was added 1N HCl 5.0 equiv.) and then extracted with EtOAc (5 mL). The organic layers were wasted with water, brine, and dried over Na₂SO₄. The residue was re-dissolved in a small amount of EtOAc and crystallized to obtain the desired products. ¹H NMR (400 MHz, CDCl₃) δ ppm. 7.67 (s, 2H), 7.51 (m, 2H), 7.29 (t, 1H), 6.94 (d, 1H), 6.70 (d, 1H), 3.26 (m, 4H), 3.25 (m, 4H), 3.08 (t, 2H), 2.76 (t, 2H).

### EXAMPLE 187

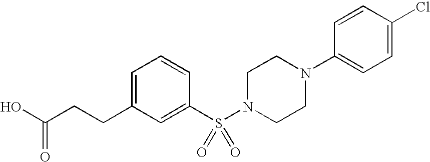

### 3-{3-[4-(4-Chloro-phenyl)-piperazine-1-sulfonyl]-phenyl}-propionic acid

The compound of Example 187 was synthesized according to the procedure described above in Example 186. ¹H NMR (400 MHz, CDCl₃) δ ppm. 7.69 (s, 2H), 7.51 (m, 2H), 7.22 (d, 2H), 6.82 (d, 2H), 3.23 (m, 4H), 3.18 (m, 4H), 3.08 (t, 2H), 2.75 (t, 2H).

### EXAMPLE 188

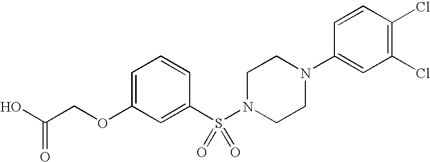

Examples 188 was prepared according to Scheme XXI.

Step 1

To a solution of 3-methoxybenzene sulfonyl chloride (531 mg, 2.57 mmol, 1.0 equiv.) in THF (8 mL), was added the corresponding piperazine (2.57 mmol, 1.0 equiv.), followed by Et₃N (5.14 mmol, 2.0 equiv.). Formation of precipitation was observed, and reaction occurred instantly as shown by TLC. The reaction mixture was stirred at room temperature for 1 hour. The solid was removed by filtration. The filtrate was concentrated under nitrogen to give the desired product.

Step 2

A solution of the product of step 1 above in DCM (5 mL) was cooled to −78° C. Under N₂ atmosphere, boron tribromide (516 μL, 5.46 mmol, 3 equiv.) was added to the solution. The resulting reaction mixture was stirred at −78° C. for 1 hour. The reaction flask was removed from the acetone/dry ice bath and then placed in an ice bath to warm to 0° C. with stirring for another 0.5 hour. The reaction flask was removed from the ice bath to warm to room temperature with stirring with additional 2 hours. The reaction mixture was slowly poured into an ice bath (200 mL), and the pH was adjusted to pH=10 with 1N NaOH. The white solid was filtered to afforded the desired product.

Step 3

To a solution of the product of step 2 above (1.5 mmol, 1 equiv.) in CH₃CN (5 mL) was added ethyl bromoacetate (3.0 mmol, 2 equiv.), followed by CsCO₃ (3.0 mmol, 2 equiv.). The reaction mixture was stirred at 60° C. for 5 hours. The reaction mixture was cooled to room temperature. The solvent was evaporated away under reduced pressure. The residue was taken in by EtOAc and washed with water, brine and dried over Na₂SO₄. Removal of solvent afforded the desired product.

Step 4

The product of Step 3 was dissolved in 3 mL of THF/MeOH (1:3), followed by addition of 1N LiOH (5.0 equiv.). The resulting mixture was stirred at 50° C. for 3 hours. The organic solvent was evaporated under N₂ and residue was diluted with water (2 mL). The aqueous layer was partitioned with ethyl ether (2 mL). After removal of organic layer, the aqueous layer was neutralized by 1N HCl (5.0 equiv.) and then extracted with EtOAc (5 mL). The organic layer was washed with water, brine, and dried over Na₂SO₄. Removal of solvent afforded {3-[4[(3,4-Dichloro-phenyl)-piperazine-1-sulfonyl]-phenoxy}-acetic acid. ¹H NMR (400 MHz, CDCl₃) δ ppm. 7.51 (t, 1H), 7.45 (d, 1H), 7.33 (s, 1H), 7.30 (d, 1H), 7.20 (d, 1H), 6.93 (s, 1H), 6.72 (d, 1H), 4.74 (s, 2H), 3.85 (s, 3H), 3.24 (m, 4H), 3.18 (m, 4H).

### EXAMPLE 189

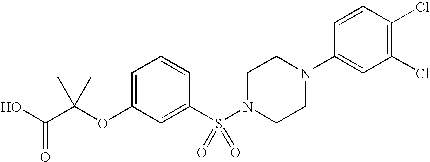

### 2-{3-[4-(3,4-Dichloro-phenyl)-piperazine-1-sulfonyl]-phenoxy}-2-methyl-propionic acid

Prepared as in Example 188. ¹H NMR (400 MHz, CDCl₃) δ ppm. 7.43 (m, 2H), 7.29 (t, 2H), 7.09 (d, 1H), 6.93 (s, 1H), 6.71 (d, 1H), 3.25 (m, 4H), 3.18 (m, 4H), 1.30 (s, 3H), 1.27 (s, 3H).

### EXAMPLE 190

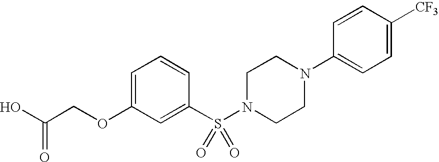

### {3-[4-(4-Trifluoromethyl-phenyl)-piperazine-1-sulfonyl]-phenoxy}-acetic acid

Prepared as in Example 188. ¹H NMR (400 MHz, CDCl₃) δ ppm. 7.46 (d, 2H), 7.43 (t, 2H), 7.35 (s, 1H), 7.20(d, 1H), 6.92 (d, 2H), 4.74 (s, 2H), 3.38 (m, 4H), 3.21 (m, 4H).

### EXAMPLE 191

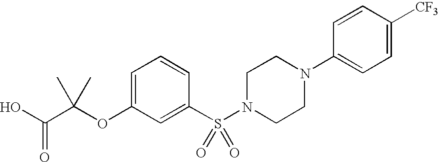

### 2-Methyl-2-{3-[4-(4-trifluoromethyl-phenyl)-piperazine-1-sulfonyl]-phenoxy}-propionic acid

Prepared as in Example 188. ¹H NMR (400 MHz, CDCl₃) δ ppm. 7.51 (d, 2H), 7.44 (t, 2H), 7.31 (s, 1H), 7.04 (d, 1H), 6.92 (d, 2H), 3.69 (m, 4H), 3.18 (m, 4H), 1.66 (s, 6H).

### EXAMPLE 192

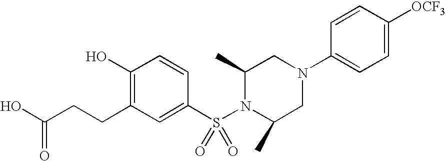

### 3-{5-[2,6-Dimethyl-4-(4-trifluoromethoxy-phenyl)-piperazine-1-sulfonyl]-2-hydroxy-phenyl}-propionic acid

Step 1

2-Oxo-chroman-6-sulfonyl chloride. To chlorosulfonic acid (3.5 mL) at 0° C. was added dihydrocoumarin (4.5 g, 3.84 mL, 30 mmol) via addition funnel dropwise over 20 minutes. After the addition was complete, the reaction mixture was warmed up to room temperature and stirred for 2 h. The mixture was carefully poured over ice water. The resulting emulsion was rinsed into a separatory funnel and extracted with ethyl acetate (3×50 mL), dried over Na₂SO₄ and concentrated in vacuo to give the title compound (3.2 g 43%). This material was used directly without further purification. ¹H NMR (400 MHz, CDCl₃) δ 7.99-7.94 (m, 2H), 7.28-7.26 (m, 1H), 3.16 (t, 2H), 2.89 (dd, 2H).

Step 2

3-{5-[2,6-Dimethyl-4-(4-trifluoromethoxy-phenyl)-piperazine-1-sulfonyl]-2-hydroxy-phenyl}-propionic acid. To a solution of 2-Oxo-chroman-6-sulfonyl-chloride (0.09 g, 0.36 mmol) from step 1 in 3.6 mL acetonitrile was added 3,5-Dimethyl-1-(4-trifluoromethoxy-phenyl)-piperazine (0.1 g, 0.36 mmol), followed by addition of solid K₂CO₃ (0.15 g, 1.1 mmol). This mixture was heated to 55° C. and stirred overnight. MeOH (0.5 mL) was added and the mixture was stirred at room temperature for 4 h. Solids were removed by filtration and the filtrate was concentrated in vacuo. The residue was purified by column chromatography (0-10% MeOH in CH₂Cl₂) to give the title compound (0.092 g). ¹H NMR (400 MHz, CDCl₃) δ 7.62 (d, 2H), 7.54 (dd, 1H), 7.07 (d, 1H), 6.88-6.84 (m, 2H), 4.14-4.09 (m, 2H), 3.31-3.27 (m, 2H), 2.91 (t, 2H), 2.58-2.52 (m, 4H), 1.42 (d, 6H).

### EXAMPLE 193

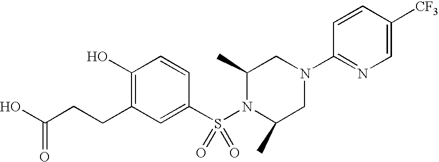

### 3-{5-[2,6-Dimethyl-4-(5-trifluoromethyl-pyridin-2-yl)-piperazine-1-sulfonyl]-2-hydroxy-phenyl}-propionic acid

The compound 3-1-5-[2,6-Dimethyl4-(5-trifluoromethyl-pyridin-2-yl)-piperazine-1-sulfonyl]-2-hydroxy-phenyl)-propionic acid was synthesized as outlined in Example 192 using 2-Oxo-chroman-6-sulfonyl chloride and 3,5-Dimethyl-1-(5-trifluoromethyl-pyridin-2-yl)-piperazine. ¹H NMR (400 MHz, DMSO) δ 8.32 (s, 1H), 7.74 (dd, 1H), 7.48-7.42 (m, 2H), 6.85 (d, 1H), 6.76 (d, 1H), 4.12-4.08 (m, 4H), 2.85 (dd, 2H), 2.70 (t, 2H), 2.32 (t, 2H), 1.2 (d, 6H).

### EXAMPLE 194

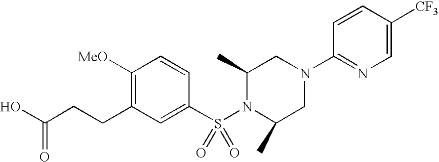

### 3-{5-[2,6-Dimethyl-4-(5-trifluoromethyl-pyridin-2-yl)-piperazine-1-sulfonyl]-2-methoxy-phenyl}-propionic acid

To a solution of the product of Example 193 (0.01 g, 0.02 mmol) in 1:1 THF/MeOH (0.4 mL) was added TMSCHN₂ (30 μL of a 2M solution in ether, 0.06 mmol). The mixture was stirred at room temperature for 2 h and 1N aqueous solution of LiOH (60 μL, 0.06 mmol) was added. The mixture was stirred at room temperature overnight. The reaction mixture was quenched with acidic Dowex resin, solids were removed by filtration, and the filtrate concentrated in vacuo. The residue was purified by column chromatography (0-10% MeOH in CH₂Cl₂) affording 0.003 g product. ¹H NMR (400 MHz, CD₃OD) δ 8.25 (s, 1H), 7.71-7.62 (m, 3H), 7.01 (d, 1H), 6.70 (d, 1H), 4.17 (br, 2H), 3.97 (dd, 2H), 3.88 (s, 3H), 3.06 (dd, 2H), 2.93-2.89 (m, 2H), 2.58-2.55 (m, 2H), 1.35 (d, 6H).

### EXAMPLE 195

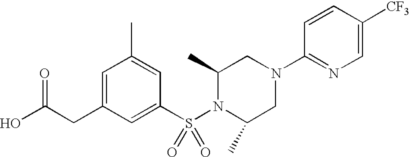

### {3-[2,6(S,S)-Dimethyl-4-(5-trifluoromethyl-pyridin-2-yl)-piperazine-1-sulfonyl]-5-methyl-phenyl}-acetic acid

The compound of Example 195 was synthesized as outlined in Example 19 (steps 2 and 3) by using (3-Chlorosulfonyl-5-methyl-phenyl)-acetic acid methyl ester and (S,S)-3,5-dimethyl-1-(5-trifluoromethyl-pyridin-2-yl)-piperazine. ¹H NMR (400 MHz, CD₃OD) δ 8.27 (br, s, 1H), 7.67-7.62 (m, 2H), 7.50 (s, 1H), 7.23 (s, 1H), 6.61 (d, 1H), 4.22-4.17 (m, 2H), 3.78 (dd, 2H), 3.48 (dd, 2H), 2.30 (s, 3H), 1.30 (d, 6H).

### EXAMPLE 196

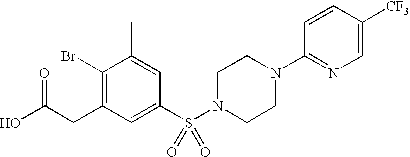

### {2-Bromo-3-methyl-5-[4-(5-trifluoromethyl-pyridin-2-yl)-piperazine-1-sulfonyl]-phenyl}-acetic acid

Step 1

N-(3,5-Dimethylphenyl)acetamide. To a solution of 3,5-dimethylbenzenamine (20 g, 165.3 mmol) in CH₂Cl₂ (200 mL) was added acetic anhydride (20.2 g, 198.0 mmol) dropwise with stirring at 0° C. To this mixture was added triethylamine (20 g, 198.0 mmol) dropwise with stirring. The resulting solution was stirred for 3 h while the temperature was maintained at 0° C. The reaction was quenched with water, extracted with CH₂Cl₂, dried over Na₂SO₄ and concentrated in vacuo to afford the title compound (28 g) as an orange solid.

Step 2

N-(4-Bromo-3,5-dimethylphenyl)acetamide. To a solution of N-(3,5-dimethylphenyl)acetamide (2.5 g, 15.3 mmol) in CH₂Cl₂ (100 mL) was added methanol (40 mL). The mixture was stirred 30 min. To the mixture was added Bu₄NBr₃ (8 g, 16.6 mmol). The resulting solution was stirred overnight at room temperature. The reaction mixture was concentrated in vacuo. To the residue was added 200 mL of H₂O. The resulting solution was extracted with EtOAc (3×100 mL) and the combined organic layers were dried over Na₂SO₄ and concentrated in vacuo to afford the title compound (3 g, 48%) as a white solid.

Step 3

4-Bromo-3,5-dimethylbenzenamine. To a solution of N-(4-bromo-3,5-dimethylphenyl)acetamide (3.0 g, 12.40 mmol) in methanol (120 mL) was added hydrochloric acid (30 mL). The resulting solution was stirred for 3 h while the temperature was maintained at reflux. The mixture was cooled and concentrated in vacuo. The pH was adjusted to 9 by addition of saturation Na₂CO₃ solution. The resulting solution was extracted with EtOAc (3×50 mL). The combined organic layers were dried over Na₂SO₄ and concentrated in vacuo to afford the title compound (2.0 g) as a white solid.

Step 4

2-Bromo-1,3-dimethyl-5-nitrobenzene. To a solution of cat. (0.36 g) in H₂O₂ (5.6 g). This was followed by the addition of a solution of 4-bromo-3,5-dimethylbenzenamine (2 g, 8.26 mmol) in methanol (8 mL) which was added dropwise with stirring, while maintaining the temperature of 0-20° C. To the mixture was added methanol (8 mL). To the above was added H₂O₂ (7.9 g) dropwise with stirring, while cooling to a temperature of 0-10° C. The resulting solution was allowed to react, with stirring, for 3 hours while the temperature was maintained at room temperature. The resulting solution was extracted three times with 50 mL of CH₂Cl₂ and the combined organic layers were dried over Na₂SO₄ and concentrated in vacuo. The residue was purified by column chromatography eluting with 1:100 EtOAc/petroleum ether to afford the title compound (1.6 g) as a white solid.

Step 5

2-Bromo-1-(bromomethyl)-3-methyl-5-nitrobenzene. To a solution of 2-bromo-1,3-dimethyl-5-nitrobenzene (1.4 g, 6.09 mmol) in CCl₄ (30 mL) was added NBS (1.3 g, 7.30 mmol) and AIBN (0.02 g). The resulting solution was stirred for 2 h while the temperature was maintained at 95° C. in an oil bath. Solids were removed by filtration. The filtrate was washed with 20 mL of 10% sodium hydroxide solution and 2×10 mL of water. The mixture was dried over Na₂SO₄ and concentrated in vacuo to afford the title compound (0.9 g) as a yellow solid.

Step 6

2-(2-Bromo-3-methyl-5-nitrophenyl)acetonitrile. To a solution of 2-bromo-1-(bromomethyl)-3-methyl-5-nitrobenzene (120 g, 32 mmol) in ethanol (200 mL) was added a solution of potassium cyanide (2.7 g, 39 mmol) in water (20 mL). The resulting solution was stirred ovemight while the temperature was maintained at reflux in an oil bath. The mixture was concentrated in vacuo. To the residue was added 200 mL of H₂O). The resulting solution was extracted with CH₂Cl₂ (3×100 mL). The combined organic layers were dried over Na₂SO₄ and concentrated in vacuo to give the title compound (3. g, 31%) as a black oil.

Step 7

2-(2-Bromo-3-methyl-5-nitrophenyl)acetic acid. To 2-(2-bromo-3-methyl-5-nitrophenyl)acetonitrile (3 g, 12.45 mmol) was added sulfuric acid (7 mL), followed by acetic acid (7mL) and water (7 mL). The resulting solution was heated at reflux overnight. The reaction mixture was cooled and then quenched with the addition of H₂O (50 mL). The resulting solution was -extracted with EtOAc (3×30 mL) and the organic layers combined and concentrated in vacuo to afford the title compound (2 g, 49%) as a brown solid.

Step 8

Methyl 2-(2-bromo-3-methyl-5-nitrophenyl)acetate. To a solution of 2-(2-bromo-3-methyl-5-nitrophenyl)acetic acid (2 g, 6.15 mmol) in MeOH (30 mL) was added sulfuric acid (1 mL). The resulting solution was heated at reflux overnight. The mixture was cooled and concentrated in vacuo. To the residue was added H₂O (20 mL). The resulting solution was extracted with EtOAc (2×20 mL) and the combined organic layers were dried over Na₂SO₄ and concentrated in vacuo to afford the title compound (2.8 g) as a black solid.

Step 9

Methyl 2-(2-bromo-3-methyl-5-aminophenyl)acetate. A mixture of methyl 2-(2-bromo-3-methyl-5-nitrophenyl)acetate (2.8 g, 10 mmol) in water (35 mL) was heated to 70° C. To the mixture was added iron (2.8 g, 50 mmol) followed by dropwise addition of acetic acid (3 g, 50 mmol) with stirring. The resulting solution was stirred for 1 h while the temperature was maintained at 95° C. in an oil bath. The resulting solution was filtered and extracted with EtOAc (3×30 mL). The combined organic layers were dried over Na₂SO₄ and concentrated in vacuo to afford the title compound (2.2 g) as a black solid.

Step 10

(2-Bromo-5-chlorosulfonyl-3-methyl-phenyl)-acetic acid methyl ester. To a solution of methyl 2-(2-bromo-3-methyl-5-aminophenyl)acetate (2 g, 80 mmol) in acetonitrile (94 mL) at 0° C. was added hydrochloric acid (4.8 g) followed by dropwise addition of acetic acid (9.2 g) and a solution of sodium nitrite (0.66 g) in water (5 mL). The solution was saturated with SO₂ and a solution of CuCl₂ (1.4 g) in water (5 mL) was added, at 0° C. The resulting solution was stirred overnight at room temperature. The reaction mixture was quenched with the addition of 50 mL of H₂O/ice. The resulting solution was extracted with EtOAc (3×50 mL) and the combined organic layers were washed with water (3×100 mL), dried over Na₂SO₄, and concentrated in vacuo. The residue was purified by column chromatography eluting with 1:100 EtOAC/PE solvent system to afford the title compound (0.5 g) as a white solid. ¹H NMR (400 MHz, CDCl₃) δ 7.89 (s, 1H), 7.85 (s, 1H), 4.00 (s, 2H), 3.82 (s, 3H), 2.63 (s, 3H).

Step 11

{2-Bromo-3-methyl-5-[4-(5-trifluoromethyl-pyridin-2-yl)-piperazine-1-sulfonyl]-phenyl}-acetic acid methyl ester. This compound was prepared as outlined in Example 19 (step 2) by using (2-Bromo-5-chlorosulfonyl-3-methyl-phenyl)-acetic acid methyl ester (0.147 g, 0.43 mmol) and 1-(5-trifluoromethyl-pyridin-2-yl)-piperazine (0.1 g, 0.43 mmol). ¹H NMR (400 MHz, CDCl) δ 8.35 (br, s, 1H), 7.62-7.60 (m, 1H), 7.54-7.50 (m, 2H), 6.59 (d, 1H), 3.87 (s, 2H), 3.76-3.74 (m, 4H), 3.70 (s, 3H), 3.13-3.10 (m, 4H), 2.49 (s, 3H).

Step 12

{2-Bromo-3-methyl-5-[4-(5-trifluoromethyl-pyridin-2-yl)-piperazine-1-sulfonyl]-phenyl}-acetic acid. This compound was prepared as outlined in Example 19 (step 3) by using 12-Bromo-3-methyl-5-[4-(5-trifluoromethyl-pyridin-2-yl)-piperazine-1-sulfonyl]-phenyl)-acetic acid methyl ester (0.040 g, 0.07 mmol) and LiOH (0.11 mL, 0.1 mmol). ¹H NMR (400 MHz, CD₃OD) δ 8.31 (br, s, 1H), 7.70-7.68 (m, 1H), 7.61 (d, 2H), 6.86 (d, 1H), 3.92 (s, 2H), 3.76-3.74 (m, 4H), 3.12-3.09 (m, 4H), 2.50 (s, 3H).

### EXAMPLE 197

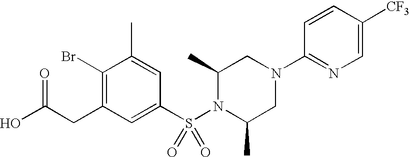

### {2-Bromo-5-[2,6-dimethyl-4-(5-trifluoromethyl-pyridin-2-yl)-piperazine-1-sulfonyl]-3-methyl-phenyl}-acetic acid

The compound of Example 197 was prepared as outlined in Example 196. ¹H NMR (400 MHz, CD₃OD) δ 8.24 (br, s, 1H), 7.70-7.61 (m, 3H), 6.67 (d, 1H), 4.24-4.20 (m, 2H), 3.90 (s, 2H), 3.88 (s, 2H), 3.17 (dd, 2H), 2.41 (s, 3H), 1.37 (d, 6H).

### EXAMPLE 198

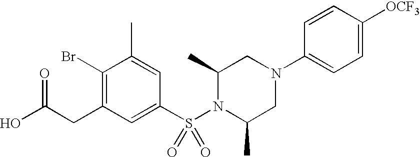

### {2-Bromo-5-[2,6-dimethyl-4-(4-trifluoromethoxy-phenyl)-piperazine-1-sulfonyl]-3-methyl-phenyl}-acetic acid

The compound of Example 198 was prepared as outlined in Example 196. ¹H NMR (400 MHz, CD₃OD) δ7.68 (s, 1H), 7.67 (s, 1H), 7.07 (d, 2H), 6.88 (d, 2H), 4.20-4.17 (m, 2H), 3.85 (s, 2H), 3.28 (m, 2H), 2.67 (dd, 2H), 2.46 (s, 3H), 1.47 (d, 6H).

### EXAMPLE 199

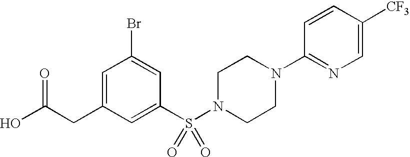

### {3-Bromo-5-[4-(5-trifluoromethyl-pyridin-2-yl)-piperazine-1-sulfonyl]-phenyl}acetic acid

Step 1

(3-Bromo-5-chlorosulfonyl-phenyl)-acetic acid methyl ester. To a solution of 3-nitrobenzoic acid (12.6 g, 75.4 mmol) in sulfuric acid (150 mL) was added silver sulfate (11.7 g, 37.5 mmol). This mixture was treated with bromine (5.5 mL). The resulting solution was stirred overnight at 130° C. The reaction mixture was cooled and quenched with the addition of 300 mL of H₂O/ice. The mixture was filtered and washed with water (3×50 mL). The pH was adjusted to 10 by the addition of Na₂CO₃ (100%). Solids were removed by filtration and the pH of the filtrate was adjusted to 2 by the addition of HCl. The desired product was isolated by filtration and washed with water (3×50 mL) to afford the title compound (14.6 g) as a white solid.

Step 2

(3-Bromo-5-nitrophenyl)ₘethanol. To sodium borohydride (1.1 g, 4.47 mmol) in tetrahydrofuran (35 mL) was added 3-bromo-5-nitrobenzoic acid (3.5 g, 88.77 mmol) in several batches, while -cooling to 0-5° C. Upon complete addition, a solution of boron trifluoride etherate (2.1 mL) in tetrahydrofuran (10 mL) was added dropwise with stirring, while cooling to a temperature of 0° C. over 30 minutes. The resulting solution was stirred for 3 h at room temperature. The reaction mixture was then quenched by the addition of 100 mL ice water. The resulting solution was extracted with EtOAc (3×100 mL) and the combined organic layers were washed with 10% Na₂CO₃ solution and water. The mixture was dried over Na₂SO₄ and concentrated in vacuo to afford the title compound (3 g, 76%) as a white solid.

Step 3

1-Bromo-3-(bromomethyl)-5-nitrobenzene. To a solution of (3-bromo-5-nitrophenyl) methanol (3 g, 12.9 mmol) in CH₂Cl₂ (40 mL) was added tribromophosphine (4.2 g, 15.5 mmol) dropwise with stirring at 0° C. The resulting solution was stirred at room temperature. The reaction mixture was then quenched by the addition ice water (200 mL). The resulting solution was extracted with CH₂Cl₂ and the combined organic layers were washed with saturated NaHCO₃ solution and water. The mixture was dried over MgSO₄ and concentrated in vacuo. The residue was purified by column chromatography eluting with a 20:1 EtOAc/PE solvent system to afford the title compound (2 g, 60%) as a yellow solid.

Step 4

2-(3-Bromo-5-nitrophenyl) acetonitrile. The compound was prepared as outlined in Example 196, Step 6.

Step 5

2-(3-Bromo-5-nitrophenyl)acetic acid. The compound was prepared as outlined in Example 196, Step 7.

Step 6

Methyl 2-(3-bromo-5-nitrophenyl)acetate. The compound was prepared as outlined in Example 196, Step 8.

Step 7

Methyl 2-(3-amino-5-bromophenyl)acetate. The compound was prepared as outlined in Example 196, Step 9.

Step 8

(3-Bromo-5-chlorosulfonyl-phenyl)-acetic acid methyl ester. The compound was prepared as outlined in Example 196, Step 10. ¹H NMR (400 MHz, CDCl₃) δ 8.05 (s, 1H), 7.86 (s, 1H), 7.79(s, 1H), 3.70 (s, 2H), 3.72 (s, 3H).

Step 9

{3-Bromo-5-[4-(5-trifluoromethyl-pyridin-2-yl)-piperazine-1-sulfonyl]-phenyl}acetic acid methyl ester. The compound was prepared as outlined in Example 196, Step 11. ¹H NMR (400 MHz, CDCl₃) δ 8.30 (s, br, 1H), 7.81-7.80 (m, 1H), 7.66-7.61 (m, 3H), 6.60 (d, 1H), 3.76 (t, 4H), 3.71 (s, 3H), 3.67 (s, 2H), 3.13 (t, 4H).

Step 10

{3-Bromo-5-[4-(5-trifuoromethyl-pyridin-2-yl)-piperazine-1-sulfonyl]-phenyl}acetic acid: The compound was prepared as outlined in Example 196, Step 12. ¹H NMR (400 MHz, CD₃OD) δ 8.32 (s, 1H), 7.82-7.77 (m, 2H), 7.71-7.68 (m, 2H), 6.85 (d, 2H), 3.75 (t, 4H), 3.74 (s, 2H), 3.12 (t, 4H).

### EXAMPLE 200

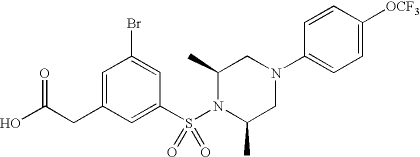

### {3-Bromo-5-[2,6-dimethyl-4-(4-trifluoromethoxy-phenyl)-piperazine-1-sulfonyl]-phenyl}-acetic acid

The compound of Example 200 was prepared as outlined in Example 199. ¹H NMR (400 MHz, CD₃OD) δ 7.89 (t, 1H), 7.79 (s, 1H), 7.70 (s, 1H), 7.08 (d, 2H), 6.88 (d, 2H), 4.20-4.17 (m, 2H), 3.71 (s, 2H), 3.33-3.31 (m, 2H), 2.65 (dd, 2H), 1.47 (d, 6H).

### EXAMPLE 201

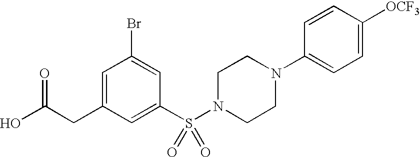

### {3-Bromo-5-[4-(4-trifluoromethoxy-phenyl)-piperazine-1-sulfonyl]-phenyl}-acetic acid

The compound of Example 201 was prepared as outlined in Example 199. ¹H NMR (400 MHz, CD₃OD) δ 7.89 (t, 1H), 7.79 (s, 1H), 7.70 (s, 1H), 7.08 (d, 2H), 6.88 (d, 2H), 4.20-4.17 (m, 2H), 3.71 (s, 2H), 3.33-3.31 (m, 2H), 2.65 (dd, 2H), 1.47 (d, 6H).

### EXAMPLE 202

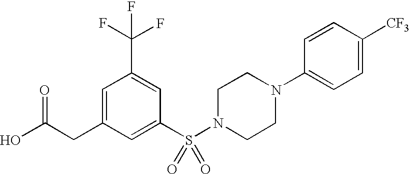

Step 1

(3-Trifluoromethyl-phenyl)-methanol. To lithium aluminum hydride (37.9 g, 1.2 mol, 1.2 equiv.) in THF (500 mL) at 0° C. was added 3-(trifluoromethyl)benzoic acid (200 g, 1.0 mol) in THF (1000 mL) dropwise at 0-10° C. The mixture was stirred overnight followed by dropwise addition of 10% sulfuric acid (500 ml) and water (1000 mL). The solution was filtrated and the filtrate was extracted with ethyl acetate (3×500 mL). The combined organic solution was washed with water (500 mL), dried over anhydrous sodium sulfate and concentrated in vacuo to give the title compound as an orange oil (180 g, 97%).

Step 2

1-Bromomethyl-3-trifluoromethyl-benzene. A solution of (3-Trifluoromethyl-phenyl)-methanol (180 g, 1.0 mol, 1.0 eqiv.) in dichloromethane (1000 mL) was cooled below 10° C. and phosphorus tribromide (360 g, 1.30 mol, 1.3 eqiv.) was added dropwise in 30 minutes. The mixture was stirred overnight and water was added dropwise until no gas was produced. The solution was washed with saturated sodium hydrogen carbonate (2×500 mL) and water (200 mL). The organic layer dried over anhydrous sodium sulfate and concentrated in vacuo to give the title compound as an brown-red liquid (163 g, 67%).

Step 3

(3-Trifluoromethyl-phenyl)-acetonitrile. To a solution of 1-Bromomethyl-3-trifluoromethyl-benzene (163 g, 0.68 mol, 1.0 eqiv.) in ethanol (1000 mL) was added potassium cyanide (54 g, 0.83 mol, 1.2 eqiv.) in water (500 mL). The mixture was heated at reflux for 1.5 h and concentrated in vacuo. The residue was extracted with ethyl acetate (3×400 mL). The combined organic solution was washed with water (3×500 mL). The solution was dried with anhydrous sodium sulfate and concentrated in vacuo to afford the title compound as yellow oil (130 g, 100%).

Step 4

(3-Trifluoromethyl-phenyl)-acetic acid. To a solution of (3-Trifluoromethyl-phenyl)-acelonitrile (90 g,

0.49 mol, 1.0 eqiv.) in 50% acetic acid (410 mL) was added concentrated sulfuric acid (205 mL) in batches. The mixture was heated at reflux for 5 h. The mixture was cooled and water (200 mL) was added. The solution was extracted with ethyl acetate (3×300 mL). The organic layer was dried over anhydrous sodium sulfate and concentrated in vacuo to give the title compound as a black solid.

Step 5

(3-Trifluoromethyl-phenyl)-acetic acid methyl ester. To a solution of (3-Trifluoromethyl-phenyl)-acetic acid in methanol (1200 mL) was added concentrated sulfuric acid (50 mL). The mixture was heated at reflux overnight. The solution was concentrated in vacuo and water (600 mL) was added to the residue. The solution was extracted with ethyl acetate (3×400 mL) and the organic layer was washed with water. The solution was dried over anhydrous sodium sulfate and purified with column chromatography (ethyl acetate: petroleum ether=1:20) to give the product (108 g, 100%) as a yellow oil.

Step 6

(3-Nitro-5-trifluoromethyl-phenyl)-acetic acid methyl ester. To a solution of (3-Trifluoromethyl-phenyl)-acetic acid methyl ester (14.9 g, 0.068 mol, 1.0 eqiv.) and Me₄NNO₃ (13.9 g, 0.102 mol, 1.5 eqiv.) in dichloromethane (100 mL) was added dropwise (CF₃SO₂)₂O (28.9 g, 0.102 mol, 1.5 eqiv.) in dichloromethane (50 mL). The mixture was stirred for 2 h at room temperature and heated to reflux overnight. The solution was neutralized with saturated sodium hydrogen carbonate and the organic layer was washed with water. The solution was dried over anhydrous magnesium sulfate and concentrated in vacuo to afford the title compound (6.1 g, 34%) as yellow oil.

Step 7

(3-Amino-5-trifluoromethyl-phenyl)-acetic acid methyl ester. A mixture of iron powder (5 g), acetic acid (2 g) and water (30 mL) was heated to reflux. To the mixture was added (3-Nitro-5-trifluoromethyl-phenyl)-acetic acid methyl ester (2.5 g, 9.5 mmol). The mixture was heated to reflux for 2 h. The solution was filtrated and the filter cake was washed with water and ethyl acetate. The filtrate was extracted with ethyl acetate (3×30 mL). The combined organic layer was dried over anhydrous magnesium sulfate and concentrated in vacuo to give brown liquid (2.3 g, 100%).

Step 8

(3-Chlorosulfonyl-5-trifluoromethyl-phenyl)-acetic acid methyl ester. To a solution of (3-amino-5-trifluoromethyl-phenyl)-acetic acid methyl ester 8 (2.3 g, 9.8 mmol) in acetonitrile (120 mL) was added acetic acid (8.2 mL). The reaction solution was cooled to 0° C. for 30 min. Concentrated hydrochloride (4.1 mL) was added followed by a sodium nitrite solution (1.5 mL, 0.9 g). The mixture reacted for 1 hour, the mixture reacted for 3-4 hours under SO₂ atmosphere. Cupric chloride hydrate (2.2 g, 2 mL) solution was added dropwise and the mixture continued to react for 3 hours under SO₂ atmosphere. TLC (ethyl acetate: petroleum ether=1:2) monitored the reaction. The solution was poured into water (500 mL) and extracted with ethyl acetate (400 mL). The organic layer was washed till the volume did not decrease and no SO₂. The organic layer was dried over anhydrous magnesium sulfate and evaporated to give red-brown crude product. Column chromatography (ethyl acetate: petroleum ether=1:10) afforded crystals (1.5 g, 48%). ¹H NMR (400 MHz, CDCl₃), δ (ppm): 8.21 (s, 1H), 8.16 (s, 1H), 7.93 (s, 1H), 4.02 (s, 2H), 3.77 (s, 3H).

Step 9

{3-Trifluoromethyl-5-[4-(4-trifluoromethyl-phenyl)-piperazine-1-sulfonyl]-phenyl}-acetic acid. The compound was synthesized according to the procedure outlined for Example 17. ¹H NMR (400 MHz, CDCl₃), δ (ppm): 7.96 (s, 1H), 7.92 (s, 1H), 7.79 (s, 1H), 7.47 (d, 2H), 6.88 (d, 2H), 3.83 (s, 2H), 3.35 (m, 4H), 3.20 (m, 4H).

### EXAMPLE 203

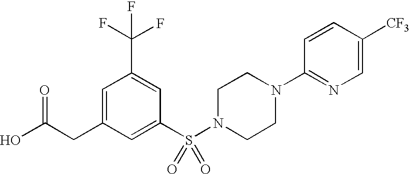

### {3-Trifluoromethyl-5-[4-(5-trifluoromethyl-pyridin-2-yl)-piperazine-1-sulfonyl]-phenyl}-acetic acid

The compound of Example 203 was synthesized according to the procedure outlined for Example 202. ¹H NMR (400 MHz, CDCl₃), δ (ppm): 8.35 (s, 1H), 7.94 (s, 1H), 7.91 (s, 1H), 7.78 (s, 1H), 7.63 (dd, 1H), 6.61 (d, 1H), 3.82 (s, 2H), 3.76 (m, 4H), 3.15 (m, 4H).

### EXAMPLE 204

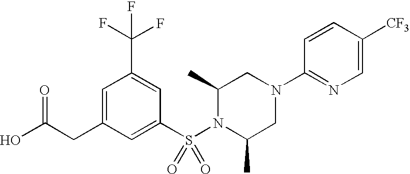

### {3-[2,6-Dimethyl-4-(5-trifluoromethyl-pyridin-2-yl)-piperazine-1-sulfonyl]-5-trifluoromethyl-phenyl}-acetic acid

The compound of Example 204 was synthesized according to the procedure outlined for Example 202. ¹H NMR (400 MHz, CDCl₃), δ (ppm): 8.31 (s, 1H), 8.01 (s, 1H), 7.97 (s, 1H), 7.66 (s, 1H), 7.58 (dd, 1H), 6.51 (d, 1H), 4.22 (m, 2H), 4.02 (d, 2H), 3.77 (s, 2H), 3.04 (dd, 2H), 1.38 (d, 6H).

### EXAMPLE 205

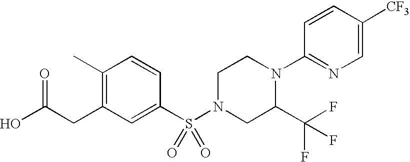

### {2-Methyl-5-[3-trifluoromethyl-4-(5-trifluoromethyl-pyridin-2-yl)-piperazine-1-sulfonyl]-phenyl}-acetic acid

The compound of Example 205 was synthesized according to the procedure outline for Example 23. ¹H NMR (400 MHz, CDCl₃), δ (ppm): 8.36 (s, 1H), 7.67 (d, 1H), 7.59 (s, 1H), 7.54 (d, 2H), 7.29 (d, 1H), 6.62 (d, 1H), 5.5 (m, 1H), 4.08 (m, 2H), 3.80 (d, 1H), 3.59 (s, 2H), 3.56 (m, 1H), 2.70 (dd, 1H), 2.49 (dt, 1H), 2.34 (s, 3H).

### EXAMPLE 206

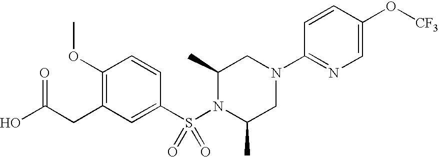

### {5-[2,6-Dimethyl-4-(5-trifluoromethoxy-pyridin-2-yl)-piperazine-1-sulfonyl]-2-methoxy-phenyl}-acetic acid

The compound of Example 206 was synthesized according to the procedure outlined for Example 75. ¹H NMR (400 MHz, CDCl₃), δ (ppm): 7.77 (dd, 1H), 7.68 (d, 1H), 6.99 (d, 2H), 6.92 (d, 1H), 6.77 (d, 2H), 4.18 (m, 2H), 3.88 (s, 3H), 3.68 (s, 2H), 3.19 (d, 2H), 2.66(dd, 2H), 1.46 (d, 6H).

### EXAMPLE 207

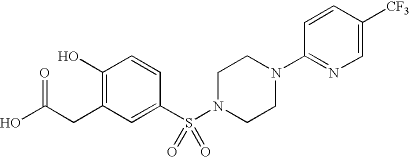

Step 1

{5-[4-(5-trifluoromethyl-pyridin-2-yl)-piperazine-1-sulfonyl]-2-hydroxy-phenyl}-acetic acid methyl ester. A solution of {5-[4-(5-trifluoromethyl-pyridin-2-yl)-piperazine-1-sulfonyl]-2-methoxy-phenyl}-acetic acid methyl ester (synthesized according to the procedure outline for Example 75, steps 1 and 2) (98.6 mg, 0.21 mmol, 1.0 equiv.) in CH₂Cl₂ (3 mL) was cooled to −78° C. To the cool solution was added BBr₃ (100 μL, 1.04 mmol, 5.0 eqiv.) with stirring. After stirring for 5 min, the cooling bath was removed and the mixture was stirred at room temperature for 1 hour. 2N NaOH (1.5 mL) was added to the reaction mixture with vigorous stirring and then the reaction mixture was adjusted to PH 3˜4 with saturated NaHCO₃. The reaction mixture was diluted with CH₂Cl₂ (20 mL) washed with water (20 mL) and brine (20 mL). The organic solution was dried over Na₂SO₄ and concentrated in vacuo to give the desired product (97 mg, 99%). ¹H NMR (400 MHz, CDCl₃), δ (ppm): 8.34 (s, 1H), 7.58 (m, 2H), 7.52 (s, 1H), 7.01 (d, 1H), 6.59 (d, 1H), 3.76 (s, 3H), 3.74 (t, 4H), 3.72 (s, 2H), 3.09 (t, 4H).

Step 2

{5-[4-(5-trifluoromethyl-pyridin-2-yl)-piperazine-1-sulfonyl]-2-hydroxy-phenyl}-acetic acid. The product of Step 1 was treated with 1N LiOH in THF/MeOH (3:1) to give desired product (95% yield). ¹H NMR (400 MHz, CD₃OD), δ (ppm): 8.25 (s, 1H), 7.63 (dd, 1H), 7.47 (s, 1H), 7.46 (d, 1H), 6.88 (d, 1H), 6.77 (d, 1H). 3.67 (m, 4H), 3.54 (s, 2H), 2.98 (m, 4H).

### EXAMPLE 208

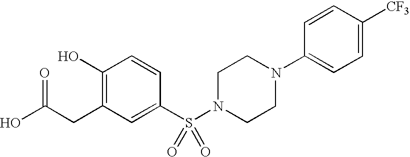

### {5-[4-(4-trifluoromethyl-phenyl)-piperazine-1-sulfonyl]-2-hydroxy-phenyl}-acetic acid

The product of Example 208 was synthesized according to the procedure outlined for Example 207. ¹H NMR (400 MHz, CD₃OD), δ (ppm): 7.59 (d, 1H), 7.55 (dd, 1H), 7.43 (d, 2H), 6.99 (d, 2H), 6.94 (d, 1H), 3.65 (s, 2H), 3.29 (m, 4H), 3.09 (m, 4H).

### EXAMPLE 209

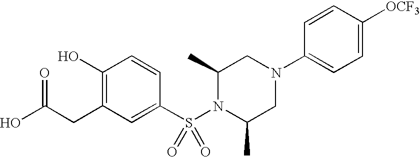

### {5-[2,6-Dimethyl-4-(4-trifluoromethoxy-phenyl)-piperazine-1-sulfonyl]-2-hydroxy-phenyl}-acetic acid

The product of Example 209 was synthesized according to the procedure outlined for Example 207. ¹H NMR (400 MHz, CD₃OD), δ (ppm): 7.67 (d, 1H), 7.60 (dd, 1H), 7.08 (d, 2H), 6.89 (m, 3H), 4.15 (m, 2H), 3.64 (s, 2H), 3.29 (d, 2H), 2.64 (dd, 2H), 1.45 (d, 6H).

### EXAMPLE 210

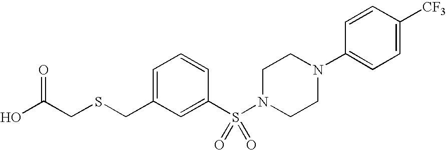

### {3-[4-(4-Trifluoromethyl-phenyl)-piperazine-1-sulfonyl]-benzylsulfanyl}-acetic acid

Step 1

3-Bromomethyl-benzenesulfonyl chloride. To solution of 3-Methyl-benzenesulfonyl chloride (5.5 g, 28.8 mmol, 1.0 equiv.) in benzene (50 mL) was added NBS (5.6 g, 31.7 mmol, 1.1 equiv.) and AIBN (47 mg, 0.29 mmol, 0.01 equiv.) with stirring. The resulting mixture was heated to reflux for 2 h. The reaction mixture was cooled to room temperature and diluted with ethyl acetate (200 mL). The diluted mixture was washed with water (2×100 mL), brine and dried over Na₂SO₄. After removal of solvent, the crude product was purified by chromatography to give 2.6 g of desired product (33%). ¹H NMR (400 MHz, CDCl₃), δ (ppm): 8.06 (s, 1H), 7.98 (d, 1H), 7.78 (d, 1H), 7.63 (t, 1H), 4.54 (s, 2H).

Step 2

1-(3-Bromomethyl-benzenesulfonyl)-4-(4-trifluoromethyl-phenyl)-piperazine. To a solution of 3-Bromomethyl-benzenesulfonyl chloride (2.6 g, 9.65 mmol, 1.0 equiv.) and 1-(4-Trifluoromethyl-phenyl)-piperazine (2.2 g, 9.7 mmol, 1.0 equiv.) in THF (20 mL) was added Et₃N (1.34 mL, 9.65 mmol, 1.0 equiv.). The resulting mixture was stirred at room temperature for 3 h and then diluted with ethyl acetate (100 mL). The diluted mixture was washed with water (2×50 mL), brine and dried over Na₂SO₄. After removal of solvent, the crude product was purified by chromatography to give 4.08 g of desired product (99%). ¹H NMR (400 MHz, CDCl₃), δ (ppm): 7.81 (t, 1H), 7.71 (dt, 1H), 7.65 (d, 1H), 7.55 (t, 1H), 7.47 (d, 2H), 6.88 (d, 2H), 4.53 (s, 2H), 3.35 (m, 4H), 3.19 (m, 4H).

Step 3

{3-[4-(4-Trifluoromethyl-phenyl)-piperazine-1-sulfonyl]-benzylsulfanyl}-acetic acid methyl ester To solution of 1-(3-Bromomethyl-benzenesulfonyl)-4-(4-trifluoromethyl-phenyl)-piperazine (306 mg, 0.80 mmol, 1.0 equiv.) and Mercapto-acetic acid methyl ester (127 mg, 1.20 mmol, 1.5 equiv.) in THF (20 mL) was added Et₃N (0.17 mL, 1.20 mmol, 1.5 equiv.). The resulting mixture was stirred at room temperature overnight and then diluted with ethyl acetate (100 mL). The diluted mixture was washed with water (2×50 mL), brine and dried over Na₂SO₄. After removal of solvent, the crude product was purified by chromatography to give the desired product (248 mg, 63%). ¹H NMR (400 MHz, CDCl₃), δ (ppm): 7.77 (t, 1H), 7.69 (dt, 1H), 7.62 (d, 1H), 7.52 (t, 1H), 7.47 (d, 2H), 6.88 (d, 2H), 3.88 (s, 2H), 3.72(s, 3H), 3.35 (m, 4H), 3.18 (m, 4H), 3.07 (s, 2H).

Step 4

{3-[4-(4-Trifluoromethyl-phenyl)-piperazine-1-sulfonyl]-benzylsulfanyl}-acetic acid. {3-[4-(4-Trifluoromethyl-phenyl)-piperazine-1-sulfonyl]-benzylsulfanyl}-acetic acid methyl ester was treated with 1N LiOH in THF/MeOH (3:1) to give desired product (40%). ¹H NMR (400 MHz, CD₃OD), δ (ppm): 7.82 (s, 1H), 7.73 (d, 1H), 7.66 (d, 1H), 7.57 (t, 1H), 7.50 (d, 2H), 6.92 (d, 2H), 3.97 (s, 2H), 3.38 (m, 4H), 3.20 (m, 4H), 3.12 (s, 2H).

### EXAMPLE 211

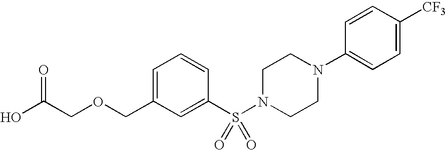

Step 1

{3-[4-(4-Trifluoromethyl-phenyl)-piperazine-1-sulfonyl]-benzyloxy}-acetic acid methyl ester. To a solution of 1-(3-Bromomethyl-benzenesulfonyl)-4-(4-trifluoromethyl-phenyl)-piperazine (1.1 g, 2.9 mmol) and Hydroxy-acetic acid methyl ester (2.6 g, 29.2 mmol) in THF (30 mL) was added NaH (60% in mineral oil) (1.2 g, 29.2 mmol). The resulting mixture was stirred at room temperature for 16 h and then diluted with ethyl acetate (200 mL). The diluted mixture was washed with water (2×50 mL), brine and dried over Na₂SO₄. After removal of solvent, the crude product was purified by chromatography to give the desired product (1.06 g, 77%). ¹H NMR (400 MHz, CDCl₃), δ (ppm): 7.83 (s, 1H), 7.76 (d, 1H), 7.69 (d, 1H), 7.59 (t,. 1H), 7.50 (d, 2H), 6.90 (d, 2H), 4.75 (s, 2H), 4.22 (s, 2H), 3.82 (s, 3H), 3.37 (m, 4H), 3.21 (m, 4H).

Step 2

{3-[4-(4-Trifluoromethyl-phenyl)-piperazine-1-sulfonyl]-benzyloxy}-acetic acid. {3-[4-(4-Trifluoromethyl-phenyl)-piperazine-1-sulfonyl]-benzyloxy}-acetic acid methyl ester was treated with 1N LiOH in THF/MeOH (3:1) to give the desired product (50% yield). ¹H NMR (400 MHz, CD₃OD), δ (ppm): 7.84 (s, 1H), 7.77 (d, 1H), 7.67 (d, 1H), 7.60 (t, 1H), 7.50 (d, 2H), 6.90 (d, 2H), 4.76 (s, 2H), 4.26 (s, 2H), 3.38 (m, 4H), 3.21 (m, 4H).

### EXAMPLE 212

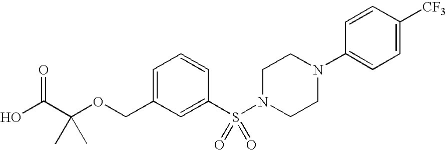

### 2-Methyl-2-{3-[4-(4-trifluoromethyl-phenyl)-piperazine-1-sulfonyl]-benzyloxy}-propionic acid

The product of Example 212 was synthesized according to the procedure outlined for Example 203. ¹H NMR (400 MHz, CD₃OD), δ (ppm): 7.83 (s, 1H), 7.75 (d, 1H), 7.70 (d, 2H), 7.59 (t, 1H), 7.50 (d, 2H), 6.91 (d, 2H), 4.65 (s, 2H), 3.38 (m, 4H), 3.21 (m, 4H), 1.62 (s, 6H).

### EXAMPLE 213

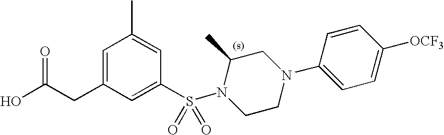

### {3-Methyl-5-[2-(S)-methyl-4-(4-trifluoromethoxy-phenyl)-piperazine-1-sulfonyl]phenyl}-acetic acid

The compound of Example 213 was synthesized according to the procedure in Example 68 using (3-chlorosulfonyl-5-methyl-phenyl)-acetic acid methyl ester. ¹H NMR (400 MHz, CD₃OH) δ 7.62 (s, 1H), 7.58 (s, 1H), 7.37 (s, 1H), 7.09 (d, 2H), 6.90 (d, 2H), 4.24-4.15 (m, 1H), 3.77 (d, 1H), 3.68 (s, 2H), 3.43 (d, 1H), 3.41-3.36 (m, 2H), 2.82 (dd, 1H), 2.68 (td, 1H), 2.41 (s, 3H), 1.21 (d 3H); LCMS 472.9 (M+1)⁺.

### EXAMPLE 214

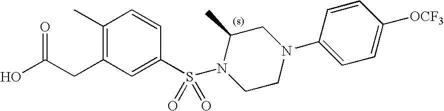

### {2-Methyl-5-[2-(S)-methyl-4-(4-trifluoromethoxy-phenyl)-piperazine-1-sulfonyl]-phenyl}-acetic acid

The compound of Example 214 was synthesized according to the procedure in Example 68. ¹H NMR (400 MHz, CD₃OH) δ 7.72 (d, 1H), 7.64 (dd, 1H), 7.39 (d, 1H), 7.09 (d, 2H), 6.90 (d, 2H), 4.32-4.14 (m, 1H), 3.80-3.75 (m, 1H), 3.74 (s, 2H), 3.50-3.42 (m, 1H), 3.40-3.30 (m, 2H), 2.81 (dd, 1H), 2.70-2.64 (m, 1H), 2.38 (s, 3H), 1.20 (d, 3H); LCMS 473.5 (M+1)⁺.

### EXAMPLE 215

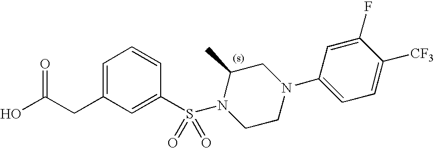

### {3-[4-(3-Fluoro-4-trifluoromethyl-phenyl)-2-(S)-methyl-piperazine-1-sulfonyl]-phenyl}-acetic acid

The compound of Example 215 was synthesized according to the procedure in Example 68 using (3-chlorosulfonyl-phenyl)-acetic acid methyl ester. ¹H NMR (400 MHz, CD₃OH) δ 7.82 (s, 1H), 7.80-7.75 (m, 1H), 7.53-7.50 (m, 2H), 7.38 (t, 1H), 6.67 (d, 2H), 4.26-4.16 (m, 1H), 3.80-3.74 (m, 1H), 3.72 s, 2H), 3.67-3.63 (m, 1H), 3.57-3.51 (m, 1H), 3.42-3.36 (m, 1H), 3.02 (dd, 1H), 2.86 (td, 1H), 1.16 (d, 3H); LCMS 461.5 (M+1)⁺.

### EXAMPLE 216

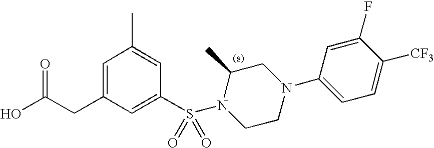

### {3-[4-(3-Fluoro-4-trifluoromethyl-phenyl)-2-(S)-methyl-piperazine-1-sulfonyl]-5-methyl-phenyl}-acetic acid

The compound of Example 216 was synthesized according to the procedure in Example 68 using (3-chlorosulfonyl-5-methyl-phenyl)-acetic acid methyl ester. ¹H NMR (400 MHz, CD₃OH) δ 7.58 (s, 1H), 7.55 (s, 1H), 7.36 (t, 1H), 7.31 (s, 1H), 6.67-6.63 (m, 1H), 6.62 (s, 1H), 4.25-4.15 (m, 1H), 3.80-3.73 (m, 1H), 3.65 (s, 2H), 3.62-3.54 (m, 1H), 3.55-3.47 (m, 1H), 3.45-3.30 (m, 1H), 3.06 (dd, 1H), 2.88 (td, 1H), 2.37 (s, 3H), 1.16 (d, 3H); LCMS 475.5 (M+1)⁺.

### EXAMPLE 217

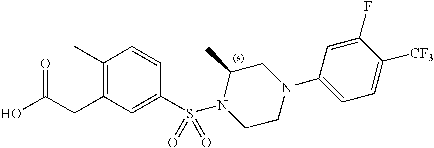

### {5-[4-(3-Fluoro-4-trifluoromethyl-phenyl)-2-(S)-methyl-piperazine-1-sulfonyl]-2-methyl-phenyl}-acetic acid

The compound of Example 217 was synthesized according to the procedure in Example 68. ¹H NMR (400 MHz, CD₃OH) δ 7.71 (s, 1H), 7.68-7.58 (m, 1H), 7.38-1.32 (m, 2H), 6.66-6.62 (m, 2H), 4.23-4.17 (m, 1H), 3.80-3.70 (m, 1H), 3.72 (s, 2H), 3.64-3.55 (m, 1H), 3.54-3.45 (m, 1H), 3.42-3.32 (m, 1H), 3.06 (dd, 2H), 2.88 (td, 2H), 2.35 (s, 3H), 1.17 (d, 3H); LCMS 475.5 (M+1)⁺.

### EXAMPLE 218

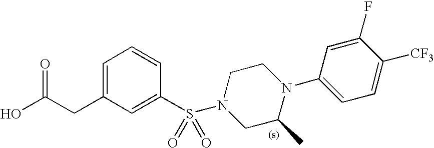

### {3-[4-(3-Fluoro-4-trifluorometbyl-phenyl)-3-(S)-methyl-piperazine-1-sulfonyl]-phenyl}-acetic acid

The compound Example 218 was synthesized according to the procedure in Example 90 using (3-chlorosulfonyl-phenyl)-acetic acid methyl ester. ¹H NMR (400 MHz, CD₃OH) δ 7.75 (s, 1H), 7.71-7.68 (m, 1H), 7.62-7.55 (m, 2H), 7.41 (t, 1H), 6.75 (s, 1H), 6.72 (s, 1H), 4.26-4.18 (m, 1H), 3.82-3.76 (m, 1H), 3.76 (s, 2H), 3.63-3.55 (m, 2H), 3.30-3.15 (m, 1H), 2.61 (dd, 1H), 2.46(td, 1H), 1.20 (d, 3H); LCMS 461.5 (M+1)⁺.

### EXAMPLE 219

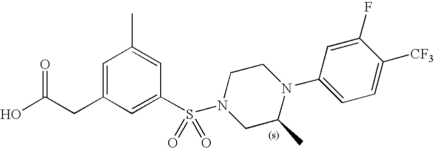

### {3-[4-(3-Fluoro-4-trifluoromethyl-phenyl)-3-(S)-methyl-piperazine-1-sulfonyl]-5-methyl-phenyl)}-acetic acid

The compound of Example 219 was synthesized according to the procedure in Example 90 using (3-Chlorosulfonyl-5-methyl-phenyl)-acetic acid. ¹H NMR (400 MHz, CD₃OH) δ 7.53 (s, 1H), 7.51 (s, 1H), 7.46-7.35 (m, 2H), 6.73 (s, 1H), 6.72 (s, 1H), 4.25-4.18 (m, 1H), 3.80-3.73 (m, 1H), 3.71 (s, 2H), 3.62-3.54 (m, 2H), 3.21(td, 2H), 2.61 (dd, 1H), 2.45 (td, 1H), 2.44 s, 3H), 1.20(d, 3H); LCMS 475.5 (M+1)⁺.

### EXAMPLE 220

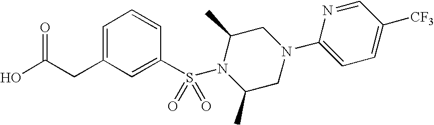

### {3-[2,6-Dimethyl-4-(5-trifluoromethyl-pyridin-2-yl)-piperazine-1-sulfonyl]-phenyl}-acetic acid

The compound of Example 220 was synthesized according to the procedure in Example 68 using (3-chlorosulfonyl-phenyl)-acetic acid methyl ester. ¹H NMR (400 MHz, CD₃OH) δ 8.27 (s, 1H), 7.84 (s, 1H), 7.78-7.73 (m, 1H), 7.65 (dd, 1H), 7.51-7.47 (m, 2H), 6.75 (d, 1H), 4.23-4.20 (m, 2H), 4.09 (d, 2H), 3.70 (s, 2H), 3.01 (dd, 2H), 1.36 (d, 6H); LCMS 457.7 (M+1)⁺.

### EXAMPLE 221

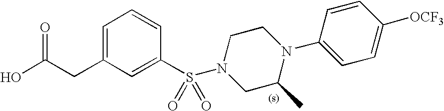

### {3-[3-(s)-Methyl-4-(4-trifluoromethoxy-phenyl)-piperazine-1-sulfonyl]-phenyl}-acetic acid

The compound of Example 221 was synthesized according to the procedure in Example 90 using (3-chlorosulfonyl-phenyl)-acetic acid methyl ester. ¹H NMR (400 MHz, CD₃OH) δ 7.75 (s, 1H), 7.71-7.68 (m, 1H), 7.64-7.55 (m, 2H), 7.11 (d, 2H), 6.96-6.92 (m, 2H), 3.95-3.91 (m, 1H), 3.76 (s, 2H), 3.63-3.55 (m, 1H), 3.38-3.32 (m, 1H), 3.28-3.24 (m, 1H), 3.18-3.12 (m, 1H), 2.80 (dd, 1H), 2.67-2.61 (m, 1H), 1.05 (d, 3H); LCMS 459.5 (M+1)⁺.

### EXAMPLE 222

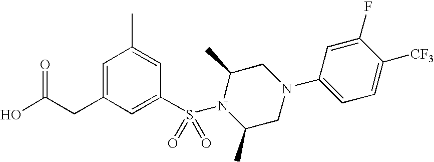

### {3-[4-(3-Fluoro-4-trifluoromethoxy-phenyl)-2,6-dimethyl-piperazine-1-sulfonyl]-5-methyl-phenyl}-acetic acid

The compound Example 222 was synthesized according to the procedure in Example 68 using (3-chlorosulfonyl-5-methyl-phenyl)-acetic acid methyl ester. ¹H NMR (400 MHz, CD₃OH) δ 7.61 (s, 1H), 7.56 (s, 1H), 7.35 (t, 1H), 7.29 (s, 1H), 6.65-6.55 (m, 2H), 4.21-4.18 (m, 2H), 3.65 (s, 2H), 3.45 (dd, 2 H), 2.92 (dd, 2H), 2.36 (s, 2H), 1.42 (d, 6H); LCMS 488.9 (M+1)⁺.

### EXAMPLE 223

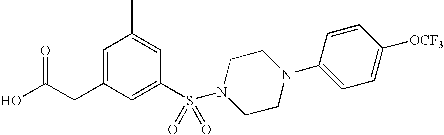

### {3-Methyl-5-[4-(4-trifluoromethoxy-phenyl)-piperazine-1-sulfonyl]-phenyl}-acetic acid

The compound of Example 223 was synthesized according to the procedure in Example 68 using (3-chlorosulfonyl-5-methyl-phenyl)-acetic acid methyl ester. ¹H NMR (400 MHz, CD₃OH) δ 7.54 (s, 1H), 7.53 (s, 1H), 7.44 (s, 1H) 7.11 (d, 2H), 6.97 (d, 2H), 3.72 (s, 2H), 3.23-3.21 (m, 4H), 3.14-3.12 (m, 4H), 2.45 (s, 3H); LCMS 459.5 (M+1)⁺.

### EXAMPLE 224

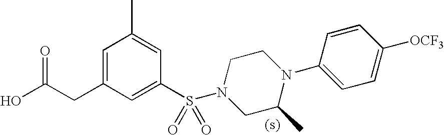

### {3-Methyl-5-[3-(s)-methyl-4-(4-trifluoromethoxy-phenyl)-piperazine-1-sulfonyl]-phenyl}-acetic acid

The compound of Example 224 was synthesized according to the procedure in Example 90 using (3-chlorosulfonyl-5-methyl-phenyl)-acetic acid methyl ester. ¹H NMR (400 MHz, CD₃OH) δ 7.54 (s, 1H), 7.50 (s, 1H), 7.44 (s, 1H), 7.11 (d, 2H), 6.98-6.92 (m, 2H), 3.97-3.93 (m, 1H), 3.69 (s, 2H), 3.61-3.53 (m, 1H), 3.38-3.32 (m, 1H), 3.29-3.23 (m, 1H), 3.20-3.10 (m, 1H), 2.80 (dd, 1H), 2.64 (td, 1H), 2.44 (s, 3H), 1.06 (d, 3H); LCMS 473.5 (M+1)⁺.

### EXAMPLE 225

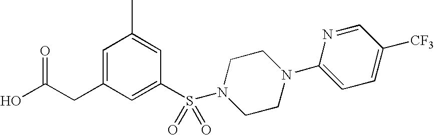

### {3-Methyl-5-[4-5-trifluoromethyl-pyridin-2-yl)-piperazine-1-sulfonyl]-phenyl}-acetic acid

The compound of Example 225 was synthesized according to the procedure in Example 3 using (3-chlorosulfonyl-5-methyl-phenyl)-acetic acid methyl ester. ¹H NMR (400 MHz, CD3OH) δ 8.31 (s, 1H), 7.70 (dd, 1H), 7.52 (d, 2H), 7.41 (s, 1H), 6.85 (d, 1H), 3.78-3.72 (m, 4H), 3.69 (s, 2H), 3.09-3.00 (m, 4H), 2.43 (s, 3H); LCMS 444.3 (M+1)⁺.

### EXAMPLE 226

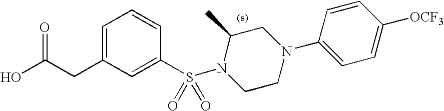

### {3-[2-(s)-Methyl-4-(4-trifluoromethoxy-phenyl)-piperazine-1-sulfonyl]-phenyl}-acetic acid

The compound of Example 226 was synthesized according to the procedure in Example 68 using (3-chlorosulfonyl-phenyl)-acetic acid methyl ester. ¹H NMR (400 MHz, CD₃OH) δ 7.83 (s, 1H), 7.77 (d, 1H), 7.59-7.49 (m, 2H), 7.09 (d, 2H), 6.98-6.86 (m, 2H), 4.25-4.18 (m, 1H), 3.81-3.74 (m, 1H), 3.75 (s, 2H), 3.50-3.45 (m, 1H), 3.43-3.33 (m, 2H), 2.81 (dd, 1H), 2.67 (td, 1H), 1.21 (d, 3H); LCMS 458.5 (M+1)⁺.

### EXAMPLE 227

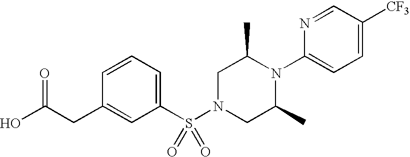

### 2-(3-(-3,5-Dimethyl-4-(5-(trifluoromethyl)pyridine-2-yl)piperazin-1-ylsulfonyl)phenyl)acetic acid

The compound of Example 227 was synthesized following the procedure in Example 90. ¹H NMR (CD₃OD) δ ppm. 8.34 (s, 1H), 7.75 (s, 1H), 7.71 (m, 2H), 7.58 (m, 2H), 6.76(d, 1H), 4.61 (m, 2H), 3.75 (s, 2H), 3.68 (d, 2H), 2.51 (dd, 2H), 1.33 (d, 6H).

### EXAMPLE 228

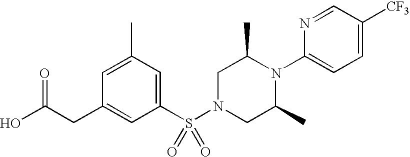

### 2-(3-(-3,5-Dimethyl-4-(5-(trifluoromethyl)pyridine-2-yl)piperazin-1-ylsulfonyl)-5-methylphenyl)acetic acid

The compound of Example 228 was synthesized following the procedure in Example 90. ¹H NMR (CD₃OD) δ ppm. 8.35 (s, 1H), 7.70 (dd, 1H), 7.54 (s, 1H), 7.51 (s, 1H), 7.42 (s, 1H), 6.77 (d, 1H), 4.60 (m, 2H), 3.68 (s, 2H), 2.51 (dd, 2H), 2.43 (s, 3H), 1.33 (d, 6H).

### EXAMPLE 229

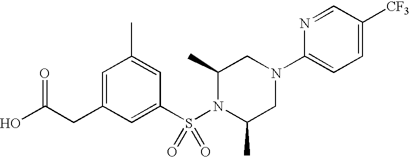

### {3-[2,6-Dimethyl-4-(5-trifluoromethyl-pyridin-2-yl)-piperazine-1-sulfonyl]-5-methyl-phenyl}-acetic acid

The compound of Example 229 was synthesized according to the procedure outlined in Example 68 using (3-chlorosulfonyl-5-methyl-phenyl) acetic acid methyl ester. ¹H NMR (400 MHz, CD₃OD) δ 8.25 (s, 1H), 7.64 (dd, 1H), 7.62 (s, 1H), 7.56 (s, 1H), 7.29 (s, 1H), 6.71 (d, 1H), 4.23-4.19 (m, 2H), 3.99 (d, 2H), 3.66 (s, 2H), 3.09 (dd, 2H), 2.36 (s, 3H), 1.36 (d, 6H); LCMS 472.3(M+1)⁺.

### EXAMPLE 230

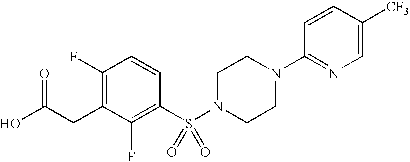

### {2,6-Difluoro-3-[4-(5-trifluoromethyl-pyridin-2-yl)-piperazine-1-sulfonyl]-phenyl}-acetic acid

The compound of Example 230 was synthesized according to the procedure outlined in Example 3 steps 3-4 using (3-chlorosulfonyl-2,6-difluoro-phenyl)-acetic acid methyl ester and 1-(5-trifluoromethyl-pyridin-2-yl)-piperazine. ¹H NMR (400 MHz, CD₃OD) δ 8.33 (s, 1H), 7.88-7.81 (m, 1H), 7.71 (dd, 1H), 7.19 (t, 1H), 6.88 (d, 1H), 3.77-3.74 (m, 6H), 3.28-3.25 (m, 4H); LCMS 466.4 (M+1)⁺.

### EXAMPLE 231

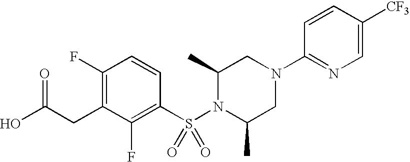

### {3-[2,6-Dimethyl-4-(5-trifluoromethyl-pyridin-2-yl)-piperazine-1-sulfonyl]-2,6-difluoro-phenyl}-acetic acid

The compound of Example 231 was synthesized according to the procedure outlined in Example 19 using (3-chlorosulfonyl-2,6-difluoro-phenyl)-acetic acid methyl ester. ¹H NMR (400 MHz, CD₃OD) δ 8.33 (s, 1H), 7.94-7.88 (m, 1H), 7.69 (dd, 1H), 7.16 (t, 1H), 6.86 (d, 1H), 4.27 (d, 2H), 4.20-4.10 (m, 2H), 3.75 (s, 2H), 3.03 (dd, 2H), 1.39 (d, 6H); LCMS 494.5 (M+1)⁺.

### EXAMPLE 232

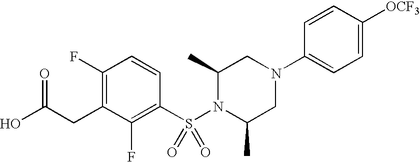

### {3-[2,6-Dimethyl-4-(4-trifluoromethoxy-phenyl)-piperazine-1-sulfonyl]-2,6-difluoro-phenyl}-acetic acid

The compound of Example 232 was synthesized according to the procedure outlined in Example 68 using (3-chlorosulfonyl-2,6-difluoro-phenyl)-acetic acid methyl ester. ¹H NMR (400 MHz, CD₃OD) 7.94-7.87 (m, 1H), 7.16 (t, 1H), 7.10 (d, 2H), 6.94 (d, 2H), 4.18-4.10 (m, 2H), 3.75 (s, 2H), 3.38 (d, 2H), 2.72 (dd, 2H), 1.52 (d, 6H); LCMS 508.9 (M+1)⁺.

### EXAMPLE 233

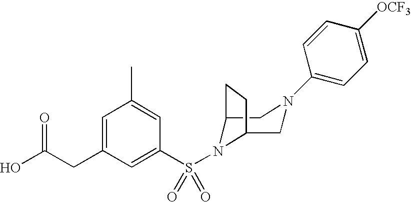

### {3-Methyl-5-[3-(4-trifluoromethoxy-phenyl)-3,8-diaza-bicyclo[3.2.1]octane-8-sulfonyl]-phenyl}-acetic acid

The compound of Example 233 was synthesized according to the procedure outlined in Example 90 using (3-chlorosulfonyl-5-methyl-phenyl) acetic acid methyl ester. ¹H NMR (400 MHz, CD₃OD) 7.66 (d, 2H), 7.41 (s, 1H), 7.09 (d, 2H), 6.88 (d, 2H), 4.35-4.32 (m, 2H), 3.69 (s, 2H), 3.53 (dd, 2H), 2.96 (d, 2H), 2.43 (s, 3H), 1.72-1.68 (m, 2H), 1.52-1.45 (m, 2H); LCMS 484.9 (M+1)⁺.

### BIOLOGICAL ASSAYS OF THE COMPOUNDS OF THE INVENTION

Compounds of Examples 1-233 were assayed to measure their biological activity with respect to their EC₅₀ values and efficacy for modulating PPAR-alpha, PPAR-gamma, and PPAR-delta as set forth in Table 3.

TABLE 3
Biological Activity

| | PPAR alpha | PPAR delta | PPAR gamma |
|---|---|---|---|
| | A > 100 μM | A > 100 μM | A > 100 μM |
| | B = 5-100 μM | B = 5-100 μM | B = 5-100 μM |
| Example # | C = <5 μM | C = <5 μM | C = <5 μM |
| 1 | A | C | A |
| 2 | A | B | A |
| 3 | B | C | B |
| 4 | B | C | B |
| 5 | A | C | B |
| 6 | B | C | B |
| 7 | B | C | B |
| 8 | B | C | C |
| 9 | C | C | C |
| 10 | A | B | A |
| 11 | A | B | A |
| 12 | A | B | B |
| 13 | A | B | A |
| 14 | B | C | C |
| 15 | B | C | B |
| 16 | A | B | C |
| 17 | B | C | B |
| 18 | C | C | C |
| 19 | B | C | B |
| 20 | A | C | A |
| 21 | A | C | B |
| 22 | B | C | C |
| 23 | B | C | C |
| 24 | A | B | A |
| 25 | A | B | A |
| 26 | A | B | A |
| 27 | A | B | B |
| 28 | A | C | A |
| 29 | A | C | A |
| 30 | B | C | B |
| 31 | C | C | B |
| 32 | C | B | B |
| 33 | C | C | C |
| 34 | C | C | B/C |
| 35 | B | C | B |
| 36 | B | C | C |
| 37 | A | C | A |
| 38 | A | C | C |
| 39 | A | C | C |
| 40 | A | B | A |
| 41 | A | C | B |
| 42 | A | C | B |
| 43 | A | A | A |
| 44 | C | C | C |
| 45 | B | C | B |
| 46 | A | A | B |
| 47 | A | B | B |
| 48 | A | B/C | B |
| 49 | A | B | B |
| 50 | A | B | B |
| 51 | B | C | C |
| 52 | A | A | A |
| 53 | A | B | B |
| 54 | C | C | C |
| 55 | B | C | B |
| 56 | A | C | B |
| 57 | B | C | C |
| 58 | B | C | C |
| 59 | C | C | C |
| 61 | A | C | C |
| 62 | A | C | B/C |
| 63 | A | C | B |
| 64 | A | A | B |
| 66 | B | C | A |
| 67 | A | A | A |
| 68 | C | C | B |
| 69 | B | C | C |
| 70 | C | C | C |
| 72 | A | B | A |
| 73 | A | B | A |
| 74 | A | B | A |
| 75 | A | C | B |
| 76 | A | C | B |
| 77 | A | C | B |
| 78 | A | C | A |
| 79 | A | C | B |
| 80 | A | C | B |
| 81 | A | B | B |
| 82 | A | B | A |
| 83 | A | C | A |
| 84 | A | A | A |
| 85 | A | B | A |
| 86 | A | A | A |
| 87 | C | C | B |
| 88 | A | A | A |
| 89 | A | A | A |
| 90 | C | C | B |
| 91 | A | A | A |
| 92 | C | C | A |
| 93 | C | C | B |
| 94 | B | C | C |
| 95 | A | C | A |
| 96 | C | C | A |
| 97 | C | C | A |
| 98 | C | C | A |
| 99 | C | C | B |
| 100 | B | C | B |
| 101 | B | C | B |
| 102 | B | C | B |
| 103 | B | C | B |
| 104 | B | C | C |
| 105 | A | C | C |
| 106 | A | C | A |
| 107 | A | C | A |
| 108 | B | C | B |
| 109 | B | C | A |
| 110 | B | C | B |
| 111 | B | C | B |
| 112 | B | C | A |
| 113 | B | C | A |
| 114 | B | C | A |
| | PPAR alpha | PPAR delta | PPAR gamma |
| | A > 100 μM | A > 100 μM | A > 100 μM |
| | B = 5-100 μM | B = 5-100 μM | B = 5-100 μM |
| Example # | C < 5μM | C = < 5μM | C < 5 μM |
| 115 | B | C | B |
| 116 | A | C | B |
| 117 | A | C | B |
| 118 | A | B | B |
| 119 | B | B | B |
| 120 | A | A | A |
| 121 | A | A | A |
| 122 | A | B | A |
| 123 | A | B | B |
| 124 | A | B | B |
| 125 | B | B | B |
| 126 | B | B | C |
| 127 | A | A | A |
| 128 | B | B | B |
| 129 | A | A | A |
| 130 | A | A | B |
| 131 | A | B | B |
| 132 | A | A | B |
| 133 | A | A | A |
| 134 | A | A | B |
| 135 | A | B | A |
| 136 | A | A | A |
| 137 | A | A | A |
| 138 | A | A | B |
| 139 | A | B | B |
| 140 | A | A | A |
| 141 | A | B | A |
| 142 | A | A | A |
| 143 | B | B | A |
| 144 | A | B | B |
| 145 | A | B | A |
| 146 | A | A | B |
| 147 | A | C | B |
| 148 | A | A | A |
| 149 | A | A | B |
| 150 | A | B | B |
| 151 | A | A | B |
| 152 | A | A | B |
| 153 | A | A | A |
| 154 | A | A | B |
| 155 | A | A | A |
| 156 | A | B | B |
| 157 | A | A | A |
| 158 | A | B | A |
| 159 | A | A | A |
| 160 | A | A | B |
| 161 | A | A | B |
| 162 | A | A | A |
| 163 | A | A | A |
| 164 | A | B | B |
| 165 | A | C | B |
| 166 | B | C | B |
| 167 | A | C | B |
| 168 | A | B | B |
| 169 | A | B | B |
| 170 | A | B | A |
| 171 | A | B | B |
| 172 | A | A | A |
| 173 | A | B | A |
| 174 | A | A | A |
| 175 | B | C | B |
| 176 | A | C | B |
| 177 | A | B | B |
| 178 | A | B | B |
| 179 | A | A | A |
| 180 | A | B | B |
| 181 | A | B | B |
| 182 | B | C | C |
| 183 | B | C | B |
| 184 | A | B | A |
| 185 | A | B | B |
| 186 | A | C | B |
| 187 | A | C | B |
| 188 | A | B | A |
| 189 | A | A | B |
| 190 | A | B | A |
| 191 | B | B | B |
| 192 | A | B | A |
| 193 | A | B | A |
| 194 | A | C | A |
| 195 | B | C | B |
| 196 | B | C | C |
| 197 | A | C | B |
| 198 | B | C | B |
| 199 | B | C | B |
| 200 | B | C | B |
| 201 | B | C | B |
| 202 | B | C | B |
| 203 | B | C | B |
| 204 | A | C | A |
| 205 | B | C | B |
| 206 | A | C | B |
| 207 | B | C | B |
| 208 | B | C | A |
| 209 | A | B | A |
| 210 | B | B | B |
| 211 | A | A | A |
| 212 | B | A | B |
| 213 | B | C | B |
| 214 | B | C | B |
| 215 | B | C | B |
| 216 | B | C | B |
| 217 | B | C | B |
| 218 | C | C | B |
| 219 | B | C | B |
| 220 | B | C | A |
| 221 | B | C | B |
| 222 | B | C | A |
| 223 | B | C | B |
| 224 | B | C | B |
| 225 | B | C | C |
| 226 | B | C | B |
| 227 | B | C | C |
| 228 | B | C | C |
| 229 | A | C | A |
| 230 | B | C | B |
| 231 | A | B | A |
| 232 | A | C | A |
| 233 | B | C | B |

Those of skill in the art will appreciate that the compounds and uses disclosed herein can be used as PPAR modulators, providing a therapeutic effect.

One skilled in the art will appreciate that these methods and compounds are and may be adapted to carry out the objects and obtain the ends and advantages mentioned, as well as those inherent therein. The methods, procedures, and compounds described herein are exemplary and are not intended as limitations on the scope of the invention. Changes therein and other uses will occur to those skilled in the art which are encompassed within the spirit of the invention and are defined by the scope of the claims.

It will be apparent to one skilled in the art that varying substitutions and modifications may be made to the invention disclosed herein without departing from the scope and spirit of the invention.

Those skilled in the ail recognize that the aspects and embodiments of the invention set forth herein may be practiced separate from each other or in conjunction with each other. Therefore, combinations of separate embodiments are within the scope of the invention as claimed herein.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that the use of such terms and expressions indicates the exclusion of equivalents of the features shown and described or portions thereof. It is recognized that various modifications are possible within the scope of the invention claimed. Thus, it should be understood that although the present invention has been specifically disclosed by certain embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are within the following claims.

1. A compound having the structure of Formula (I) 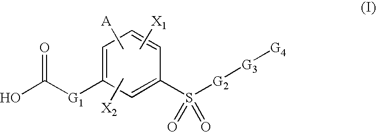
or a pharmaceutically acceptable N-oxide, pharmaceutically acceptable prodrug, pharmaceutically acceptable metabolite, pharmaceutically acceptable salt, pharmaceutically acceptable ester, pharmaceutically acceptable amide, or pharmaceutically acceptable solvate thereof;
wherein:
G₁ is selected from the group consisting of CR₁R₂)ₙ—, -Z(CR₁R₂)ₙ—, —(CR₁R₂)ₙZ-, and —(CR₁R₂)ₙZ(CR₁R₂)ₛ—, wherein Z is O, S, or NR₃; n is 1-5; r and s are each independently 0 or 1 wherein each R₁ and each R₂ are each independently hydrogen, halogen, optionally substituted lower alkyl, optionally substituted lower heteroalkyl, optionally substituted lower alkoxy, or together may form an optionally substituted cycloalkyl; r and s are not both 0; each R₃ is selected from the group consisting of hydrogen, optionally substituted lower alkyl, and optionally substituted heteroalkyl; A, X₁ and X₂ are each independently selected from the group consisting of hydrogen, optionally substituted lower alkyl, optionally substituted cycloalkyl, halogen, optionally substituted heteroalkyl, optionally substituted cycloheteroalkyl, optionally substituted lower alkynyl, perhaloalkyl, perhaloalkoxy, hydroxy, optionally substituted lower alkoxy, nitro, cyano, and NH₂;
G₂ is a 5, 6, or 7-membered cyclic moiety having the structure 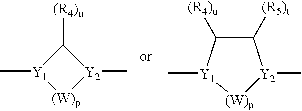
wherein Y₁ is C—R₆ or N and Y₂ is C—R₆ or N;
each R₄ and each R₅ are each independently selected from the group consisting of hydrogen, optionally substituted lower alkyl, halogen, lower perhaloalkyl, hydroxy, optionally substituted heteroalkyl, optionally substituted cycloalkyl, optionally substituted lower alkoxy, nitro, cyano, lower perhaloalkoxy, NH₂,
and —C(O)—O—R₁₁ wherein R₁₁ is hydrogen or optionally substituted lower alkyl, provided that R₄ is not hydroxy or NH₂ when Y₁ is N and R₅ is not hydroxy or NH₂ when Y₂ is N;
W is independently selected from the group consisting of —CR₇R₈—, and a moiety —CR₇— joined together with Y₁ or Y₂ by a double bond;
R₆ is selected from the group consisting of hydrogen, optionally substituted lower alkyl, hydroxy, and lower perhaloalkyl, or is null when Y₁ or Y₂ is joined to W by a double bond;
each u is 1 or 2, and each t is 1 or 2, provided that when both Y₁ and Y₂ are N, one of R₄ or R₅ may be taken together with one of W to form an optionally substituted 1- or 2-carbon bridge moiety;
each R₇ and each R₈ are each independently selected from the group consisting of hydrogen, optionally substituted lower alkyl, optionally substituted cycloalkyl, optionally substituted heteroalkyl, hydroxy, optionally substituted lower alkoxy, cyano, halogen, lower perhaloalkyl, NH₂, and a moiety which taken together with R₄ and R₅ forms a 1 or 2 carbon bridge, provided that R₇ and R₈ are not hydroxy or NH₂ when attached to a ring carbon atom adjacent to a ring nitrogen atom;
p is 1, 2 or 3, provided that the G₂ moiety comprises a 5, 6 or 7-membered ring;
G₃ is selected from the group consisting of a bond, a double bond, —(CR₉R₁₀)ₘ, carbonyl, and —(CR₉R₁₀)ₘCR₉═CR₁₀—, wherein m is 0, 1, or 2, and wherein each R₉ and each R₁₀ is independently hydrogen, optionally substituted lower alkyl, optionally substituted lower alkoxy, optionally substituted aryl, lower perhaloalkyl, cyano, and nitro; and
G₄ is selected from the group consisting of hydrogen, optionally substituted aryl, optionally substituted heteroaryl, optionally substituted cycloalkyl, optionally substituted cycloheteroalkyl, optionally substituted cycloalkenyl, optionally substituted fused aryl, optionally substituted fused heteroaryl, and optionally substituted fused cycloalkyl;
provided that when G₄ is said optionally substituted cycloheteroalkyl, said optional substitutents are non-cyclic; and further provided that when G₃ is a bond, G₄ may be covalently linked to G₂.

2. The compound of claim 1 wherein G₁ is —CR₁R₂)ₙ—.

3. The compound of claim 2, wherein each R₁ and each R₂ are each independently selected from the group consisting of hydrogen, methyl, ethyl, and propyl, or together may form a cyclopropyl, cyclobutyl, cyclopentyl, or cyclohexyl.

4. The compound of claim 3, wherein each R₁ and each R₂ are each hydrogen.

5. The compound of claim 2 wherein n=1.

6. The compound of claim 5 wherein G₁ is —CH₂— and A is selected from the group consisting of lower alkyl, optionally substituted cycloalkyl, optionally substituted cycloheteroalkyl, hydroxy, NH₂, and optionally substituted heteroalkyl wherein said heteroalkyl is attached to the phenyl ring at a carbon atom and said heteroalkyl contains at least one heteroatom selected from the group consisting of O, N, and S.

7. The compound of claim 1 having a structural formula selected from the group consisting of:

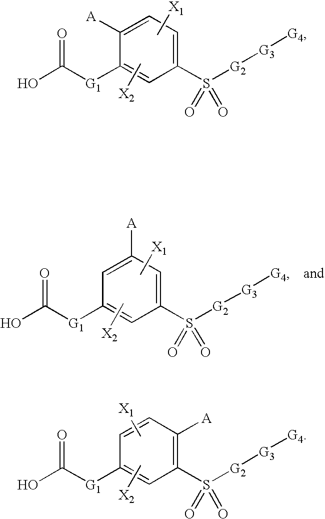

8. The compound of claim 7 wherein A is selected from the group consisting of optionally substituted lower alkyl, optionally substituted cycloalkyl, halogen, optionally substituted heteroalkyl, optionally substituted cycloheteroalkyl, lower perhaloalkyl, hydroxy, and NH₂.

9. The compound of claim 8 wherein A is selected from the group consisting of lower alkyl, optionally substituted cycloalkyl, optionally substituted cycloheteroalkyl, hydroxy, NH₂, and optionally substituted heteroalkyl wherein said heteroalkyl is attached to the phenyl ring at a carbon atom and said heteroalkyl contains at least one heteroatom selected from the group consisting of O, N, and S.

10. The compound of claim 9 wherein A is selected from the group consisting of lower alkyl and said optionally substituted heteroalkyl.

11. The compound of claim 1 wherein A, X₁, and X₂ are each independently selected from the group consisting of hydrogen, optionally substituted lower alkyl, lower perhaloalkyl, and halogen.

12. The compound of claim 11, wherein at least one of A, X₁, and X₂ is methyl.

13. The compound of claim 1 wherein G₂ is selected from the group consisting of: 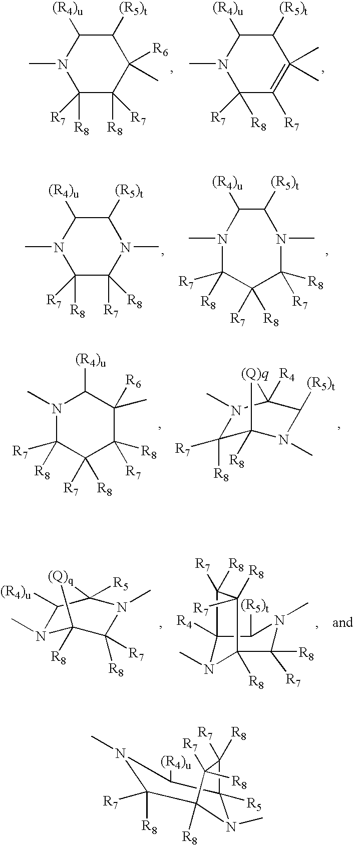
wherein each R₄, each R₅, each R₇ and each R₈ are each independently selected from the group consisting of hydrogen, optionally substituted lower alkyl, halogen, lower perhaloalkyl, hydroxy, optionally substituted lower alkoxy, nitro, cyano, carboxy, and NH₂, or together may form an optionally substituted cycloalkyl, provided that R₄, R₅, R₇ and R₈ are not hydroxy or NH₂ when attached to a ring carbon atom adjacent to a ring nitrogen atom;
each Q is each independently —CR₇R₈—; and
q is 1 or 2.

14. The compound of claim 13 wherein A is selected from the group consisting of lower alkyl, optionally substituted cycloalkyl, optionally substituted cycloheteroalkyl, hydroxy, NH₂, and optionally substituted heteroalkyl wherein said heteroalkyl is attached to the phenyl ring at a carbon atom and said heteroalkyl contains at least one heteroatom selected from the group consisting of O, N, and S.

15. The compound of claim 1 wherein p is 2; each W is CR₇R₈ or is a moiety —CR₇— joined to Y₂ by a double bond; and Y₁ is N.

16. The compound of claim 15 wherein each W is —CR₇R₈—, and Y₂ is —N.

17. The compound of claim 1 wherein said G₂ comprises at least one chiral center.

18. The compound of claim 1 having a structural formula selected from the group consisting of:

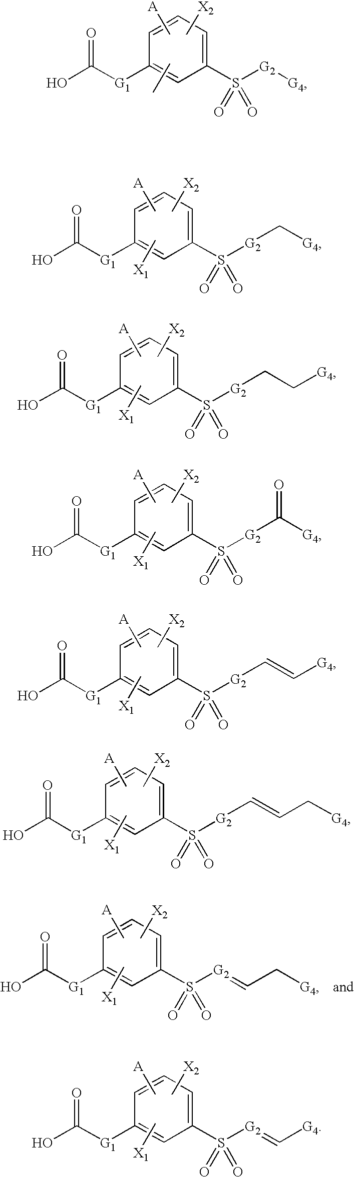

19. The compound of claim 1 wherein G₃ is a bond.

20. The compound of claim 1 wherein G₄ is optionally substituted aryl, optionally substituted heteroaryl, optionally substituted fused aryl or optionally substituted fused heteroaryl.

21. The compound of claim 20 wherein G₄ has a structural formula selected from the group consisting of: 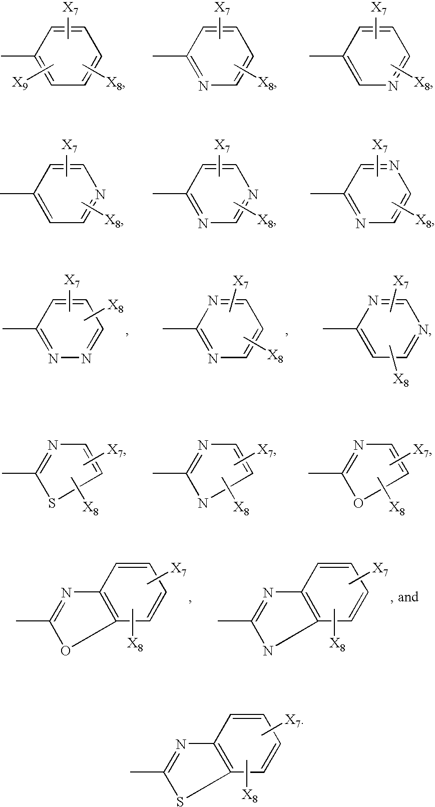
wherein each X₇, each X₈, and each X₉ are each independently selected from the group consisting of hydrogen, optionally substituted lower alkyl, optionally substituted lower alkynyl, halogen, optionally substituted lower heteroalkyl, lower perhaloalkyl, hydroxy, optionally substituted lower alkoxy, lower perhaloalkoxy, nitro, cyano, NH₂, and —CO₂R₁₂, where R₁₂ is selected from the group consisting of optionally substituted lower alkyl and H; further provided that when X₇ and X₈ are present at adjacent ring positions of G₄, X₇ and X₈ may together form an optionally substituted aryl, heteroaryl, aliphatic or heteroaliphatic ring.

22. The compound of claim 21 wherein X₇ is selected from the group consisting of halogen, lower perhaloalkyl and lower perhaloalkoxy and X₈ is selected from the group consisting of hydrogen, halogen, optionally substituted lower alkyl, lower perhaloalkyl and lower perhaloalkoxy.

23. The compound of claim 1 wherein the compound is an hPPAR-delta modulator.

24. The compound of claim 23 wherein the compound is a selective hPPAR-delta modulator.

25. The compound of claim 23 wherein the compound modulates hPPAR-delta having an EC₅₀ value less than 5 μM as measured by a functional cell assay.

26. A compound having a structural formula selected from the group consisting of: 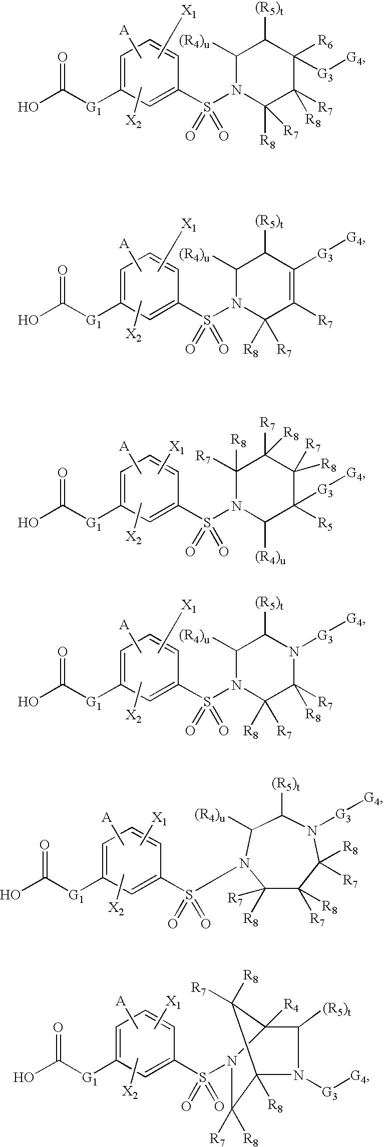 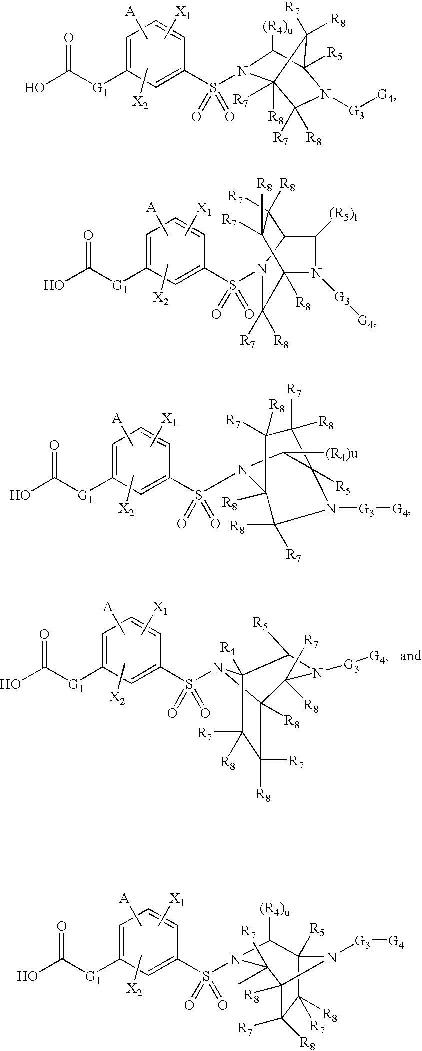
or a pharmaceutically acceptable N-oxide, pharmaceutically acceptable prodrug, pharmaceutically acceptable metabolite, pharmaceutically acceptable salt, pharmaceutically acceptable ester, pharmaceutically acceptable amide, or pharmaceutically accept able solvate thereof;
wherein:
G₁ is —CR₁R₂)ₙ— wherein n is 1 to 5 and each R₁ and each R₂ are each independently hydrogen, fluoro, optionally substituted lower alkyl, optionally substituted lower heteroalkyl, optionally substituted lower alkoxy, and lower perhaloalkyl or together may form an optionally substituted cycloalkyl;
A, X₁ and X₂ are each independently selected from the group consisting of hydrogen, optionally substituted lower alkyl, optionally substituted cycloalkyl, halogen, optionally substituted heteroalkyl, optionally substituted cycloheteroalkyl, optionally substituted lower alkynyl, perhaloalkyl, perhaloalkoxy, hydroxy, optionally substituted lower alkoxy, nitro, cyano, and NH₂;
each R₄, each R₅, each R₇, and each R₈ are each independently selected from the group consisting of hydrogen, optionally substituted lower alkyl, halogen, lower perhaloalkyl, hydroxy, optionally substituted heteroalkyl, optionally substituted cycloalkyl, optionally substituted lower alkoxy, nitro, cyano, lower perhaloalkoxy, NH₂, and —C(O)—OR₁₁, wherein R₁₁ is hydrogen or optionally substituted lower alkyl;
R₆ is selected from the group consisting of hydrogen, optionally substituted lower alkyl, hydroxy, and C₁₋₄ perhaloalkyl;
u is 1 or 2; t is 1 or 2;
G₃ is selected from the group consisting of a bond, a double bond, —CR₉R₁₀)ₘ—, carbonyl, and —(CR₉R₁₀)ₘCR₉═CR₁₀, wherein m is 0, 1, or 2, and wherein each R₉ and each R₁₀ is independently hydrogen, optionally substituted lower alkyl, optionally substituted lower alkoxy, optionally substituted aryl, lower perhaloalkyl, cyano, and nitro; and
G₄ is selected from the group consisting of optionally substituted aryl, optionally substituted heteroaryl, optionally substituted cycloalkyl, optionally substituted cycloheteroalkyl, optionally substituted cycloalkenyl, optionally substituted fused aryl, optionally substituted fused heteroaryl, and optionally substituted fused cycloalkyl;
provided that when G₄ is said optionally substituted cycloheteroalkyl, said optional substitutents are non-cyclic; and further provided that when G₃ is a bond, G₄ may be covalently linked to G₂.

27. The compound of claim 26 wherein A is selected from the group consisting of optionally substituted lower alkyl, optionally substituted cycloalkyl, halogen, optionally substituted heteroalkyl, optionally substituted cycloheteroalkyl, lower perhaloalkyl, hydroxy, and NH₂.

28. The compound of claim 27 wherein A is selected from the group consisting of lower alkyl, optionally substituted cycloalkyl, optionally substituted cycloheteroalkyl, hydroxy, NH₂, and optionally substituted heteroalkyl wherein said heteroalkyl is attached to the phenyl ring at a carbon atom and said heteroalkyl contains at least one heteroatom selected from the group consisting of O, N, and S.

29. The compound of claim 28 wherein A is selected from the group consisting of lower alkyl and said optionally substituted heteroalkyl.

30. The compound of claim 26, wherein A, X₁ and X₂ are each independently selected from the group consisting of hydrogen, optionally substituted lower alkyl, halogen, optionally substituted lower heteroalkyl, perhaloalkyl, perhaloalkoxy, and optionally substituted lower alkoxy.

31. The compound of claim 30, wherein A, X₁ and X₂ are each independently selected from the group consisting of hydrogen and methyl and at least one of A, X₁ and X₂ is methyl.

32. The compound of claim 26, wherein n=1.

33. The compound of claim 32, wherein R₁ and R₂ are each independently selected from the group consisting of hydrogen, lower alkyl, or together may form an optionally substituted cycloalkyl.

34. The compound of claim 33, wherein R₁ and R₂ are each hydrogen.

35. The compound of claim 26 having the structure

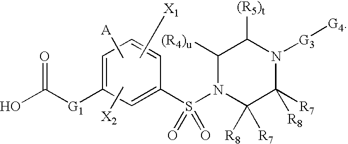

36. The compound of claim 35, wherein at least one of R₄, R₅, R₇, and R₈ is not hydrogen.

37. The compound of claim 36, wherein said at least one of R₄, R₅, R₇, and R₈ is lower alkyl.

38. The compound of claim 37, wherein said at least one of R₄, R₅, R₇, and R₈ is methyl.

39. The compound of claim 35, wherein at least two of R₄, R₅, R₇, and R₈ are methyl.

40. The compound of claim 39, wherein the at least two of R₄, R₅, R₇, and R₈ which are methyl are oriented cis to each other.

41. The compound of claim 35, wherein R₄ and R₇ are methyl and are attached to the piperazine ring at the 2 and 6 positions.

42. The compound of claim 41, wherein the R₄ and R₇ methyl groups are oriented cis to each other.

43. The compound of claim 35, wherein R₄ and R₅ are methyl.

44. The compound of claim 43, wherein the R₄ and R₅ methyl groups are oriented cis to each other.

45. The compound of claim 39, wherein the at least two of R₄, R₅, R₇, and R₈ which are methyl are oriented cis to each other.

46. The compound of claim 35, wherein G₃ is a bond.

47. The compound of claim 35, wherein G₄ has a structural formula selected from the group consisting of: 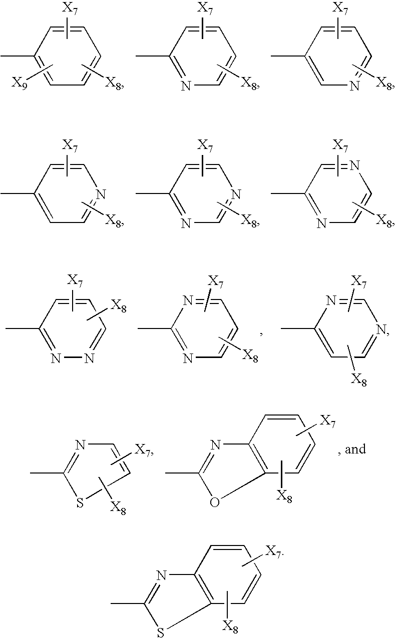
wherein each X₇, X₈ and X₉ are each independently selected from the group consisting of hydrogen, optionally substituted lower alkyl, halogen, lower perhaloalkyl, hydroxy, optionally substituted lower alkoxy, lower perhaloalkoxy, nitro, cyano, NH₂, and CO₂R₁₂ where R₁₂ is optionally substituted lower alkyl and H;
X₇ and X₈, if present on adjacent sites of G₄, may together form an aryl, heteroaryl, aliphatic or heteroaliphatic ring.

48. The compound of claim 47, wherein G₃ is a bond.

49. The compound of claim 26 wherein the compound is an hPPAR-delta modulator.

50. The compound of claim 49 wherein the compound is a selective hPPAR-delta modulator.

51. The compound of claim 49 wherein the compound modulates hPPAR-delta having an EC₅₀ value less than 5 μM as measured by a functional cell assay.

52. A compound having the structure 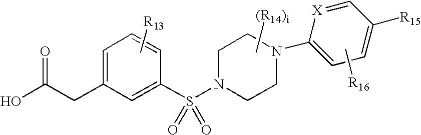
or a pharmaceutically acceptable N-oxide, pharmaceutically acceptable prodrug, pharmaceutically acceptable metabolite, pharmaceutically acceptable salt, pharmaceutically acceptable ester, pharmaceutically acceptable amide, or pharmaceutically acceptable solvate thereof;
wherein:
X is C or N;
R₁₃ is selected from the group consisting of hydrogen, C₁-C₄ alkyl, and singly or multiply fluoro substituted C₁-C₄ alkyl;
each R₁₄ is selected from the group consisting of hydrogen, C₁-C₃ alkyl;
i is 0, 1, or 2;
R₁₅ is selected from the group consisting of halogen, perhalomethyl, and perhalomethoxy; and
R₁₆ is selected from the group consisting of hydrogen, halogen, lower alkyl and lower alkoxy.

53. The compound of claim 52 wherein R₁₃ is selected from the group consisting of hydrogen, methyl, perfluoromethyl, difluoromethyl and —CH₂—CF₃.

54. The compound of claim 52 wherein R₁₄ is selected from the group consisting of hydrogen, methyl, ethyl, and isopropyl.

55. The compound of claim 54 wherein i=2 and R₁₄ is selected from the group consisting of methyl.

56. The compound of claim 55 wherein the two R₁₄ moieties are oriented cis to each other.

57. The compound of claim 56 wherein the two R₁₄ moieties are attached to the piperazine ring at the 2 and 6 positions.

58. The compound of claim 56 wherein the two R₁₄ moieties are attached to the piperazine ring at the 2 and 3 positions.

59. The compound of claim 54 wherein R₁₃ is selected from the group consisting of hydrogen, methyl, perfluoromethyl, difluoromethyl and —CH₂—CF₃.

60. The compound of claim 52 wherein R₁₅ is selected from the group consisting of halogen, perfluoromethyl, and perfluoromethoxy.

61. The compound of claim 60 wherein R₁₃ is selected from the group consisting of hydrogen, methyl, perfluoromethyl, difluoromethyl and —CH₂—CF₃.

62. The compound of claim 52 wherein the compound is an hPPAR-delta modulator.

63. The compound of claim 62 wherein the compound is a selective hPPAR-delta modulator.

64. The compound of claim 62 wherein the compound modulates hPPAR-delta having an EC₅₀ value less than 5 μM as measured by a functional cell assay.

65. A compound having a structure, or a pharmaceutically acceptable N-oxide, pharmaceutically acceptable prodrug, pharmaceutically acceptable metabolite, pharmaceutically acceptable salt, pharmaceutically acceptable ester, pharmaceutically acceptable amide, or pharmaceutically acceptable solvate thereof, wherein the structure is selected from the group consisting of:

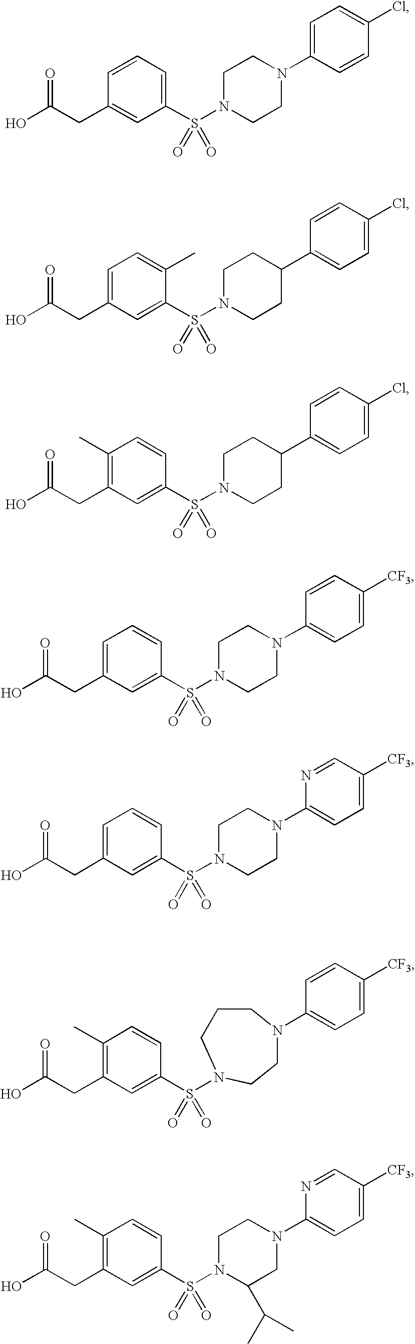

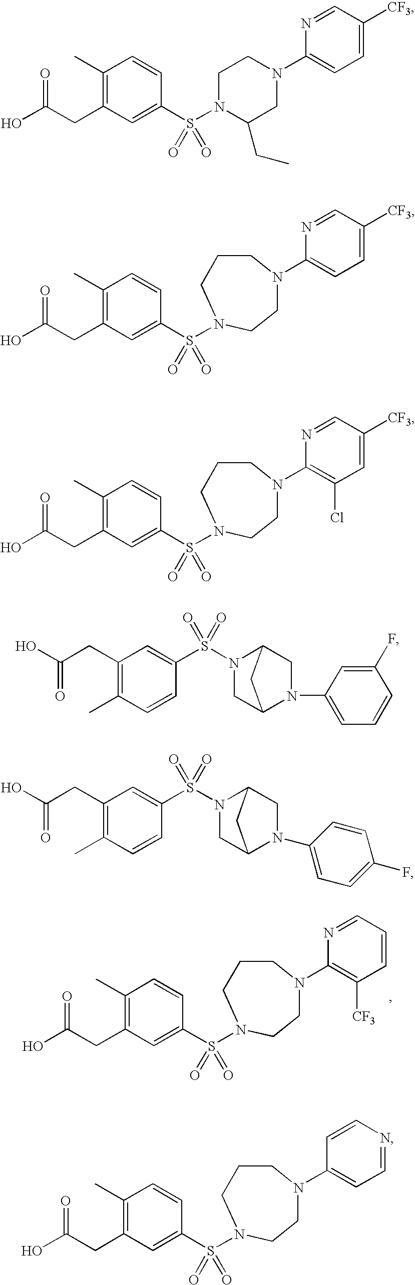

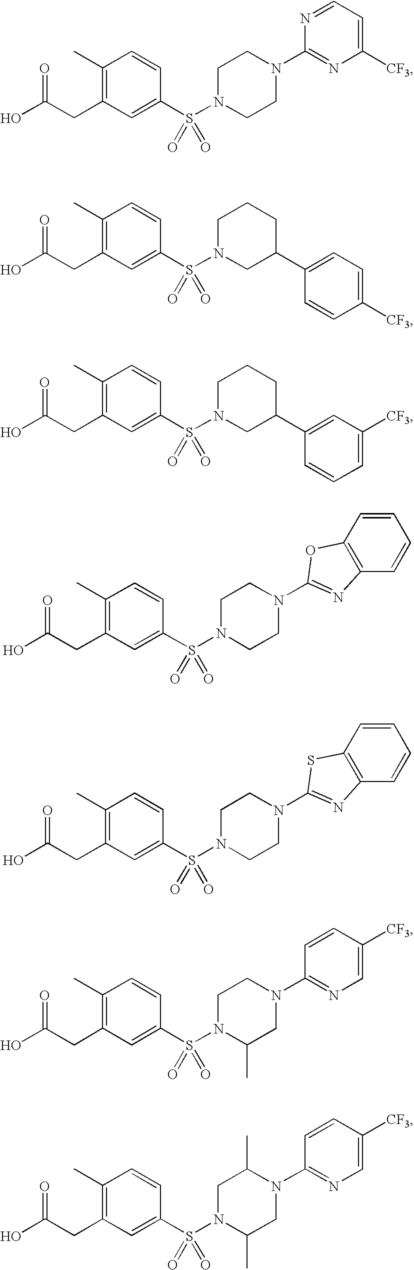

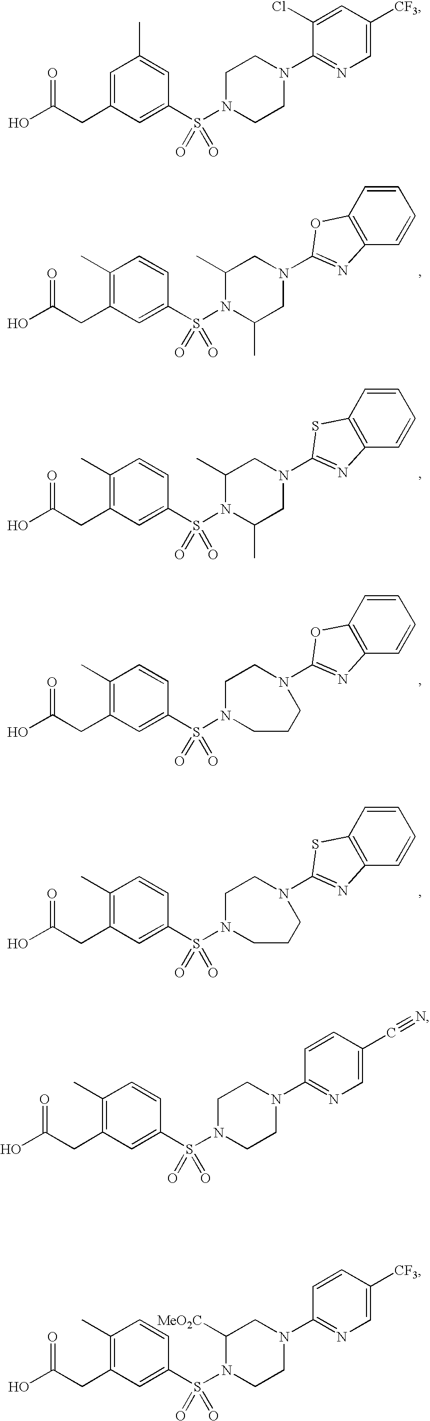

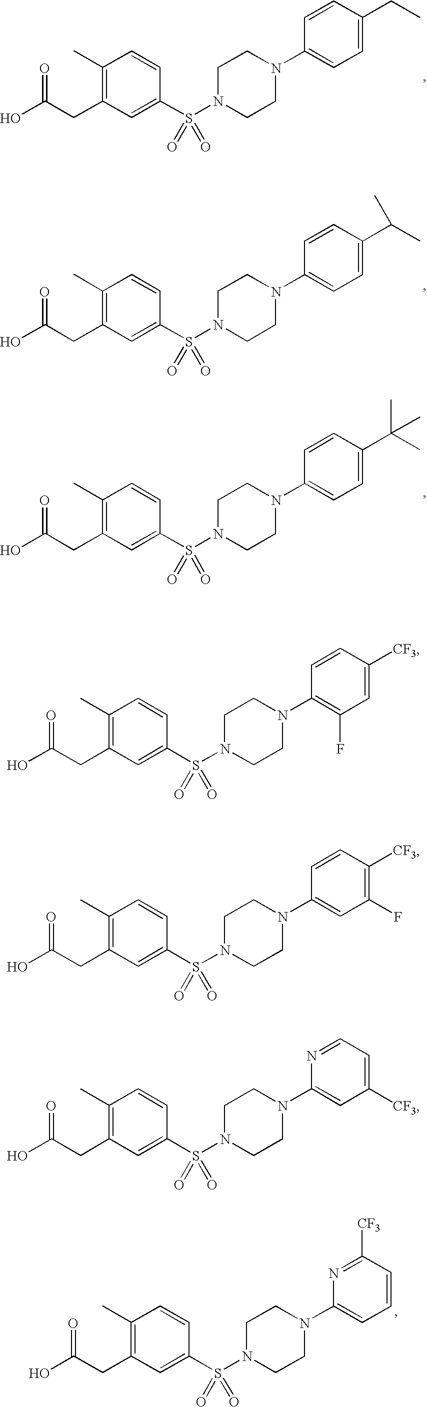

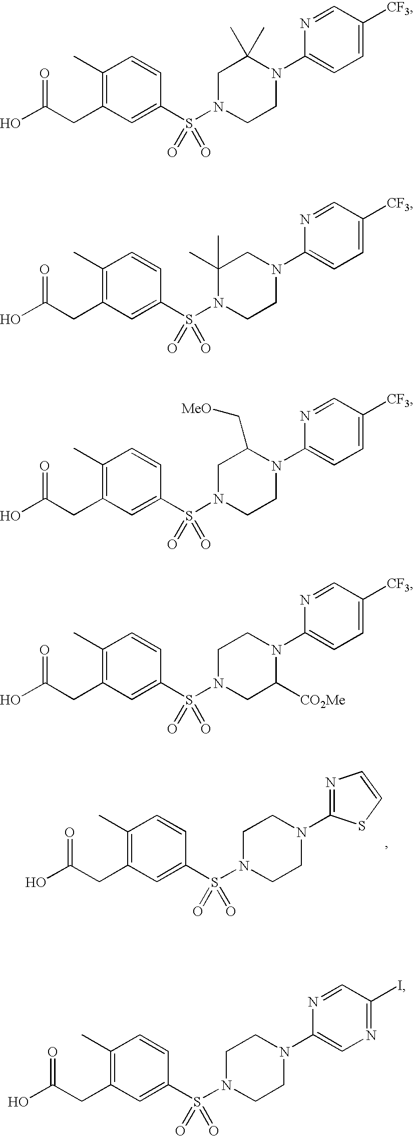

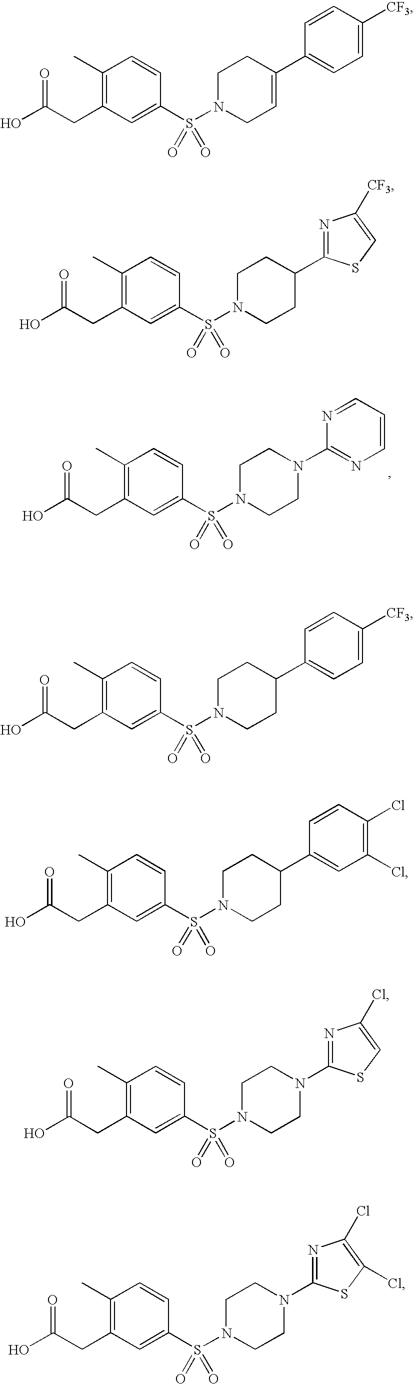

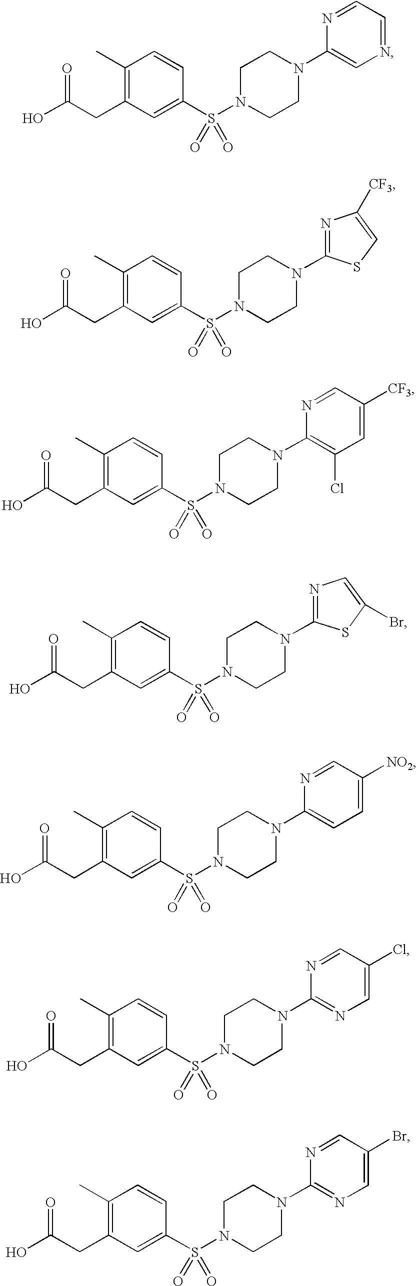

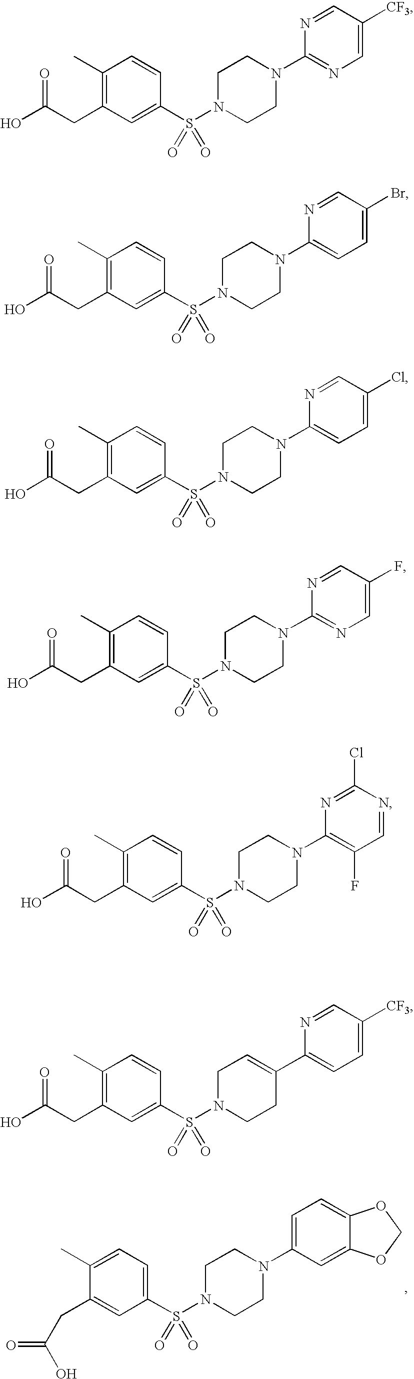

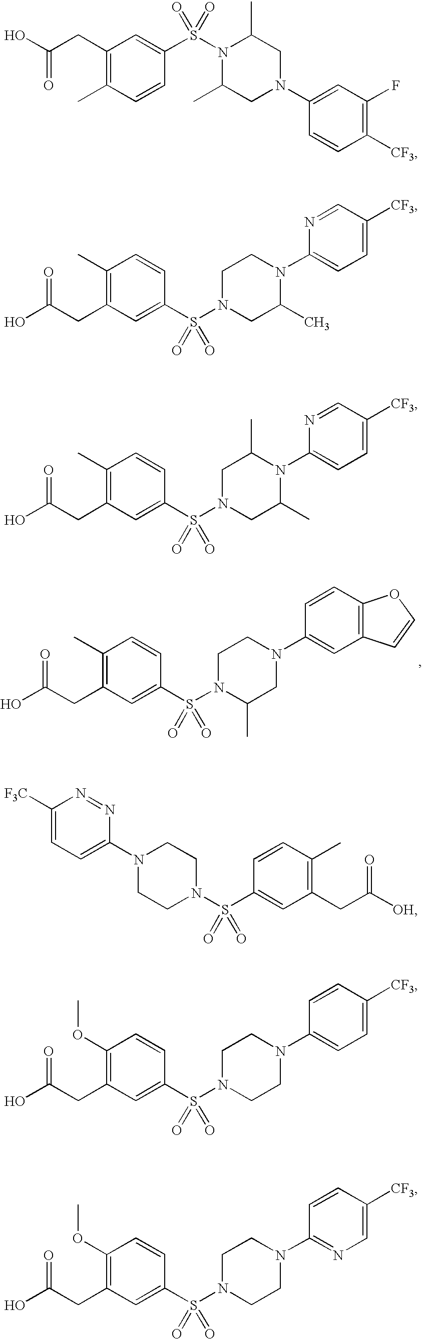

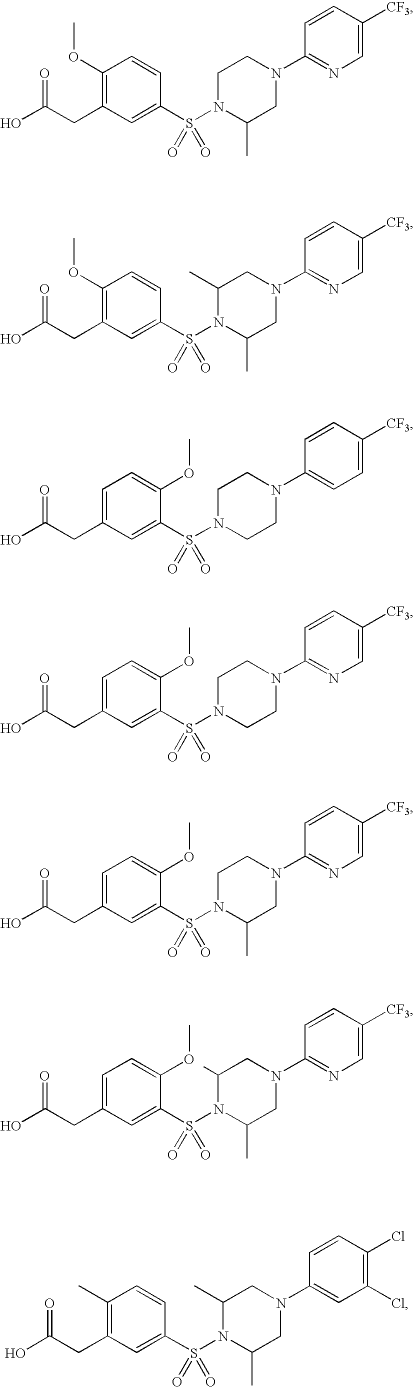

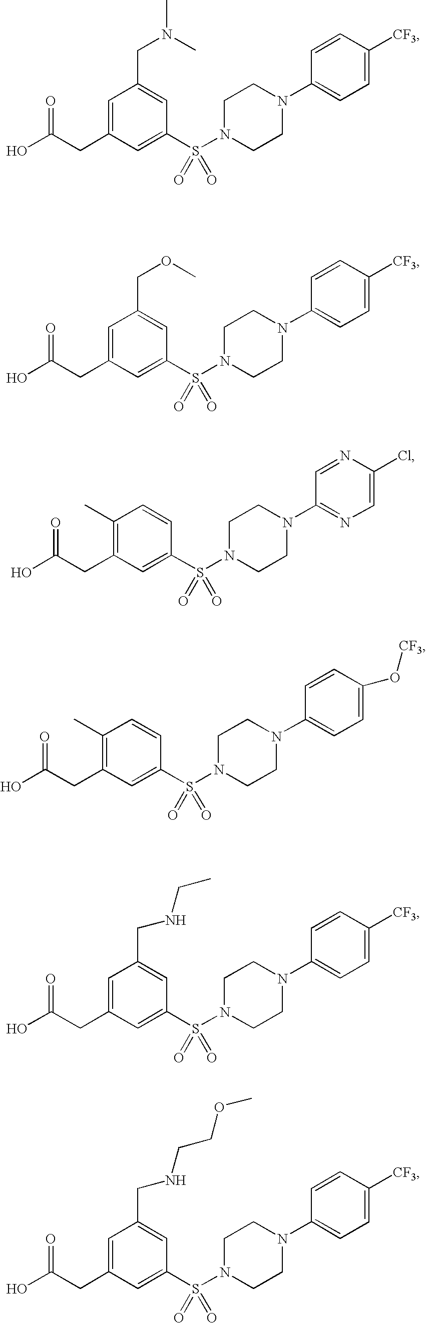

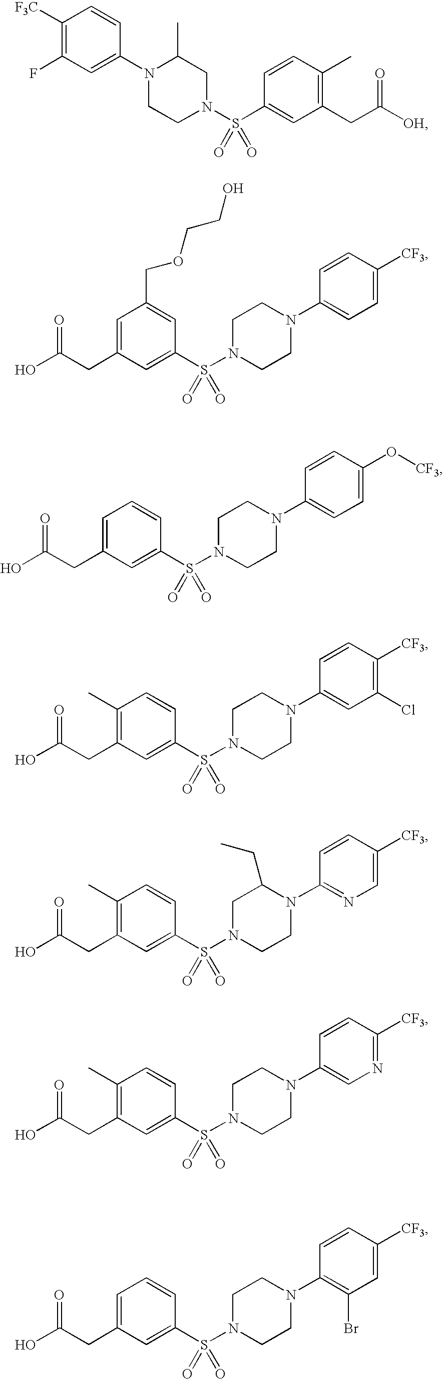

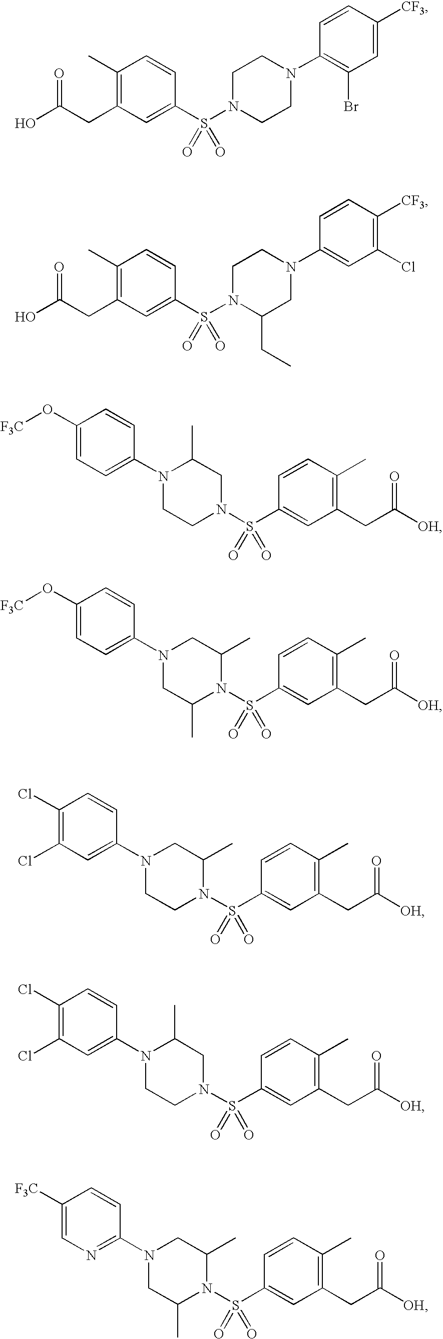

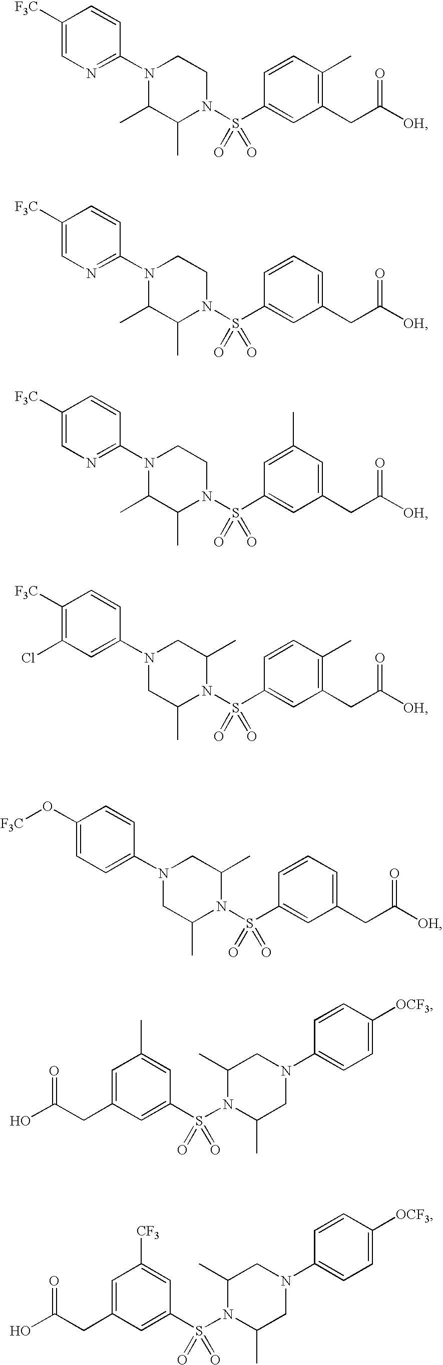

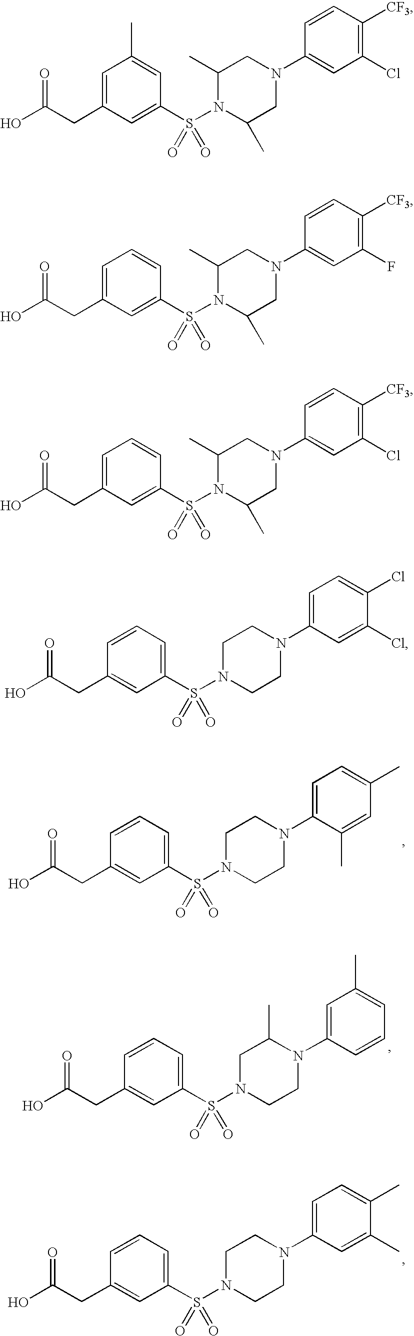

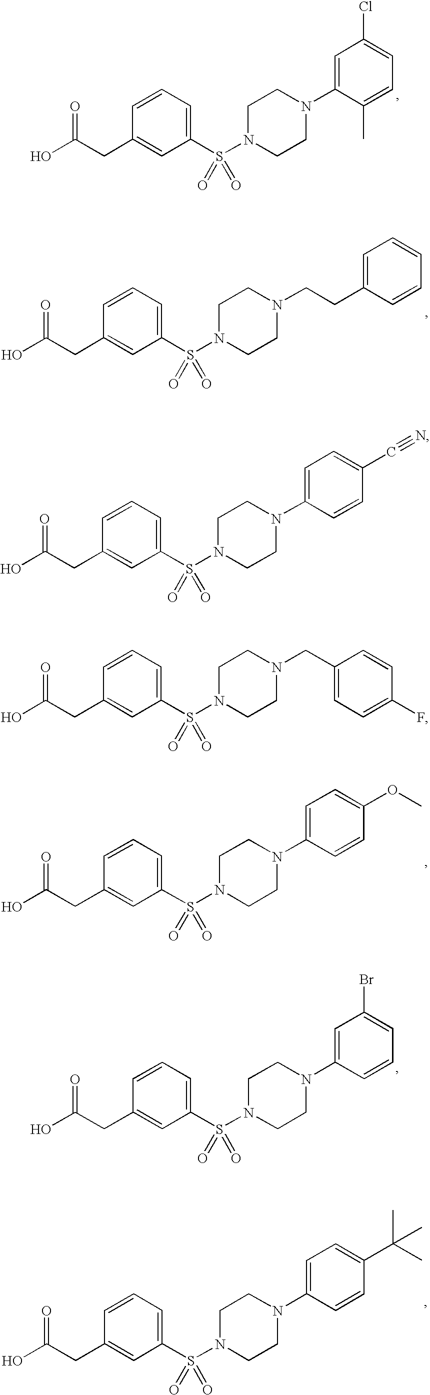

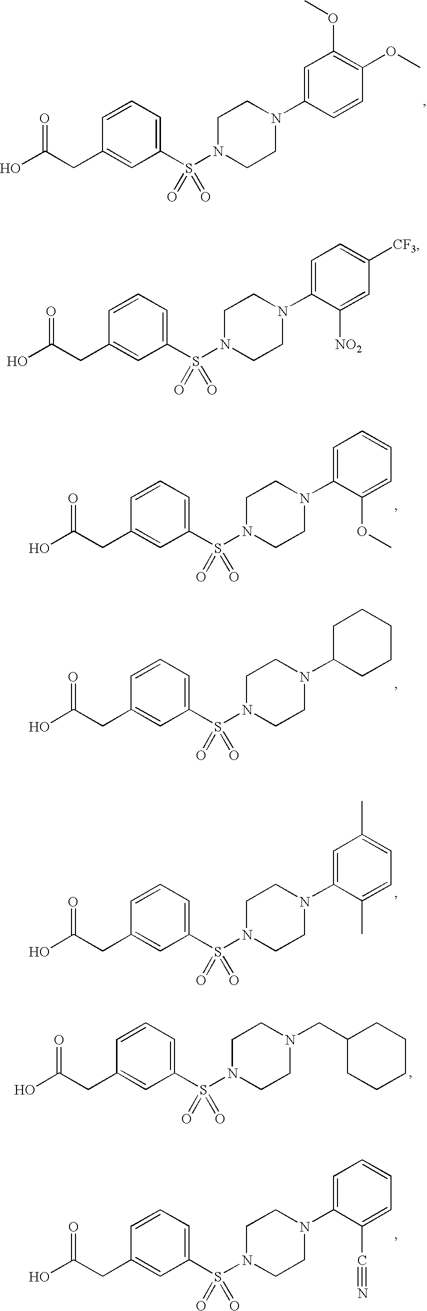

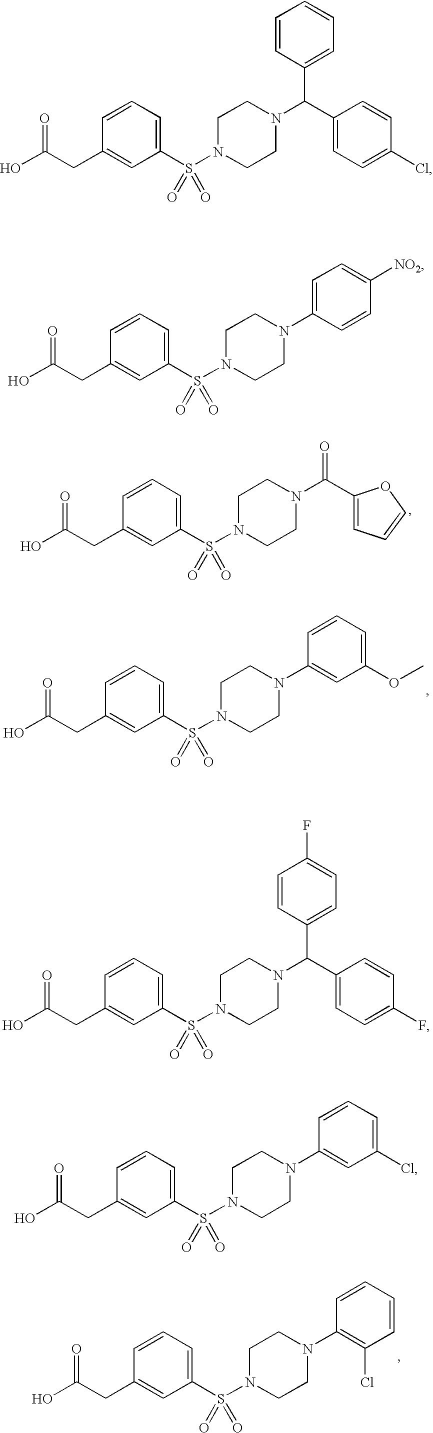

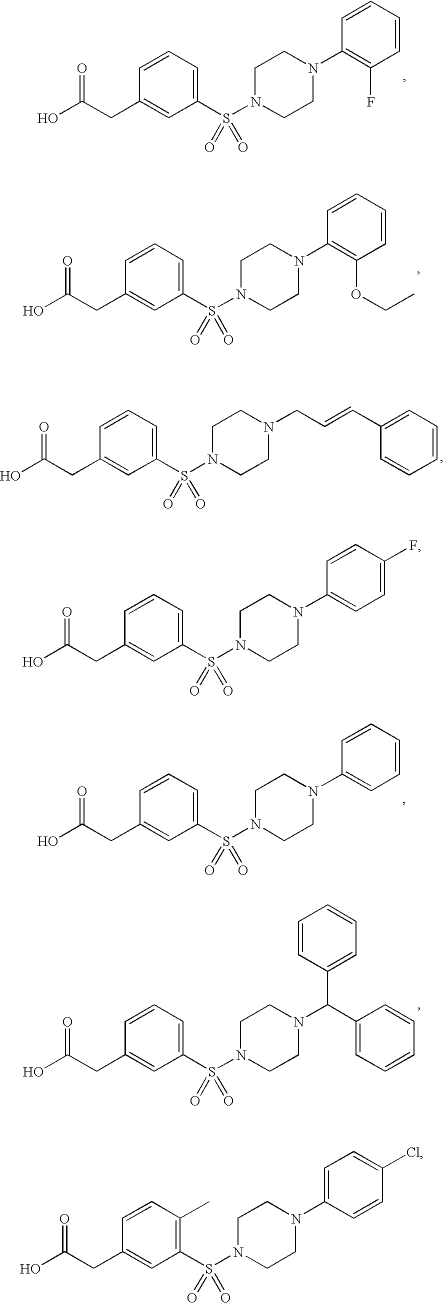

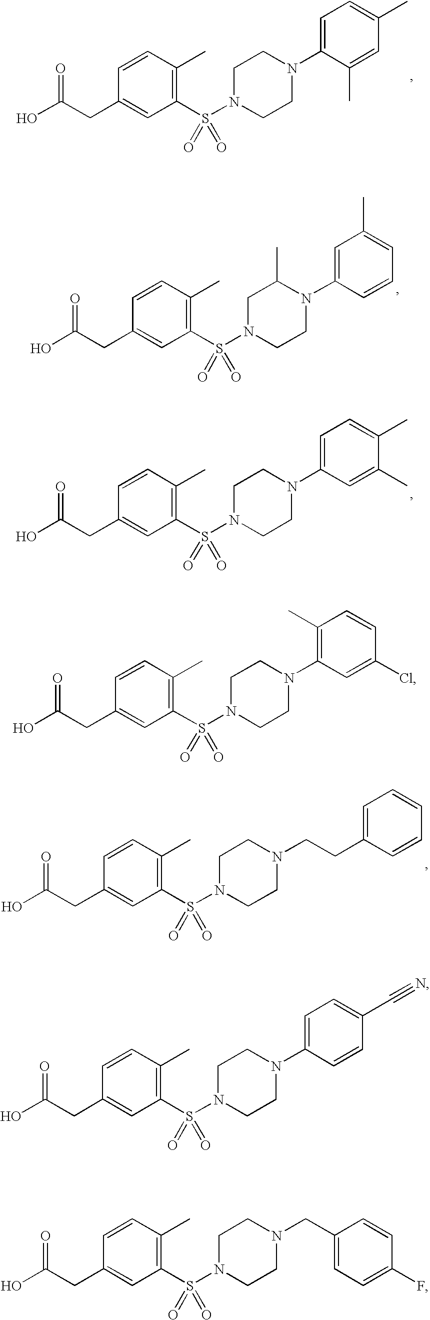

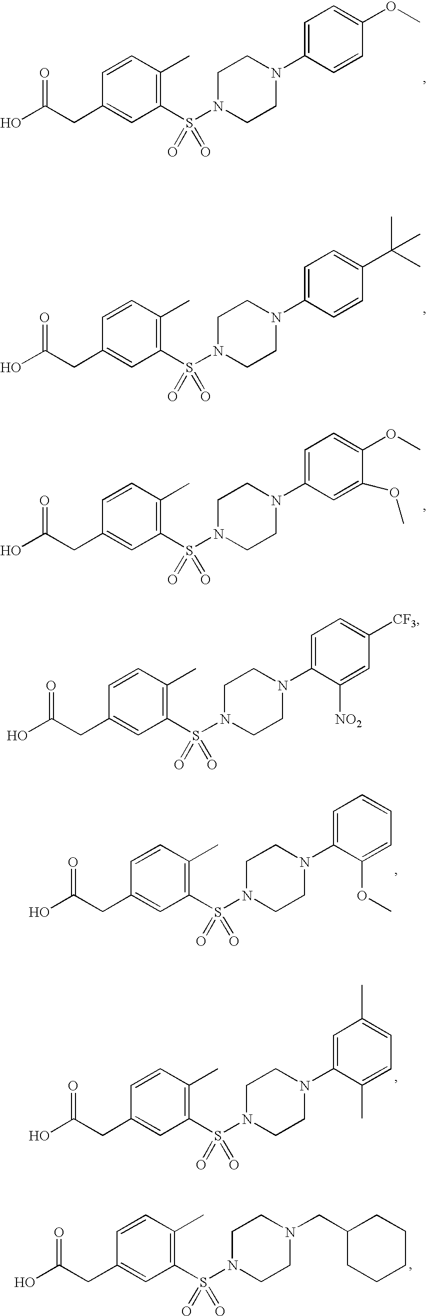

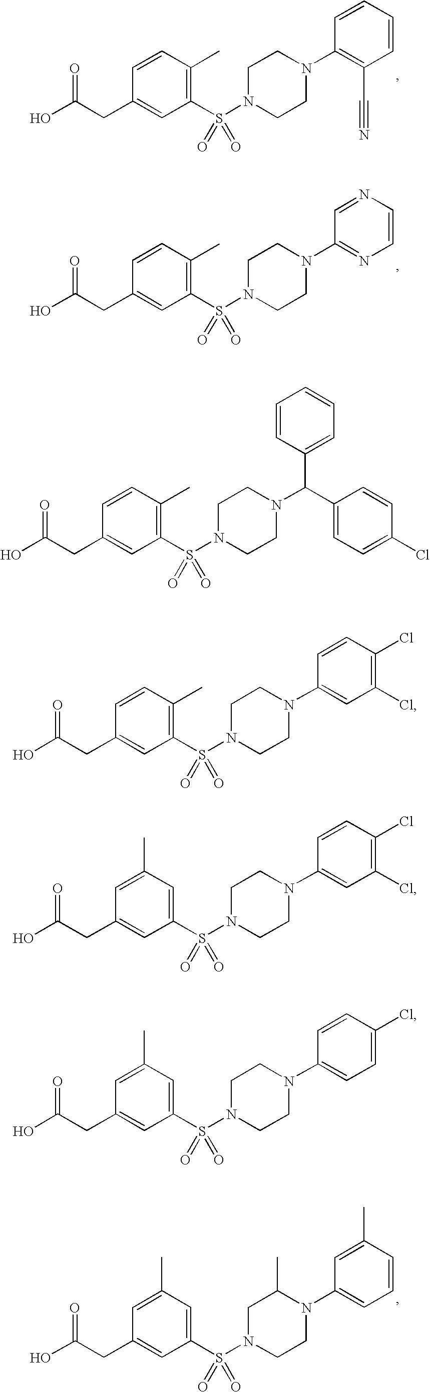

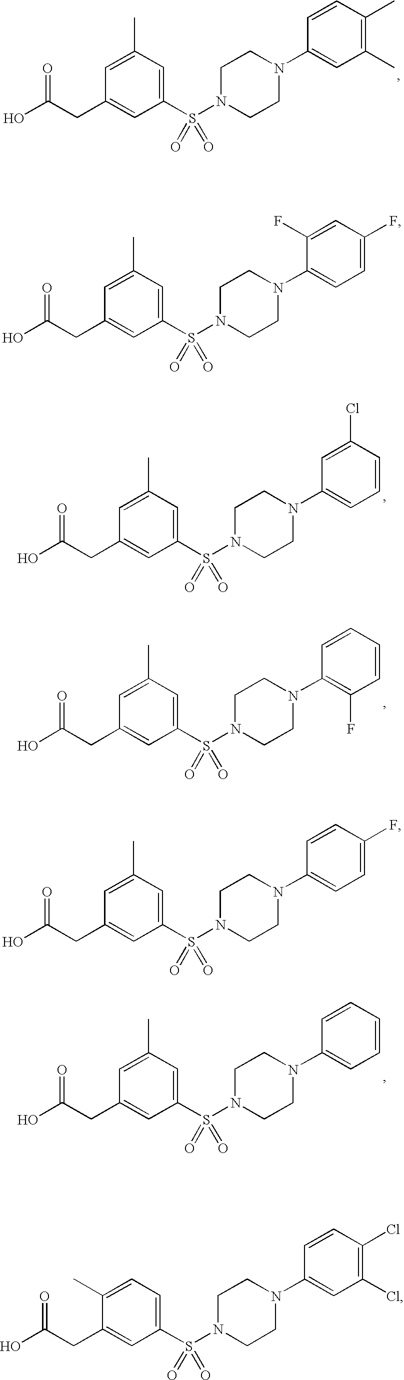

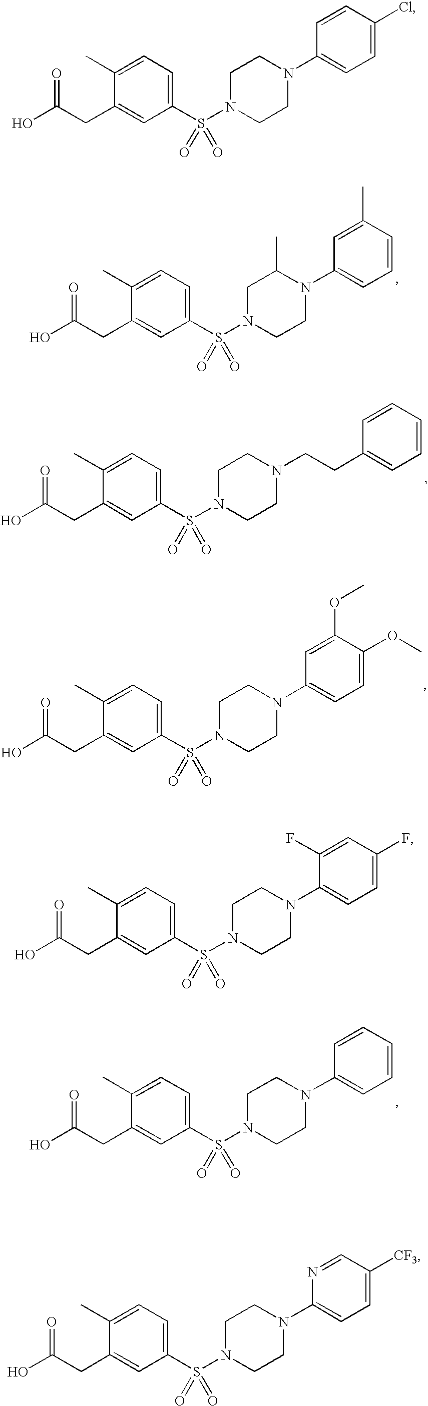

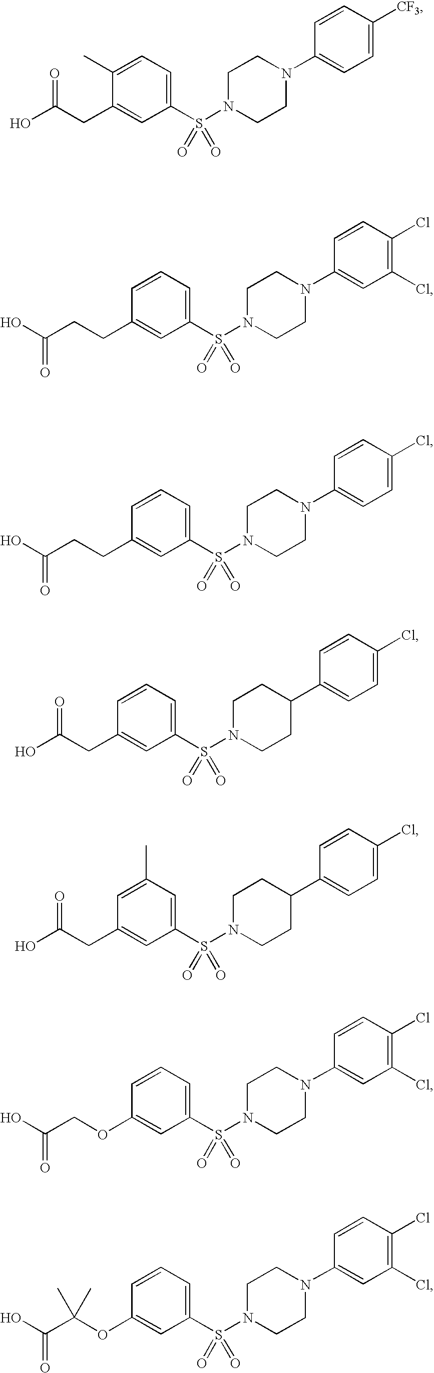

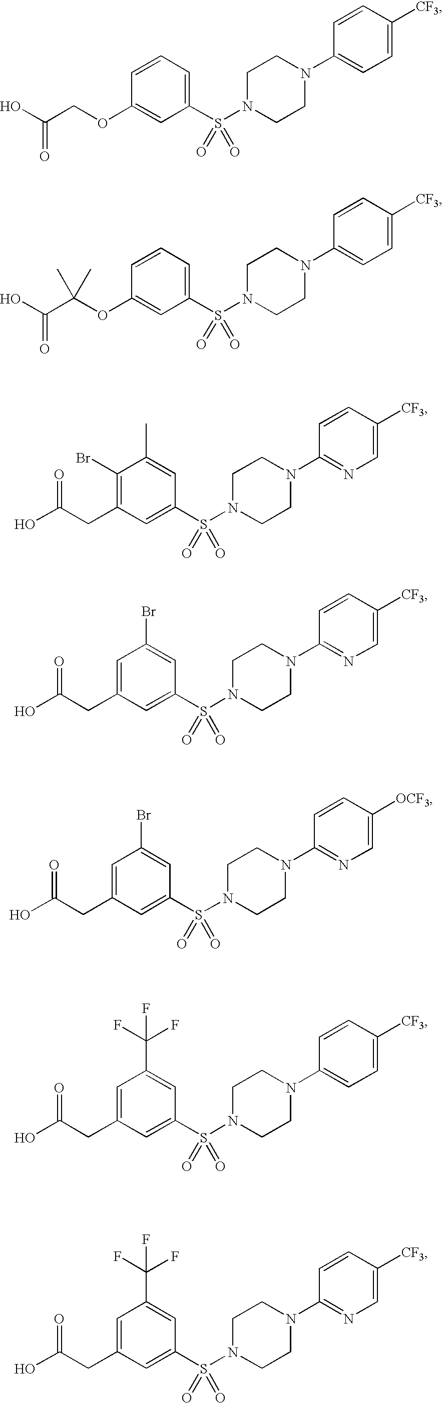

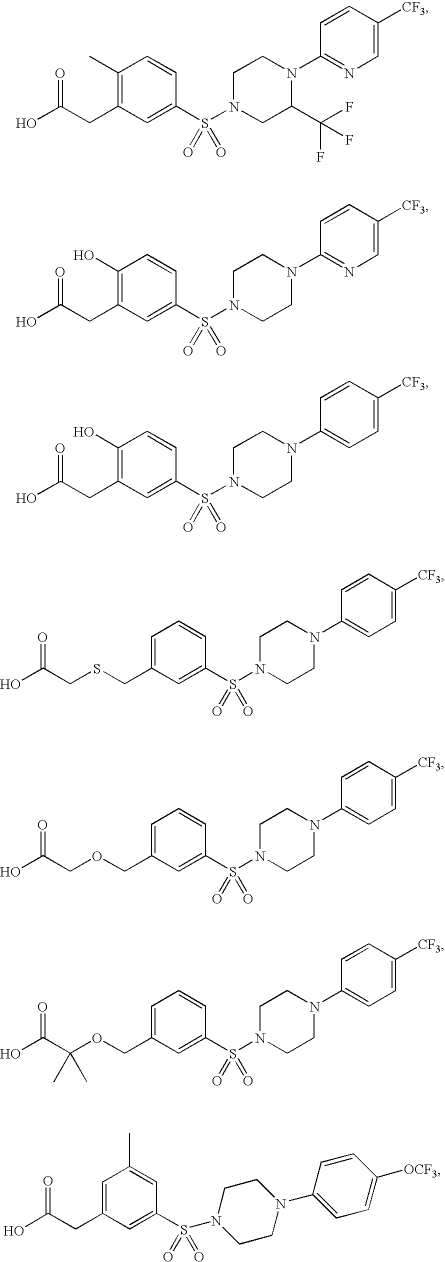

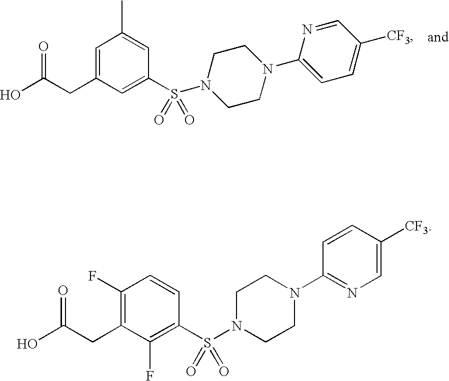

66. A compound having a structure, or a pharmaceutically acceptable N-oxide, pharmaceutically, acceptable prodrug, pharmaceutically acceptable metabolite, pharmaceutically acceptable salt, pharmaceutically acceptable ester, pharmaceutically acceptable amide, or pharmaceutically acceptable solvate thereof, wherein the structure is selected from the group consisting of:

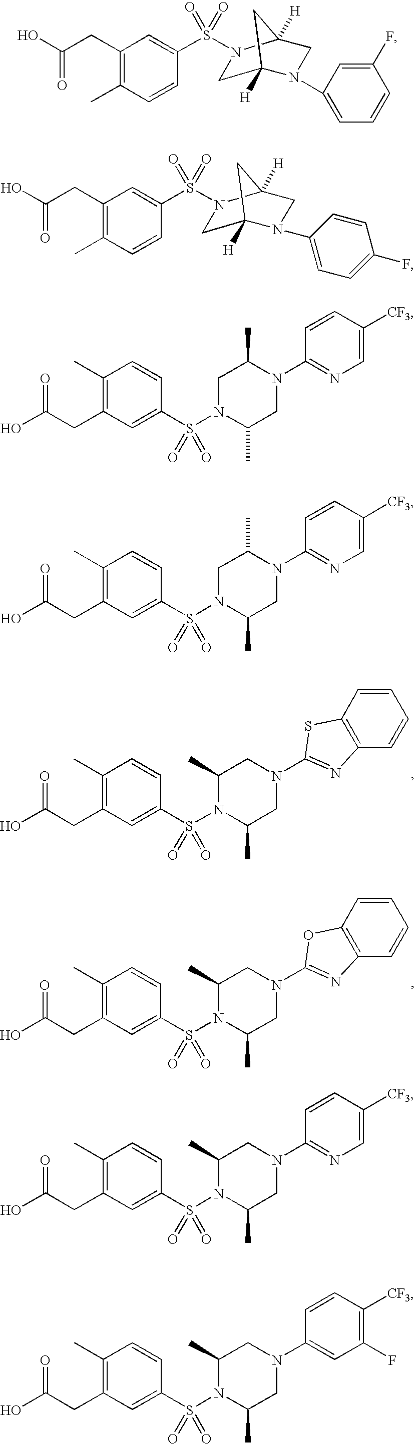

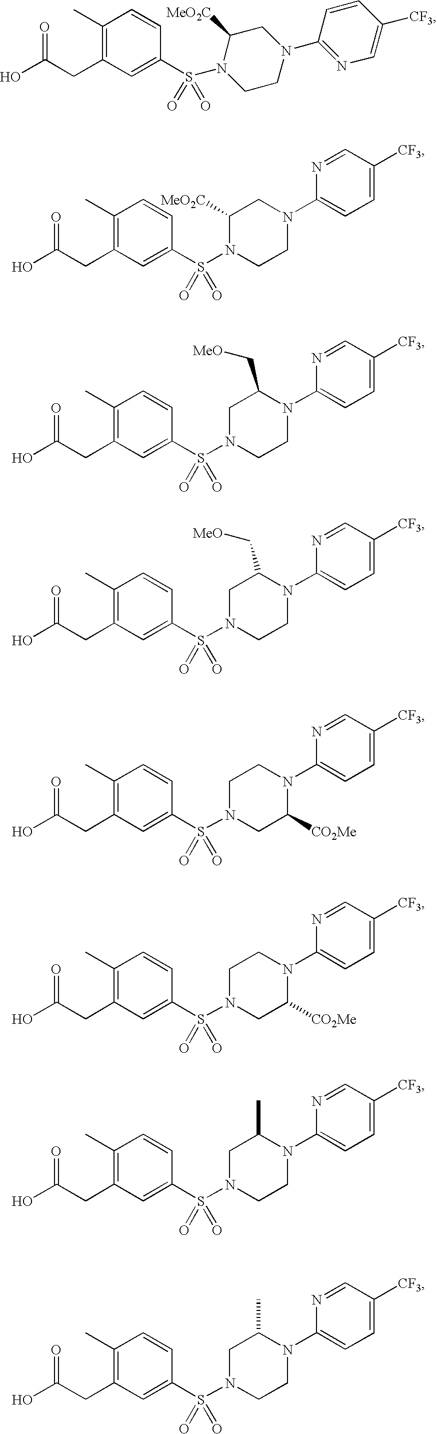

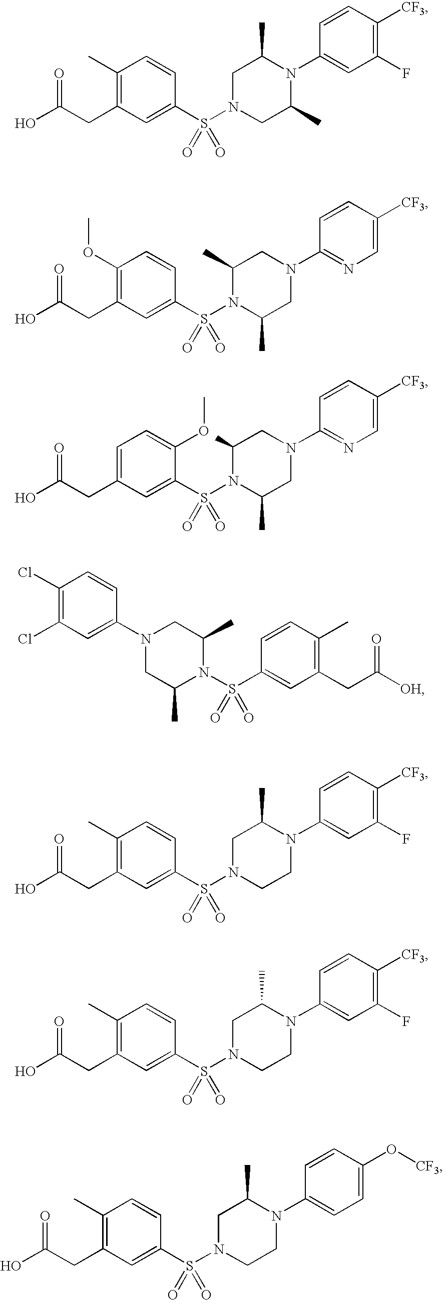

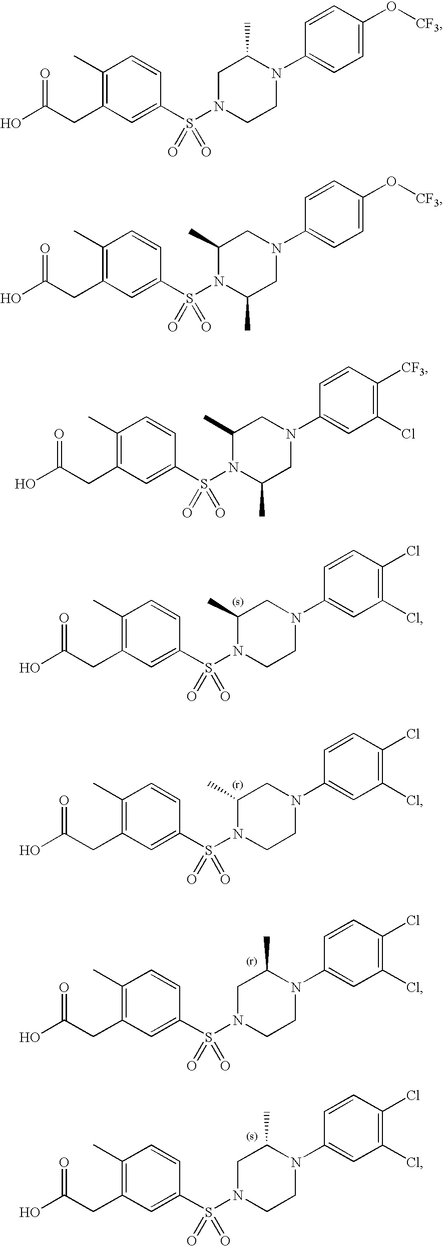

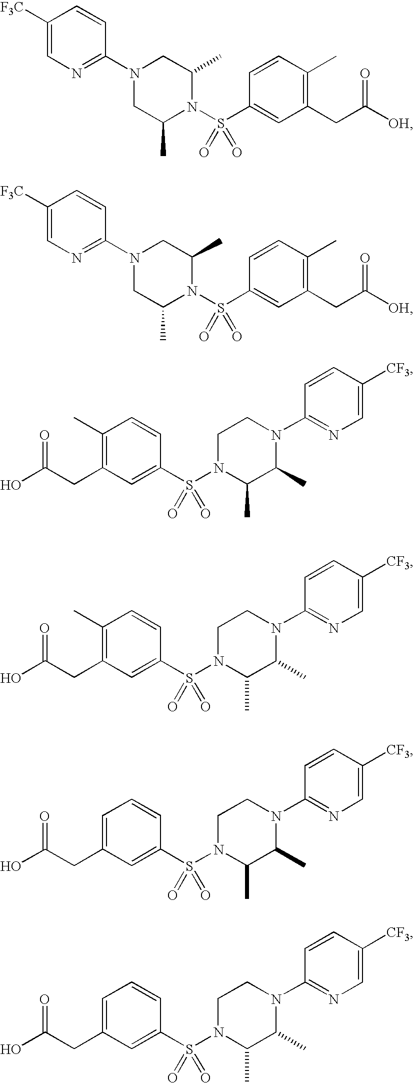

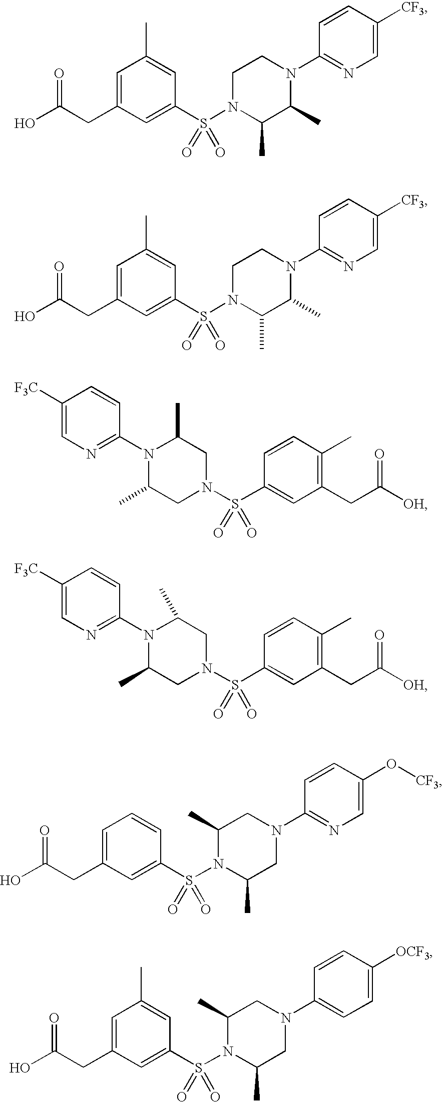

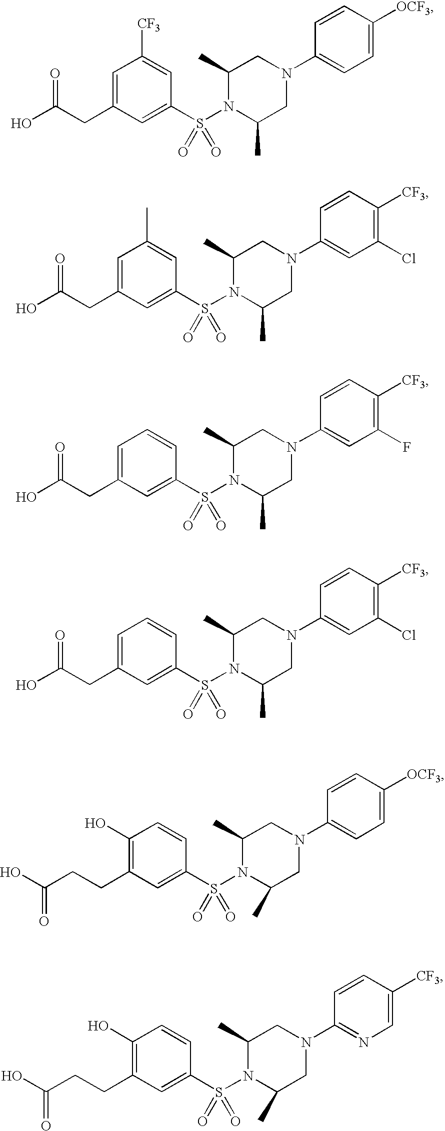

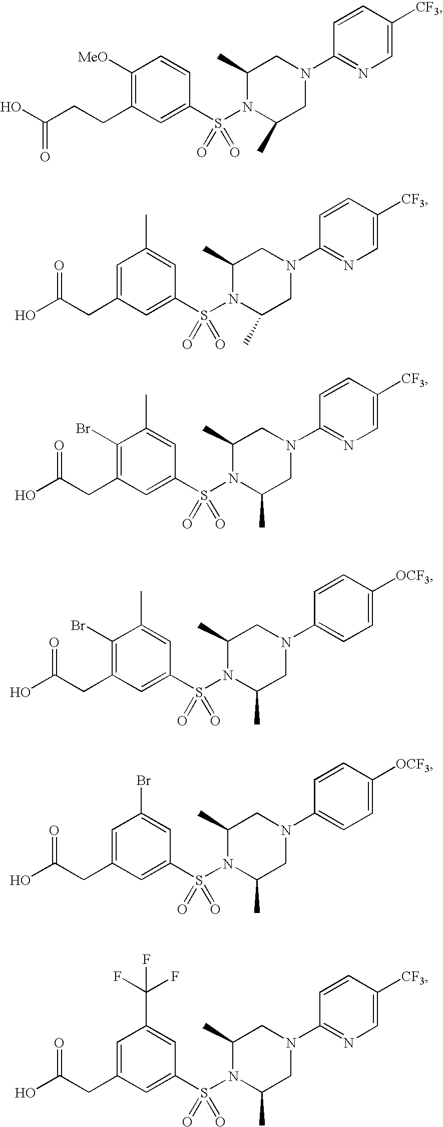

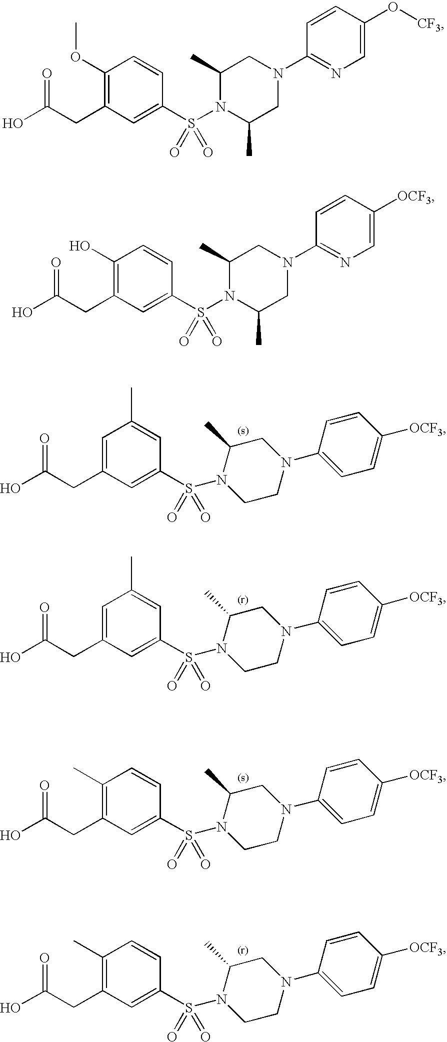

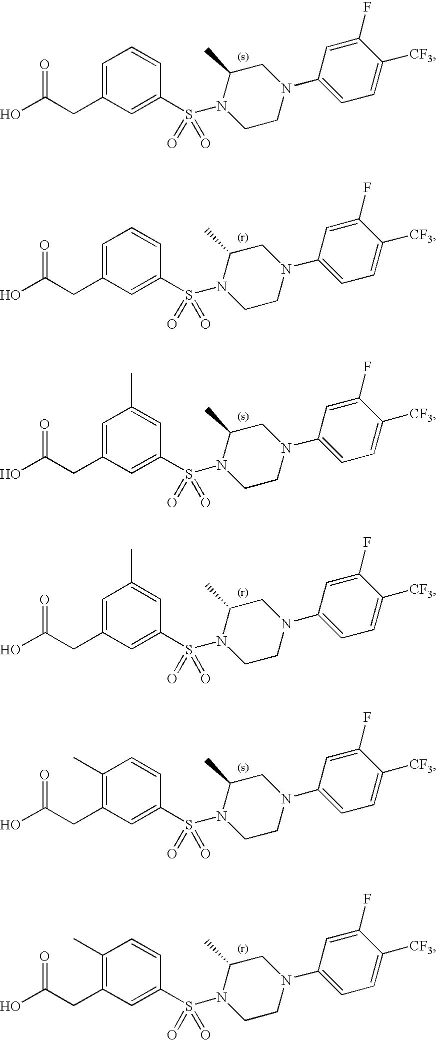

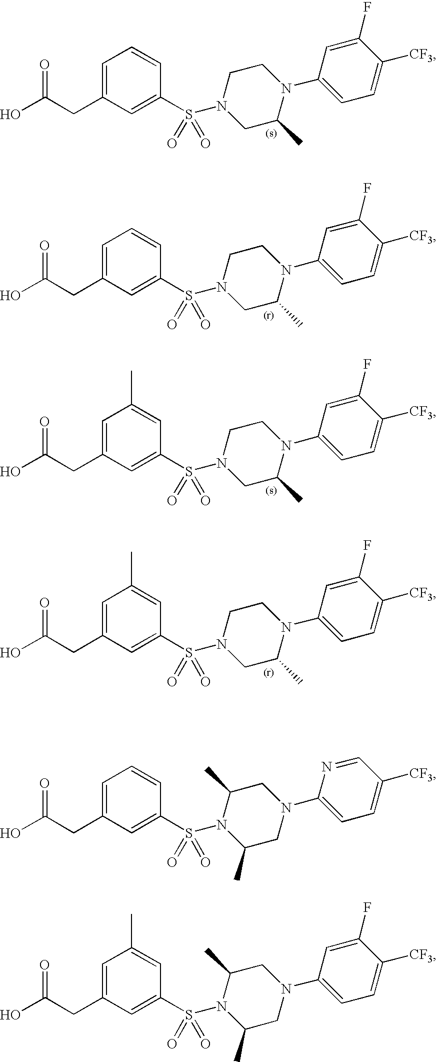

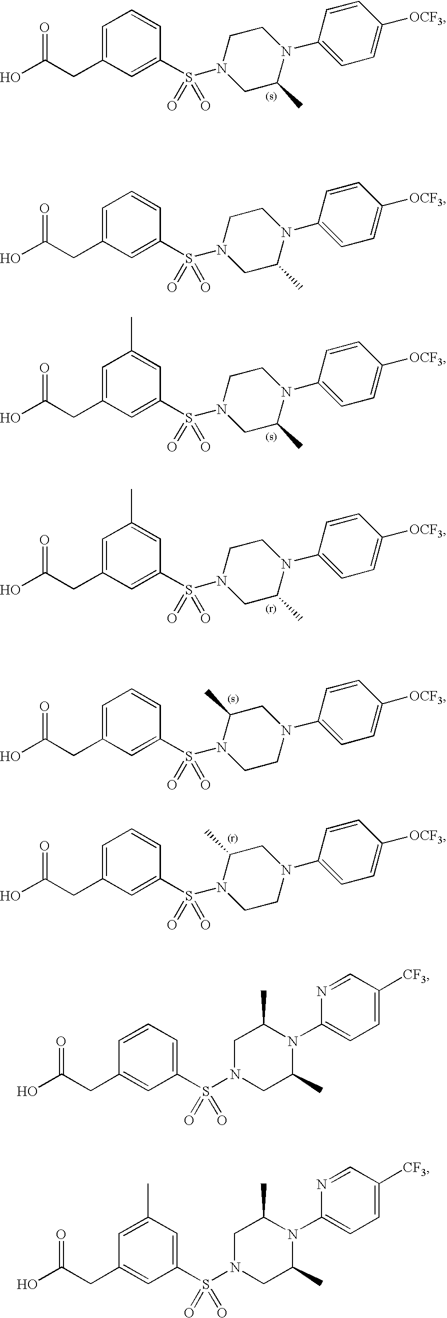

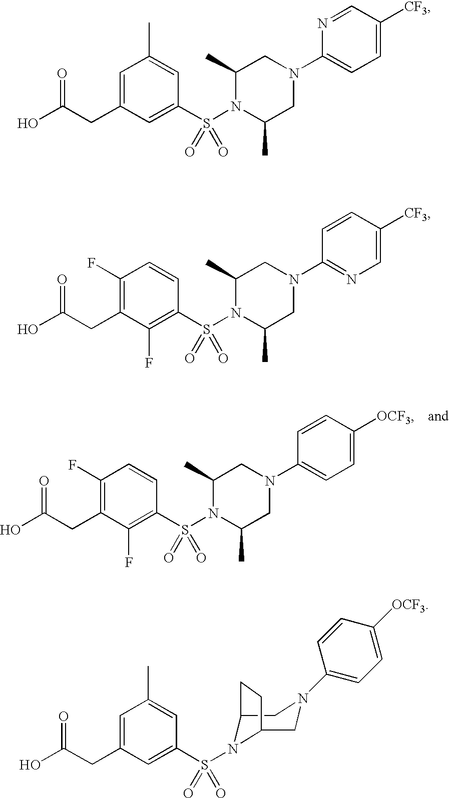

67. A compound having the structure A-B-C, or a pharmaceutically acceptable N-oxide, pharmaceutically acceptable prodrug, pharmaceutically acceptable metabolite, pharmaceutically acceptable salt, pharmaceutically acceptable ester, pharmaceutically acceptable amide, or pharmaceutically acceptable solvate thereof,
wherein:
A is selected from the group consisting of 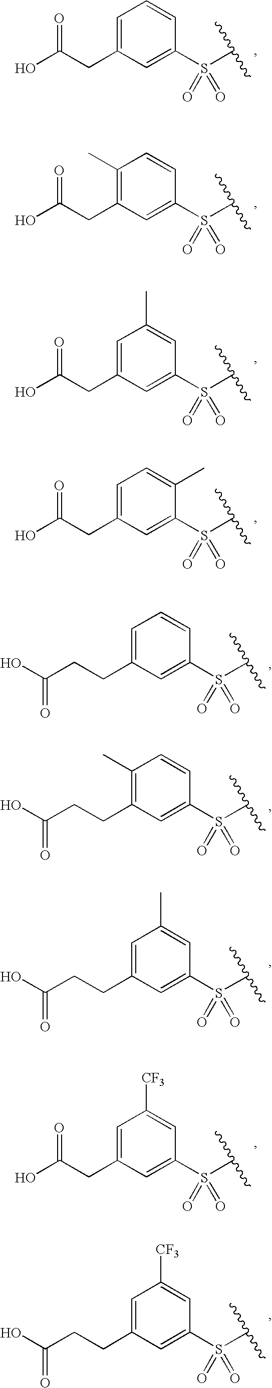 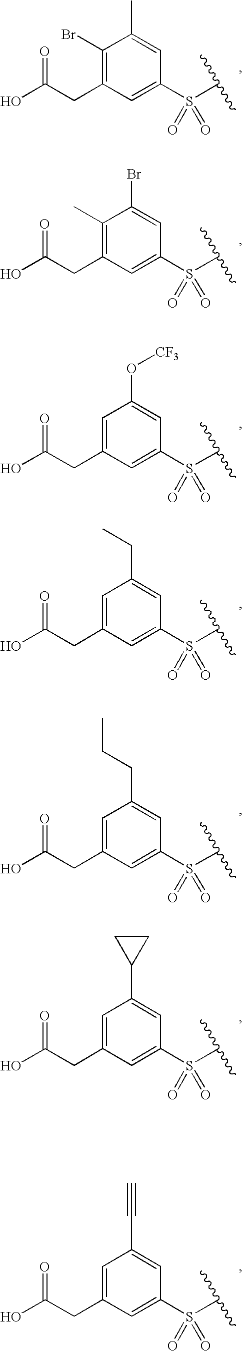 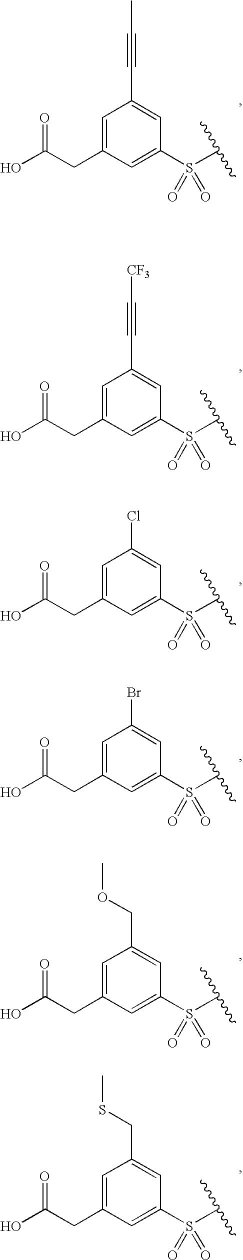 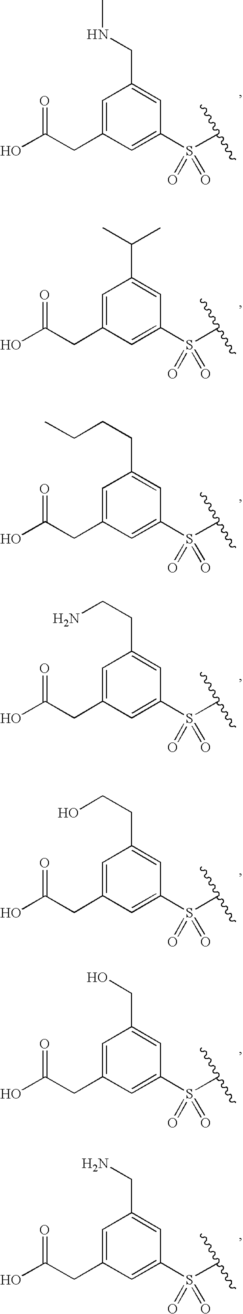 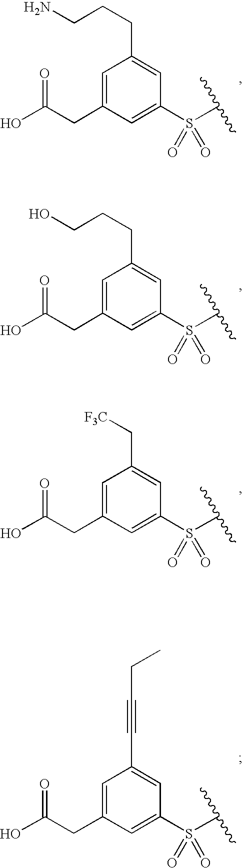
B is selected from the group consisting of: 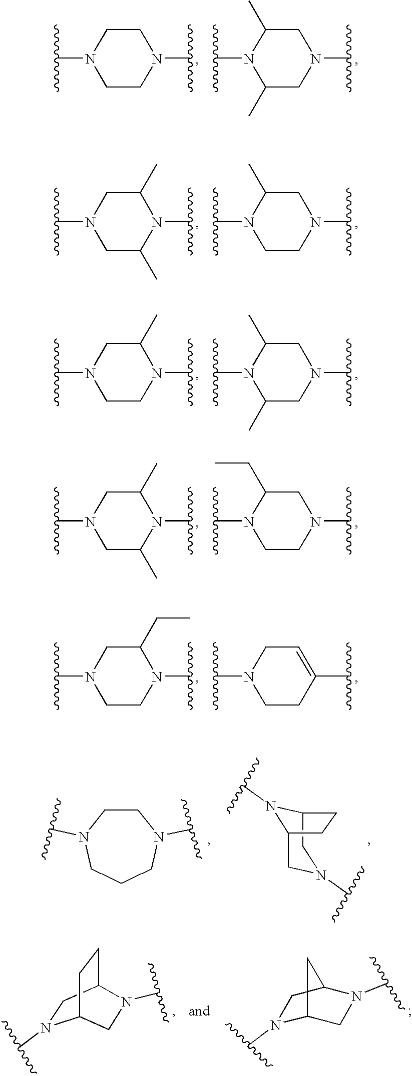
C is selected from the group consisting of:

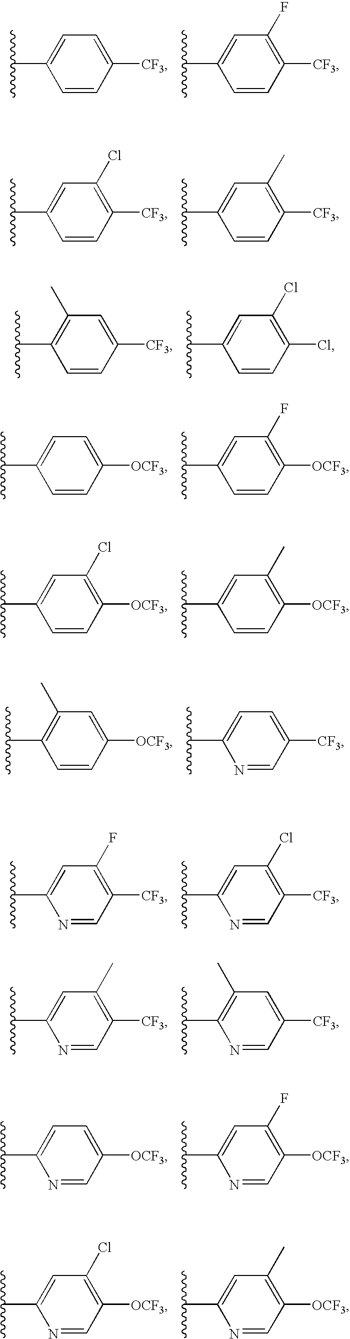

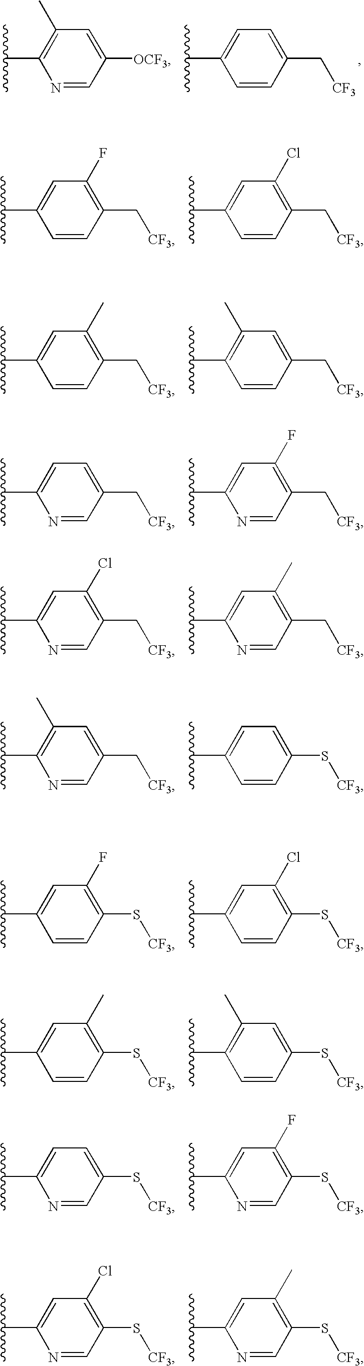

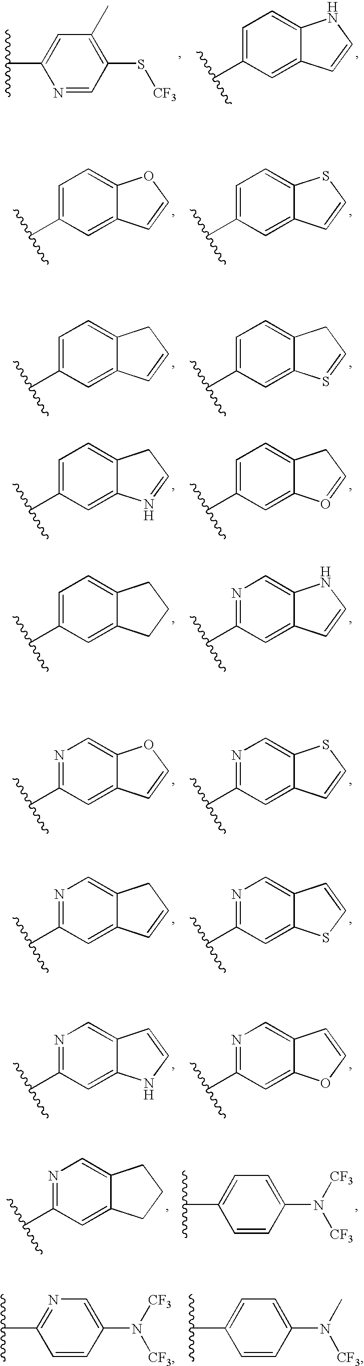

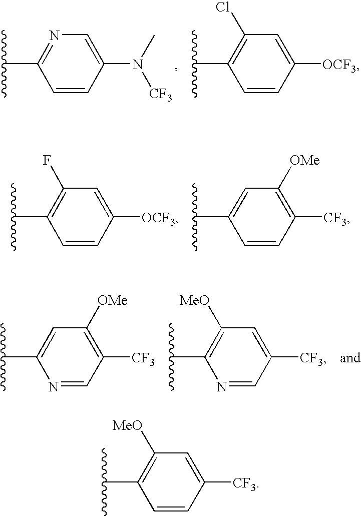

68. The compound of claim 67 wherein:
B is selected from the group consisting of:

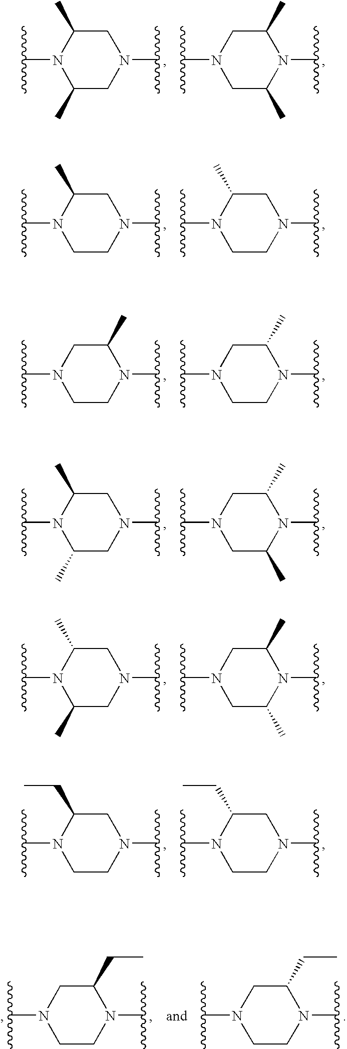

69. A pharmaceutical composition comprising the compound of claim 1.

70. The pharmaceutical composition of claim 69 further comprising a pharmaceutically acceptable diluent or carrier.

71. A compound having the structure of Formula (I) 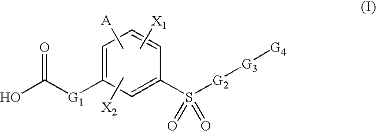
or a pharmaceutically acceptable N-oxide, pharmaceutically acceptable prodrug, pharmaceutically acceptable metabolite, pharmaceutically acceptable salt, pharmaceutically acceptable ester, pharmaceutically acceptable amide, or pharmaceutically acceptable solvate thereof;
wherein:
G₁ is selected from the group consisting of —(CR₁R₂)ₙ—, -Z(CR₁R₂)ₙ—, —(CR₁R₂)ₙZ-, and —(CR₁R₂)ᵣZ(CR₁R₂)ₛ—, wherein Z is O, S, or NR₃;
n is 1-5; r and s are each independently 0 or 1 wherein each R₁ and each R₂ are each independently hydrogen, halogen, optionally substituted lower alkyl, optionally substituted lower heteroalkyl, optionally substituted lower alkoxy, or together may form an optionally substituted cycloalkyl; r and s are not both 0; each R₃ is selected from the group consisting of hydrogen, optionally substituted lower alkyl, and optionally substituted heteroalkyl;
A, X₁ and X₂ are each independently selected from the group consisting of hydrogen, optionally substituted lower alkyl, optionally substituted cycloalkyl, halogen, optionally substituted heteroalkyl, optionally substituted cycloheteroalkyl, optionally substituted lower alkynyl, perhaloalkyl, perhaloalkoxy, hydroxy, optionally substituted lower alkoxy, nitro, cyano, and NH₂;
G₂ is a 5, 6, or 7-membered cyclic moiety having the structure 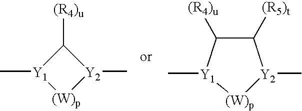
wherein Y₁ is C—R₆ or N and Y₂ is C—R₆ or N;
each R₄ and each R₁ are each independently selected from the group consisting of hydrogen, optionally substituted lower alkyl, halogen, lower perhaloalkyl, hydroxy, optionally substituted heteroalkyl, optionally substituted cycloalkyl, optionally substituted lower alkoxy, nitro, cyano, lower perhaloalkoxy, NH₂,
and —C(O)—O—R₁₁ wherein R₁₁ is hydrogen or optionally substituted lower alkyl, provided that R₄ is not hydroxy or NH₂ when Y₁ is N and R₅ is not hydroxy or NH₂ when Y₂ is N;
W is independently selected from the group consisting of —CR₇R₈—, and a moiety —CR₇— joined together with Y₁ or Y₂ by a double bond;
R₆ is selected from the group consisting of hydrogen, optionally substituted lower alkyl, hydroxy, and lower perhaloalkyl, or is null when Y₁ or Y₂ is joined to W by a double bond; each u is 1 or 2, and each t is 1 or 2, provided that when both Y₁ and Y₂ are N, one of R₄ or R₅ may be taken together with one of W to form an optionally substituted 1- or 2-carbon bridge moiety;
each R₇ and each R₈ are each independently selected from the group consisting of hydrogen, optionally substituted lower alkyl, optionally substituted cycloalkyl, optionally substituted heteroalkyl, hydroxy, optionally substituted lower alkoxy, cyano, halogen, lower perhaloalkyl, NH₂, and a moiety which taken together with R₄ and R₅ forms a 1 or 2 carbon bridge, provided that R₇ and R₈ are not hydroxy or NH₂ when attached to a ring carbon atom adjacent to a ring nitrogen atom;
p is 1, 2 or 3, provided that the G₂ moiety comprises a 5, 6 or 7-membered ring;
G₃ is selected from the group consisting of a bond, a double bond, —CR₉R₁₀)ₘ—, carbonyl, and —(CR₉R₁₀)ₘCR₉═CR₁₀—, wherein m is 0, 1, or 2, and wherein each R₉ and each R₁₀ is independently hydrogen, optionally substituted lower alkyl, optionally substituted lower alkoxy, optionally substituted aryl, lower perhaloalkyl, cyano, and nitro; and
G₄ is selected from the group consisting of hydrogen, optionally substituted aryl, optionally substituted heteroaryl, optionally substituted cycloalkyl, optionally substituted cycloheteroalkyl, optionally substituted cycloalkenyl, optionally substituted fused aryl, optionally substituted fused heteroaryl, and optionally substituted fused cycloalkyl; provided that when G₃ is a bond, G₄ may be covalently linked to G₂.

72. The compound of claim 71 wherein the compound is an hPPAR-delta modulator.

73. The compound of claim 72 for use in the treatment of a disease or condition ameliorated by the modulation of a hPPAR-delta.

74. A pharmaceutical composition comprising the compound of claim 72.

75. The pharmaceutical composition of claim 74 further comprising a pharmaceutically acceptable diluent or carrier.

76. The pharmaceutical composition of claim 74 for use in the treatment of a disease or condition ameliorated by the modulation of a hPPAR-delta.

77. The compound of claim 73 wherein said hPPAR-delta mediated disease or condition is selected from the group consisting of dyslipidemia, metabolic syndrome X, heart failure, hypercholesteremia, cardiovascular disease, type II diabetes mellitus, type 1 diabetes, insulin resistance hyperlipidemia, obesity, anorexia bulimia, inflammation, a wound requiring healing, and anorexia nervosa.

78. The compound of claim 72 for use in the manufacture of a medicament for the prevention or treatment of a disease or condition ameliorated by the modulation of a hPPAR-delta.

79. A method for raising HDL in a subject comprising the administration of a therapeutic amount of the compound of claim 72.

80. Use of the compound of claim 72 for the manufacture of a medicament for the raising of HDL in a patient in need thereof.

81. A method for treating Type 2 diabetes, decreasing insulin resistance or lowering blood pressure in a subject comprising the administration of a therapeutic amount of a compound of claim 72.

82. Use of the compound of claim 72 for the manufacture of a medicament for the treatment of Type 2 diabetes, decreasing insulin resistance or lowering blood pressure in a patient in need thereof.

83. A method for decreasing LDLc in a subject comprising the administration of a therapeutic amount of a compound of claim 72.

84. Use of the compound of claim 72 for the manufacture of a medicament for decreasing LDLc in a patient in need thereof.

85. A method for shifting LDL particle size from small dense to normal LDL in a subject comprising the administration of a therapeutic amount of the compound of claim 72.

86. Use of the compound of claim 72 for the manufacture of a medicament for shifting LDL particle size from small dense to normal LDL in a patient in need thereof.

87. A method for treating atherosclerotic diseases including vascular disease, coronary heart disease, cerebrovascular disease and peripheral vessel disease in a subject comprising the administration of a therapeutic amount of the compound of claim 72.

88. Use of the compound of claim 72 for the manufacture of a medicament for the treatment of atherosclerotic diseases including vascular disease, coronary heart disease, cerebrovascular disease and peripheral vessel disease in a patient in need thereof.

89. A method for treating inflammatory diseases, including rheumatoid arthritis, asthma, osteoarthritis and autoimmune disease in a subject comprising the administration of a therapeutic amount of the compound of claim 72.

90. Use of the compound of claim 72 for the manufacture of a medicament for the treatment of inflammatory diseases, including rheumatoid arthritis, asthma, osteoarthritis and autoimmune disease in a patient in need thereof.

91. A method of treatment of a hPPAR-delta mediated disease or condition comprising administering a therapeutically effective amount of the compound of claim 72.

92. A method of modulating a peroxisome proliferator-activated receptor (PPAR) function comprising contacting said PPAR with a compound of claim 71 and monitoring a change in cell phenotype, cell proliferation, activity of said PPAR, or binding of said PPAR with a natural binding partner.

93. The method of claim 92, wherein said PPAR is selected from the group consisting of PPAR-alpha, PPAR-delta, and PPAR-gamma.

94. A method of treating a disease or condition comprising identifying a patient in need thereof, and administering a therapeutically effective amount of a compound of claim 71 to said patient wherein said disease or condition is selected from the group consisting of obesity, diabetes, hyperinsulinemia, metabolic syndrome X, polycystic ovary syndrome, climacteric, disorders associated with oxidative stress, inflammatory response to tissue injury, pathogenesis of emphysema, ischemia-associated organ injury, doxorubicin-induced cardiac injury, drug-induced hepatotoxicity, atherosclerosis, hypertoxic lung injury, and a wound requiring healing.

95. The compound of claim 71, wherein the compound modulates a peroxisome proliferator-activated receptor (PPAR) function.

96. The compound of claim 95, wherein said PPAR is selected from the group consisting of PPARα, PPARδ, and PPARγ.

97. The compound of claim 95 for use in the treatment of a disease or condition ameliorated by the modulation of a PPAR.

98. The compound of claim 97 wherein said disease or condition is dyslipidemia, metabolic syndrome X, heart failure, hypercholesteremia, cardiovascular disease, type II diabetes mellitus, type 1 diabetes, insulin resistance hyperlipidemia, obesity, anorexia bulimia, inflammation, anorexia nervosa and a wound requiring healing.

99. The compound or composition of claim 97 wherein said PPAR is selected from the group consisting of PPARα, PPARδ, and PPARγ.

100. The compound of claim 71 for use in the manufacture of a medicament for the prevention or treatment of disease or condition ameliorated by the modulation of a PPAR.

101. The compound of claim 100, wherein said PPAR is selected from the group consisting of PPARα, PPARδ, and PPARγ.
